SEQUENCE LISTING (13 sequences)

SEQ ID NO: 1 - RNA, 22 nt, Homo sapiens
ugagaugaagcacuguagcuca

SEQ ID NO: 2 - RNA, 106 nt, Homo sapiens
gcgcagcgcccugucucccagccugaggugcagugcugcaucucuggucaguugggagucugagaugaagcacuguagcucaggaagagagaaguuguucugcagc

SEQ ID NO: 3 - RNA, 83 nt, Homo sapiens
ugucucccagcccaaggugcagugcugcaucucuggucaguugugagucugagaugaagcacuguagcucgggaagggggaau

SEQ ID NO: 4 - RNA, 99 nt, Homo sapiens
gaucuacagucgucuggcccgcggugcagugcugcaucucuggucaacugggagucugagaugaagcacuguagcucgggaggacaacacugucagcuc

SEQ ID NO: 5 - RNA, 105 nt, Homo sapiens
gcggagcgccugucucccagccugaggugcagugcugcaucucuggucaguugggagucugagaugaagcacuguagcucaggaagggagaagauguucugcagc

SEQ ID NO: 6 - RNA, 106 nt, Homo sapiens
gcgcagcgcccugucucccagccugaggugcagugcugcaucucuggucaguugggagucugagaugaagcacuguagcucaggaagagagaaguuuuucugcagc

SEQ ID NO: 7 - RNA, 106 nt, Homo sapiens; identical to SEQ ID NO: 2

SEQ ID NO: 8 - RNA, 106 nt, Homo sapiens; identical to SEQ ID NO: 2

SEQ ID NO: 9 - RNA, 99 nt, Homo sapiens; identical to SEQ ID NO: 4

SEQ ID NO: 10 - RNA, 106 nt, Homo sapiens; identical to SEQ ID NO: 2

SEQ ID NO: 11 - RNA, 106 nt, Homo sapiens; identical to SEQ ID NO: 6

SEQ ID NO: 12 - RNA, 63 nt, Homo sapiens
cccgaggugcagugcugcaucucuggucaguugugagucugagaugaagcacuguagcucggg

SEQ ID NO: 13 - RNA, 63 nt, Homo sapiens
ccugaggugcagugcugcaucucuggucaguugggagucugagaugaagcacuguagcucagg

(19) United States
(12) Patent Application Publication
(10) Pub. No.: US 2009/0232893 A1
(43) Pub. Date: Sep. 17, 2009

(54) miR-143 REGULATED GENES AND PATHWAYS AS TARGETS FOR THERAPEUTIC INTERVENTION

(76) Inventors: Andreas G. Bader, Austin, TX (US); Mike W. Byrom, Austin, TX (US); Charles D. Johnson, Austin, TX (US); David Brown, Austin, TX (US)

Correspondence Address: Fullbright & Jaworski L.L.P., 600 Congress Avenue, Suite 2400, Austin, TX, 78701, US

(21) Appl. No.: 12/125,412

(22) Filed: May 22, 2008

Related U.S. Application Data
(60) Provisional application No. 60/939,573, filed on May 22, 2007.

(51) Int. Cl. A61K 9/14; C12N 5/02; A61K 31/7088; C12Q 1/68

(52) U.S. Cl. 424/489; 435/375; 514/44 R; 435/6

(57) ABSTRACT

The present invention concerns methods and compositions for identifying genes or genetic pathways modulated by miR-143, using miR-143 to modulate a gene or gene pathway, using this profile in assessing the condition of a patient and/or treating the patient with an appropriate miRNA.

This application claims Priority to U.S. Provisional Patent Application Ser. No. 60/939,573, filed May 22, 2007 and PCT application No. PCT/US07/78859 filed Sep. 19, 2007, each of which are hereby incorporated by reference in their entirety.

### BACKGROUND OF THE INVENTION

I. Field of the Invention

The present invention relates to the fields of molecular biology and medicine. More specifically, the invention relates to methods and compositions for the treatment of diseases or conditions that are affected by miR-143 microRNAs, microRNA expression, and genes and cellular pathways directly and indirectly modulated by such.

II. Background

In 2001, several groups used a cloning method to isolate and identify a large group of “microRNAs” (miRNAs) from C. elegans, Drosophila, and humans (Lagos-Quintana et al., 2001; Lau et al., 2001; Lee and Ambros, 2001). Several hundred miRNAs have been identified in plants and animals—including humans—that do not appear to have endogenous siRNAs. Thus, while similar to siRNAs, miRNAs are distinct.

miRNAs thus far observed have been approximately 21-22 nucleotides in length, and they arise from longer precursors transcribed from non-protein-encoding genes. See review of Carrington et al. (2003). The precursors form structures that fold back on themselves in self-complementary regions; they are then processed by the nuclease Dicer (in animals) or DCL1 (in plants) to generate the short double-stranded miRNA. One of the miRNA strands is incorporated into a complex of proteins and miRNA called the RNA-induced silencing complex (RISC). The miRNA guides the RISC complex to a target mRNA, which is then cleaved or translationally silenced, depending on the degree of sequence complementarity of the miRNA to its target mRNA. Currently, it is believed that perfect or nearly perfect complementarity leads to mRNA degradation, as is most commonly observed in plants. In contrast, imperfect base pairing, as is primarily found in animals, leads to translational silencing. However, recent data suggest additional complexity (Bagga et al., 2005; Lim et al., 2005), and mechanisms of gene silencing by miRNAs remain under intense study.

Recent studies have shown that expression levels of numerous miRNAs are associated with various cancers (reviewed in Esquela-Kerscher and Slack, 2006; Calin and Croce, 2006). miRNAs have also been implicated in regulating cell growth and cell and tissue differentiation—cellular processes that are associated with the development of cancer.

The inventors previously demonstrated that hsa-miR-143 is involved with the regulation of numerous cell activities that represent intervention points for cancer therapy and for therapy of other diseases and disorders (U.S. patent application Ser. No. 11/141,707 filed May 31, 2005 and Ser. No. 11/273,640 filed Nov. 14, 2005, each of which are incorporated herein by reference in their entirety). Upon evaluation of 24 different human tissues, hsa-miR-143 was found to be preferentially expressed in human prostate and colon tissue samples. The inventors observed that hsa-miR-143 expression is lower in many human cancer tumor samples including lung, colon, breast, bladder, and thyroid tumors, than in normal cells from the same patients. Overexpression of hsa-miR-143 in human leukemia cells (Jurkat) increased proliferation of those cells. The inventors also found hsa-miR-143 to be up-regulated in brain tissues of Alzheimer's patients. Other investigators have also observed that miR-143 is down-regulated in colorectal tumors when compared with matched normal samples (Michael et al., 2003; Akao et al., 2006) and that miR-143 may be involved in the differentiation of human adipocytes (fat storage cells) (Esau et al., 2004).

Bioinformatics analyses suggest that any given miRNA may bind to and alter the expression of up to several hundred different genes. In addition, a single gene may be regulated by several miRNAs. Thus, each miRNA may regulate a complex interaction among genes, gene pathways, and gene networks. Mis-regulation or alteration of these regulatory pathways and networks, involving miRNAs, are likely to contribute to the development of disorders and diseases such as cancer. Although bioinformatics tools are helpful in predicting miRNA binding targets, all have limitations. Because of the imperfect complementarity with their target binding sites, it is difficult to accurately predict the mRNA targets of miRNAs with bioinformatics tools alone. Furthermore, the complicated interactive regulatory networks among miRNAs and target genes make it difficult to accurately predict which genes will actually be mis-regulated in response to a given miRNA.

Correcting gene expression errors by manipulating miRNA expression or by repairing miRNA mis-regulation represent promising methods to repair genetic disorders and cure diseases like cancer. A current, disabling limitation of this approach is that, as mentioned above, the details of the regulatory pathways and networks that are affected by any given miRNA, including miR-143, remain largely unknown. This represents a significant limitation for treatment of cancers in which miR-143 may play a role. A need exists to identify the genes, genetic pathways, and genetic networks that are regulated by or that may regulate hsa-miR-143 expression.

### SUMMARY OF THE INVENTION

The present invention provides additional compositions and methods by identifying genes that are direct targets for miR-143 regulation or that are indirect or downstream targets of regulation following the miR-143-mediated modification of another gene(s) expression. Furthermore, the invention describes gene, disease, and/or physiologic pathways and networks influenced by miR-143 and its family members. In certain aspects, compositions of the invention are administered to a subject having, suspected of having, or at risk of developing a metabolic, an immunologic, an infectious, a cardiovascular, a digestive, an endocrine, an ocular, a genitourinary, a blood, a musculoskeletal, a nervous system, a congenital, a respiratory, a skin, or a cancerous disease or condition.

In particular aspects, a subject or patient may be selected for treatment based on expression and/or aberrant expression of one or more miRNA or mRNA. In a further aspect, a subject or patient may be selected for treatment based on aberrations in one or more biologic or physiologic pathway(s), including aberrant expression of one or more gene associated with a pathway, or the aberrant expression of one or more protein encoded by one or more gene associated with a pathway. In still a further aspect, a subject or patient may be selected based on aberrations in miRNA expression, or biologic and/or physiologic pathway(s). A subject may be assessed for sensitivity, resistance, and/or efficacy of a therapy or treatment regime based on the evaluation and/or analysis of miRNA or mRNA expression or lack thereof. A subject may be evaluated for amenability to certain therapy prior to, during, or after administration of one or therapy to a subject or patient. Typically, evaluation or assessment may be done by analysis of miRNA and/or mRNA, as well as combination of other assessment methods that include but are not limited to histology, immunohistochemistry, blood work, etc.

In some embodiments, an infectious disease or condition includes a bacterial, viral, parasite, or fungal infection. Many of these genes and pathways are associated with various cancers and other diseases. Cancerous conditions include, but are not limited to astrocytoma, acute myelogenous leukemia, acute lymphoblastic leukemia, anaplastic large cell lymphoma, B-cell lymphoma, breast carcinoma, bladder carcinoma, cervical carcinoma, chronic lymphoblastic leukemia, colorectal carcinoma, endometrial carcinoma, glioma, glioblastoma, gastric carcinoma, hepatocellular carcinoma, Hodgkin lymphoma, leukemia, medulloblastoma, melanoma, mantle cell lymphoma, multiple myeloma, myeloma, non-Hodgkin lymphoma, lung carcinoma, non-small cell lung carcinoma, oligodendroglioma, ovarian carcinoma, esophageal carcinoma, osteosarcoma, pancreatic carcinoma, prostate carcinoma, renal cell carcinoma, small cell lung carcinoma, squamous cell carcinoma of the head and neck, thyroid carcinoma, or testicular tumor wherein the modulation of one or more gene is sufficient for a therapeutic response. Typically, a cancerous condition is an aberrant hyperproliferative condition associated with the uncontrolled growth or inability to undergo cell death, including apoptosis. In certain aspects the cancerous condition is lung carcinoma, such asadenocarcinoma, squamous cell carcinoma, large cell carcinoma, or bronchioalveolar carcinoma.

The present invention provides methods and compositions for identifying genes that are direct targets for miR-143 regulation or that are downstream targets of regulation following the miR-143-mediated modification of upstream gene expression. Furthermore, the invention describes gene pathways and networks that are influenced by miR-143 expression in biological samples. Many of these genes and pathways are associated with various cancers and other diseases. The altered expression or function of miR-143 in cells would lead to changes in the expression of these key genes and contribute to the development of disease. Introducing miR-143 (for diseases where the miRNA is down-regulated) or a miR-143 inhibitor (for diseases where the miRNA is up-regulated) into disease cells or tissues would result in a therapeutic response. The identities of key genes that are regulated directly or indirectly by miR-143 and the disease with which they are associated are provided herein. In certain aspects a cell may be an endothelial, a mesothelial, an epithelial, stromal, or mucosal cell. The cell can be, but is not limited to brain, a neuronal, a blood, an esophageal, a lung, a cardiovascular, a liver, a breast, a bone, a thyroid, a glandular, an adrenal, a pancreatic, a stomach, a intestinal, a kidney, a bladder, a prostate, a uterus, an ovarian, a testicular, a splenic, a skin, a smooth muscle, a cardiac muscle, or a striated muscle cell. In certain aspects, the cell, tissue, or target may not be defective in miRNA expression yet may still respond therapeutically to expression or over expression of a miRNA. miR-143 could be used as a therapeutic target for any of these diseases. In certain embodiments miR-143 can be used to modulate the activity of miR-143 in a subject, organ, tissue, or cell.

A cell, tissue, or subject may be a cancer cell, a cancerous tissue, harbor cancerous tissue, or be a subject or patient diagnosed or at risk of developing a disease or condition. In certain aspects a cancer cell is a neuronal, glial, lung, liver, brain, breast, bladder, blood, leukemic, lymphoid, colon, endometrial, stomach, skin, ovarian, fat, bone, cervical, esophageal, pancreatic, prostate, kidney, testicular, intestinal, colorectal, or thyroid cell. In still a further aspect cancer includes, but is not limited to astrocytoma, acute myelogenous leukemia, acute lymphoblastic leukemia, anaplastic large cell lymphoma, B-cell lymphoma, breast carcinoma, bladder carcinoma, cervical carcinoma, chronic lymphoblastic leukemia, colorectal carcinoma, endometrial carcinoma, glioma, glioblastoma, gastric carcinoma, hepatocellular carcinoma, Hodgkin lymphoma, leukemia, medulloblastoma, melanoma, mantle cell lymphoma, multiple myeloma, myeloma, non-Hodgkin lymphoma, lung carcinoma, non-small cell lung carcinoma, oligodendroglioma, ovarian carcinoma, esophageal carcinoma, osteosarcoma, pancreatic carcinoma, prostate carcinoma, renal cell carcinoma, small cell lung carcinoma, squamous cell carcinoma of the head and neck, thyroid carcinoma, or testicular tumor

Embodiments of the invention include methods of modulating gene expression, or biologic or physiologic pathways in a cell, a tissue, or a subject comprising administering to the cell, tissue, or subject an amount of an isolated nucleic acid or mimetic thereof comprising a miR-143 nucleic acid, mimetic, or inhibitor in an amount sufficient to modulate the expression of a gene positively or negatively modulated by a miR-143 miRNA. A “miR-143 nucleic acid sequence” or “miR-143 inhibitor” includes the full length precursor of miR-143, or complement thereof, as well as 5, 6, 7, 8, 9, 10, 11, 12, 13, 14, 15, 16, 17, 18, 19, 20, 21, 22, 23, 24, 25, 26, 27, 28, 29 or more nucleotides of a precursor miRNA or its processed sequence, or complement thereof, including all ranges and integers there between. In certain embodiments, the miR-143 nucleic acid sequence or miR-143 inhibitor contains the full-length processed miRNA sequence or complement thereof and is referred to as the “miR-143 full-length processed nucleic acid sequence” or “miR-143 full-length processed inhibitor sequence.” In still further aspects, the miR-143 nucleic acid comprises at least 5, 6, 7, 8, 9, 10, 11, 12, 13, 14, 15, 16, 17, 18, 19, 20, 21, 22, 232, 24, 25, 50 nucleotide (including all ranges and integers there between) segment or complementary segment of miR-143 that is at least 75, 80, 85, 90, 95, 98, 99 or 100% identical to SEQ ID NO:1 to SEQ ID NO:13. The general term miR-143 includes all members of the miR-143 family that share at least part of a mature miR-143 sequence (UGAGAUGAAGCACUGUAGCUCA (SEQ ID NO:1)) or a complement thereof.

A “miR-143 nucleic acid sequence” includes the full length precursor of miR-143 and other family members that include

lla-mir-143 (MI0002552)

(SEQ ID NO: 2)

GCGCAGCGCCCUGUCUCCCAGCCUGAGGUGCAGUGCUGCAUCUCUGGUCA
GUUGGGAGUCUGAGAUGAAGCACUGUAGCUCAGGAAGAGAGAAGUUGUUC
UGCAGC;
xtr-mir-143 (MI0004937)

(SEQ ID NO: 3)

UGUCUCCCAGCCCAAGGUGCAGUGCUGCAUCUCUGGUCAGUUGUGAGUCU
GAGAUGAAGCACUGUAGCUCGGGAAGGGGGAAU;
dre-mir-143-2 (MI0002008)

(SEQ ID NO: 4)

GAUCUACAGUCGUCUGGCCCGCGGUGCAGUGCUGCAUCUCUGGUCAACUG
GGAGUCUGAGAUGAAGCACUGUAGCUCGGGAGGACAACACUGUCAGCUC;
rno-mir-143 (MI0000916)

(SEQ ID NO: 5)

GCGGAGCGCCUGUCUCCCAGCCUGAGGUGCAGUGCUGCAUCUCUGGUCAG
UUGGGAGUCUGAGAUGAAGCACUGUAGCUCAGGAAGGGAGAAGAUGUUCU
GCAGC;
ptr-mir-143 (MI0002549)

(SEQ ID NO: 6)

GCGCAGCGCCCUGUCUCCCAGCCUGAGGUGCAGUGCUGCAUCUCUGGUCA
GUUGGGAGUCUGAGAUGAAGCACUGUAGCUCAGGAAGAGAGAAGUUUUUC
UGCAGC;
ppy-mir-143 (MI0002551)

(SEQ ID NO: 7)

GCGCAGCGCCCUGUCUCCCAGCCUGAGGUGCAGUGCUGCAUCUCUGGUCA
GUUGGGAGUCUGAGAUGAAGCACUGUAGCUCAGGAAGAGAGAAGUUGUUC
UGCAGC;
ggo-mir-143 (MI0002550)

(SEQ ID NO: 8)

GCGCAGCGCCCUGUCUCCCAGCCUGAGGUGCAGUGCUGCAUCUCUGGUCA
GUUGGGAGUCUGAGAUGAAGCACUGUAGCUCAGGAAGAGAGAAGUUGUUC
UGCAGC;
dre-mir-143-1 (MI0002007)

(SEQ ID NO: 9)

GAUCUACAGUCGUCUGGCCCGCGGUGCAGUGCUGCAUCUCUGGUCAACUG
GGAGUCUGAGAUGAAGCACUGUAGCUCGGGAGGACAACACUGUCAGCUC;
hsa-mir-143 (MI0000459)

(SEQ ID NO: 10)

GCGCAGCGCCCUGUCUCCCAGCCUGAGGUGCAGUGCUGCAUCUCUGGUCA
GUUGGGAGUCUGAGAUGAAGCACUGUAGCUCAGGAAGAGAGAAGUUGUUC
UGCAGC;
ppa-mir-143 (MI0002553)

(SEQ ID NO: 11)

GCGCAGCGCCCUGUCUCCCAGCCUGAGGUGCAGUGCUGCAUCUCUGGUCA
GUUGGGAGUCUGAGAUGAAGCACUGUAGCUCAGGAAGAGAGAAGUUUUUC
UGCAGC;
mdo-mir-143 (MI0005302)

(SEQ ID NO: 12)

CCCGAGGUGCAGUGCUGCAUCUCUGGUCAGUUGUGAGUCUGAGAUGAAGC
ACUGUAGCUCGGG;
mmu-mir-143 (MI0000257)

(SEQ ID NO: 13)

CCUGAGGUGCAGUGCUGCAUCUCUGGUCAGUUGGGAGUCUGAGAUGAAGC
ACUGUAGCUCAGG.

In certain aspects, a nucleic acid or mimetic of the present invention will comprise 5, 6, 7, 8, 9, 10, 11, 12, 13, 14, 15, 16, 17, 18, 19, 20, 21, 22, 23, 24, 25, 26, 27, 28, 29 or more nucleotides of the precursor miRNA or its processed sequence, including all ranges and integers there between. In certain embodiments, the miR-143 nucleic acid sequence contains the full-length processed miRNA sequence and is referred to as the “miR-143 full-length processed nucleic acid sequence.” In still further aspects, a miR-143 comprises at least one 5, 6, 7, 8, 9, 10, 11, 12, 13, 14, 15, 16, 17, 18, 19, 20, 21, 22, 23, 24, 25, 50 nucleotide (including all ranges and integers there between) segment of miR-143 that is at least 75, 80, 85, 90, 95, 98, 99 or 100% identical to SEQ ID NOs provided herein.

In specific embodiments, a miR-143 or miR-143 inhibitor containing nucleic acid is hsa-miR-143 or hsa-miR-143 inhibitor, or a variation thereof. In a further aspect, a miR-143 nucleic acid or miR-143 inhibitor can be administered with 1, 2, 3, 4, 5, 6, 7, 8, 9, 10 or more miRNAs or miRNA inhibitors. miRNA or its complement can be administer concurrently, in sequence or in an ordered progression. In certain aspects, a miR-143 or miR-143 inhibitor can be administered in combination with one or more of let-7, miR-15a, miR-16, miR-20, miR-21, miR-26a, miR-31, miR-34a, miR-126, miR-145, miR-147, miR-188, miR-200b, miR-200c, miR-215, miR-216, miR-292-3p, and/or miR-331. All or combinations of miRNAs or inhibitors thereof may be administered in a single formulation. Administration may be before, during or after a second therapy.

miR-143 nucleic acids or complement thereof may also include various heterologous nucleic acid sequence, i.e., those sequences not typically found operatively coupled with miR-143 in nature, such as promoters, enhancers, and the like. The miR-143 nucleic acid is a recombinant nucleic acid, and can be a ribonucleic acid or a deoxyribonucleic acid. The recombinant nucleic acid may comprise a miR-143 or miR-143 inhibitor expression cassette, i.e., a nucleic acid segment that expresses a nucleic acid when introduce into an environment containing components for nucleic acid synthesis. In a further aspect, the expression cassette is comprised in a viral, or plasmid DNA vector or other therapeutic nucleic acid vector or delivery vehicle, including liposomes and the like. In certain aspects, viral vectors can be administered at 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴ pfu or viral particle (vp).

In a particular aspect, the miR-143 nucleic acid or miR-143 inhibitor is a synthetic nucleic acid. Moreover, nucleic acids of the invention may be fully or partially synthetic. In still further aspects, a nucleic acid of the invention or a DNA encoding such can be administered at 0.001, 0.01, 0.1, 1, 10, 20, 30, 40, 50, 100, 200, 400, 600, 800, 1000, 2000, to 4000 μg or mg, including all values and ranges there between. In yet a further aspect, nucleic acids of the invention, including synthetic nucleic acid, can be administered at 0.001, 0.01, 0.1, 1, 10, 20, 30, 40, 50, 100, to 200 μg or mg per kilogram (kg) of body weight. Each of the amounts described herein may be administered over a period of time, including 0.5, 1, 2, 3, 4, 5, 6, 7, 8, 9, 10, minutes, hours, days, weeks, months or years, including all values and ranges there between.

In certain embodiments, administration of the composition(s) can be enteral or parenteral. In certain aspects, enteral administration is oral. In further aspects, parenteral administration is intralesional, intravascular, intracranial, intrapleural, intratumoral, intraperitoneal, intramuscular, intralymphatic, intraglandular, subcutaneous, topical, intrabronchial, intratracheal, intranasal, inhaled, or instilled. Compositions of the invention may be administered regionally or locally and not necessarily directly into a lesion.

In certain aspects, the gene or genes modulated comprises 1, 2, 3, 4, 5, 6, 7, 8, 9, 10, 11, 12, 13, 14, 15, 20, 25, 30, 35, 40, 45, 50, 100, 150, 200 or more genes or combinations of genes identified in Tables 1, 3, 4, and/or 5. In still further aspects, the gene or genes modulated may exclude 1, 2, 3, 4, 5, 6, 7, 8, 9, 10, 11, 12, 13, 14, 15, 20, 25, 30, 35, 40, 45, 50, 100, 150, 175 or more genes or combinations of genes identified in Tables 1, 3, 4, and/or 5. Modulation includes modulating transcription, mRNA levels, mRNA translation, and/or protein levels in a cell, tissue, or organ. In certain aspects the expression of a gene or level of a gene product, such as mRNA or encoded protein, is down-regulated or up-regulated. In a particular aspect the gene modulated comprises or is selected from (and may even exclude) 1, 2, 3, 4, 5, 6, 7, 8, 9, 10, 11, 12, 13, 14, 15, 16, 17, 18, 19, 20, 21, 22, 23, 24, 25, 26. 27, 28, or all of the genes identified in Tables 1, 3, 4, and/or 5, or any combinations thereof. In certain embodiments a gene modulated or selected to be modulated is from Table 1. In further embodiments a gene modulated or selected to be modulated is from Table 3. In still further embodiments a gene modulated or selected to be modulated is from Table 4. In yet further embodiments a gene modulated or selected to be modulated is from Table 5. Embodiments of the invention may also include obtaining or assessing a gene expression profile or miRNA profile of a target cell prior to selecting the mode of treatment, e.g., administration of a miR-143 nucleic acid, inhibitor of miR-143, or mimetics thereof. The database content related to nucleic acids and genes designated by an accession number or a database submission are incorporated herein by reference as of the filing date of this application. In certain aspects of the invention one or more miRNA or miRNA inhibitor may modulate a single gene. In a further aspect, one or more genes in one or more genetic, cellular, or physiologic pathways can be modulated by one or more miRNAs or complements thereof, including miR-143 nucleic acids and miR-143 inhibitors in combination with other miRNAs.

miR-143 nucleic acids may also include various heterologous nucleic acid sequence, i.e., those sequences not typically found operatively coupled with miR-143 in nature, such as promoters, enhancers, and the like. The miR-143 nucleic acid is a recombinant nucleic acid, and can be a ribonucleic acid or a deoxyribonucleic acid. The recombinant nucleic acid may comprise a miR-143 expression cassette. In a further aspect, the expression cassette is comprised in a viral, or plasmid DNA vector or other therapeutic nucleic acid vector or delivery vehicle, including liposomes and the like. In a particular aspect, the miR-143 nucleic acid is a synthetic nucleic acid. Moreover, nucleic acids of the invention may be fully or partially synthetic.

TABLE 1
Genes with increased (positive values) or decreased (negative values)
expression following transfection of human cancer cells with
pre-miR hsa-miR-143.

| Gene | | |
|---|---|---|
| Symbol | RefSeq Transcript ID | Δ log₂ |
| AKAP12 | NM_005100 /// NM_144497 | 0.725245496 |
| ANKRD46 | NM_198401 | 0.791492237 |
| ANXA6 | NM_001155 /// NM_004033 | 0.727214714 |
| ARL2BP | NM_012106 | 0.800772424 |
| ASNA1 | NM_004317 | −1.07942093 |
| ATP6V1A | NM_001690 | −1.126127932 |
| ATXN1 | NM_000332 | 0.850968582 |
| AXL | NM_001699 /// NM_021913 | 1.156039698 |
| BCL2L1 | NM_001191 /// NM_138578 | −0.821265359 |
| CCND1 | NM_053056 | −0.938024465 |
| CCNG1 | NM_004060 /// NM_199246 | 0.862627632 |
| CLIC4 | NM_013943 | 0.825614765 |
| CXCL1 | NM_001511 | 0.938115811 |
| CXCL2 | NM_002089 | 0.706326327 |
| DAZAP2 | NM_014764 | −0.916764957 |
| DCP2 | NM_152624 | 0.797770229 |
| DDAH1 | NM_012137 | 0.765730627 |
| DDX3Y | NM_004660 | 0.848651105 |
| DICER1 | NM_030621 /// NM_177438 | 0.929848609 |
| DSC2 | NM_004949 /// NM_024422 | 0.902830281 |
| FLJ13910 | NM_022780 | 0.866839654 |
| GALC | NM_000153 | −1.161432175 |
| GATM | NM_001482 | −1.970548228 |
| GOLPH2 | NM_016548 /// NM_177937 | −1.126884613 |
| GREB1 | NM_014668 /// NM_033090 /// | 0.755673527 |
| | NM_148903 | |
| GREM1 | NM_013372 | 1.051739161 |
| HIPK2 | NM_022740 | −0.904313564 |
| HIPK3 | NM_005734 | 0.826433357 |
| IFIH1 | NM_022168 | 0.706653845 |
| IGFBP3 | NM_000598 /// NM_001013398 | −0.809607512 |
| IL32 | NM_001012631 /// NM_001012632 /// | 0.757126883 |
| | NM_001012633 /// NM_001012634 /// | |
| | NM_001012635 | |
| IL6ST | NM_002184 /// NM_175767 | 0.751854493 |
| IL8 | NM_000584 | 1.104016175 |
| INSIG1 | NM_005542 /// NM_198336 /// | 0.875027481 |
| | NM_198337 | |
| LEPR | NM_001003679 /// NM_001003680 /// | 0.797930372 |
| | NM_002303 | |
| LMO4 | NM_006769 | −1.012706499 |
| LOC137886 | XM_059929 | −0.752855433 |
| MCL1 | NM_021960 /// NM_182763 | 0.761759353 |
| MGC5618 | — | 0.797855581 |
| MTUS1 | NM_001001924 /// NM_001001925 /// | 0.70655 |
| | NM_001001927 /// NM_001001931 /// | |
| | NM_020749 | |
| NID1 | NM_002508 | 1.090976167 |
| NT5E | NM_002526 | 0.878049429 |
| PDCD2 | NM_002598 /// NM_144781 | −0.723484401 |
| PDCD4 | NM_014456 /// NM_145341 | 0.728228239 |
| PDK4 | NM_002612 | 0.961974975 |
| PELI1 | NM_020651 | 0.768582445 |
| PMCH | NM_002674 | 0.790936704 |
| PROSC | NM_007198 | −1.645677869 |
| PTPN12 | NM_002835 | 0.769808986 |
| RAB11FIP1 | NM_001002233 /// NM_001002814 /// | −0.83733308 |
| | NM_025151 | |
| RAB2 | NM_002865 | 0.827382805 |
| RBL1 | NM_002895 /// NM_183404 | −1.302328709 |
| RDX | NM_002906 | 0.760806942 |
| RECK | NM_021111 | 1.103484746 |
| RHEB | NM_005614 | 0.825468322 |
| RHOB | NM_004040 | 0.921813933 |
| RHOBTB1 | NM_001032380 /// NM_014836 /// | 0.744478582 |
| | NM_198225 | |
| RP2 | NM_006915 | 0.822851399 |
| SERPINE1 | NM_000602 | −0.856846452 |
| SLC11A2 | NM_000617 | 0.716682705 |
| SLC30A1 | NM_021194 | −0.841163945 |
| SLC35B1 | NM_005827 | −1.07644709 |
| TAF10 | NM_006284 | −1.695883532 |
| TBC1D2 | NM_018421 | −0.746279363 |
| TGFBR2 | NM_001024847 /// NM_003242 | 0.854509353 |
| TMEM45A | NM_018004 | −0.748492283 |
| TMF1 | NM_007114 | −0.939693594 |
| TNC | NM_002160 | 0.86901183 |
| TNRC9 | XM_049037 | 0.740367787 |
| TRA1 | NM_003299 | 0.875188144 |
| TTMP | NM_024616 | 0.844059608 |
| TXN | NM_003329 | 0.92541735 |
| UGT1A8 /// | NM_019076 /// NM_021027 | −0.961897449 |
| UGT1A9 | | |
| WASPIP | NM_003387 | 1.04160055 |
| WDR50 | NM_016001 | −1.049152791 |
| WEE1 | NM_003390 | 0.722369746 |

A further embodiment of the invention is directed to methods of modulating a cellular pathway comprising administering to the cell an amount of an isolated nucleic acid comprising a miR-143 nucleic acid sequence or a miR-143 inhibitor. A cell, tissue, or subject may be a cancer cell, a cancerous tissue or harbor cancerous tissue, or a cancer patient. The database content related to all nucleic acids and genes designated by an accession number or a database submission are incorporated herein by reference as of the filing date of this application. In certain aspects, a composition of the invention is a pharmaceutical formulation such a lipid, nanoparticle, microparticle and the like that are typically biocompatible and/or biodegradable.

A further embodiment of the invention is directed to methods of modulating a cellular pathway comprising administering to the cell an amount of an isolated nucleic acid comprising a miR-143 nucleic acid sequence in an amount sufficient to modulate the expression, function, status, or state of a cellular pathway, in particular those pathways described in Table 2 or the pathways known to include one or more genes from Table 1, 3, 4, and/or 5. Modulation of a cellular pathway includes, but is not limited to modulating the expression of one or more gene(s). Modulation of a gene can include inhibiting the function of an endogenous miRNA or providing a functional miRNA to a cell, tissue, or subject. Modulation refers to the expression levels or activities of a gene or its related gene product (e.g., mRNA) or protein, e.g., the mRNA levels may be modulated or the translation of an mRNA may be modulated. Modulation may increase or up regulate a gene or gene product or it may decrease or down regulate a gene or gene product (e.g., protein levels or activity).

Still a further embodiment includes methods of administering an miRNA or mimic thereof, and/or treating a subject or patient having, suspected of having, or at risk of developing a pathological condition comprising one or more of step (a) administering to a patient or subject an amount of an isolated nucleic acid comprising a miR-143 nucleic acid sequence or a miR-143 inhibitor in an amount sufficient to modulate expression of a cellular pathway; and (b) administering a second therapy, wherein the modulation of the cellular pathway sensitizes the patient or subject, or increases the efficacy of a second therapy. An increase in efficacy can include a reduction in toxicity, a reduced dosage or duration of the second therapy, or an additive or synergistic effect. A cellular pathway may include, but is not limited to one or more pathway described in Table 2 below or a pathway that is know to include one or more genes of Tables 1, 3, 4, and/or 5. The second therapy may be administered before, during, and/or after the isolated nucleic acid or miRNA or inhibitor is administered

A second therapy can include administration of a second miRNA or therapeutic nucleic acid such as a siRNA or antisense oligonucleotide, or may include various standard therapies, such as pharmaceuticals, chemotherapy, radiation therapy, drug therapy, immunotherapy, and the like. Embodiments of the invention may also include the determination or assessment of gene expression or gene expression profile for the selection of an appropriate therapy. In a particular aspect, a second therapy is a chemotherapy. A chemotherapy can include, but is not limited to paclitaxel, cisplatin, carboplatin, doxorubicin, oxaliplatin, larotaxel, taxol, lapatinib, docetaxel, methotrexate, capecitabine, vinorelbine, cyclophosphamide, gemcitabine, amrubicin, cytarabine, etoposide, camptothecin, dexamethasone, dasatinib, tipifarnib, bevacizumab, sirolimus, temsirolimus, everolimus, lonafarnib, cetuximab, erlotinib, gefitinib, imatinib mesylate, rituximab, trastuzumab, nocodazole, sorafenib, sunitinib, bortezomib, alemtuzumab, gemtuzumab, tositumomab or ibritumomab.

Embodiments of the invention include methods of treating a subject with a disease or condition comprising one or more of the steps of (a) determining an expression profile of one or more genes selected from Table 1, 3, 4, and/or 5; (b) assessing the sensitivity of the subject to therapy based on the expression profile; (c) selecting a therapy based on the assessed sensitivity; and (d) treating the subject using a selected therapy. Typically, the disease or condition will have as a component, indicator, or resulting mis-regulation of one or more gene of Table 1, 3, 4, and/or 5.

In certain aspects, 2, 3, 4, 5, 6, 7, 8, 9, 10, or more miRNA may be used in sequence or in combination. For instance, any combination of miR-143 or a miR-143 inhibitor with another miRNA Further embodiments include the identification and assessment of an expression profile indicative of miR-143 status in a cell or tissue comprising expression assessment of one or more gene from Table 1, 3, 4, and/or 5, or any combination thereof.

The term “miRNA” is used according to its ordinary and plain meaning and refers to a microRNA molecule found in eukaryotes that is involved in RNA-based gene regulation. See, e.g., Carrington et al., 2003, which is hereby incorporated by reference. The term can be used to refer to the single-stranded RNA molecule processed from a precursor or in certain instances the precursor itself.

In some embodiments, it may be useful to know whether a cell expresses a particular miRNA endogenously or whether such expression is affected under particular conditions or when it is in a particular disease state. Thus, in some embodiments of the invention, methods include assaying a cell or a sample containing a cell for the presence of one or more marker gene or mRNA or other analyte indicative of the expression level of a gene of interest. Consequently, in some embodiments, methods include a step of generating an RNA profile for a sample. The term “RNA profile” or “gene expression profile” refers to a set of data regarding the expression pattern for one or more gene or genetic marker in the sample (e.g., a plurality of nucleic acid probes that identify one or more markers from Tables 1, 3, 4, and/or 5); it is contemplated that the nucleic acid profile can be obtained using a set of RNAs, using for example nucleic acid amplification or hybridization techniques well know to one of ordinary skill in the art. The difference in the expression profile in the sample from the patient and a reference expression profile, such as an expression profile from a normal or non-pathologic sample, is indicative of a pathologic, disease, or cancerous condition. A nucleic acid or probe set comprising or inhibitor can be selected based on observing two given miRNAs share a set of target genes or pathways listed in Tables 1, 2, 4 and/or 5 that are altered in a particular disease or condition. These two miRNAs may result in an improved therapy (e.g., reduced toxicity, greater efficacy, prolong remission, or other improvements in a subjects condition), result in an increased efficacy, an additive efficacy, or a synergistic efficacy providing an additional or an improved therapeutic response. Without being bound by any particular theory, synergy of two miRNA can be a consequence of regulating the same genes or related genes (related by a common pathway or biologic end result) more effectively (e.g., due to distinct binding sites on the same target or related target(s)) and/or a consequence of regulating different genes, but all of which have been implicated in a disease or condition.

In certain aspects, miR-143 or a miR-143 inhibitor and let-7 can be administered to patients with acute myeloid leukemia, breast carcinoma, bladder carcinoma, cervical carcinoma, colorectal carcinoma, endometrial carcinoma, glioma, glioblastoma, gastric carcinoma, hepatocellular carcinoma, Hodgkin lymphoma, leukemia, melanoma, myxofibrosarcoma, multiple myeloma, neuroblastoma, non-Hodgkin lymphoma, non-small cell lung carcinoma, ovarian carcinoma, esophageal carcinoma, pancreatic carcinoma, prostate carcinoma, squamous cell carcinoma of the head and neck, thyroid carcinoma, or urothelial carcinoma.

Further aspects include administering miR-143 or a miR-143 inhibitor and miR-15 to patients with astrocytoma, acute myeloid leukemia, breast carcinoma, bladder carcinoma, cervical carcinoma, colorectal carcinoma, endometrial carcinoma, glioma, glioblastoma, gastric carcinoma, hepatocellular carcinoma, Hodgkin lymphoma, melanoma, mantle cell lymphoma, myxofibrosarcoma, multiple myeloma, neuroblastoma, non-Hodgkin lymphoma, non-small cell lung carcinoma, ovarian carcinoma, esophageal carcinoma, osteosarcoma, pancreatic carcinoma, prostate carcinoma, squamous cell carcinoma of the head and neck, or thyroid carcinoma.

In still further aspects, miR-143 or a miR-143 inhibitor and miR-16 are administered to patients with astrocytoma, breast carcinoma, bladder carcinoma, colorectal carcinoma, endometrial carcinoma, glioblastoma, gastric carcinoma, hepatocellular carcinoma, Hodgkin lymphoma, melanoma, mantle cell lymphoma, myxofibrosarcoma, multiple myeloma, non-small cell lung carcinoma, ovarian carcinoma, esophageal carcinoma, pancreatic carcinoma, prostate carcinoma, squamous cell carcinoma of the head and neck, or thyroid carcinoma.

Aspects of the invention include methods where miR-143 or a miR-143 inhibitor and miR-20 are administered to patients with astrocytoma, acute myeloid leukemia, breast carcinoma, bladder carcinoma, colorectal carcinoma, endometrial carcinoma, glioma, glioblastoma, gastric carcinoma, hepatocellular carcinoma, melanoma, mantle cell lymphoma, neuroblastoma, non-small cell lung carcinoma, ovarian carcinoma, esophageal carcinoma, pancreatic carcinoma, prostate carcinoma, or squamous cell carcinoma of the head and neck.

In a further aspect, miR-143 or a miR-143 inhibitor and miR-21 are administered to patients with astrocytoma, acute myeloid leukemia, breast carcinoma, bladder carcinoma, cervical carcinoma, colorectal carcinoma, endometrial carcinoma, glioma, glioblastoma, gastric carcinoma, hepatocellular carcinoma, Hodgkin lymphoma, leukemia, melanoma, mantle cell lymphoma, multiple myeloma, non-Hodgkin lymphoma, non-small cell lung carcinoma, ovarian carcinoma, esophageal carcinoma, osteosarcoma, pancreatic carcinoma, prostate carcinoma, squamous cell carcinoma of the head and neck, or thyroid carcinoma.

In still further aspects, miR-143 or a miR-143 inhibitor and miR-26a are administered to patients with acute myeloid leukemia, breast carcinoma, bladder carcinoma, cervical carcinoma, colorectal carcinoma, glioma, glioblastoma, gastric carcinoma, hepatocellular carcinoma, leukemia, melanoma, multiple myeloma, neuroblastoma, non-Hodgkin lymphoma, non-small cell lung carcinoma, ovarian carcinoma, esophageal carcinoma, osteosarcoma, pancreatic carcinoma, or prostate carcinoma.

In yet further aspects, miR-143 or a miR-143 inhibitor and miR-34a are administered to patients with astrocytoma, acute myeloid leukemia, breast carcinoma, bladder carcinoma, cervical carcinoma, colorectal carcinoma, endometrial carcinoma, glioma, glioblastoma, gastric carcinoma, hepatocellular carcinoma, Hodgkin lymphoma, leukemia, melanoma, mantle cell lymphoma, multiple myeloma, non-Hodgkin lymphoma, non-small cell lung carcinoma, ovarian carcinoma, esophageal carcinoma, osteosarcoma, pancreatic carcinoma, prostate carcinoma, squamous cell carcinoma of the head and neck, thyroid carcinoma, or urothelial carcinoma.

In certain aspects, miR-143 or a miR-143 inhibitor and miR-126 are administered to patients with astrocytoma, acute myeloid leukemia, breast carcinoma, bladder carcinoma, cervical carcinoma, colorectal carcinoma, endometrial carcinoma, glioma, glioblastoma, gastric carcinoma, hepatocellular carcinoma, Hodgkin lymphoma, leukemia, melanoma, mantle cell lymphoma, non-Hodgkin lymphoma, non-small cell lung carcinoma, ovarian carcinoma, esophageal carcinoma, osteosarcoma, pancreatic carcinoma, prostate carcinoma, squamous cell carcinoma of the head and neck, or thyroid carcinoma.

In still a further aspect, miR-143 or a miR-143 inhibitor and miR-147 are administered to patients with astrocytoma, breast carcinoma, bladder carcinoma, cervical carcinoma, colorectal carcinoma, endometrial carcinoma, esophageal squamous cell carcinoma, glioma, glioblastoma, gastric carcinoma, hepatocellular carcinoma, Hodgkin lymphoma, leukemia, lipoma, melanoma, mantle cell lymphoma, myxofibrosarcoma, multiple myeloma, non-Hodgkin lymphoma, non-small cell lung carcinoma, ovarian carcinoma, esophageal carcinoma, osteosarcoma, pancreatic carcinoma, prostate carcinoma, squamous cell carcinoma of the head and neck, or thyroid carcinoma.

In yet another aspect, miR-143 or a miR-143 inhibitor and miR-188 are administered to patients with astrocytoma, acute myeloid leukemia, breast carcinoma, bladder carcinoma, cervical carcinoma, colorectal carcinoma, endometrial carcinoma, esophageal squamous cell carcinoma, glioma, glioblastoma, gastric carcinoma, hepatocellular carcinoma, leukemia, melanoma, multiple myeloma, non-Hodgkin lymphoma, non-small cell lung carcinoma, ovarian carcinoma, esophageal carcinoma, pancreatic carcinoma, prostate carcinoma, squamous cell carcinoma of the head and neck, or thyroid carcinoma.

In other aspects, miR-143 or a miR-143 inhibitor and miR-215 are administered to patients with astrocytoma, acute myeloid leukemia, breast carcinoma, bladder carcinoma, cervical carcinoma, colorectal carcinoma, endometrial carcinoma, esophageal squamous cell carcinoma, glioma, glioblastoma, gastric carcinoma, hepatocellular carcinoma, Hodgkin lymphoma, leukemia, lipoma, melanoma, mantle cell lymphoma, myxofibrosarcoma, multiple myeloma, neuroblastoma, non-Hodgkin lymphoma, non-small cell lung carcinoma, ovarian carcinoma, esophageal carcinoma, osteosarcoma, pancreatic carcinoma, prostate carcinoma, squamous cell carcinoma of the head and neck, thyroid carcinoma, or urothelial carcinoma.

In certain aspects, miR-143 or a miR-143 inhibitor and miR-216 are administered to patients with astrocytoma, breast carcinoma, cervical carcinoma, colorectal carcinoma, endometrial carcinoma, glioma, glioblastoma, gastric carcinoma, hepatocellular carcinoma, Hodgkin lymphoma, leukemia, non-Hodgkin lymphoma, non-small cell lung carcinoma, ovarian carcinoma, esophageal carcinoma, osteosarcoma, prostate carcinoma, or squamous cell carcinoma of the head and neck.

In a further aspect, miR-143 or a miR-143 inhibitor and miR-292-3p are administered to patients with astrocytoma, acute myeloid leukemia, breast carcinoma, bladder carcinoma, cervical carcinoma, colorectal carcinoma, endometrial carcinoma, glioma, glioblastoma, gastric carcinoma, hepatocellular carcinoma, leukemia, lipoma, melanoma, myxofibrosarcoma, multiple myeloma, neuroblastoma, non-Hodgkin lymphoma, non-small cell lung carcinoma, ovarian carcinoma, esophageal carcinoma, osteosarcoma, pancreatic carcinoma, prostate carcinoma, squamous cell carcinoma of the head and neck, thyroid carcinoma, or urothelial carcinoma.

In still a further aspect, miR-143 or a miR-143 inhibitor and miR-331 are administered to patients with astrocytoma, acute myeloid leukemia, breast carcinoma, bladder carcinoma, cervical carcinoma, colorectal carcinoma, endometrial carcinoma, glioma, glioblastoma, gastric carcinoma, hepatocellular carcinoma, leukemia, melanoma, myxofibrosarcoma, multiple myeloma, neuroblastoma, non-Hodgkin lymphoma, ovarian carcinoma, esophageal carcinoma, osteosarcoma, pancreatic carcinoma, prostate carcinoma, squamous cell carcinoma of the head and neck, or thyroid carcinoma.

In yet a further aspect, miR-143 or a miR-143 inhibitor and miR-200b/c are administered to patients with breast carcinoma, cervical carcinoma, colorectal carcinoma, glioma, glioblastoma, gastric carcinoma, hepatocellular carcinoma, leukemia, lipoma, multiple myeloma, non-small cell lung carcinoma, ovarian carcinoma, esophageal carcinoma, osteosarcoma, pancreatic carcinoma, prostate carcinoma, squamous cell carcinoma of the head and neck, or thyroid carcinoma.

It is contemplated that when miR-143 or a miR-143 inhibitor is given in combination with one or more other miRNA molecules, the two different miRNAs or inhibitors may be given at the same time or sequentially. In some embodiments, therapy proceeds with one miRNA or inhibitor and that therapy is followed up with therapy with the other miRNA or inhibitor 1, 2, 3, 4, 5, 6, 7, 8, 9, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4, 5 weeks, or 1, 2, 3, 4, 5, 6, 7, 8, 9, 10, 11, or 12 months or any such combination later.

Further embodiments include the identification and assessment of an expression profile indicative of miR-143 status in a cell or tissue comprising expression assessment of one or more gene from Table 1, 3, 4, and/or 5, or any combination thereof.

The term “miRNA” is used according to its ordinary and plain meaning and refers to a microRNA molecule found in eukaryotes that is involved in RNA-based gene regulation. See, e.g., Carrington et al., 2003, which is hereby incorporated by reference. The term can be used to refer to the single-stranded RNA molecule processed from a precursor or in certain instances the precursor itself or a mimetic thereof.

In some embodiments, it may be useful to know whether a cell expresses a particular miRNA endogenously or whether such expression is affected under particular conditions or when it is in a particular disease state. Thus, in some embodiments of the invention, methods include assaying a cell or a sample containing a cell for the presence of one or more miRNA marker gene or mRNA or other analyte indicative of the expression level of a gene of interest. Consequently, in some embodiments, methods include a step of generating an RNA profile for a sample. The term “RNA profile” or “gene expression profile” refers to a set of data regarding the expression pattern for one or more gene or genetic marker in the sample (e.g., a plurality of nucleic acid probes that identify one or more markers or genes from Tables 1, 3, 4, and/or 5); it is contemplated that the nucleic acid profile can be obtained using a set of RNAs, using for example nucleic acid amplification or hybridization techniques well know to one of ordinary skill in the art. The difference in the expression profile in the sample from a patient and a reference expression profile, such as an expression profile from a normal or non-pathologic sample, or a digitized reference, is indicative of a pathologic, disease, or cancerous condition. In certain aspects the expression profile is an indicator of a propensity to or probability of (i.e., risk factor for a disease or condition) developing such a condition(s). Such a risk or propensity may indicate a treatment, increased monitoring, prophylactic measures, and the like. A nucleic acid or probe set may comprise or identify a segment of a corresponding mRNA and may include all or part of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 100, 200, 500, or more segments, including any integer or range derivable there between, of a gene or genetic marker, or a nucleic acid, mRNA or a probe representative thereof that is listed in Tables 1, 3, 4, and/or 5 or identified by the methods described herein.

Certain embodiments of the invention are directed to compositions and methods for assessing, prognosing, or treating a pathological condition in a patient comprising measuring or determining an expression profile of one or more miRNA or marker(s) in a sample from the patient, wherein a difference in the expression profile in the sample from the patient and an expression profile of a normal sample or reference expression profile is indicative of pathological condition and particularly cancer (e.g., In certain aspects of the invention, the miRNAs, cellular pathway, gene, or genetic marker is or is representative of one or more pathway or marker described in Table 1, 2, 3, 4, and/or 5, including any combination thereof.

Aspects of the invention include diagnosing, assessing, or treating a pathologic condition or preventing a pathologic condition from manifesting. For example, the methods can be used to screen for a pathological condition; assess prognosis of a pathological condition; stage a pathological condition; assess response of a pathological condition to therapy; or to modulate the expression of a gene, genes, or related pathway as a first therapy or to render a subject sensitive or more responsive to a second therapy. In particular aspects, assessing the pathological condition of the patient can be assessing prognosis of the patient. Prognosis may include, but is not limited to an estimation of the time or expected time of survival, assessment of response to a therapy, and the like. In certain aspects, the altered expression of one or more gene or marker is prognostic for a patient having a pathologic condition, wherein the marker is one or more of Table 1, 3, 4, and/or 5, including any combination thereof.

TABLE 2
Significantly affected functional cellular pathways following
hsa-miR-143 over-expression in human cancer cells.

| Number | |
|---|---|
| of Genes | Pathway Functions |
| 9 | Cellular Movement, Hematological System Development and |
| | Function, Immune Response |
| 2 | Gene Expression, Cellular Growth and Proliferation, |
| | Developmental Disorder |

TABLE 3
Predicted target genes of hsa-miR-143 for Ref Seq ID reference - Pruitt et al., 2005.

| Gene | RefSeq | |
|---|---|---|
| Symbol | Transcript ID | Description |
| 76P | NM_014444 | gamma tubulin ring complex protein (76p gene) |
| AACS | NM_023928 | acetoacetyl-CoA synthetase |
| AADACL1 | NM_020792 | arylacetamide deacetylase-like 1 |
| AARSL | NM_020745 | alanyl-tRNA synthetase like |
| ABAT | NM_000663 | 4-aminobutyrate aminotransferase precursor |
| ABCA1 | NM_005502 | ATP-binding cassette, sub-family A member 1 |
| ABCB11 | NM_003742 | ATP-binding cassette, sub-family B (MDR/TAP), |
| ABCB9 | NM_203445 | ATP-binding cassette, sub-family B (MDR/TAP), |
| ABCC1 | NM_004996 | ATP-binding cassette, sub-family C, member 1 |
| ABCC13 | NM_172024 | ATP-binding cassette protein C13 isoform b |
| ABCC3 | NM_020038 | ATP-binding cassette, sub-family C, member 3 |
| ABCC4 | NM_005845 | ATP-binding cassette, sub-family C, member 4 |
| ABCG4 | NM_022169 | ATP-binding cassette, subfamily G, member 4 |
| ABCG5 | NM_022436 | sterolin 1 |
| ABHD14A | NM_015407 | abhydrolase domain containing 14A |
| ABHD14B | NM_032750 | abhydrolase domain containing 14B |
| ABHD8 | NM_024527 | abhydrolase domain containing 8 |
| ABLIM1 | NM_001003407 | actin-binding LIM protein 1 isoform b |
| ABR | NM_001092 | active breakpoint cluster region-related |
| ABTB2 | NM_145804 | ankyrin repeat and BTB (POZ) domain containing |
| ACACB | NM_001093 | acetyl-Coenzyme A carboxylase beta |
| ACADSB | NM_001609 | acyl-Coenzyme A dehydrogenase, short/branched |
| ACCN1 | NM_001094 | amiloride-sensitive cation channel 1, neuronal |
| ACE | NM_152831 | angiotensin I converting enzyme isoform 3 |
| ACE2 | NM_021804 | angiotensin I converting enzyme 2 precursor |
| ACIN1 | NM_014977 | apoptotic chromatin condensation inducer 1 |
| ACOXL | NM_018308 | acyl-Coenzyme A oxidase-like |
| ACP1 | NM_004300 | acid phosphatase 1 isoform c |
| ACSL6 | NM_001009185 | acyl-CoA synthetase long-chain family member 6 |
| ACTL8 | NM_030812 | actin like protein |
| ACTN2 | NM_001103 | actinin, alpha 2 |
| ACTR8 | NM_022899 | actin-related protein 8 |
| ACVR1B | NM_004302 | activin A type IB receptor isoform a precursor |
| ACY1L2 | NM_001010853 | hypothetical protein LOC135293 |
| ADAM10 | NM_001110 | ADAM metallopeptidase domain 10 |
| ADAM12 | NM_003474 | ADAM metallopeptidase domain 12 isoform 1 |
| ADAM9 | NM_001005845 | ADAM metallopeptidase domain 9 isoform 2 |
| ADAMTS1 | NM_006988 | ADAM metallopeptidase with thrombospondin type 1 |
| ADAMTS3 | NM_014243 | ADAM metallopeptidase with thrombospondin type 1 |
| ADAMTS4 | NM_005099 | ADAM metallopeptidase with thrombospondin type 1 |
| ADAMTSL1 | NM_052866 | ADAMTS-like 1 isoform 2 |
| ADAR | NM_001025107 | adenosine deaminase, RNA-specific isoform d |
| ADARB1 | NM_001033049 | RNA-specific adenosine deaminase B1 isoform 4 |
| ADAT1 | NM_012091 | adenosine deaminase, tRNA-specific 1 |
| ADCY1 | NM_021116 | brain adenylate cyclase 1 |
| ADCY2 | NM_020546 | adenylate cyclase 2 |
| ADCY6 | NM_015270 | adenylate cyclase 6 isoform a |
| ADCY9 | NM_001116 | adenylate cyclase 9 |
| ADD2 | NM_001617 | adducin 2 isoform a |
| ADD3 | NM_001121 | adducin 3 (gamma) isoform b |
| ADI1 | NM_018269 | membrane-type 1 matrix metalloproteinase |
| ADIPOQ | NM_004797 | adiponectin precursor |
| ADORA3 | NM_000677 | adenosine A3 receptor isoform 2 |
| ADRA2B | NM_000682 | alpha-2B-adrenergic receptor |
| ADSSL1 | NM_152328 | adenylosuccinate synthase-like 1 isoform 2 |
| AFAP | NM_021638 | actin filament associated protein |
| AFF1 | NM_005935 | myeloid/lymphoid or mixed-lineage leukemia |
| AFF2 | NM_002025 | fragile X mental retardation 2 |
| AFG3L2 | NM_006796 | AFG3 ATPase family gene 3-like 2 |
| AGBL4 | NM_032785 | hypothetical protein LOC84871 |
| AGMAT | NM_024758 | agmatine ureohydrolase (agmatinase) |
| AGPAT1 | NM_006411 | 1-acylglycerol-3-phosphate O-acyltransferase 1 |
| AGPAT3 | NM_020132 | 1-acylglycerol-3-phosphate O-acyltransferase 3 |
| AGPAT4 | NM_001012733 | 1-acylglycerol-3-phosphate O-acyltransferase 4 |
| AGR2 | NM_006408 | anterior gradient 2 homolog |
| AGRN | NM_198576 | agrin |
| AHCTF1 | NM_015446 | transcription factor ELYS |
| AHCYL1 | NM_006621 | S-adenosylhomocysteine hydrolase-like 1 |
| AICDA | NM_020661 | activation-induced cytidine deaminase |
| AIF1 | NM_004847 | allograft inflammatory factor 1 isoform 2 |
| AIG1 | NM_016108 | androgen-induced 1 |
| AIPL1 | NM_001033054 | aryl hydrocarbon receptor interacting |
| AIRE | NM_000383 | autoimmune regulator AIRE isoform 1 |
| AK1 | NM_000476 | adenylate kinase 1 |
| AK3 | NM_016282 | adenylate kinase 3 |
| AKAP11 | NM_144490 | A-kinase anchor protein 11 isoform 2 |
| AKAP13 | NM_006738 | A-kinase anchor protein 13 isoform 1 |
| AKAP6 | NM_004274 | A-kinase anchor protein 6 |
| AKT1 | NM_001014431 | v-akt murine thymoma viral oncogene homolog 1 |
| ALB | NM_000477 | albumin precursor |
| ALDH3A2 | NM_000382 | aldehyde dehydrogenase 3A2 isoform 2 |
| ALDH5A1 | NM_001080 | aldehyde dehydrogenase 5A1 precursor, isoform 2 |
| ALKBH4 | NM_017621 | hypothetical protein LOC54784 |
| ALPL | NM_000478 | tissue non-specific alkaline phosphatase |
| ALS2 | NM_020919 | alsin |
| ALX3 | NM_006492 | aristaless-like homeobox 3 |
| AMDHD1 | NM_152435 | hypothetical protein LOC144193 |
| AMFR | NM_001144 | autocrine motility factor receptor |
| AMICA1 | NM_153206 | adhesion molecule, interacts with CXADR antigen |
| AMMECR1 | NM_001025580 | AMMECR1 protein isoform 2 |
| AMOTL1 | NM_130847 | angiomotin like 1 |
| AMPD2 | NM_004037 | adenosine monophosphate deaminase 2 (isoform L) |
| AMT | NM_000481 | aminomethyltransferase (glycine cleavage system |
| AMZ1 | NM_133463 | archaemetzincin-1 |
| ANGEL1 | NM_015305 | angel homolog 1 |
| ANGPTL1 | NM_004673 | angiopoietin-like 1 precursor |
| ANGPTL2 | NM_012098 | angiopoietin-like 2 precursor |
| ANGPTL7 | NM_021146 | angiopoietin-like 7 |
| ANKH | NM_054027 | ankylosis, progressive homolog |
| ANKRD12 | NM_015208 | ankyrin repeat domain 12 |
| ANKRD13 | NM_033121 | ankyrin repeat domain 13 |
| ANKRD20A3 | NM_001012419 | hypothetical protein LOC441425 |
| ANKRD25 | NM_015493 | ankyrin repeat domain 25 |
| ANKRD28 | NM_015199 | ankyrin repeat domain 28 |
| ANKRD29 | NM_173505 | ankyrin repeat domain 29 |
| ANKRD41 | NM_152363 | ankyrin repeat domain 41 |
| ANKRD50 | NM_020337 | ankyrin repeat domain 50 |
| ANKS6 | NM_173551 | sterile alpha motif domain containing 6 |
| ANXA3 | NM_005139 | annexin A3 |
| ANXA9 | NM_003568 | annexin A9 |
| AOC2 | NM_001158 | amine oxidase, copper containing 2 isoform a |
| AP2B1 | NM_001030006 | adaptor-related protein complex 2, beta 1 |
| AP3D1 | NM_003938 | adaptor-related protein complex 3, delta 1 |
| AP3M1 | NM_012095 | adaptor-related protein complex 3, mu 1 subunit |
| APAF1 | NM_001160 | apoptotic protease activating factor isoform b |
| APOA1BP | NM_144772 | apolipoprotein A-I binding protein precursor |
| APOA5 | NM_052968 | apolipoprotein AV |
| APOBEC3A | NM_145699 | phorbolin 1 |
| APOBEC3F | NM_145298 | apolipoprotein B mRNA editing enzyme, catalytic |
| APOBEC4 | NM_203454 | apolipoprotein B mRNA editing enzyme, catalytic |
| APOL1 | NM_003661 | apolipoprotein L1 isoform a precursor |
| APOL6 | NM_030641 | apolipoprotein L6 |
| APOLD1 | NM_030817 | apolipoprotein L domain containing 1 |
| APPL | NM_012096 | adaptor protein containing pH domain, PTB domain |
| APTX | NM_175069 | aprataxin isoform b |
| AQP10 | NM_080429 | aquaporin 10 |
| AQP2 | NM_000486 | aquaporin 2 |
| AQP3 | NM_004925 | aquaporin 3 |
| ARCN1 | NM_001655 | archain |
| ARFGAP3 | NM_014570 | ADP-ribosylation factor GTPase activating |
| ARFIP2 | NM_012402 | ADP-ribosylation factor interacting protein 2 |
| ARHGAP18 | NM_033515 | Rho GTPase activating protein 18 |
| ARHGAP20 | NM_020809 | Rho GTPase activating protein 20 |
| ARHGAP25 | NM_001007231 | Rho GTPase activating protein 25 isoform a |
| ARHGAP26 | NM_015071 | GTPase regulator associated with the focal |
| ARHGAP28 | NM_001010000 | Rho GTPase activating protein 28 isoform a |
| ARHGAP9 | NM_032496 | Rho GTPase activating protein 9 |
| ARHGDIB | NM_001175 | Rho GDP dissociation inhibitor (GDI) beta |
| ARHGEF1 | NM_004706 | Rho guanine nucleotide exchange factor 1 isoform |
| ARHGEF7 | NM_003899 | Rho guanine nucleotide exchange factor 7 isoform |
| ARID3B | NM_006465 | AT rich interactive domain 3B (BRIGHT-like) |
| ARID5B | NM_032199 | AT rich interactive domain 5B (MRF1-like) |
| ARL15 | NM_019087 | ADP-ribosylation factor related protein 2 |
| ARL3 | NM_004311 | ADP-ribosylation factor-like 3 |
| ARL6 | NM_032146 | ADP-ribosylation factor-like 6 |
| ARL6IP2 | NM_022374 | ADP-ribosylation factor-like 6 interacting |
| ARMC5 | NM_024742 | armadillo repeat containing 5 |
| ARMC8 | NM_014154 | armadillo repeat containing 8 isoform 1 |
| ARNT | NM_001668 | aryl hydrocarbon receptor nuclear translocator |
| ARRDC4 | NM_183376 | arrestin domain containing 4 |
| ARSD | NM_001669 | arylsulfatase D isoform a precursor |
| ARTS-1 | NM_016442 | type 1 tumor necrosis factor receptor shedding |
| ASAM | NM_024769 | adipocyte-specific adhesion molecule |
| ASB4 | NM_145872 | ankyrin repeat and SOCS box-containing protein 4 |
| ASB6 | NM_017873 | ankyrin repeat and SOCS box-containing 6 isoform |
| ASCC3 | NM_006828 | activating signal cointegrator 1 complex subunit |
| ASL | NM_000048 | argininosuccinate lyase isoform 1 |
| ASPH | NM_004318 | aspartate beta-hydroxylase isoform a |
| ASTN | NM_004319 | astrotactin isoform 1 |
| ASXL1 | NM_015338 | additional sex combs like 1 |
| ASXL2 | NM_018263 | additional sex combs like 2 |
| ATCAY | NM_033064 | caytaxin |
| ATF3 | NM_001030287 | activating transcription factor 3 isoform 1 |
| ATG10 | NM_031482 | APG10 autophagy 10-like |
| ATG12 | NM_004707 | APG12 autophagy 12-like |
| ATG9A | NM_024085 | APG9 autophagy 9-like 1 |
| ATG9B | NM_173681 | nitric oxide synthase 3 antisense |
| ATHL1 | NM_025092 | hypothetical protein LOC80162 |
| ATM | NM_000051 | ataxia telangiectasia mutated protein isoform 1 |
| ATOH8 | NM_032827 | atonal homolog 8 |
| ATP10A | NM_024490 | ATPase, Class V, type 10A |
| ATP11B | NM_014616 | ATPase, Class VI, type 11B |
| ATP11C | NM_001010986 | ATPase, Class VI, type 11C isoform b |
| ATP1A2 | NM_000702 | Na+/K+-ATPase alpha 2 subunit proprotein |
| ATP1A3 | NM_152296 | Na+/K+-ATPase alpha 3 subunit |
| ATP2B2 | NM_001001331 | plasma membrane calcium ATPase 2 isoform a |
| ATP6AP1 | NM_001183 | ATPase, H+ transporting, lysosomal accessory |
| ATP6V0E | NM_003945 | ATPase, H+ transporting, lysosomal, V0 subunit |
| ATP6V1A | NM_001690 | ATPase, H+ transporting, lysosomal 70 kD, V1 |
| ATP6V1C2 | NM_144583 | vacuolar H+ ATPase C2 isoform b |
| ATP6V1F | NM_004231 | ATPase, H+ transporting, lysosomal 14 kD, V1 |
| ATP8A1 | NM_006095 | ATPase, aminophospholipid transporter (APLT), |
| ATPBD4 | NM_080650 | ATP binding domain 4 |
| ATPIF1 | NM_178191 | ATPase inhibitory factor 1 isoform 3 precursor |
| ATXN1 | NM_000332 | ataxin 1 |
| AVPR1B | NM_000707 | arginine vasopressin receptor 1B |
| AZGP1 | NM_001185 | alpha-2-glycoprotein 1, zinc |
| B3GNT6 | NM_138706 | UDP-GlcNAc:betaGal |
| B4GALT1 | NM_001497 | UDP-Gal:betaGlcNAc beta 1,4- |
| B4GALT5 | NM_004776 | UDP-Gal:betaGlcNAc beta 1,4- |
| BAAT | NM_001701 | bile acid Coenzyme A: amino acid |
| BACE1 | NM_012104 | beta-site APP-cleaving enzyme 1 isoform A |
| BACH1 | NM_001011545 | BTB and CNC homology 1 isoform b |
| BACH2 | NM_021813 | BTB and CNC homology 1, basic leucine zipper |
| BAG1 | NM_004323 | BCL2-associated athanogene isoform 1L |
| BAG3 | NM_004281 | BCL2-associated athanogene 3 |
| BAG5 | NM_001015048 | BCL2-associated athanogene 5 isoform b |
| BAGE4 | NM_181704 | B melanoma antigen family, member 4 |
| BARHL2 | NM_020063 | BarH-like 2 |
| BAT2D1 | NM_015172 | HBxAg transactivated protein 2 |
| BATF2 | NM_138456 | basic leucine zipper transcription factor, |
| BAZ2A | NM_013449 | bromodomain adjacent to zinc finger domain, 2A |
| BBC3 | NM_014417 | BCL2 binding component 3 |
| BBS1 | NM_024649 | Bardet-Biedl syndrome 1 |
| BBS5 | NM_152384 | Bardet-Biedl syndrome 5 |
| BCAN | NM_198427 | brevican isoform 2 |
| BCAP29 | NM_001008406 | B-cell receptor-associated protein BAP29 isoform |
| BCAP31 | NM_005745 | B-cell receptor-associated protein 31 |
| BCL2 | NM_000633 | B-cell lymphoma protein 2 alpha isoform |
| BCL3 | NM_005178 | B-cell CLL/lymphoma 3 |
| BCORL1 | NM_021946 | BCL6 co-repressor-like 1 |
| BCR | NM_004327 | breakpoint cluster region isoform 1 |
| BDH2 | NM_020139 | 3-hydroxybutyrate dehydrogenase, type 2 |
| BET1L | NM_016526 | blocked early in transport 1 homolog (S. |
| BFAR | NM_016561 | apoptosis regulator |
| BGN | NM_001711 | biglycan preproprotein |
| BHLHB9 | NM_030639 | basic helix-loop-helix domain containing, class |
| BHMT2 | NM_017614 | betaine-homocysteine methyltransferase 2 |
| BICD1 | NM_001003398 | bicaudal D homolog 1 isoform 2 |
| BIRC1 | NM_004536 | baculoviral IAP repeat-containing 1 |
| BIRC2 | NM_001166 | baculoviral IAP repeat-containing protein 2 |
| BIRC4 | NM_001167 | baculoviral IAP repeat-containing protein 4 |
| BIRC4BP | NM_017523 | XIAP associated factor-1 isoform 1 |
| BIRC5 | NM_001012270 | baculoviral IAP repeat-containing protein 5 |
| BLMH | NM_000386 | bleomycin hydrolase |
| BLOC1S2 | NM_001001342 | biogenesis of lysosome-related organelles |
| BLR1 | NM_001716 | Burkitt lymphoma receptor 1 isoform 1 |
| BLZF1 | NM_003666 | basic leucine zipper nuclear factor 1 |
| BMPR1A | NM_004329 | bone morphogenetic protein receptor, type IA |
| BMPR2 | NM_001204 | bone morphogenetic protein receptor type II |
| BOK | NM_032515 | BCL2-related ovarian killer |
| BOLA2 | NM_001031833 | BolA-like protein 2 isoform b |
| BOLL | NM_033030 | boule isoform 2 |
| BPNT1 | NM_006085 | 3′(2′), 5′-bisphosphate nucleotidase 1 |
| BRCA1 | NM_007306 | breast cancer 1, early onset isoform |
| BRD2 | NM_005104 | bromodomain containing protein 2 |
| BRD4 | NM_014299 | bromodomain-containing protein 4 isoform short |
| BSN | NM_003458 | bassoon protein |
| BTBD14B | NM_052876 | transcriptional repressor NAC1 |
| BTBD15 | NM_014155 | BTB (POZ) domain containing 15 |
| BTBD4 | NM_025224 | BTB (POZ) domain containing 4 |
| BTBD6 | NM_033271 | BTB domain protein BDPL |
| BTF3L4 | NM_152265 | transcription factor BTF3-like |
| BTG2 | NM_006763 | B-cell translocation gene 2 |
| BTN1A1 | NM_001732 | butyrophilin, subfamily 1, member A1 |
| BTN2A1 | NM_007049 | butyrophilin, subfamily 2, member A1 isoform 1 |
| BTN2A2 | NM_006995 | butyrophilin, subfamily 2, member A2 isoform a |
| BTN3A2 | NM_007047 | butyrophilin, subfamily 3, member A2 precursor |
| BTNL8 | NM_024850 | butyrophilin-like 8 short form |
| BTRC | NM_003939 | beta-transducin repeat containing protein |
| BVES | NM_007073 | blood vessel epicardial substance |
| C10orf10 | NM_007021 | fasting induced gene |
| C10orf104 | NM_173473 | hypothetical protein LOC119504 |
| C10orf111 | NM_153244 | hypothetical protein LOC221060 |
| C10orf114 | NM_001010911 | hypothetical protein LOC399726 |
| C10orf12 | NM_015652 | hypothetical protein LOC26148 |
| C10orf129 | NM_207321 | hypothetical protein LOC142827 |
| C10orf38 | NM_001010924 | hypothetical protein LOC221061 |
| C10orf39 | NM_194303 | hypothetical protein LOC282973 |
| C10orf42 | NM_138357 | hypothetical protein LOC90550 |
| C10orf46 | NM_153810 | hypothetical protein LOC143384 |
| C10orf53 | NM_182554 | hypothetical protein LOC282966 |
| C10orf54 | NM_022153 | hypothetical protein LOC64115 |
| C10orf56 | NM_153367 | hypothetical protein LOC219654 |
| C10orf65 | NM_138413 | hypothetical protein LOC112817 |
| C10orf83 | NM_178832 | hypothetical protein LOC118812 |
| C10orf99 | NM_207373 | hypothetical protein LOC387695 |
| C11orf1 | NM_022761 | hypothetical protein LOC64776 |
| C11orf17 | NM_182901 | chromosome 11 open reading frame 17 |
| C11orf45 | NM_145013 | hypothetical protein LOC219833 |
| C11orf46 | NM_152316 | hypothetical protein LOC120534 |
| C11orf49 | NM_001003676 | hypothetical protein LOC79096 isoform 1 |
| C11orf54 | NM_014039 | hypothetical protein LOC28970 |
| C11orf55 | NM_207428 | hypothetical protein LOC399879 |
| C11orf69 | NM_152314 | hypothetical protein LOC120196 |
| C12orf22 | NM_030809 | TGF-beta induced apoptosis protein 12 |
| C12orf29 | NM_001009894 | hypothetical protein LOC91298 |
| C12orf31 | NM_032338 | hypothetical protein LOC84298 |
| C12orf41 | NM_017822 | hypothetical protein LOC54934 |
| C12orf5 | NM_020375 | chromosome 12 open reading frame 5 |
| C12orf59 | NM_153022 | hypothetical protein LOC120939 |
| C13orf3 | NM_145061 | hypothetical protein LOC221150 |
| C14orf103 | NM_018036 | hypothetical protein LOC55102 |
| C14orf11 | NM_018453 | hypothetical protein LOC55837 |
| C14orf115 | NM_018228 | hypothetical protein LOC55237 |
| C14orf143 | NM_145231 | hypothetical protein LOC90141 |
| C14orf150 | NM_001008726 | hypothetical protein LOC112840 |
| C14orf162 | NM_020181 | chromosome 14 open reading frame 162 |
| C14orf43 | NM_194278 | hypothetical protein LOC91748 |
| C14orf58 | NM_017791 | hypothetical protein LOC55640 |
| C14orf8 | NM_173846 | chromosome 14 open reading frame 8 |
| C15orf15 | NM_016304 | ribosomal protein L24-like |
| C15orf20 | NM_025049 | DNA helicase homolog PIF1 |
| C15orf27 | NM_152335 | hypothetical protein LOC123591 |
| C15orf38 | NM_182616 | hypothetical protein LOC348110 |
| C15orf39 | NM_015492 | hypothetical protein LOC56905 |
| C15orf42 | NM_152259 | leucine-rich repeat kinase 1 |
| C16orf53 | NM_024516 | hypothetical protein LOC79447 |
| C16orf54 | NM_175900 | hypothetical protein LOC283897 |
| C16orf58 | NM_022744 | hypothetical protein LOC64755 |
| C17orf28 | NM_030630 | hypothetical protein LOC283987 |
| C17orf42 | NM_024683 | hypothetical protein LOC79736 |
| C17orf45 | NM_152350 | hypothetical protein LOC125144 |
| C17orf53 | NM_024032 | hypothetical protein LOC78995 |
| C17orf56 | NM_144679 | hypothetical protein LOC146705 |
| C17orf59 | NM_017622 | hypothetical protein LOC54785 |
| C17orf69 | NM_152466 | hypothetical protein LOC147081 |
| C18orf1 | NM_001003674 | hypothetical protein LOC753 isoform gamma 1 |
| C18orf24 | NM_145060 | hypothetical protein LOC220134 |
| C18orf25 | NM_001008239 | chromosome 18 open reading frame 25 isoform b |
| C18orf45 | NM_032933 | hypothetical protein LOC85019 |
| C19orf10 | NM_019107 | chromosome 19 open reading frame 10 |
| C19orf23 | NM_152480 | hypothetical protein LOC148046 |
| C19orf35 | NM_198532 | hypothetical protein LOC374872 |
| C19orf39 | NM_175871 | hypothetical protein LOC126074 |
| C19orf4 | NM_012109 | brain-specific membrane-anchored protein |
| C1orf106 | NM_018265 | hypothetical protein LOC55765 |
| C1orf107 | NM_014388 | hypothetical protein LOC27042 |
| C1orf108 | NM_024595 | hypothetical protein LOC79647 |
| C1orf109 | NM_017850 | hypothetical protein LOC54955 |
| C1orf115 | NM_024709 | hypothetical protein LOC79762 |
| C1orf116 | NM_023938 | specifically androgen-regulated protein |
| C1orf117 | NM_182623 | hypothetical protein LOC348487 |
| C1orf119 | NM_020141 | hypothetical protein LOC56900 |
| C1orf130 | NM_001010980 | hypothetical protein LOC400746 |
| C1orf135 | NM_024037 | hypothetical protein LOC79000 |
| C1orf140 | NM_001010913 | hypothetical protein LOC400804 |
| C1orf144 | NM_015609 | putative MAPK activating protein PM20, PM21 |
| C1orf145 | NM_001025495 | hypothetical protein LOC574407 |
| C1orf149 | NM_022756 | hypothetical protein LOC64769 |
| C1orf151 | NM_001032363 | chromosome 1 open reading frame 151 protein |
| C1orf157 | NM_182579 | hypothetical protein LOC284573 |
| C1orf162 | NM_174896 | hypothetical protein LOC128346 |
| C1orf166 | NM_024544 | hypothetical protein LOC79594 |
| C1orf172 | NM_152365 | hypothetical protein LOC126695 |
| C1orf173 | NM_001002912 | hypothetical protein LOC127254 |
| C1orf183 | NM_019099 | hypothetical protein LOC55924 isoform 1 |
| C1orf187 | NM_198545 | chromosome 1 open reading frame 187 |
| C1orf21 | NM_030806 | chromosome 1 open reading frame 21 |
| C1orf36 | NM_183059 | chromosome 1 open reading frame 36 |
| C1orf38 | NM_004848 | basement membrane-induced gene isoform 1 |
| C1orf45 | NM_001025231 | hypothetical protein LOC448834 |
| C1orf49 | NM_032126 | hypothetical protein LOC84066 |
| C1orf52 | NM_198077 | hypothetical protein LOC148423 |
| C1orf53 | NM_001024594 | hypothetical protein LOC388722 |
| C1orf56 | NM_017860 | hypothetical protein LOC54964 |
| C1orf61 | NM_006365 | transcriptional activator of the c-fos promoter |
| C1orf66 | NM_015997 | hypothetical protein LOC51093 |
| C1orf69 | NM_001010867 | hypothetical protein LOC200205 |
| C1orf74 | NM_152485 | hypothetical protein LOC148304 |
| C1orf76 | NM_173509 | hypothetical protein MGC16664 |
| C1orf80 | NM_022831 | hypothetical protein LOC64853 |
| C1orf83 | NM_153035 | hypothetical protein LOC127428 |
| C1orf95 | NM_001003665 | hypothetical protein LOC375057 |
| C1orf96 | NM_145257 | hypothetical protein LOC126731 |
| C1QTNF1 | NM_030968 | C1q and tumor necrosis factor related protein 1 |
| C1RL | NM_016546 | complement component 1, r subcomponent-like |
| C20orf108 | NM_080821 | hypothetical protein LOC116151 |
| C20orf11 | NM_017896 | chromosome 20 open reading frame 11 |
| C20orf111 | NM_016470 | oxidative stress responsive 1 |
| C20orf12 | NM_018152 | hypothetical protein LOC55184 |
| C20orf28 | NM_015417 | hypothetical protein LOC25876 |
| C20orf29 | NM_018347 | hypothetical protein LOC55317 |
| C20orf4 | NM_015511 | hypothetical protein LOC25980 |
| C20orf42 | NM_017671 | chromosome 20 open reading frame 42 |
| C20orf43 | NM_016407 | hypothetical protein LOC51507 |
| C20orf44 | NM_018244 | basic FGF-repressed Zic binding protein isoform |
| C20orf98 | NM_024958 | hypothetical protein LOC80023 |
| C21orf114 | NM_001012707 | hypothetical protein LOC378826 |
| C21orf24 | NM_001001789 | hypothetical protein LOC400866 |
| C21orf29 | NM_144991 | chromosome 21 open reading frame 29 |
| C21orf62 | NM_019596 | hypothetical protein LOC56245 |
| C21orf69 | NM_058189 | chromosome 21 open reading frame 69 |
| C21orf93 | NM_145179 | hypothetical protein LOC246704 |
| C22orf13 | NM_031444 | chromosome 22 open reading frame 13 |
| C22orf18 | NM_001002876 | proliferation associated nuclear element 1 |
| C22orf25 | NM_152906 | hypothetical protein LOC128989 |
| C22orf9 | NM_001009880 | hypothetical protein LOC23313 isoform b |
| C2orf11 | NM_144629 | hypothetical protein LOC130132 |
| C2orf15 | NM_144706 | hypothetical protein LOC150590 |
| C2orf17 | NM_024293 | hypothetical protein LOC79137 |
| C2orf18 | NM_017877 | hypothetical protein LOC54978 |
| C2orf27 | NM_013310 | hypothetical protein LOC29798 |
| C2orf37 | NM_025000 | hypothetical protein LOC80067 |
| C3orf17 | NM_001025072 | hypothetical protein LOC25871 isoform b |
| C3orf21 | NM_152531 | hypothetical protein LOC152002 |
| C3orf23 | NM_001029839 | hypothetical protein LOC285343 isoform 2 |
| C3orf34 | NM_032898 | hypothetical protein LOC84984 |
| C4orf13 | NM_001030316 | hypothetical protein LOC84068 isoform a |
| C5orf21 | NM_032042 | hypothetical protein LOC83989 |
| C5orf24 | NM_152409 | hypothetical protein LOC134553 |
| C5orf4 | NM_016348 | hypothetical protein LOC10826 isoform 1 |
| C6orf130 | NM_145063 | hypothetical protein LOC221443 |
| C6orf149 | NM_020408 | hypothetical protein LOC57128 |
| C6orf15 | NM_014070 | STG protein |
| C6orf155 | NM_024882 | hypothetical protein LOC79940 |
| C6orf157 | NM_198920 | hypothetical protein LOC90025 |
| C6orf165 | NM_178823 | hypothetical protein LOC154313 isoform 2 |
| C6orf201 | NM_206834 | hypothetical protein LOC404220 |
| C6orf205 | NM_001010909 | hypothetical protein LOC394263 |
| C6orf69 | NM_173562 | hypothetical protein LOC222658 |
| C6orf96 | NM_017909 | hypothetical protein LOC55005 |
| C6orf97 | NM_025059 | hypothetical protein LOC80129 |
| C7 | NM_000587 | complement component 7 precursor |
| C7orf34 | NM_178829 | hypothetical protein LOC135927 |
| C7orf38 | NM_145111 | hypothetical protein LOC221786 |
| C8orf1 | NM_004337 | hypothetical protein LOC734 |
| C8orf17 | NM_020237 | MOST-1 protein |
| C8orf44 | NM_019607 | hypothetical protein LOC56260 |
| C8orf51 | NM_024035 | hypothetical protein LOC78998 |
| C9orf106 | NM_001012715 | hypothetical protein LOC414318 |
| C9orf128 | NM_001012446 | hypothetical protein LOC392307 |
| C9orf140 | NM_178448 | hypothetical protein LOC89958 |
| C9orf152 | NM_001012993 | hypothetical protein LOC401546 |
| C9orf163 | NM_152571 | hypothetical protein LOC158055 |
| C9orf25 | NM_147202 | hypothetical protein LOC203259 |
| C9orf27 | NM_021208 | chromosome 9 open reading frame 27 |
| C9orf42 | NM_138333 | hypothetical protein LOC116224 |
| C9orf5 | NM_032012 | hypothetical protein LOC23731 |
| C9orf50 | NM_199350 | hypothetical protein LOC375759 |
| C9orf58 | NM_001002260 | chromosome 9 open reading frame 58 isoform 2 |
| C9orf65 | NM_138818 | hypothetical protein LOC158471 |
| C9orf89 | NM_032310 | chromosome 9 open reading frame 89 |
| C9orf91 | NM_153045 | hypothetical protein LOC203197 |
| CA12 | NM_001218 | carbonic anhydrase XII isoform 1 precursor |
| CA2 | NM_000067 | carbonic anhydrase II |
| CABLES2 | NM_031215 | Cdk5 and Abl enzyme substrate 2 |
| CACHD1 | NM_020925 | cache domain containing 1 |
| CACNA1E | NM_000721 | calcium channel, voltage-dependent, alpha 1E |
| CACNA2D2 | NM_001005505 | calcium channel, voltage-dependent, alpha |
| CACNA2D3 | NM_018398 | calcium channel, voltage-dependent, alpha |
| CACNG4 | NM_014405 | voltage-dependent calcium channel gamma-4 |
| CALCB | NM_000728 | calcitonin-related polypeptide, beta |
| CALD1 | NM_004342 | caldesmon 1 isoform 2 |
| CALM3 | NM_005184 | calmodulin 3 |
| CALML4 | NM_033429 | calmodulin-like 4 isoform 2 |
| CALN1 | NM_001017440 | calneuron 1 |
| CALR | NM_004343 | calreticulin precursor |
| CAMK2A | NM_015981 | calcium/calmodulin-dependent protein kinase IIA |
| CAMK2D | NM_172127 | calcium/calmodulin-dependent protein kinase II |
| CAMK2G | NM_001222 | calcium/calmodulin-dependent protein kinase II |
| CAMKK1 | NM_032294 | calcium/calmodulin-dependent protein kinase 1 |
| CAMKK2 | NM_006549 | calcium/calmodulin-dependent protein kinase |
| CAMLG | NM_001745 | calcium modulating ligand |
| CAMSAP1 | NM_015447 | calmodulin regulated spectrin-associated protein |
| CAND1 | NM_018448 | TIP120 protein |
| CAPN11 | NM_007058 | calpain 11 |
| CAPN3 | NM_212464 | calpain 3 isoform g |
| CAPZB | NM_004930 | F-actin capping protein beta subunit |
| CARKL | NM_013276 | carbohydrate kinase-like |
| CASC2 | NM_178816 | cancer susceptibility candidate 2 isoform 1 |
| CASC3 | NM_007359 | cancer susceptibility candidate 3 |
| CASKIN2 | NM_020753 | cask-interacting protein 2 |
| CASP2 | NM_032982 | caspase 2 isoform 1 preproprotein |
| CASP8 | NM_001228 | caspase 8 isoform A |
| CASQ2 | NM_001232 | cardiac calsequestrin 2 |
| CAST1 | NM_015576 | cytomatrix protein p110 |
| CBFA2T2 | NM_001032999 | core-binding factor, runt domain, alpha subunit |
| CBFB | NM_001755 | core-binding factor, beta subunit isoform 2 |
| CBL | NM_005188 | Cas-Br-M (murine) ecotropic retroviral |
| CBLL1 | NM_024814 | Cas-Br-M (murine) ecotropic retroviral |
| CBX7 | NM_175709 | chromobox homolog 7 |
| CC2D1B | NM_032449 | coiled-coil and C2 domain containing 1B |
| CCBL1 | NM_004059 | cytoplasmic cysteine conjugate-beta lyase |
| CCBP2 | NM_001296 | chemokine binding protein 2 |
| CCDC102B | NM_024781 | hypothetical protein LOC79839 |
| CCDC14 | NM_022757 | coiled-coil domain containing 14 |
| CCDC21 | NM_022778 | coiled-coil domain containing 21 |
| CCDC25 | NM_001031708 | coiled-coil domain containing 25 isoform 1 |
| CCDC33 | NM_182791 | hypothetical protein LOC80125 |
| CCDC49 | NM_017748 | hypothetical protein LOC54883 |
| CCDC58 | NM_001017928 | hypothetical protein LOC131076 |
| CCDC68 | NM_025214 | CTCL tumor antigen se57-1 |
| CCDC72 | NM_015933 | hypothetical protein LOC51372 |
| CCDC93 | NM_019044 | hypothetical protein LOC54520 |
| CCDC94 | NM_018074 | hypothetical protein LOC55702 |
| CCDC97 | NM_052848 | hypothetical protein LOC90324 |
| CCDC98 | NM_139076 | coiled-coil domain containing 98 |
| CCKAR | NM_000730 | cholecystokinin A receptor |
| CCL18 | NM_002988 | small inducible cytokine A18 precursor |
| CCL22 | NM_002990 | small inducible cytokine A22 precursor |
| CCL4L1 | NM_001001435 | chemokine (C-C motif) ligand 4-like 1 precursor |
| CCL4L2 | NM_207007 | chemokine (C-C motif) ligand 4-like 2 precursor |
| CCL7 | NM_006273 | chemokine (C-C motif) ligand 7 precursor |
| CCND1 | NM_053056 | cyclin D1 |
| CCND2 | NM_001759 | cyclin D2 |
| CCNT2 | NM_001241 | cyclin T2 isoform a |
| CCPG1 | NM_004748 | cell cycle progression 1 isoform 1 |
| CCR1 | NM_001295 | chemokine (C-C motif) receptor 1 |
| CCR2 | NM_000647 | chemokine (C-C motif) receptor 2 isoform A |
| CCR6 | NM_004367 | chemokine (C-C motif) receptor 6 |
| CCT5 | NM_012073 | chaperonin containing TCP1, subunit 5 (epsilon) |
| CD109 | NM_133493 | CD109 |
| CD164L2 | NM_207397 | CD164 sialomucin-like 2 |
| CD22 | NM_001771 | CD22 antigen |
| CD244 | NM_016382 | CD244 natural killer cell receptor 2B4 |
| CD276 | NM_001024736 | CD276 antigen isoform a |
| CD28 | NM_006139 | CD28 antigen |
| CD300C | NM_006678 | CD300C antigen |
| CD300LG | NM_145273 | triggering receptor expressed on myeloid cells |
| CD34 | NM_001025109 | CD34 antigen isoform a |
| CD3D | NM_000732 | CD3D antigen, delta polypeptide (TiT3 complex) |
| CD4 | NM_000616 | CD4 antigen precursor |
| CD40 | NM_152854 | CD40 antigen isoform 2 precursor |
| CD44 | NM_000610 | CD44 antigen isoform 1 precursor |
| CD47 | NM_001025079 | CD47 molecule isoform 3 precursor |
| CD53 | NM_000560 | CD53 antigen |
| CD80 | NM_005191 | CD80 antigen (CD28 antigen ligand 1, B7-1 |
| CD82 | NM_001024844 | CD82 antigen isoform 2 |
| CD84 | NM_003874 | CD84 antigen (leukocyte antigen) |
| CD8A | NM_001768 | CD8 antigen alpha polypeptide isoform 1 |
| CD93 | NM_012072 | CD93 antigen precursor |
| CDAN1 | NM_138477 | codanin 1 |
| CDC25A | NM_001789 | cell division cycle 25A isoform a |
| CDC25B | NM_004358 | cell division cycle 25B isoform 2 |
| CDC42BPA | NM_003607 | CDC42-binding protein kinase alpha isoform B |
| CDC42SE1 | NM_020239 | CDC42 small effector 1 |
| CDCA5 | NM_080668 | cell division cycle associated 5 |
| CDGAP | NM_020754 | Cdc42 GTPase-activating protein |
| CDH1 | NM_004360 | cadherin 1, type 1 preproprotein |
| CDH17 | NM_004063 | cadherin 17 precursor |
| CDH3 | NM_001793 | cadherin 3, type 1 preproprotein |
| CDH5 | NM_001795 | cadherin 5, type 2 preproprotein |
| CDK2AP1 | NM_004642 | CDK2-associated protein 1 |
| CDK5R2 | NM_003936 | cyclin-dependent kinase 5, regulatory subunit 2 |
| CDK5RAP3 | NM_025197 | CDK5 regulatory subunit associated protein 3 |
| CDK6 | NM_001259 | cyclin-dependent kinase 6 |
| CDKAL1 | NM_017774 | CDK5 regulatory subunit associated protein |
| CDON | NM_016952 | surface glycoprotein, Ig superfamily member |
| CDR2L | NM_014603 | paraneoplastic antigen |
| CDRT1 | NM_006382 | CMT1A duplicated region transcript 1 |
| CDRT4 | NM_173622 | hypothetical protein LOC284040 |
| CDX1 | NM_001804 | caudal type homeo box transcription factor 1 |
| CEACAM5 | NM_004363 | carcinoembryonic antigen-related cell adhesion |
| CELSR1 | NM_014246 | cadherin EGF LAG seven-pass G-type receptor 1 |
| CELSR2 | NM_001408 | cadherin EGF LAG seven-pass G-type receptor 2 |
| CELSR3 | NM_001407 | cadherin EGF LAG seven-pass G-type receptor 3 |
| CENTA2 | NM_018404 | centaurin-alpha 2 protein |
| CENTD1 | NM_015230 | centaurin delta 1 isoform a |
| CENTG1 | NM_014770 | centaurin, gamma 1 |
| CEP135 | NM_025009 | centrosome protein 4 |
| CEP192 | NM_018069 | hypothetical protein LOC55125 isoform 2 |
| CEP350 | NM_014810 | centrosome-associated protein 350 |
| CFD | NM_001928 | complement factor D preproprotein |
| CG018 | NM_052818 | hypothetical protein LOC90634 |
| CGN | NM_020770 | cingulin |
| CGNL1 | NM_032866 | cingulin-like 1 |
| CHD5 | NM_015557 | chromodomain helicase DNA binding protein 5 |
| CHD6 | NM_032221 | chromodomain helicase DNA binding protein 6 |
| CHKA | NM_001277 | choline kinase alpha isoform a |
| CHKB | NM_152253 | choline/ethanolamine kinase isoform b |
| CHML | NM_001821 | choroideremia-like Rab escort protein 2 |
| CHPF | NM_024536 | chondroitin polymerizing factor |
| CHRNB1 | NM_000747 | nicotinic acetylcholine receptor beta 1 subunit |
| CHRNB2 | NM_000748 | cholinergic receptor, nicotinic, beta |
| CHRNG | NM_005199 | cholinergic receptor, nicotinic, gamma |
| CHST10 | NM_004854 | HNK-1 sulfotransferase |
| CHST13 | NM_152889 | carbohydrate (chondroitin 4) sulfotransferase |
| CHST3 | NM_004273 | carbohydrate (chondroitin 6) sulfotransferase 3 |
| CHST4 | NM_005769 | carbohydrate (N-acetylglucosamine 6-O) |
| CHURC1 | NM_145165 | churchill domain containing 1 |
| CIAPIN1 | NM_020313 | cytokine induced apoptosis inhibitor 1 |
| CIAS1 | NM_004895 | cryopyrin isoform a |
| CIDEA | NM_001279 | cell death-inducing DFFA-like effector a isoform |
| CIR | NM_004882 | CBF1 interacting corepressor |
| CIT | NM_007174 | citron |
| CITED4 | NM_133467 | Cbp/p300-interacting transactivator, with |
| CLASP1 | NM_015282 | CLIP-associating protein 1 |
| CLCN6 | NM_001286 | chloride channel 6 isoform ClC-6a |
| CLEC12A | NM_138337 | myeloid inhibitory C-type lectin-like receptor |
| CLEC12B | NM_205852 | macrophage antigen h |
| CLEC4E | NM_014358 | C-type lectin domain family 4, member E |
| CLEC4F | NM_173535 | C-type lectin, superfamily member 13 |
| CLEC5A | NM_013252 | C-type lectin, superfamily member 5 |
| CLIC4 | NM_013943 | chloride intracellular channel 4 |
| CLN5 | NM_006493 | ceroid-lipofuscinosis, neuronal 5 |
| CLN6 | NM_017882 | CLN6 protein |
| CLN8 | NM_018941 | CLN8 protein |
| CLPS | NM_001832 | colipase preproprotein |
| CLYBL | NM_138280 | citrate lyase beta like |
| CMYA5 | NM_153610 | cardiomyopathy associated 5 |
| CNDP2 | NM_018235 | CNDP dipeptidase 2 (metallopeptidase M20 |
| CNGA2 | NM_005140 | cyclic nucleotide gated channel alpha 2 |
| CNGA3 | NM_001298 | cyclic nucleotide gated channel alpha 3 |
| CNGB1 | NM_001297 | cyclic nucleotide gated channel beta 1 |
| CNNM1 | NM_020348 | cyclin M1 |
| CNNM3 | NM_017623 | cyclin M3 isoform 1 |
| CNOT4 | NM_013316 | CCR4-NOT transcription complex, subunit 4 |
| CNP | NM_033133 | 2′,3′-cyclic nucleotide 3′ phosphodiesterase |
| CNTD1 | NM_173478 | hypothetical protein LOC124817 |
| CNTD2 | NM_024877 | hypothetical protein LOC79935 |
| CNTNAP2 | NM_014141 | cell recognition molecule Caspr2 precursor |
| COG4 | NM_015386 | component of oligomeric golgi complex 4 |
| COG5 | NM_006348 | component of oligomeric golgi complex 5 isoform |
| COL12A1 | NM_004370 | collagen, type XII, alpha 1 long isoform |
| COL18A1 | NM_030582 | alpha 1 type XVIII collagen isoform 1 precursor |
| COL1A1 | NM_000088 | alpha 1 type I collagen preproprotein |
| COL21A1 | NM_030820 | collagen, type XXI, alpha 1 precursor |
| COL24A1 | NM_152890 | collagen, type XXIV, alpha 1 |
| COL4A4 | NM_000092 | alpha 4 type IV collagen precursor |
| COL4A5 | NM_000495 | alpha 5 type IV collagen isoform 1, precursor |
| COL5A2 | NM_000393 | alpha 2 type V collagen preproprotein |
| COL5A3 | NM_015719 | collagen, type V, alpha 3 preproprotein |
| COL9A1 | NM_001851 | alpha 1 type IX collagen isoform 1 precursor |
| COL9A2 | NM_001852 | alpha 2 type IX collagen |
| COMMD2 | NM_016094 | COMM domain containing 2 |
| COMMD5 | NM_014066 | hypertension-related calcium-regulated gene |
| COMMD7 | NM_053041 | COMM domain containing 7 |
| COPA | NM_004371 | coatomer protein complex, subunit alpha |
| COPZ1 | NM_016057 | coatomer protein complex, subunit zeta 1 |
| COQ5 | NM_032314 | hypothetical protein LOC84274 |
| COQ9 | NM_020312 | hypothetical protein LOC57017 |
| CORIN | NM_006587 | corin |
| CORO1B | NM_001018070 | coronin, actin binding protein, 1B |
| CORO2B | NM_006091 | coronin, actin binding protein, 2B |
| COTL1 | NM_021149 | coactosin-like 1 |
| COVA1 | NM_006375 | cytosolic ovarian carcinoma antigen 1 isoform a |
| COX4NB | NM_006067 | neighbor of COX4 |
| COX7A2L | NM_004718 | cytochrome c oxidase subunit VIIa polypeptide 2 |
| CP110 | NM_014711 | CP110 protein |
| CPAMD8 | NM_015692 | C3 and PZP-like, alpha-2-macroglobulin domain |
| CPB2 | NM_001872 | plasma carboxypeptidase B2 isoform a |
| CPD | NM_001304 | carboxypeptidase D precursor |
| CPLX2 | NM_001008220 | complexin 2 |
| CPM | NM_001005502 | carboxypeptidase M precursor |
| CPNE3 | NM_003909 | copine III |
| CPOX | NM_000097 | coproporphyrinogen oxidase |
| CPSF2 | NM_017437 | cleavage and polyadenylation specific factor 2 |
| CPSF3L | NM_032179 | related to CPSF subunits 68 kDa isoform 2 |
| CRAMP1L | NM_020825 | Crm, cramped-like |
| CREB1 | NM_004379 | cAMP responsive element binding protein 1 |
| CREB3L2 | NM_194071 | cAMP responsive element binding protein 3-like |
| CREB5 | NM_001011666 | cAMP responsive element binding protein 5 |
| CREBL2 | NM_001310 | cAMP responsive element binding protein-like 2 |
| CREG2 | NM_153836 | cellular repressor of E1A-stimulated genes 2 |
| CRELD1 | NM_001031717 | cysteine-rich with EGF-like domains 1 isoform 1 |
| CRISPLD2 | NM_031476 | cysteine-rich secretory protein LCCL domain |
| CRK | NM_005206 | v-crk sarcoma virus CT10 oncogene homolog |
| CRLF3 | NM_015986 | cytokine receptor-like factor 3 |
| CRNKL1 | NM_016652 | crooked neck-like 1 protein |
| CRSP2 | NM_004229 | cofactor required for Sp1 transcriptional |
| CRSP7 | NM_004831 | cofactor required for Sp1 transcriptional |
| CRTC3 | NM_022769 | transducer of regulated CREB protein 3 |
| CRX | NM_000554 | cone-rod homeobox protein |
| CSDC2 | NM_014460 | RNA-binding protein pippin |
| CSF1 | NM_172212 | colony stimulating factor 1 isoform a precursor |
| CSF2RA | NM_006140 | colony stimulating factor 2 receptor alpha chain |
| CSMD1 | NM_033225 | CUB and Sushi multiple domains 1 |
| CSNK1G1 | NM_001011664 | casein kinase 1, gamma 1 isoform L |
| CSNK1G3 | NM_001031812 | casein kinase 1, gamma 3 isoform 2 |
| CSNK2A1 | NM_001895 | casein kinase II alpha 1 subunit isoform a |
| CSPG3 | NM_004386 | chondroitin sulfate proteoglycan 3 (neurocan) |
| CSRP3 | NM_003476 | cysteine and glycine-rich protein 3 |
| CSTB | NM_000100 | cystatin B |
| CTAGE1 | NM_172241 | cutaneous T-cell lymphoma-associated antigen 1 |
| CTDSP2 | NM_005730 | nuclear LIM interactor-interacting factor 2 |
| CTF1 | NM_001330 | cardiotrophin 1 |
| CTGF | NM_001901 | connective tissue growth factor |
| CTH | NM_001902 | cystathionase isoform 1 |
| CTLA4 | NM_005214 | cytotoxic T-lymphocyte-associated protein 4 |
| CTNNBIP1 | NM_001012329 | catenin, beta interacting protein 1 |
| CTNND1 | NM_001331 | catenin (cadherin-associated protein), delta 1 |
| CTSB | NM_001908 | cathepsin B preproprotein |
| CTSC | NM_148170 | cathepsin C isoform b precursor |
| CTSD | NM_001909 | cathepsin D preproprotein |
| CTSS | NM_004079 | cathepsin S preproprotein |
| CTTN | NM_005231 | cortactin isoform a |
| CTXN1 | NM_206833 | cortexin 1 |
| CUBN | NM_001081 | cubilin |
| CUGBP2 | NM_001025076 | CUG triplet repeat, RNA binding protein 2 |
| CUL3 | NM_003590 | cullin 3 |
| CUL5 | NM_003478 | Vasopressin-activated calcium-mobilizing |
| CWF19L1 | NM_018294 | CWF19-like 1, cell cycle control |
| CX3CL1 | NM_002996 | chemokine (C—X3—C motif) ligand 1 |
| CXCL12 | NM_000609 | chemokine (C—X—C motif) ligand 12 (stromal |
| CXCL14 | NM_004887 | small inducible cytokine B14 precursor |
| CXCL9 | NM_002416 | small inducible cytokine B9 precursor |
| CXorf21 | NM_025159 | hypothetical protein LOC80231 |
| CXorf23 | NM_198279 | hypothetical protein LOC256643 |
| CXorf34 | NM_024917 | hypothetical protein LOC79979 |
| CXorf38 | NM_144970 | hypothetical protein LOC159013 |
| CXorf53 | NM_001018055 | BRCA1/BRCA2-containing complex subunit 36 |
| CXXC5 | NM_016463 | CXXC finger 5 |
| CXXC6 | NM_030625 | CXXC finger 6 |
| CYB561D1 | NM_182580 | cytochrome b-561 domain containing 1 |
| CYB5B | NM_030579 | cytochrome b5 outer mitochondrial membrane |
| CYB5D1 | NM_144607 | hypothetical protein LOC124637 |
| CYBRD1 | NM_024843 | cytochrome b reductase 1 |
| CYCS | NM_018947 | cytochrome c |
| CYFIP2 | NM_014376 | cytoplasmic FMR1 interacting protein 2 |
| CYLC2 | NM_001340 | cylicin 2 |
| CYLD | NM_015247 | ubiquitin carboxyl-terminal hydrolase CYLD |
| CYLN2 | NM_003388 | cytoplasmic linker 2 isoform 1 |
| CYP11B1 | NM_000497 | cytochrome P450, family 11, subfamily B, |
| CYP11B2 | NM_000498 | cytochrome P450, subfamily XIB polypeptide 2 |
| CYP1A2 | NM_000761 | cytochrome P450, family 1, subfamily A, |
| CYP26B1 | NM_019885 | cytochrome P450, family 26, subfamily b, |
| CYP2B6 | NM_000767 | cytochrome P450, family 2, subfamily B, |
| CYP2C9 | NM_000771 | cytochrome P450, family 2, subfamily C, |
| CYP8B1 | NM_004391 | cytochrome P450, family 8, subfamily B, |
| D2HGDH | NM_152783 | D-2-hydroxyglutarate dehydrogenase |
| DAB2 | NM_001343 | disabled homolog 2 |
| DAPK1 | NM_004938 | death-associated protein kinase 1 |
| DAPK2 | NM_014326 | death-associated protein kinase 2 |
| DBF4 | NM_006716 | activator of S phase kinase |
| DBT | NM_001918 | dihydrolipoamide branched chain transacylase |
| DCAKD | NM_024819 | dephospho-CoA kinase domain containing |
| DCAMKL1 | NM_004734 | doublecortin and CaM kinase-like 1 |
| DCLRE1C | NM_001033855 | artemis protein isoform a |
| DCST2 | NM_144622 | hypothetical protein LOC127579 |
| DCTD | NM_001012732 | dCMP deaminase isoform a |
| DCTN4 | NM_016221 | dynactin 4 (p62) |
| DCTN5 | NM_032486 | dynactin 4 |
| DCX | NM_000555 | doublecortin isoform a |
| DDAH1 | NM_012137 | dimethylarginine dimethylaminohydrolase 1 |
| DDEFL1 | NM_017707 | development and differentiation enhancing |
| DDI1 | NM_001001711 | hypothetical protein LOC414301 |
| DDI2 | NM_032341 | DNA-damage inducible protein 2 |
| DDIT4L | NM_145244 | DNA-damage-inducible transcript 4-like |
| DDR1 | NM_001954 | discoidin domain receptor family, member 1 |
| DDX11 | NM_004399 | DEAD/H (Asp-Glu-Ala-Asp/His) box polypeptide 11 |
| DDX17 | NM_006386 | DEAD box polypeptide 17 isoform p82 |
| DDX23 | NM_004818 | DEAD (Asp-Glu-Ala-Asp) box polypeptide 23 |
| DEDD2 | NM_133328 | death effector domain-containing DNA binding |
| DEFA3 | NM_005217 | defensin, alpha 3 preproprotein |
| DEFA6 | NM_001926 | defensin, alpha 6 preproprotein |
| DEGS1 | NM_003676 | degenerative spermatocyte homolog 1, lipid |
| DENND1C | NM_024898 | hypothetical protein LOC79958 |
| DENND2C | NM_198459 | DENN/MADD domain containing 2C |
| DERA | NM_015954 | 2-deoxyribose-5-phosphate aldolase homolog |
| DERL3 | NM_001002862 | derlin-3 protein isoform b |
| DFFA | NM_213566 | DNA fragmentation factor, 45 kDa, alpha |
| DFFB | NM_001004285 | DNA fragmentation factor, 40 kD, beta |
| DGKB | NM_004080 | diacylglycerol kinase, beta isoform 1 |
| DGKQ | NM_001347 | diacylglycerol kinase, theta |
| DHCR24 | NM_014762 | 24-dehydrocholesterol reductase precursor |
| DHDDS | NM_024887 | dehydrodolichyl diphosphate synthase isoform a |
| DHFR | NM_000791 | dihydrofolate reductase |
| DHRS7B | NM_015510 | hypothetical protein LOC25979 |
| DHRS9 | NM_005771 | NADP-dependent retinol dehydrogenase/reductase |
| DHTKD1 | NM_018706 | dehydrogenase E1 and transketolase domain |
| DHX30 | NM_138614 | DEAH (Asp-Glu-Ala-His) box polypeptide 30 |
| DHX37 | NM_032656 | DEAH (Asp-Glu-Ala-His) box polypeptide 37 |
| DIAPH1 | NM_005219 | diaphanous 1 |
| DIDO1 | NM_033081 | death inducer-obliterator 1 isoform c |
| DIP13B | NM_018171 | DIP13 beta |
| DIP2B | NM_173602 | hypothetical protein LOC57609 |
| DIP2C | NM_014974 | hypothetical protein LOC22982 |
| DIRAS1 | NM_145173 | small GTP-binding tumor suppressor 1 |
| DIRAS2 | NM_017594 | Di-Ras2 |
| DIRC1 | NM_052952 | hypothetical protein LOC116093 |
| DISC1 | NM_001012957 | disrupted in schizophrenia 1 isoform Lv |
| DIXDC1 | NM_033425 | DIX domain containing 1 isoform b |
| DJ122O8.2 | NM_020466 | hypothetical protein LOC57226 |
| DKFZP434B0335 | NM_015395 | hypothetical protein LOC25851 |
| DKFZp434I1020 | NM_194295 | hypothetical protein LOC196968 |
| DKFZp547H025 | NM_020161 | hypothetical protein LOC56918 |
| DKFZp564K142 | NM_032121 | implantation-associated protein |
| DKFZp686K16132 | NM_001012987 | hypothetical protein LOC388957 |
| DKFZp686O24166 | NM_001009913 | hypothetical protein LOC374383 |
| DKFZp761B107 | NM_173463 | hypothetical protein LOC91050 |
| DKFZp761E198 | NM_138368 | hypothetical protein LOC91056 |
| DKFZp779B1540 | NM_001010903 | hypothetical protein LOC389384 |
| DLC1 | NM_006094 | deleted in liver cancer 1 isoform 2 |
| DLEC1 | NM_007335 | deleted in lung and esophageal cancer 1 isoform |
| DLG3 | NM_021120 | synapse-associated protein 102 |
| DLGAP2 | NM_004745 | discs large-associated protein 2 |
| DLX1 | NM_178120 | distal-less homeobox 1 isoform 1 |
| DMBX1 | NM_147192 | diencephalon/mesencephalon homeobox 1 isoform b |
| DMTF1 | NM_021145 | cyclin D binding myb-like transcription factor |
| DNAH11 | NM_003777 | dynein, axonemal, heavy polypeptide 11 |
| DNAJA4 | NM_018602 | DnaJ (Hsp40) homolog, subfamily A, member 4 |
| DNAJC11 | NM_018198 | DnaJ (Hsp40) homolog, subfamily C, member 11 |
| DNAJC14 | NM_032364 | dopamine receptor interacting protein |
| DNAJC18 | NM_152686 | DnaJ (Hsp40) homolog, subfamily C, member 18 |
| DNAL4 | NM_005740 | dynein light chain 4, axonemal |
| DNASE1L1 | NM_001009932 | deoxyribonuclease I-like 1 precursor |
| DNASE2 | NM_001375 | deoxyribonuclease II, lysosomal precursor |
| DNMT3A | NM_022552 | DNA cytosine methyltransferase 3 alpha isoform |
| DOC2B | NM_003585 | double C2-like domains, beta |
| DOCK1 | NM_001380 | dedicator of cytokinesis 1 |
| DOCK2 | NM_004946 | dedicator of cytokinesis 2 |
| DOCK3 | NM_004947 | dedicator of cytokinesis 3 |
| DOCK9 | NM_015296 | dedicator of cytokinesis 9 |
| DPCR1 | NM_080870 | diffuse panbronchiolitis critical region 1 |
| DPF3 | NM_012074 | D4, zinc and double PHD fingers, family 3 |
| DPY19L2 | NM_173812 | hypothetical protein LOC283417 |
| DPYSL3 | NM_001387 | dihydropyrimidinase-like 3 |
| DQX1 | NM_133637 | DEAQ box polypeptide 1 (RNA-dependent ATPase) |
| DSCAM | NM_206887 | Down syndrome cell adhesion molecule isoform |
| DTNA | NM_001390 | dystrobrevin alpha isoform 1 |
| DTNB | NM_021907 | dystrobrevin, beta isoform 1 |
| DTWD2 | NM_173666 | DTW domain containing 2 |
| DTX1 | NM_004416 | deltex homolog 1 |
| DTX3L | NM_138287 | deltex 3-like |
| DUSP13 | NM_001007271 | muscle-restricted dual specificity phosphatase |
| DUSP4 | NM_001394 | dual specificity phosphatase 4 isoform 1 |
| DUT | NM_001025248 | dUTP pyrophosphatase isoform 1 precursor |
| DUX1 | NM_012146 | double homeobox, 1 |
| DUXA | NM_001012729 | hypothetical protein LOC503835 |
| DVL3 | NM_004423 | dishevelled 3 |
| DYNC2LI1 | NM_016008 | dynein 2 light intermediate chain isoform 1 |
| DYRK1B | NM_004714 | dual-specificity tyrosine-(Y)-phosphorylation |
| DZIP1 | NM_014934 | DAZ interacting protein 1 isoform 1 |
| E2F2 | NM_004091 | E2F transcription factor 2 |
| E2F3 | NM_001949 | E2F transcription factor 3 |
| EAF1 | NM_033083 | ELL associated factor 1 |
| EARS2 | NM_133451 | hypothetical protein LOC124454 |
| EBI3 | NM_005755 | Epstein-Barr virus induced gene 3 precursor |
| ECM2 | NM_001393 | extracellular matrix protein 2 precursor |
| ECOP | NM_030796 | EGFR-coamplified and overexpressed protein |
| EDA2R | NM_021783 | X-linked ectodysplasin receptor |
| EDARADD | NM_080738 | EDAR-associated death domain isoform B |
| EDEM1 | NM_014674 | ER degradation enhancer, mannosidase alpha-like |
| EDG4 | NM_004720 | endothelial differentiation, lysophosphatidic |
| EDN3 | NM_000114 | endothelin 3 isoform 1 preproprotein |
| EEF2K | NM_013302 | elongation factor-2 kinase |
| EFCAB5 | NM_001033562 | EF-hand calcium binding domain 5 isoform 2 |
| EFEMP1 | NM_004105 | EGF-containing fibulin-like extracellular matrix |
| EFNA1 | NM_004428 | ephrin A1 isoform a precursor |
| EFNA3 | NM_004952 | ephrin A3 |
| EFNB3 | NM_001406 | ephrin-B3 precursor |
| EFS | NM_005864 | embryonal Fyn-associated substrate isoform 1 |
| EGFR | NM_201284 | epidermal growth factor receptor isoform d |
| EGLN1 | NM_022051 | egl nine homolog 1 |
| EGR1 | NM_001964 | early growth response 1 |
| EHD2 | NM_014601 | EH-domain containing 2 |
| EIF2AK2 | NM_002759 | eukaryotic translation initiation factor 2-alpha |
| EIF2AK3 | NM_004836 | eukaryotic translation initiation factor 2-alpha |
| EIF2AK4 | NM_001013703 | eukaryotic translation initiation factor 2 alpha |
| EIF2C1 | NM_012199 | eukaryotic translation initiation factor 2C, 1 |
| EIF4EBP2 | NM_004096 | eukaryotic translation initiation factor 4E |
| EIF4ENIF1 | NM_019843 | eukaryotic translation initiation factor 4E |
| EIF5 | NM_001969 | eukaryotic translation initiation factor 5 |
| ELAC1 | NM_018696 | elaC homolog 1 |
| ELF4 | NM_001421 | E74-like factor 4 (ets domain transcription |
| ELF5 | NM_001422 | E74-like factor 5 ESE-2b |
| ELK1 | NM_005229 | ELK1 protein |
| ELK4 | NM_021795 | ELK4 protein isoform b |
| ELMO1 | NM_014800 | engulfment and cell motility 1 isoform 1 |
| ELMO2 | NM_133171 | engulfment and cell motility 2 |
| ELMOD1 | NM_018712 | ELMO domain containing 1 |
| ELOF1 | NM_032377 | elongation factor 1 homolog (ELF1, S. |
| ELOVL5 | NM_021814 | homolog of yeast long chain polyunsaturated |
| ELOVL6 | NM_024090 | ELOVL family member 6, elongation of long chain |
| EME1 | NM_152463 | essential meiotic endonuclease 1 homolog 1 |
| EMID1 | NM_133455 | EMI domain containing 1 |
| EMP1 | NM_001423 | epithelial membrane protein 1 |
| EMR2 | NM_013447 | egf-like module containing, mucin-like, hormone |
| ENAH | NM_001008493 | enabled homolog isoform a |
| ENAM | NM_031889 | enamelin |
| ENO1 | NM_001428 | enolase 1 |
| ENPP1 | NM_006208 | ectonucleotide pyrophosphatase/phosphodiesterase |
| ENPP5 | NM_021572 | ectonucleotide pyrophosphatase/phosphodiesterase |
| ENPP6 | NM_153343 | ectonucleotide pyrophosphatase/phosphodiesterase |
| ENSA | NM_207043 | endosulfine alpha isoform 2 |
| ENTPD3 | NM_001248 | ectonucleoside triphosphate diphosphohydrolase |
| EP400 | NM_015409 | E1A binding protein p400 |
| EPB41 | NM_004437 | erythrocyte membrane protein band 4.1 |
| EPB41L5 | NM_020909 | erythrocyte membrane protein band 4.1 like 5 |
| EPHA2 | NM_004431 | ephrin receptor EphA2 |
| EPHA3 | NM_005233 | ephrin receptor EphA3 isoform a precursor |
| EPHB4 | NM_004444 | ephrin receptor EphB4 precursor |
| EPM2AIP1 | NM_014805 | EPM2A interacting protein 1 |
| EPO | NM_000799 | erythropoietin precursor |
| ERBB3 | NM_001982 | erbB-3 isoform 1 precursor |
| ERGIC1 | NM_020462 | endoplasmic reticulum-golgi intermediate |
| ESAM | NM_138961 | endothelial cell adhesion molecule |
| ESRRG | NM_001438 | estrogen-related receptor gamma isoform 1 |
| ET | NM_024311 | hypothetical protein LOC79157 |
| ETV1 | NM_004956 | ets variant gene 1 |
| ETV3 | NM_005240 | ets variant gene 3 |
| ETV6 | NM_001987 | ets variant gene 6 |
| EVC | NM_153717 | Ellis van Creveld syndrome protein |
| EXOC2 | NM_018303 | Sec5 protein |
| EXOC4 | NM_021807 | SEC8 protein isoform a |
| EXTL3 | NM_001440 | Reg receptor |
| EYA2 | NM_005244 | eyes absent 2 isoform a |
| EZH1 | NM_001991 | enhancer of zeste homolog 1 |
| F11R | NM_016946 | F11 receptor isoform a precursor |
| F13A1 | NM_000129 | coagulation factor XIII A1 subunit precursor |
| F2R | NM_001992 | coagulation factor II receptor precursor |
| F2RL1 | NM_005242 | coagulation factor II (thrombin) receptor-like 1 |
| F2RL3 | NM_003950 | coagulation factor II (thrombin) receptor-like 3 |
| FADS2 | NM_004265 | fatty acid desaturase 2 |
| FADS6 | NM_178128 | fatty acid desaturase domain family, member 6 |
| FAIM2 | NM_012306 | Fas apoptotic inhibitory molecule 2 |
| FAM100B | NM_182565 | hypothetical protein LOC283991 |
| FAM102A | NM_203305 | early estrogen-induced gene 1 protein isoform b |
| FAM102B | NM_001010883 | hypothetical protein LOC284611 |
| FAM104A | NM_032837 | hypothetical protein LOC84923 |
| FAM106A | NM_024974 | hypothetical protein LOC80039 |
| FAM107A | NM_007177 | downregulated in renal cell carcinoma |
| FAM107B | NM_031453 | hypothetical protein LOC83641 |
| FAM111A | NM_022074 | hypothetical protein LOC63901 |
| FAM117A | NM_030802 | C/EBP-induced protein |
| FAM11A | NM_032508 | family with sequence similarity 11, member A |
| FAM19A1 | NM_213609 | family with sequence similarity 19 (chemokine |
| FAM20B | NM_014864 | family with sequence similarity 20, member B |
| FAM36A | NM_198076 | family with sequence similarity 36, member A |
| FAM3B | NM_058186 | family with sequence similarity 3, member B |
| FAM40A | NM_033088 | hypothetical protein LOC85369 |
| FAM43A | NM_153690 | hypothetical protein LOC131583 |
| FAM53B | NM_014661 | hypothetical protein LOC9679 |
| FAM55C | NM_145037 | hypothetical protein LOC91775 |
| FAM5B | NM_021165 | BMP/retinoic acid-inducible neural-specific |
| FAM60A | NM_021238 | family with sequence similarity 60, member A |
| FAM62C | NM_031913 | family with sequence similarity 62 (C2 domain |
| FAM71C | NM_153364 | hypothetical protein LOC196472 |
| FAM81A | NM_152450 | hypothetical protein LOC145773 |
| FAM83E | NM_017708 | hypothetical protein LOC54854 |
| FAM83F | NM_138435 | hypothetical protein LOC113828 |
| FAM83H | NM_198488 | hypothetical protein LOC286077 |
| FAM89B | NM_152832 | Mouse Mammary Turmor Virus Receptor homolog 1 |
| FAM98B | NM_173611 | hypothetical protein LOC283742 |
| FANCC | NM_000136 | Fanconi anemia, complementation group C |
| FANCD2 | NM_033084 | Fanconi anemia complementation group D2 isoform |
| FATE1 | NM_033085 | fetal and adult testis expressed transcript |
| FBS1 | NM_022452 | fibrosin 1 |
| FBXL11 | NM_012308 | F-box and leucine-rich repeat protein 11 |
| FBXO16 | NM_172366 | F-box only protein 16 |
| FBXO21 | NM_015002 | F-box only protein 21 isoform 2 |
| FBXO27 | NM_178820 | F-box protein 27 |
| FBXO31 | NM_024735 | F-box protein 31 |
| FBXO34 | NM_017943 | F-box only protein 34 |
| FBXO44 | NM_001014765 | F-box protein 44 isoform 1 |
| FBXO9 | NM_012347 | F-box only protein 9 isoform 1 |
| FBXW11 | NM_012300 | F-box and WD-40 domain protein 1B isoform C |
| FBXW8 | NM_012174 | F-box and WD-40 domain protein 8 isoform 2 |
| FCER2 | NM_002002 | Fc fragment of IgE, low affinity II, receptor |
| FCGR3A | NM_000569 | Fc fragment of IgG, low affinity IIIa, receptor |
| FCGR3B | NM_000570 | low affinity immunoglobulin gamma Fc region |
| FCHSD1 | NM_033449 | FCH and double SH3 domains 1 |
| FCMD | NM_006731 | fukutin |
| FEM1C | NM_020177 | feminization 1 homolog a |
| FGA | NM_021871 | fibrinogen, alpha polypeptide isoform alpha |
| FGD6 | NM_018351 | FYVE, RhoGEF and PH domain containing 6 |
| FGF1 | NM_000800 | fibroblast growth factor 1 (acidic) isoform 1 |
| FGF19 | NM_005117 | fibroblast growth factor 19 precursor |
| FGFR1 | NM_023107 | fibroblast growth factor receptor 1 isoform 5 |
| FHIT | NM_002012 | fragile histidine triad gene |
| FIS | NM_175616 | hypothetical protein LOC202299 |
| FKBP10 | NM_021939 | FK506 binding protein 10, 65 kDa |
| FKBP1A | NM_000801 | FK506-binding protein 1A |
| FKBP5 | NM_004117 | FK506 binding protein 5 |
| FKBP9 | NM_007270 | FK506 binding protein 9 |
| FKBP9L | NM_182827 | FK506 binding protein 9-like |
| FKRP | NM_024301 | fukutin-related protein |
| FKSG44 | NM_031904 | FKSG44 protein |
| FLJ10159 | NM_018013 | hypothetical protein LOC55084 |
| FLJ10324 | NM_018059 | hypothetical protein LOC55698 |
| FLJ10357 | NM_018071 | hypothetical protein LOC55701 |
| FLJ10490 | NM_018111 | hypothetical protein LOC55150 |
| FLJ10803 | NM_018224 | hypothetical protein LOC55744 |
| FLJ10815 | NM_018231 | amino acid transporter |
| FLJ11021 | NM_023012 | hypothetical protein LOC65117 isoform a |
| FLJ11151 | NM_018340 | hypothetical protein LOC55313 |
| FLJ11171 | NM_018348 | hypothetical protein LOC55783 |
| FLJ11259 | NM_018370 | hypothetical protein LOC55332 |
| FLJ11292 | NM_018382 | hypothetical protein LOC55338 |
| FLJ11806 | NM_024824 | nuclear protein UKp68 isoform 1 |
| FLJ12505 | NM_024749 | hypothetical protein LOC79805 |
| FLJ12681 | NM_022773 | hypothetical protein LOC64788 |
| FLJ12700 | NM_024910 | hypothetical protein LOC79970 |
| FLJ12949 | NM_023008 | hypothetical protein LOC65095 isoform 1 |
| FLJ13197 | NM_024614 | hypothetical protein LOC79667 |
| FLJ14001 | NM_024677 | hypothetical protein LOC79730 |
| FLJ14213 | NM_024841 | hypothetical protein LOC79899 |
| FLJ14397 | NM_032779 | hypothetical protein LOC84865 |
| FLJ14816 | NM_032845 | hypothetical protein LOC84931 |
| FLJ14834 | NM_032849 | hypothetical protein LOC84935 |
| FLJ20032 | NM_017628 | hypothetical protein LOC54790 |
| FLJ20035 | NM_017631 | hypothetical protein LOC55601 |
| FLJ20160 | NM_017694 | hypothetical protein LOC54842 |
| FLJ20186 | NM_207514 | differentially expressed in FDCP 8 isoform 1 |
| FLJ20297 | NM_017751 | hypothetical protein LOC55627 isoform 1 |
| FLJ20581 | NM_017888 | hypothetical protein LOC54988 |
| FLJ20582 | NM_014106 | hypothetical protein LOC54989 |
| FLJ20628 | NM_017910 | hypothetical protein LOC55006 |
| FLJ20701 | NM_017933 | hypothetical protein LOC55022 |
| FLJ20758 | NM_017952 | hypothetical protein LOC55037 |
| FLJ20972 | NM_025030 | hypothetical protein LOC80098 |
| FLJ21865 | NM_022759 | endo-beta-N-acetylglucosaminidase |
| FLJ21963 | NM_024560 | hypothetical protein LOC79611 |
| FLJ22795 | NM_025084 | hypothetical protein LOC80154 |
| FLJ23322 | NM_024955 | hypothetical protein LOC80020 |
| FLJ23834 | NM_152750 | hypothetical protein LOC222256 |
| FLJ25996 | NM_001001699 | hypothetical protein LOC401109 |
| FLJ26175 | NM_001001668 | hypothetical protein LOC388566 |
| FLJ27365 | NM_207477 | hypothetical protein LOC400931 |
| FLJ31222 | NM_207388 | hypothetical protein LOC388387 |
| FLJ31568 | NM_152509 | hypothetical protein LOC150244 |
| FLJ31875 | NM_182531 | hypothetical protein LOC197320 |
| FLJ32011 | NM_182516 | hypothetical protein LOC148930 |
| FLJ32130 | NM_152458 | hypothetical protein LOC146540 |
| FLJ32312 | NM_144709 | hypothetical protein LOC150962 |
| FLJ32447 | NM_153038 | hypothetical protein LOC151278 |
| FLJ32569 | NM_152491 | hypothetical protein LOC148811 |
| FLJ32894 | NM_144667 | hypothetical protein LOC144360 |
| FLJ32926 | NM_144577 | hypothetical protein LOC93233 |
| FLJ32955 | NM_153041 | hypothetical protein LOC150596 |
| FLJ33387 | NM_182526 | hypothetical protein LOC161145 |
| FLJ33860 | NM_173644 | hypothetical protein LOC284756 |
| FLJ34931 | NM_001029883 | hypothetical protein LOC388939 |
| FLJ35409 | NM_001001688 | hypothetical protein LOC400765 |
| FLJ35429 | NM_001003807 | hypothetical protein LOC285830 |
| FLJ35740 | NM_147195 | FLJ35740 protein |
| FLJ35773 | NM_152599 | hypothetical protein LOC162387 |
| FLJ35880 | NM_153264 | hypothetical protein LOC256076 |
| FLJ36268 | NM_207511 | hypothetical protein LOC401563 |
| FLJ36492 | NM_182568 | hypothetical protein LOC284047 |
| FLJ36874 | NM_152716 | hypothetical protein LOC219988 |
| FLJ37927 | NM_152623 | hypothetical protein LOC166979 |
| FLJ38288 | NM_173632 | hypothetical protein LOC284309 |
| FLJ38663 | NM_152269 | hypothetical protein LOC91574 |
| FLJ38973 | NM_153689 | hypothetical protein LOC205327 |
| FLJ38991 | NM_001033760 | mitochondrial COX18 isoform 5 |
| FLJ39370 | NM_152400 | hypothetical protein LOC132720 |
| FLJ39531 | NM_207445 | hypothetical protein LOC400360 |
| FLJ39743 | NM_182562 | hypothetical protein LOC283777 |
| FLJ40142 | NM_207435 | hypothetical protein LOC400073 |
| FLJ40852 | NM_173677 | hypothetical protein LOC285962 |
| FLJ41423 | NM_001001679 | hypothetical protein LOC399886 |
| FLJ41733 | NM_207473 | hypothetical protein LOC400870 |
| FLJ41841 | NM_207499 | hypothetical protein LOC401263 |
| FLJ41993 | NM_001001694 | hypothetical protein LOC400935 |
| FLJ42102 | NM_001001680 | hypothetical protein LOC399923 |
| FLJ42418 | NM_001001695 | hypothetical protein LOC400941 |
| FLJ42953 | NM_207474 | hypothetical protein LOC400892 |
| FLJ43339 | NM_207380 | hypothetical protein LOC388115 |
| FLJ43505 | NM_207468 | hypothetical protein LOC400823 |
| FLJ43582 | NM_207412 | hypothetical protein LOC389649 |
| FLJ43879 | NM_001001698 | hypothetical protein LOC401039 |
| FLJ43980 | NM_001004299 | hypothetical protein LOC124149 |
| FLJ44691 | NM_198506 | hypothetical protein LOC345193 |
| FLJ45079 | NM_001001685 | hypothetical protein LOC400624 |
| FLJ45121 | NM_207451 | hypothetical protein LOC400556 |
| FLJ45139 | NM_001001692 | hypothetical protein LOC400867 |
| FLJ45202 | NM_207507 | hypothetical protein LOC401508 |
| FLJ45422 | NM_001004349 | hypothetical protein LOC441140 |
| FLJ45645 | NM_198557 | hypothetical protein LOC375287 |
| FLJ45684 | NM_207462 | hypothetical protein LOC400666 |
| FLJ45831 | NM_001001684 | hypothetical protein LOC400576 |
| FLJ45850 | NM_207395 | hypothetical protein LOC388569 |
| FLJ45909 | NM_198445 | hypothetical protein LOC126432 |
| FLJ45910 | NM_207390 | hypothetical protein LOC388512 |
| FLJ45964 | NM_207483 | hypothetical protein LOC401040 |
| FLJ46010 | NM_001001703 | hypothetical protein LOC401191 |
| FLJ46026 | NM_207458 | hypothetical protein LOC400627 |
| FLJ46154 | NM_198462 | FLJ46154 protein |
| FLJ46230 | NM_207463 | hypothetical protein LOC400679 |
| FLJ46257 | NM_001001693 | hypothetical protein LOC400932 |
| FLJ46266 | NM_207430 | hypothetical protein LOC399949 |
| FLJ46347 | NM_001005303 | hypothetical protein LOC389064 |
| FLJ46363 | NM_207434 | hypothetical protein LOC400002 |
| FLJ46365 | NM_207504 | hypothetical protein LOC401459 |
| FLJ46481 | NM_207405 | hypothetical protein LOC389197 |
| FLJ46688 | NM_001004330 | hypothetical protein LOC440107 |
| FLJ46831 | NM_207426 | forkhead box I2 |
| FLJ46838 | NM_001007546 | hypothetical protein LOC440865 |
| FLJ90757 | NM_001004336 | hypothetical protein LOC440465 |
| FLOT1 | NM_005803 | flotillin 1 |
| FLOT2 | NM_004475 | flotillin 2 |
| FLT1 | NM_002019 | fms-related tyrosine kinase 1 (vascular |
| FLT4 | NM_182925 | fms-related tyrosine kinase 4 isoform 1 |
| FLYWCH1 | NM_032296 | FLYWCH-type zinc finger 1 isoform a |
| FMNL3 | NM_175736 | formin-like 3 isoform 1 |
| FMO4 | NM_002022 | flavin containing monooxygenase 4 |
| FMOD | NM_002023 | fibromodulin precursor |
| FN1 | NM_002026 | fibronectin 1 isoform 3 preproprotein |
| FNDC1 | NM_032532 | fibronectin type III domain containing 1 |
| FNDC5 | NM_153756 | fibronectin type III domain containing 5 |
| FNDC8 | NM_017559 | hypothetical protein LOC54752 |
| FNTB | NM_002028 | farnesyltransferase, CAAX box, beta |
| FOSB | NM_006732 | FBJ murine osteosarcoma viral oncogene homolog |
| FOSL2 | NM_005253 | FOS-like antigen 2 |
| FOXJ2 | NM_018416 | forkhead box J2 |
| FOXJ3 | NM_014947 | forkhead box J3 |
| FOXK2 | NM_181430 | forkhead box K2 isoform 2 |
| FOXO1A | NM_002015 | forkhead box O1A |
| FOXP1 | NM_032682 | forkhead box P1 isoform 1 |
| FRMD1 | NM_024919 | FERM domain containing 1 |
| FRMPD2 | NM_001017929 | FERM and PDZ domain containing 2 isoform 2 |
| FSCN1 | NM_003088 | fascin 1 |
| FSD1L | NM_207647 | fibronectin type III and SPRY domain containing |
| FST | NM_006350 | follistatin isoform FST317 precursor |
| FSTL4 | NM_015082 | follistatin-like 4 |
| FTSJ1 | NM_012280 | FtsJ homolog 1 isoform a |
| FUNDC2 | NM_023934 | FUN14 domain containing 2 |
| FUSIP1 | NM_006625 | FUS interacting protein (serine-arginine rich) 1 |
| FUT2 | NM_000511 | fucosyltransferase 2 (secretor status included) |
| FUT4 | NM_002033 | fucosyltransferase 4 |
| FUT6 | NM_000150 | fucosyltransferase 6 (alpha (1, 3) |
| FXYD3 | NM_005971 | FXYD domain containing ion transport regulator 3 |
| FYCO1 | NM_024513 | FYVE and coiled-coil domain containing 1 |
| FZD1 | NM_003505 | frizzled 1 |
| GAB2 | NM_012296 | GRB2-associated binding protein 2 isoform b |
| GABARAPL1 | NM_031412 | GABA(A) receptor-associated protein like 1 |
| GABBR1 | NM_001470 | gamma-aminobutyric acid (GABA) B receptor 1 |
| GABRA4 | NM_000809 | gamma-aminobutyric acid A receptor, alpha 4 |
| GABRB3 | NM_000814 | gamma-aminobutyric acid (GABA) A receptor, beta |
| GABRE | NM_004961 | gamma-aminobutyric acid (GABA) A receptor, |
| GABRG1 | NM_173536 | gamma-aminobutyric acid A receptor, gamma 1 |
| GABRG2 | NM_000816 | gamma-aminobutyric acid A receptor, gamma 2 |
| GABRR2 | NM_002043 | gamma-aminobutyric acid (GABA) receptor, rho 2 |
| GALC | NM_000153 | galactosylceramidase isoform a precursor |
| GALM | NM_138801 | galactose mutarotase (aldose 1-epimerase) |
| GALNT13 | NM_052917 | UDP-N-acetyl-alpha-D-galactosamine:polypeptide |
| GALNT3 | NM_004482 | polypeptide N-acetylgalactosaminyltransferase 3 |
| GALNT6 | NM_007210 | polypeptide N-acetylgalactosaminyltransferase 6 |
| GALNTL2 | NM_054110 | UDP-N-acetyl-alpha-D-galactosamine:polypeptide |
| GALT | NM_000155 | galactose-1-phosphate uridylyltransferase |
| GAPVD1 | NM_015635 | GTPase activating protein and VPS9 domains 1 |
| GARNL1 | NM_014990 | GTPase activating Rap/RanGAP domain-like 1 |
| GARNL4 | NM_015085 | GTPase activating Rap/RanGAP domain-like 4 |
| GAS2L1 | NM_152237 | growth arrest-specific 2 like 1 isoform b |
| GAS7 | NM_003644 | growth arrest-specific 7 isoform a |
| GATA4 | NM_002052 | GATA binding protein 4 |
| GATAD1 | NM_021167 | GATA zinc finger domain containing 1 |
| GATM | NM_001482 | glycine amidinotransferase (L-arginine:glycine |
| GATS | NM_178831 | opposite strand transcription unit to STAG3 |
| GCLM | NM_002061 | glutamate-cysteine ligase regulatory protein |
| GCM1 | NM_003643 | glial cells missing homolog a |
| GCNT1 | NM_001490 | beta-1,3-galactosyl-O-glycosyl-glycoprotein |
| GCNT2 | NM_001491 | glucosaminyl (N-acetyl) transferase 2, |
| Gcom1 | NM_001018097 | GRINL1A combined protein isoform 8 |
| GDAP2 | NM_017686 | ganglioside induced differentiation associated |
| GDF10 | NM_004962 | growth differentiation factor 10 precursor |
| GDF6 | NM_001001557 | growth differentiation factor 6 |
| GDPD4 | NM_182833 | glycerophosphodiester phosphodiesterase domain |
| Gene_symbol | hsa-miR-143 targets | Gene_name |
| GFOD1 | NM_018988 | glucose-fructose oxidoreductase domain |
| GFOD2 | NM_030819 | hypothetical protein LOC81577 |
| GFPT1 | NM_002056 | glucosamine-fructose-6-phosphate |
| GFPT2 | NM_005110 | glutamine-fructose-6-phosphate transaminase 2 |
| GGA2 | NM_015044 | ADP-ribosylation factor binding protein 2 |
| GGT6 | NM_153338 | gamma-glutamyltransferase 6 homolog |
| GGTL3 | NM_178025 | gamma-glutamyltransferase-like 3 isoform b |
| GHR | NM_000163 | growth hormone receptor precursor |
| GIF | NM_005142 | gastric intrinsic factor (vitamin B synthesis) |
| GIMAP6 | NM_001007224 | GTPase, IMAP family member 6 isoform 3 |
| GIT2 | NM_014776 | G protein-coupled receptor kinase-interactor 2 |
| GJC1 | NM_152219 | gap junction protein, chi 1, 31.9 kDa (connexin |
| GLB1L | NM_024506 | galactosidase, beta 1-like |
| GLDC | NM_000170 | glycine dehydrogenase (decarboxylating; glycine |
| GLI3 | NM_000168 | GLI-Kruppel family member GLI3 |
| GLP1R | NM_002062 | glucagon-like peptide 1 receptor |
| GLT25D2 | NM_015101 | glycosyltransferase 25 domain containing 2 |
| GLYATL2 | NM_145016 | hypothetical protein LOC219970 |
| GMEB2 | NM_012384 | glucocorticoid modulatory element binding |
| GMFB | NM_004124 | glia maturation factor, beta |
| GNA15 | NM_002068 | guanine nucleotide binding protein (G protein), |
| GNAI1 | NM_002069 | guanine nucleotide binding protein (G protein), |
| GNAL | NM_002071 | guanine nucleotide binding protein (G protein), |
| GNAS | NM_016592 | guanine nucleotide binding protein, alpha |
| GNB3 | NM_002075 | guanine nucleotide-binding protein, beta-3 |
| GNB4 | NM_021629 | guanine nucleotide-binding protein, beta-4 |
| GNB5 | NM_006578 | guanine nucleotide-binding protein, beta-5 |
| GNG12 | NM_018841 | G-protein gamma-12 subunit |
| GNG4 | NM_004485 | guanine nucleotide binding protein (G protein), |
| GNG7 | NM_052847 | guanine nucleotide binding protein (G protein), |
| GNL3 | NM_014366 | guanine nucleotide binding protein-like 3 |
| GNPNAT1 | NM_198066 | glucosamine-phosphate N-acetyltransferase 1 |
| GNS | NM_002076 | glucosamine (N-acetyl)-6-sulfatase precursor |
| GOLGA | NM_018652 | golgin-like protein |
| GOLGA1 | NM_002077 | golgin 97 |
| GOLGA4 | NM_002078 | golgi autoantigen, golgin subfamily a, 4 |
| GOLPH2 | NM_016548 | golgi phosphoprotein 2 |
| GORASP1 | NM_031899 | Golgi reassembly stacking protein 1 |
| GOSR1 | NM_001007024 | golgi SNAP receptor complex member 1 isoform 3 |
| GOT1 | NM_002079 | aspartate aminotransferase 1 |
| GOT2 | NM_002080 | aspartate aminotransferase 2 precursor |
| GP5 | NM_004488 | glycoprotein V (platelet) |
| GP6 | NM_016363 | glycoprotein VI (platelet) |
| GPA33 | NM_005814 | transmembrane glycoprotein A33 precursor |
| GPC1 | NM_002081 | glypican 1 precursor |
| GPC2 | NM_152742 | glypican 2 |
| GPIAP1 | NM_005898 | membrane component chromosome 11 surface marker |
| GPR109A | NM_177551 | G protein-coupled receptor 109A |
| GPR109B | NM_006018 | G protein-coupled receptor 109B |
| GPR135 | NM_022571 | G protein-coupled receptor 135 |
| GPR176 | NM_007223 | putative G protein coupled receptor |
| GPR180 | NM_180989 | G protein-coupled receptor 180 precursor |
| GPR26 | NM_153442 | G protein-coupled receptor 26 |
| GPR62 | NM_080865 | G protein-coupled receptor 62 |
| GPR83 | NM_016540 | G protein-coupled receptor 83 |
| GPRC5A | NM_003979 | G protein-coupled receptor, family C, group 5, |
| GPRC5B | NM_016235 | G protein-coupled receptor, family C, group 5, |
| GPSM3 | NM_022107 | G-protein signalling modulator 3 (AGS3-like, C. |
| GPX3 | NM_002084 | plasma glutathione peroxidase 3 precursor |
| GRAMD1A | NM_020895 | hypothetical protein LOC57655 |
| GRAMD2 | NM_001012642 | hypothetical protein LOC196996 |
| GRHL2 | NM_024915 | transcription factor CP2-like 3 |
| GRIA2 | NM_000826 | glutamate receptor, ionotropic, AMPA 2 |
| GRIN2B | NM_000834 | N-methyl-D-aspartate receptor subunit 2B |
| GRINL1A | NM_001018103 | glutamate receptor, ionotropic, N-methyl |
| GRIPAP1 | NM_020137 | GRIP1 associated protein 1 isoform 1 |
| GRK1 | NM_002929 | rhodopsin kinase |
| GSDMDC1 | NM_024736 | gasdermin domain containing 1 |
| GSTA4 | NM_001512 | glutathione S-transferase A4 |
| GSTM4 | NM_147149 | glutathione S-transferase M4 isoform 3 |
| GTF2I | NM_001518 | general transcription factor II, i isoform 4 |
| GTPBP1 | NM_004286 | GTP binding protein 1 |
| GTPBP3 | NM_032620 | GTP binding protein 3 (mitochondrial) isoform V |
| GUSBL2 | NM_206910 | hypothetical protein LOC375513 isoform 2 |
| H2AFY2 | NM_018649 | core histone macroH2A2.2 |
| H2BFWT | NM_001002916 | H2B histone family, member W, testis-specific |
| H6PD | NM_004285 | hexose-6-phosphate dehydrogenase precursor |
| HABP2 | NM_004132 | hyaluronan binding protein 2 |
| HAGHL | NM_207112 | hydroxyacylglutathione hydrolase-like isoform 1 |
| HAPLN4 | NM_023002 | brain link protein 2 |
| HAS3 | NM_005329 | hyaluronan synthase 3 isoform a |
| HBS1L | NM_006620 | HBS1-like |
| hCAP-H2 | NM_152299 | kleisin beta isoform 2 |
| HCCS | NM_005333 | holocytochrome c synthase (cytochrome c |
| HCG9 | NM_005844 | hypothetical protein LOC10255 |
| HCP1 | NM_080669 | heme carrier protein 1 |
| HDAC4 | NM_006037 | histone deacetylase 4 |
| HDAC7A | NM_015401 | histone deacetylase 7A isoform a |
| HECA | NM_016217 | headcase |
| HECTD1 | NM_015382 | HECT domain containing 1 |
| HECW2 | NM_020760 | HECT, C2 and WW domain containing E3 ubiquitin |
| HEMK1 | NM_016173 | HemK methyltransferase family member 1 |
| HES2 | NM_019089 | hairy and enhancer of split homolog 2 |
| HFE | NM_000410 | hemochromatosis protein isoform 1 precursor |
| HGF | NM_001010934 | hepatocyte growth factor isoform 5 precursor |
| HGS | NM_004712 | hepatocyte growth factor-regulated tyrosine |
| HHAT | NM_018194 | hedgehog acyltransferase |
| HHLA2 | NM_007072 | HERV-H LTR-associating 2 |
| HIATL1 | NM_032558 | hypothetical protein LOC84641 |
| HIG2 | NM_013332 | hypoxia-inducible protein 2 |
| HIGD2A | NM_138820 | HIG1 domain family, member 2A |
| HIP1 | NM_005338 | huntingtin interacting protein 1 |
| HIPK1 | NM_181358 | homeodomain-interacting protein kinase 1 isoform |
| HIST1H4E | NM_003545 | H4 histone family, member J |
| HK2 | NM_000189 | hexokinase 2 |
| HKDC1 | NM_025130 | hexokinase domain containing 1 |
| HKR2 | NM_181846 | GLI-Kruppel family member HKR2 |
| HLA-A | NM_002116 | major histocompatibility complex, class I, A |
| HLA-B | NM_005514 | major histocompatibility complex, class I, B |
| HLA-C | NM_002117 | major histocompatibility complex, class I, C |
| HLA-DOA | NM_002119 | major histocompatibility complex, class II, DO |
| HLA-DPA1 | NM_033554 | major histocompatibility complex, class II, DP |
| HLA-DPB1 | NM_002121 | major histocompatibility complex, class II, DP |
| HLA-DQA2 | NM_020056 | major histocompatibility complex, class II, DQ |
| HLA-DQB1 | NM_002123 | major histocompatibility complex, class II, DQ |
| HLA-E | NM_005516 | major histocompatibility complex, class I, E |
| HLF | NM_002126 | hepatic leukemia factor |
| HMBS | NM_000190 | hydroxymethylbilane synthase isoform 1 |
| HMG2L1 | NM_001003681 | high-mobility group protein 2-like 1 isoform b |
| HMGA1 | NM_002131 | high mobility group AT-hook 1 isoform b |
| HMGA2 | NM_001015886 | high mobility group AT-hook 2 isoform c |
| HMGB1 | NM_002128 | high-mobility group box 1 |
| HMGCS2 | NM_005518 | 3-hydroxy-3-methylglutaryl-Coenzyme A synthase 2 |
| HMMR | NM_012484 | hyaluronan-mediated motility receptor isoform a |
| HN1 | NM_001002033 | hematological and neurological expressed 1 |
| HNF4A | NM_000457 | hepatocyte nuclear factor 4 alpha isoform b |
| HNMT | NM_001024074 | histamine N-methyltransferase isoform 2 |
| HNRPA0 | NM_006805 | heterogeneous nuclear ribonucleoprotein A0 |
| HOXA5 | NM_019102 | homeobox A5 |
| HOXB13 | NM_006361 | homeobox B13 |
| HOXB9 | NM_024017 | homeobox B9 |
| HOXC5 | NM_018953 | homeobox C5 |
| HPCAL4 | NM_016257 | hippocalcin-like protein 4 |
| HPS5 | NM_007216 | Hermansky-Pudlak syndrome 5 isoform b |
| HPSE | NM_006665 | heparanase |
| HR | NM_005144 | hairless protein isoform a |
| HRB | NM_004504 | HIV-1 Rev binding protein |
| HRH4 | NM_021624 | histamine H4 receptor |
| HS2ST1 | NM_012262 | heparan sulfate 2-O-sulfotransferase 1 |
| HS3ST2 | NM_006043 | heparan sulfate D-glucosaminyl |
| HSBP1 | NM_001537 | heat shock factor binding protein 1 |
| HSD17B1 | NM_000413 | hydroxysteroid (17-beta) dehydrogenase 1 |
| HSDL2 | NM_032303 | hydroxysteroid dehydrogenase like 2 |
| HSH2D | NM_032855 | hematopoietic SH2 domain containing |
| HSPB7 | NM_014424 | heat shock 27 kDa protein family, member 7 |
| HSPBP1 | NM_012267 | hsp70-interacting protein |
| HSPC065 | NM_014157 | hypothetical protein LOC29070 |
| HTR2C | NM_000868 | 5-hydroxytryptamine (serotonin) receptor 2C |
| HTR3A | NM_000869 | 5-hydroxytryptamine (serotonin) receptor 3A |
| HTR3B | NM_006028 | 5-hydroxytryptamine (serotonin) receptor 3B |
| HTR4 | NM_000870 | serotonin 5-HT4 receptor isoform b |
| HTR6 | NM_000871 | 5-hydroxytryptamine (serotonin) receptor 6 |
| HTR7 | NM_000872 | 5-hydroxytryptamine receptor 7 isoform a |
| HUNK | NM_014586 | hormonally upregulated Neu-associated kinase |
| HYOU1 | NM_006389 | oxygen regulated protein precursor |
| HYPK | NM_016400 | Huntingtin interacting protein K |
| IAPP | NM_000415 | islet amyloid polypeptide precursor |
| IBRDC1 | NM_152553 | IBR domain containing 1 |
| ICA1 | NM_022308 | islet cell autoantigen 1 isoform 3 |
| ID4 | NM_001546 | inhibitor of DNA binding 4, dominant negative |
| IER3 | NM_003897 | immediate early response 3 isoform short |
| IFIT3 | NM_001031683 | interferon-induced protein with |
| IFIT5 | NM_012420 | interferon-induced protein with |
| IFNA14 | NM_002172 | interferon, alpha 14 |
| IFNA16 | NM_002173 | interferon, alpha 16 |
| IFNA7 | NM_021057 | interferon, alpha 7 |
| IGF1 | NM_000618 | insulin-like growth factor 1 (somatomedin C) |
| IGF2BP1 | NM_006546 | insulin-like growth factor 2 mRNA binding |
| IGF2R | NM_000876 | insulin-like growth factor 2 receptor |
| IGFBP3 | NM_000598 | insulin-like growth factor binding protein 3 |
| IGFBP5 | NM_000599 | insulin-like growth factor binding protein 5 |
| IGFL1 | NM_198541 | insulin growth factor-like family member 1 |
| IGSF4D | NM_153184 | immunoglobulin superfamily, member 4D |
| IHPK1 | NM_001006115 | inositol hexaphosphate kinase 1 isoform 2 |
| IHPK2 | NM_001005910 | inositol hexaphosphate kinase 2 isoform b |
| IHPK3 | NM_054111 | inositol hexaphosphate kinase 3 |
| IL10RA | NM_001558 | interleukin 10 receptor, alpha precursor |
| IL10RB | NM_000628 | interleukin 10 receptor, beta precursor |
| IL11RA | NM_147162 | interleukin 11 receptor, alpha isoform 2 |
| IL12RB1 | NM_153701 | interleukin 12 receptor, beta 1 isoform 2 |
| IL12RB2 | NM_001559 | interleukin 12 receptor, beta 2 precursor |
| IL13RA1 | NM_001560 | interleukin 13 receptor, alpha 1 precursor |
| IL16 | NM_004513 | interleukin 16 isoform 1 precursor |
| IL17C | NM_013278 | interleukin 17C |
| IL17RD | NM_017563 | interleukin 17 receptor D |
| IL18 | NM_001562 | interleukin 18 proprotein |
| IL1F5 | NM_012275 | interleukin 1 family, member 5 |
| IL1F9 | NM_019618 | interleukin 1 family, member 9 |
| IL1RAP | NM_002182 | interleukin 1 receptor accessory protein isoform |
| IL1RL1 | NM_003856 | interleukin 1 receptor-like 1 isoform 2 |
| IL1RN | NM_000577 | interleukin 1 receptor antagonist isoform 3 |
| IL22RA2 | NM_052962 | interleukin 22-binding protein isoform 1 |
| IL27RA | NM_004843 | class I cytokine receptor |
| IL28RA | NM_170743 | interleukin 28 receptor, alpha isoform 1 |
| IL2RA | NM_000417 | interleukin 2 receptor, alpha chain precursor |
| IL3 | NM_000588 | interleukin 3 precursor |
| IL6R | NM_181359 | interleukin 6 receptor isoform 2 precursor |
| IL8RA | NM_000634 | interleukin 8 receptor alpha |
| INCA1 | NM_213726 | inhibitor of CDK interacting with cyclin A1 |
| ING5 | NM_032329 | inhibitor of growth family, member 5 |
| INOC1 | NM_017553 | INO80 complex homolog 1 |
| INPP5E | NM_019892 | inositol polyphosphate-5-phosphatase E |
| INSL4 | NM_002195 | insulin-like 4 precursor |
| INTS2 | NM_020748 | integrator complex subunit 2 |
| IQCC | NM_018134 | IQ motif containing C |
| IQCE | NM_152558 | IQ motif containing E |
| IRAK1 | NM_001025242 | interleukin-1 receptor-associated kinase 1 |
| IRF5 | NM_002200 | interferon regulatory factor 5 isoform a |
| IRF8 | NM_002163 | interferon regulatory factor 8 |
| IRX6 | NM_024335 | iroquois homeobox protein 6 |
| ITGA11 | NM_001004439 | integrin, alpha 11 precursor |
| ITGA3 | NM_002204 | integrin alpha 3 isoform a precursor |
| ITGA5 | NM_002205 | integrin alpha 5 precursor |
| ITGA6 | NM_000210 | integrin alpha chain, alpha 6 |
| ITGAM | NM_000632 | integrin alpha M precursor |
| ITGAV | NM_002210 | integrin alpha-V precursor |
| ITM2B | NM_021999 | integral membrane protein 2B |
| ITPR1 | NM_002222 | inositol 1,4,5-triphosphate receptor, type 1 |
| JAG1 | NM_000214 | jagged 1 precursor |
| JAGN1 | NM_032492 | jagunal homolog 1 |
| JM11 | NM_033626 | hypothetical protein LOC90060 |
| JMJD2B | NM_015015 | jumonji domain containing 2B |
| JMJD2C | NM_015061 | jumonji domain containing 2C |
| JOSD1 | NM_014876 | Josephin domain containing 1 |
| JOSD3 | NM_024116 | Josephin domain containing 3 |
| JPH1 | NM_020647 | junctophilin 1 |
| JPH3 | NM_020655 | junctophilin 3 |
| JRK | NM_003724 | jerky homolog |
| K6IRS4 | NM_175053 | keratin 6 irs4 |
| KA36 | NM_182497 | type I hair keratin KA36 |
| KAL1 | NM_000216 | Kallmann syndrome 1 protein |
| KATNAL1 | NM_001014380 | katanin p60 subunit A-like 1 |
| KBTBD3 | NM_152433 | BTB and kelch domain containing 3 |
| KBTBD6 | NM_152903 | kelch repeat and BTB (POZ) domain-containing 6 |
| KBTBD8 | NM_032505 | T-cell activation kelch repeat protein |
| KCNA7 | NM_031886 | potassium voltage-gated channel, shaker-related |
| KCNB1 | NM_004975 | potassium voltage-gated channel, Shab-related |
| KCND1 | NM_004979 | potassium voltage-gated channel, Shal-related |
| KCND2 | NM_012281 | potassium voltage-gated channel, Shal-related |
| KCND3 | NM_004980 | potassium voltage-gated channel, Shal-related |
| KCNE1L | NM_012282 | potassium voltage-gated channel, Isk-related |
| KCNE3 | NM_005472 | potassium voltage-gated channel, Isk-related |
| KCNH5 | NM_172375 | potassium voltage-gated channel, subfamily H, |
| KCNH6 | NM_173092 | potassium voltage-gated channel, subfamily H, |
| KCNH7 | NM_033272 | potassium voltage-gated channel, subfamily H, |
| KCNH8 | NM_144633 | potassium voltage-gated channel, subfamily H, |
| KCNIP1 | NM_014592 | Kv channel interacting protein 1 isoform 2 |
| KCNIP2 | NM_014591 | Kv channel interacting protein 2 isoform 1 |
| KCNJ10 | NM_002241 | potassium inwardly-rectifying channel, subfamily |
| KCNJ13 | NM_002242 | potassium inwardly-rectifying channel J13 |
| KCNJ4 | NM_004981 | potassium inwardly-rectifying channel J4 |
| KCNJ5 | NM_000890 | potassium inwardly-rectifying channel J5 |
| KCNJ8 | NM_004982 | potassium inwardly-rectifying channel J8 |
| KCNK2 | NM_001017424 | potassium channel, subfamily K, member 2 isoform |
| KCNK3 | NM_002246 | potassium channel, subfamily K, member 3 |
| KCNMA1 | NM_001014797 | large conductance calcium-activated potassium |
| KCNS2 | NM_020697 | potassium voltage-gated channel, |
| KCTD10 | NM_031954 | potassium channel tetramerisation domain |
| KDELC2 | NM_153705 | KDEL (Lys-Asp-Glu-Leu) containing 2 |
| KEAP1 | NM_012289 | kelch-like ECH-associated protein 1 |
| KENAE | NM_176816 | hypothetical protein LOC202243 |
| KIAA0125 | NM_014792 | hypothetical protein LOC9834 |
| KIAA0232 | NM_014743 | hypothetical protein LOC9778 |
| KIAA0256 | NM_014701 | hypothetical protein LOC9728 |
| KIAA0265 | NM_014997 | hypothetical protein LOC23008 |
| KIAA0286 | NM_015257 | hypothetical protein LOC23306 |
| KIAA0319 | NM_014809 | KIAA0319 |
| KIAA0319L | NM_024874 | polycystic kidney disease 1-like isoform a |
| KIAA0329 | NM_014844 | hypothetical protein LOC9895 |
| KIAA0350 | NM_015226 | hypothetical protein LOC23274 |
| KIAA0355 | NM_014686 | hypothetical protein LOC9710 |
| KIAA0427 | NM_014772 | hypothetical protein LOC9811 |
| KIAA0446 | NM_014655 | hypothetical protein LOC9673 |
| KIAA0467 | NM_015284 | KIAA0467 protein |
| KIAA0494 | NM_014774 | hypothetical protein LOC9813 |
| KIAA0495 | NM_207306 | KIAA0495 |
| KIAA0513 | NM_014732 | hypothetical protein LOC9764 |
| KIAA0514 | NM_014696 | hypothetical protein LOC9721 |
| KIAA0523 | NM_015253 | hypothetical protein LOC23302 |
| KIAA0553 | NM_001002909 | hypothetical protein LOC23131 |
| KIAA0644 | NM_014817 | hypothetical protein LOC9865 |
| KIAA0652 | NM_014741 | hypothetical protein LOC9776 |
| KIAA0676 | NM_015043 | hypothetical protein LOC23061 isoform b |
| KIAA0701 | NM_001006947 | hypothetical protein LOC23074 isoform b |
| KIAA0703 | NM_014861 | calcium-transporting ATPase 2C2 |
| KIAA0738 | NM_014719 | hypothetical protein LOC9747 |
| KIAA0773 | NM_001031690 | hypothetical protein LOC9715 |
| KIAA0789 | NM_014653 | hypothetical protein LOC9671 |
| KIAA0804 | NM_001009921 | hypothetical protein LOC23355 isoform a |
| KIAA0831 | NM_014924 | hypothetical protein LOC22863 |
| KIAA0889 | NM_152257 | hypothetical protein LOC25781 |
| KIAA0892 | NM_015329 | hypothetical protein LOC23383 |
| KIAA1008 | NM_014953 | KIAA1008 |
| KIAA1012 | NM_014939 | hypothetical protein LOC22878 |
| KIAA1024 | NM_015206 | hypothetical protein LOC23251 |
| KIAA1128 | NM_018999 | granule cell antiserum positive 14 |
| KIAA1161 | NM_020702 | hypothetical protein LOC57462 |
| KIAA1166 | NM_018684 | hepatocellular carcinoma-associated antigen 127 |
| KIAA1189 | NM_001009959 | hypothetical protein LOC57471 isoform a |
| KIAA1267 | NM_015443 | hypothetical protein LOC284058 |
| KIAA1274 | NM_014431 | KIAA1274 |
| KIAA1328 | NM_020776 | hypothetical protein LOC57536 |
| KIAA1333 | NM_017769 | hypothetical protein LOC55632 |
| KIAA1446 | NM_020836 | likely ortholog of rat brain-enriched guanylate |
| KIAA1456 | NM_020844 | hypothetical protein LOC57604 |
| KIAA1467 | NM_020853 | hypothetical protein LOC57613 |
| KIAA1522 | NM_020888 | hypothetical protein LOC57648 |
| KIAA1576 | NM_020927 | hypothetical protein LOC57687 |
| KIAA1604 | NM_020943 | hypothetical protein LOC57703 |
| KIAA1622 | NM_020958 | HEAT-like repeat-containing protein isoform 2 |
| KIAA1641 | NM_020970 | hypothetical protein LOC57730 |
| KIAA1706 | NM_030636 | hypothetical protein LOC80820 |
| KIAA1715 | NM_030650 | Lunapark |
| KIAA1727 | NM_033393 | hypothetical protein LOC85462 |
| KIAA1729 | NM_053042 | hypothetical protein LOC85460 |
| KIAA1737 | NM_033426 | KIAA1737 protein |
| KIAA1853 | NM_194286 | KIAA1853 protein |
| KIAA1875 | NM_032529 | KIAA1875 protein |
| KIAA1909 | NM_052909 | hypothetical protein LOC153478 |
| KIAA1914 | NM_001001936 | KIAA1914 protein isoform 1 |
| KIAA1920 | NM_052919 | hypothetical protein LOC114817 |
| KIAA2022 | NM_001008537 | hypothetical protein LOC340533 |
| KIF1B | NM_015074 | kinesin family member 1B isoform b |
| KIF3B | NM_004798 | kinesin family member 3B |
| KIF3C | NM_002254 | kinesin family member 3C |
| KIF4A | NM_012310 | kinesin family member 4 |
| KIF9 | NM_022342 | kinesin family member 9 isoform 1 |
| KIRREL | NM_018240 | kin of IRRE like |
| KLC2 | NM_022822 | likely ortholog of kinesin light chain 2 |
| KLC3 | NM_177417 | kinesin light chain 3 |
| KLF12 | NM_007249 | Kruppel-like factor 12 isoform a |
| KLF13 | NM_015995 | Kruppel-like factor 13 |
| KLF17 | NM_173484 | zinc finger protein 393 |
| KLF5 | NM_001730 | Kruppel-like factor 5 |
| KLHDC6 | NM_207335 | hypothetical protein LOC166348 |
| KLHL20 | NM_014458 | kelch-like 20 |
| KLHL21 | NM_014851 | kelch-like 21 |
| KLHL22 | NM_032775 | kelch-like |
| KLHL24 | NM_017644 | DRE1 protein |
| KLHL25 | NM_022480 | BTB/POZ KELCH domain protein |
| KLHL26 | NM_018316 | hypothetical protein LOC55295 |
| KLHL6 | NM_130446 | kelch-like 6 |
| KLHL7 | NM_001031710 | SBBI26 protein isoform 1 |
| KLK13 | NM_015596 | kallikrein 13 precursor |
| KLK5 | NM_012427 | kallikrein 5 preproprotein |
| KLRG1 | NM_005810 | killer cell lectin-like receptor subfamily G, |
| KM-HN-1 | NM_152775 | KM-HN-1 protein |
| KNDC1 | NM_152643 | kinase non-catalytic C-lobe domain (KIND) |
| KPNA1 | NM_002264 | karyopherin alpha 1 |
| KPNA6 | NM_012316 | karyopherin alpha 6 |
| KRAS | NM_004985 | c-K-ras2 protein isoform b |
| KREMEN2 | NM_024507 | kringle-containing transmembrane protein 2 |
| KRIT1 | NM_001013406 | krev interaction trapped 1 isoform 2 |
| KRT25A | NM_181534 | keratin 25A |
| KRT2A | NM_000423 | keratin 2a |
| KRT2B | NM_015848 | cytokeratin 2 |
| KRT4 | NM_002272 | keratin 4 |
| KRTAP1-1 | NM_030967 | keratin associated protein 1-1 |
| KRTAP4-14 | NM_033059 | keratin associated protein 4-14 |
| KRTAP4-4 | NM_032524 | keratin associated protein 4.4 |
| KRTAP9-2 | NM_031961 | keratin associated protein 9.2 |
| KRTAP9-3 | NM_031962 | keratin associated protein 9.3 |
| L3MBTL4 | NM_173464 | hypothetical protein LOC91133 |
| LACE1 | NM_145315 | lactation elevated 1 |
| LAMB3 | NM_000228 | laminin subunit beta 3 precursor |
| LAMC1 | NM_002293 | laminin, gamma 1 precursor |
| LANCL2 | NM_018697 | LanC lantibiotic synthetase component C-like 2 |
| LARP1 | NM_015315 | la related protein isoform 1 |
| LARP4 | NM_052879 | c-Mpl binding protein isoform a |
| LARP5 | NM_015155 | La ribonucleoprotein domain family, member 5 |
| LASP1 | NM_006148 | LIM and SH3 protein 1 |
| LASS3 | NM_178842 | hypothetical protein LOC204219 |
| LBH | NM_030915 | hypothetical protein DKFZp566J091 |
| LCT | NM_002299 | lactase-phlorizin hydrolase preproprotein |
| LDB3 | NM_007078 | LIM domain binding 3 |
| LDLR | NM_000527 | low density lipoprotein receptor precursor |
| LDLRAP1 | NM_015627 | low density lipoprotein receptor adaptor protein |
| LDOC1L | NM_032287 | hypothetical protein LOC84247 |
| LECT2 | NM_002302 | leukocyte cell-derived chemotaxin 2 precursor |
| LENEP | NM_018655 | lens epithelial protein |
| LEREPO4 | NM_018471 | erythropoietin 4 immediate early response |
| LETM1 | NM_012318 | leucine zipper-EF-hand containing transmembrane |
| LGALS8 | NM_006499 | galectin 8 isoform a |
| LHFPL2 | NM_005779 | lipoma HMGIC fusion partner-like 2 |
| LHFPL3 | NM_199000 | lipoma HMGIC fusion partner-like 3 |
| LHFPL5 | NM_182548 | lipoma HMGIC fusion partner-like 5 |
| LHX3 | NM_014564 | LIM homeobox protein 3 isoform b |
| LHX4 | NM_033343 | LIM homeobox protein 4 |
| LIAS | NM_006859 | lipoic acid synthetase isoform 1 precursor |
| LIF | NM_002309 | leukemia inhibitory factor (cholinergic |
| LIFR | NM_002310 | leukemia inhibitory factor receptor precursor |
| LILRB1 | NM_006669 | leukocyte immunoglobulin-like receptor, |
| LILRB4 | NM_006847 | leukocyte immunoglobulin-like receptor, |
| LIMD1 | NM_014240 | LIM domains containing 1 |
| LIMD2 | NM_030576 | LIM domain containing 2 |
| LIMK1 | NM_002314 | LIM domain kinase 1 |
| LIMK2 | NM_005569 | LIM domain kinase 2 isoform 2a |
| LIMS2 | NM_017980 | LIM and senescent cell antigen-like domains 2 |
| LIMS3 | NM_033514 | LIM and senescent cell antigen-like domains 3 |
| LIN28 | NM_024674 | lin-28 homolog |
| LIN9 | NM_173083 | lin-9 homolog |
| LIX1 | NM_153234 | limb expression 1 |
| LLGL1 | NM_004140 | lethal giant larvae homolog 1 |
| LMNB2 | NM_032737 | lamin B2 |
| LMO4 | NM_006769 | LIM domain only 4 |
| LMO7 | NM_005358 | LIM domain only 7 |
| LMOD3 | NM_198271 | leiomodin 3 (fetal) |
| LOC116236 | NM_198147 | hypothetical protein LOC116236 |
| LOC124491 | NM_145254 | hypothetical protein LOC124491 |
| LOC129138 | NM_138797 | hypothetical protein LOC129138 |
| LOC129607 | NM_207315 | thymidylate kinase family LPS-inducible member |
| LOC130576 | NM_177964 | hypothetical protein LOC130576 |
| LOC133619 | NM_130809 | hypothetical protein LOC133619 |
| LOC144501 | NM_182507 | hypothetical protein LOC144501 |
| LOC151194 | NM_145280 | hypothetical protein LOC151194 |
| LOC152485 | NM_178835 | hypothetical protein LOC152485 |
| LOC153561 | NM_207331 | hypothetical protein LOC153561 |
| LOC158318 | NM_001024608 | hypothetical protein LOC158318 |
| LOC162427 | NM_178126 | hypothetical protein LOC162427 |
| LOC196463 | NM_173542 | hypothetical protein LOC196463 |
| LOC196752 | NM_001010864 | hypothetical protein LOC196752 |
| LOC197322 | NM_174917 | hypothetical protein LOC197322 |
| LOC201164 | NM_178836 | hypothetical protein LOC201164 |
| LOC203427 | NM_145305 | mitochondrial solute carrier protein |
| LOC221091 | NM_203422 | hypothetical protein LOC221091 |
| LOC222967 | NM_173565 | hypothetical protein LOC222967 |
| LOC283219 | NM_001029859 | hypothetical protein LOC283219 |
| LOC283537 | NM_181785 | hypothetical protein LOC283537 |
| LOC283551 | NM_001012706 | hypothetical protein LOC283551 |
| LOC284296 | NM_175908 | hypothetical protein LOC284296 |
| LOC284434 | NM_001007525 | hypothetical protein LOC284434 |
| LOC284757 | NM_001004305 | hypothetical protein LOC284757 |
| LOC286076 | NM_001024610 | hypothetical protein LOC286076 |
| LOC339524 | NM_207357 | hypothetical protein LOC339524 |
| LOC340156 | NM_001012418 | hypothetical protein LOC340156 |
| LOC342897 | NM_001001414 | similar to F-box only protein 2 |
| LOC345222 | NM_001012982 | hypothetical protein LOC345222 |
| LOC348262 | NM_207368 | hypothetical protein LOC348262 |
| LOC387856 | NM_001013635 | hypothetical protein LOC387856 |
| LOC388503 | NM_001013640 | hypothetical protein LOC388503 |
| LOC389118 | NM_001007540 | hypothetical protein LOC389118 |
| LOC389199 | NM_203423 | hypothetical protein LOC389199 |
| LOC389791 | NM_001013652 | hypothetical protein LOC389791 |
| LOC389834 | NM_001013655 | hypothetical protein LOC389834 |
| LOC392395 | NM_001013664 | hypothetical protein LOC392395 |
| LOC399706 | NM_001010910 | hypothetical protein LOC399706 |
| LOC399898 | NM_001013666 | hypothetical protein LOC399898 |
| LOC400145 | NM_001013669 | hypothetical protein LOC400145 |
| LOC400499 | NM_001013671 | hypothetical protein LOC400499 |
| LOC400657 | NM_001008234 | hypothetical protein LOC400657 |
| LOC400891 | NM_001013675 | hypothetical protein LOC400891 |
| LOC400924 | NM_001013676 | hypothetical protein LOC400924 |
| LOC400965 | NM_001013677 | hypothetical protein LOC400965 |
| LOC401137 | NM_214711 | hypothetical protein LOC401137 |
| LOC401398 | NM_001023566 | hypothetical protein LOC401398 |
| LOC401431 | NM_001008745 | hypothetical protein LOC401431 |
| LOC401507 | NM_001012278 | hypothetical protein LOC401507 |
| LOC401589 | NM_001013687 | hypothetical protein LOC401589 |
| LOC401620 | NM_001013688 | hypothetical protein LOC401620 |
| LOC401720 | NM_001013690 | hypothetical protein LOC401720 |
| LOC440313 | NM_001013704 | hypothetical protein LOC440313 |
| LOC440337 | NM_001013705 | hypothetical protein LOC440337 |
| LOC440570 | NM_001013708 | hypothetical protein LOC440570 |
| LOC440742 | NM_001013710 | hypothetical protein LOC440742 |
| LOC440925 | NM_001013712 | hypothetical protein LOC440925 |
| LOC440944 | NM_001013713 | hypothetical protein LOC440944 |
| LOC441070 | NM_001013715 | hypothetical protein LOC441070 |
| LOC441136 | NM_001013719 | hypothetical protein LOC441136 |
| LOC441268 | NM_001013725 | hypothetical protein LOC441268 |
| LOC441459 | NM_001013728 | hypothetical protein LOC441459 |
| LOC442247 | NM_001013734 | hypothetical protein LOC442247 |
| LOC504188 | NM_001013404 | hypothetical protein LOC504188 |
| LOC54103 | NM_017439 | hypothetical protein LOC54103 |
| LOC541473 | NM_001013748 | FKBP6-like |
| LOC554251 | NM_001024680 | hypothetical protein LOC554251 |
| LOC55908 | NM_018687 | hepatocellular carcinoma-associated gene TD26 |
| LOC613206 | NM_001033016 | myeloproliferative disease associated tumor |
| LOC613266 | NM_001033516 | hypothetical protein LOC613266 |
| LOC63928 | NM_022097 | hepatocellular carcinoma antigen gene 520 |
| LOC90167 | NM_194277 | hypothetical protein LOC90167 |
| LOC90639 | NM_001031617 | hypothetical protein LOC90639 |
| LOH12CR1 | NM_058169 | LOH1CR12 |
| LOXL4 | NM_032211 | lysyl oxidase-like 4 precursor |
| LPIN3 | NM_022896 | lipin 3 |
| LPP | NM_005578 | LIM domain containing preferred translocation |
| LRAT | NM_004744 | lecithin retinol acyltransferase |
| LRBA | NM_006726 | LPS-responsive vesicle trafficking, beach and |
| LRCH4 | NM_002319 | leucine-rich repeats and calponin homology (CH) |
| LRP11 | NM_032832 | low density lipoprotein receptor-related protein |
| LRP12 | NM_013437 | suppression of tumorigenicity |
| LRP2BP | NM_018409 | LRP2 binding protein |
| LRRC14 | NM_014665 | leucine rich repeat containing 14 |
| LRRC2 | NM_024512 | leucine rich repeat containing 2 |
| LRRC20 | NM_018205 | leucine rich repeat containing 20 isoform 3 |
| LRRC27 | NM_030626 | leucine rich repeat containing 27 |
| LRRC3B | NM_052953 | leucine rich repeat containing 3B |
| LRRC48 | NM_031294 | leucine rich repeat containing 48 |
| LRRC54 | NM_015516 | tsukushi |
| LRRIQ2 | NM_024548 | leucine-rich repeats and IQ motif containing 2 |
| LRRN5 | NM_006338 | leucine rich repeat neuronal 5 precursor |
| LRRTM3 | NM_178011 | leucine rich repeat transmembrane neuronal 3 |
| LSM12 | NM_152344 | hypothetical protein LOC124801 |
| LSM16 | NM_025083 | LSM16 homolog (EDC3, S. cerevisiae) |
| LTBP2 | NM_000428 | latent transforming growth factor beta binding |
| LUZP1 | NM_033631 | leucine zipper protein 1 |
| LY6H | NM_002347 | lymphocyte antigen 6 complex, locus H |
| LY86 | NM_004271 | MD-1, RP105-associated |
| LYCAT | NM_001002257 | lysocardiolipin acyltransferase isoform 2 |
| LYPLA3 | NM_012320 | lysophospholipase 3 (lysosomal phospholipase |
| LYSMD1 | NM_212551 | LysM, putative peptidoglycan-binding, domain |
| LYSMD4 | NM_152449 | hypothetical protein LOC145748 |
| LYZ | NM_000239 | lysozyme precursor |
| LZTR2 | NM_033127 | regucalcin gene promotor region related protein |
| LZTS1 | NM_021020 | leucine zipper, putative tumor suppressor 1 |
| M6PR | NM_002355 | cation-dependent mannose-6-phosphate receptor |
| M6PRBP1 | NM_005817 | mannose 6 phosphate receptor binding protein 1 |
| MAB21L1 | NM_005584 | mab-21-like protein 1 |
| MAF | NM_001031804 | v-maf musculoaponeurotic fibrosarcoma oncogene |
| MAGEA8 | NM_005364 | melanoma antigen family A, 8 |
| MAGEA9 | NM_005365 | melanoma antigen family A, 9 |
| MAGEL2 | NM_019066 | MAGE-like protein 2 |
| MAGI2 | NM_012301 | membrane associated guanylate kinase, WW and PDZ |
| MALL | NM_005434 | mal, T-cell differentiation protein-like |
| MAN1C1 | NM_020379 | mannosidase, alpha, class 1C, member 1 |
| MANEA | NM_024641 | mannosidase, endo-alpha |
| MAP1B | NM_005909 | microtubule-associated protein 1B isoform 1 |
| MAP3K3 | NM_002401 | mitogen-activated protein kinase kinase kinase 3 |
| MAP3K7 | NM_003188 | mitogen-activated protein kinase kinase kinase 7 |
| MAP4K1 | NM_007181 | mitogen-activated protein kinase kinase kinase |
| MAPK1 | NM_002745 | mitogen-activated protein kinase 1 |
| MAPK14 | NM_001315 | mitogen-activated protein kinase 14 isoform 1 |
| MAPK3 | NM_002746 | mitogen-activated protein kinase 3 isoform 1 |
| MAPK7 | NM_002749 | mitogen-activated protein kinase 7 isoform 1 |
| MAPKAPK2 | NM_004759 | mitogen-activated protein kinase-activated |
| MAPKBP1 | NM_014994 | mitogen-activated protein kinase binding protein |
| MAPT | NM_005910 | microtubule-associated protein tau isoform 2 |
| MARCH3 | NM_178450 | membrane-associated ring finger (C3HC4) 3 |
| MARCH5 | NM_017824 | ring finger protein 153 |
| MARCKS | NM_002356 | myristoylated alanine-rich protein kinase C |
| MARK3 | NM_002376 | MAP/microtubule affinity-regulating kinase 3 |
| MARVELD1 | NM_031484 | MARVEL domain containing 1 |
| MARVELD3 | NM_052858 | MARVEL domain containing 3 isoform 2 |
| MAS1 | NM_002377 | MAS1 oncogene |
| MASP1 | NM_001879 | mannan-binding lectin serine protease 1 isoform |
| MAT1A | NM_000429 | methionine adenosyltransferase I, alpha |
| MATN2 | NM_002380 | matrilin 2 isoform a precursor |
| MBD3 | NM_003926 | methyl-CpG binding domain protein 3 |
| MBNL3 | NM_018388 | muscleblind-like 3 isoform G |
| MCART6 | NM_001012755 | hypothetical protein LOC401612 |
| MCCC2 | NM_022132 | methylcrotonoyl-Coenzyme A carboxylase 2 (beta) |
| MCF2 | NM_005369 | MCF.2 cell line derived transforming sequence |
| MCFD2 | NM_139279 | multiple coagulation factor deficiency 2 |
| MCL1 | NM_021960 | myeloid cell leukemia sequence 1 isoform 1 |
| MCM4 | NM_005914 | minichromosome maintenance protein 4 |
| MCM8 | NM_032485 | minichromosome maintenance protein 8 isoform 1 |
| MDFIC | NM_199072 | MyoD family inhibitor domain containing isoform |
| MDGA1 | NM_153487 | MAM domain containing |
| MECP2 | NM_004992 | methyl CpG binding protein 2 |
| MED12L | NM_053002 | mediator of RNA polymerase II transcription, |
| MEF2C | NM_002397 | MADS box transcription enhancer factor 2, |
| MEF2D | NM_005920 | MADS box transcription enhancer factor 2, |
| MEGF10 | NM_032446 | MEGF10 protein |
| MEP1A | NM_005588 | meprin A, alpha (PABA peptide hydrolase) |
| METT5D1 | NM_152636 | methyltransferase 5 domain containing 1 |
| METTL5 | NM_014168 | methyltransferase like 5 |
| MFAP3 | NM_005927 | microfibrillar-associated protein 3 |
| MFI2 | NM_033316 | melanoma-associated antigen p97 isoform 2, |
| MFN2 | NM_014874 | mitofusin 2 |
| MFSD4 | NM_181644 | hypothetical protein DKFZp761N1114 |
| MGAM | NM_004668 | maltase-glucoamylase |
| MGC10334 | NM_001029885 | hypothetical protein LOC80772 |
| MGC11102 | NM_032325 | hypothetical protein LOC84285 |
| MGC13379 | NM_016499 | hypothetical protein LOC51259 |
| MGC15875 | NM_032921 | hypothetical protein LOC85007 isoform 1 |
| MGC16028 | NM_052873 | hypothetical protein LOC112752 |
| MGC16703 | NM_145042 | hypothetical protein LOC113691 |
| MGC20470 | NM_145053 | hypothetical protein LOC143630 |
| MGC23280 | NM_144683 | hypothetical protein LOC147015 |
| MGC24039 | NM_144973 | hypothetical protein LOC160518 |
| MGC26694 | NM_178526 | hypothetical protein LOC284439 |
| MGC26718 | NM_001029999 | hypothetical protein LOC440482 |
| MGC26733 | NM_144992 | hypothetical protein LOC200403 |
| MGC27121 | NM_001001343 | hypothetical protein LOC408263 |
| MGC2752 | NM_023939 | hypothetical protein LOC65996 |
| MGC29891 | NM_144618 | GA repeat binding protein, beta 2 |
| MGC29898 | NM_145048 | hypothetical protein LOC133015 |
| MGC3207 | NM_001031727 | hypothetical protein LOC84245 isoform 1 |
| MGC33214 | NM_153354 | hypothetical protein LOC153396 |
| MGC33530 | NM_182546 | hypothetical protein LOC222008 |
| MGC34646 | NM_173519 | hypothetical protein LOC157807 |
| MGC35295 | NM_152717 | hypothetical protein LOC219995 |
| MGC39900 | NM_194324 | hypothetical protein LOC286527 |
| MGC4562 | NM_133375 | hypothetical protein LOC115752 |
| MGC4655 | NM_033309 | hypothetical protein LOC84752 |
| MGC50273 | NM_214461 | hypothetical protein LOC408029 |
| MGC9712 | NM_152689 | hypothetical protein LOC202915 |
| MGLL | NM_001003794 | monoglyceride lipase isoform 2 |
| MIB1 | NM_020774 | mindbomb homolog 1 |
| MICAL2 | NM_014632 | microtubule associated monoxygenase, calponin |
| MICAL-L1 | NM_033386 | molecule interacting with Rab13 |
| MID1IP1 | NM_021242 | MID1 interacting G12-like protein |
| MIER3 | NM_152622 | hypothetical protein LOC166968 |
| MIPOL1 | NM_138731 | mirror-image polydactyly 1 |
| MKL1 | NM_020831 | megakaryoblastic leukemia 1 protein |
| MKL2 | NM_014048 | megakaryoblastic leukemia 2 protein |
| MKLN1 | NM_013255 | muskelin 1, intracellular mediator containing |
| MKRN3 | NM_005664 | makorin, ring finger protein, 3 |
| MLC1 | NM_015166 | megalencephalic leukoencephalopathy with |
| MLL4 | NM_014727 | myeloid/lymphoid or mixed-lineage leukemia 4 |
| MLLT3 | NM_004529 | myeloid/lymphoid or mixed-lineage leukemia |
| MLSTD2 | NM_032228 | male sterility domain containing 2 |
| MLX | NM_170607 | transcription factor-like protein 4 isoform |
| MLXIPL | NM_032951 | Williams Beuren syndrome chromosome region 14 |
| MMD2 | NM_198403 | monocyte-to-macrophage differentiation factor 2 |
| MMP14 | NM_004995 | matrix metalloproteinase 14 preproprotein |
| MMP17 | NM_016155 | matrix metalloproteinase 17 preproprotein |
| MMP19 | NM_001032360 | matrix metalloproteinase 19 isoform 2 precursor |
| MMP2 | NM_004530 | matrix metalloproteinase 2 preproprotein |
| MMP8 | NM_002424 | matrix metalloproteinase 8 preproprotein |
| MN1 | NM_002430 | meningioma 1 |
| MOBKL2A | NM_130807 | MOB-LAK |
| MOBKL2B | NM_024761 | MOB1, Mps One Binder kinase activator-like 2B |
| MOCS1 | NM_005942 | molybdenum cofactor synthesis-step 1 protein |
| MOCS2 | NM_176806 | molybdopterin synthase small subunit MOCS2A |
| MOG | NM_001008228 | myelin oligodendrocyte glycoprotein isoform |
| MON1B | NM_014940 | MON1 homolog B |
| MOSPD1 | NM_019556 | motile sperm domain containing 1 |
| MPP2 | NM_005374 | palmitoylated membrane protein 2 |
| MPPED1 | NM_001585 | hypothetical protein LOC758 |
| MPST | NM_001013436 | 3-mercaptopyruvate sulfurtransferase |
| MRAS | NM_012219 | muscle RAS oncogene homolog |
| MRO | NM_031939 | maestro |
| MRP63 | NM_024026 | mitochondrial ribosomal protein 63 |
| MRPL30 | NM_145212 | mitochondrial ribosomal protein L30 |
| MRPL41 | NM_032477 | mitochondrial ribosomal protein L41 |
| MRPL52 | NM_178336 | mitochondrial ribosomal protein L52 isoform a |
| MRPS11 | NM_022839 | mitochondrial ribosomal protein S11 isoform a |
| MRPS26 | NM_030811 | mitochondrial ribosomal protein S26 |
| MRPS33 | NM_016071 | mitochondrial ribosomal protein S33 |
| MS4A10 | NM_206893 | membrane-spanning 4-domains, subfamily A, member |
| MS4A2 | NM_000139 | membrane-spanning 4-domains, subfamily A, member |
| MS4A4A | NM_024021 | membrane-spanning 4-domains, subfamily A, member |
| MS4A7 | NM_021201 | membrane-spanning 4-domains, subfamily A, member |
| MSH3 | NM_002439 | mutS homolog 3 |
| MSI2 | NM_138962 | musashi 2 isoform a |
| MSL3L1 | NM_078628 | male-specific lethal 3-like 1 isoform d |
| MSR1 | NM_002445 | macrophage scavenger receptor 1 isoform type 2 |
| MSRB3 | NM_001031679 | methionine sulfoxide reductase B3 isoform 2 |
| MTAC2D1 | NM_152332 | membrane targeting (tandem) C2 domain containing |
| MTHFR | NM_005957 | 5,10-methylenetetrahydrofolate reductase |
| MTHFSD | NM_022764 | hypothetical protein LOC64779 |
| MTM1 | NM_000252 | myotubularin |
| MTMR12 | NM_019061 | myotubularin related protein 12 |
| MTMR2 | NM_016156 | myotubularin-related protein 2 isoform 1 |
| MTMR3 | NM_021090 | myotubularin-related protein 3 isoform c |
| MTMR9 | NM_015458 | myotubularin-related protein 9 |
| MTPN | NM_145808 | myotrophin |
| MTRR | NM_002454 | methionine synthase reductase isoform 1 |
| MUCDHL | NM_031265 | mu-protocadherin isoform 4 |
| MUM1L1 | NM_152423 | melanoma associated antigen (mutated) 1-like 1 |
| MUTED | NM_201280 | muted |
| MX2 | NM_002463 | myxovirus resistance protein 2 |
| MXD1 | NM_002357 | MAX dimerization protein 1 |
| MXD4 | NM_006454 | MAD4 |
| MYADM | NM_001020818 | myeloid-associated differentiation marker |
| MYBBP1A | NM_014520 | MYB binding protein 1a |
| MYBL2 | NM_002466 | MYB-related protein B |
| MYCL1 | NM_001033081 | 1-myc-1 proto-oncogene isoform 1 |
| MYD88 | NM_002468 | myeloid differentiation primary response gene |
| MYL2 | NM_000432 | myosin light chain 2 |
| MYL3 | NM_000258 | myosin light chain 3 |
| MYO18A | NM_078471 | myosin 18A isoform a |
| MYO1B | NM_012223 | myosin IB |
| MYO1E | NM_004998 | myosin IE |
| MYO3A | NM_017433 | myosin IIIA |
| MYO5C | NM_018728 | myosin VC |
| MYO6 | NM_004999 | myosin VI |
| MYO7A | NM_000260 | myosin VIIA |
| MYOM2 | NM_003970 | myomesin 2 |
| MYST2 | NM_007067 | MYST histone acetyltransferase 2 |
| MYST3 | NM_006766 | MYST histone acetyltransferase (monocytic |
| MYT1L | NM_015025 | myelin transcription factor 1-like |
| N4BP1 | NM_153029 | Nedd4 binding protein 1 |
| NAALADL2 | NM_207015 | N-acetylated alpha-linked acidic dipeptidase 2 |
| NAG6 | NM_022742 | hypothetical protein DKFZp434G156 |
| NAG8 | NM_014411 | nasopharyngeal carcinoma associated gene |
| NALP1 | NM_014922 | death effector filament-forming Ced-4-like |
| NALP12 | NM_144687 | PYRIN-containing APAF1-like protein 7 isoform 2 |
| NANOS1 | NM_199461 | nanos homolog 1 isoform 1 |
| NANP | NM_152667 | haloacid dehalogenase-like hydrolase domain |
| NAP1L4 | NM_005969 | nucleosome assembly protein 1-like 4 |
| NAPE-PLD | NM_198990 | N-acyl-phosphatidylethanolamine-hydrolyzing |
| NARG1 | NM_057175 | NMDA receptor regulated 1 |
| NARG1L | NM_024561 | NMDA receptor regulated 1-like protein isoform |
| NARG2 | NM_001018089 | NMDA receptor regulated 2 isoform b |
| NAT10 | NM_024662 | N-acetyltransferase-like protein |
| NAT12 | NM_001011713 | hypothetical protein LOC122830 |
| NAV3 | NM_014903 | neuron navigator 3 |
| NCAM1 | NM_181351 | neural cell adhesion molecule 1 isoform 2 |
| NCOA1 | NM_003743 | nuclear receptor coactivator 1 isoform 1 |
| NCOA6IP | NM_024831 | PRIP-interacting protein PIPMT |
| NCOA7 | NM_181782 | nuclear receptor coactivator 7 |
| NCR1 | NM_004829 | natural cytotoxicity triggering receptor 1 |
| NCSTN | NM_015331 | nicastrin precursor |
| NDE1 | NM_017668 | nuclear distribution gene E homolog 1 |
| NDEL1 | NM_001025579 | nudE nuclear distribution gene E homolog like 1 |
| NDFIP1 | NM_030571 | Nedd4 family interacting protein 1 |
| NDRG4 | NM_020465 | NDRG family member 4 |
| NDST1 | NM_001543 | N-deacetylase/N-sulfotransferase (heparan |
| NEBL | NM_006393 | nebulette sarcomeric isoform |
| NECAP1 | NM_015509 | adaptin-ear-binding coat-associated protein 1 |
| NECAP2 | NM_018090 | adaptin-ear-binding coat-associated protein 2 |
| NEDD4 | NM_006154 | neural precursor cell expressed, developmentally |
| NEDD9 | NM_182966 | neural precursor cell expressed, developmentally |
| NEIL2 | NM_145043 | nei-like 2 |
| NEK8 | NM_178170 | NIMA-related kinase 8 |
| NES | NM_006617 | nestin |
| NETO1 | NM_138999 | neuropilin- and tolloid-like protein 1 isoform 1 |
| NETO2 | NM_018092 | neuropilin- and tolloid-like protein 2 |
| NEURL | NM_004210 | neuralized-like |
| NEUROG2 | NM_024019 | neurogenin 2 |
| NF2 | NM_000268 | neurofibromin 2 isoform 1 |
| NFAM1 | NM_145912 | NFAT activation molecule 1 precursor |
| NFASC | NM_015090 | neurofascin precursor |
| NFAT5 | NM_006599 | nuclear factor of activated T-cells 5 isoform c |
| NFATC1 | NM_006162 | nuclear factor of activated T-cells, cytosolic |
| NFIC | NM_005597 | nuclear factor I/C isoform 1 |
| NFKBIL1 | NM_005007 | nuclear factor of kappa light polypeptide gene |
| NFXL1 | NM_152995 | nuclear transcription factor, X-box binding-like |
| NFYA | NM_002505 | nuclear transcription factor Y, alpha isoform 1 |
| NFYB | NM_006166 | nuclear transcription factor Y, beta |
| NGFR | NM_002507 | nerve growth factor receptor precursor |
| NHLH1 | NM_005598 | nescient helix loop helix 1 |
| NIPA1 | NM_144599 | non-imprinted in Prader-Willi/Angelman syndrome |
| NIPSNAP1 | NM_003634 | nipsnap homolog 1 |
| NKIRAS2 | NM_001001349 | NFKB inhibitor interacting Ras-like 2 |
| NKTR | NM_001012651 | natural killer-tumor recognition sequence |
| NLGN2 | NM_020795 | neuroligin 2 |
| NMNAT1 | NM_022787 | nicotinamide nucleotide adenylyltransferase 1 |
| NMT1 | NM_021079 | N-myristoyltransferase 1 |
| NMT2 | NM_004808 | glycylpeptide N-tetradecanoyltransferase 2 |
| NNAT | NM_005386 | neuronatin isoform alpha |
| NOB1 | NM_014062 | nin one binding protein |
| NOL11 | NM_015462 | nucleolar protein 11 |
| NOL6 | NM_022917 | nucleolar RNA-associated protein alpha isoform |
| NOM1 | NM_138400 | nucleolar protein with MIF4G domain 1 |
| NOVA1 | NM_002515 | neuro-oncological ventral antigen 1 isoform 1 |
| NOX1 | NM_007052 | NADPH oxidase 1 isoform long |
| NPAL3 | NM_020448 | NIPA-like domain containing 3 |
| NPAS2 | NM_002518 | neuronal PAS domain protein 2 |
| NPC1 | NM_000271 | Niemann-Pick disease, type C1 |
| NPHP1 | NM_000272 | nephrocystin isoform 1 |
| NPLOC4 | NM_017921 | nuclear protein localization 4 |
| NPR3 | NM_000908 | natriuretic peptide receptor C/guanylate cyclase |
| NPTX1 | NM_002522 | neuronal pentraxin I precursor |
| NPTXR | NM_014293 | neuronal pentraxin receptor isoform 1 |
| NQO1 | NM_000903 | NAD(P)H menadione oxidoreductase 1, |
| NR3C1 | NM_000176 | nuclear receptor subfamily 3, group C, member 1 |
| NRG1 | NM_013958 | neuregulin 1 isoform HRG-beta3 |
| NRIP1 | NM_003489 | receptor interacting protein 140 |
| NRIP2 | NM_031474 | nuclear receptor interacting protein 2 |
| NRP2 | NM_003872 | neuropilin 2 isoform 2 precursor |
| NSF | NM_006178 | N-ethylmaleimide-sensitive factor |
| NT5C2 | NM_012229 | 5′-nucleotidase, cytosolic II |
| NTRK2 | NM_001007097 | neurotrophic tyrosine kinase, receptor, type 2 |
| NUAK2 | NM_030952 | NUAK family, SNF1-like kinase, 2 |
| NUCB1 | NM_006184 | nucleobindin 1 |
| NUDT10 | NM_153183 | nudix-type motif 10 |
| NUDT12 | NM_031438 | nudix-type motif 12 |
| NUDT15 | NM_018283 | nudix-type motif 15 |
| NUDT16 | NM_152395 | nudix-type motif 16 |
| NUDT16L1 | NM_032349 | syndesmos |
| NUDT18 | NM_024815 | nudix (nucleoside diphosphate linked moiety |
| NUDT4 | NM_019094 | nudix-type motif 4 isoform alpha |
| NUMB | NM_001005743 | numb homolog isoform 1 |
| NUMBL | NM_004756 | numb homolog (Drosophila)-like |
| NUP35 | NM_001008544 | nucleoporin 35 kDa isoform b |
| NUP43 | NM_198887 | nucleoporin 43 kDa |
| NXF1 | NM_006362 | nuclear RNA export factor 1 |
| NYD-SP18 | NM_032599 | testes development-related NYD-SP18 |
| NY-REN-7 | NM_173663 | hypothetical protein LOC285596 |
| OACT2 | NM_138799 | O-acyltransferase (membrane bound) domain |
| OACT5 | NM_005768 | gene rich cluster, C3f gene |
| OAF | NM_178507 | hypothetical protein LOC220323 |
| OAS3 | NM_006187 | 2′-5′oligoadenylate synthetase 3 |
| OAZ1 | NM_004152 | ornithine decarboxylase antizyme 1 |
| OBFC2B | NM_024068 | hypothetical protein LOC79035 |
| OCRL | NM_000276 | phosphatidylinositol polyphosphate 5-phosphatase |
| OLIG1 | NM_138983 | oligodendrocyte transcription factor 1 |
| OPCML | NM_001012393 | opioid binding protein/cell adhesion |
| OPRD1 | NM_000911 | opioid receptor, delta 1 |
| OPTC | NM_014359 | opticin precursor |
| OR2H1 | NM_030883 | olfactory receptor, family 2, subfamily H, |
| OR51E2 | NM_030774 | olfactory receptor, family 51, subfamily E, |
| OR7D2 | NM_175883 | hypothetical protein LOC162998 |
| ORAOV1 | NM_153451 | oral cancer overexpressed 1 |
| ORC2L | NM_006190 | origin recognition complex, subunit 2 |
| OSBP2 | NM_030758 | oxysterol binding protein 2 isoform a |
| OSBPL2 | NM_014835 | oxysterol-binding protein-like protein 2 isoform |
| OSBPL3 | NM_015550 | oxysterol-binding protein-like protein 3 isoform |
| OSBPL7 | NM_145798 | oxysterol-binding protein-like protein 7 |
| OSCAR | NM_206817 | osteoclast-associated receptor isoform 2 |
| OTUD4 | NM_199324 | OTU domain containing 4 protein isoform 1 |
| OTUD6B | NM_016023 | OTU domain containing 6B |
| OXGR1 | NM_080818 | oxoglutarate (alpha-ketoglutarate) receptor 1 |
| P2RX2 | NM_012226 | purinergic receptor P2X2 isoform I |
| P2RX7 | NM_002562 | purinergic receptor P2X7 |
| P2RY13 | NM_023914 | purinergic receptor P2Y, G-protein coupled, 13 |
| P2RY14 | NM_014879 | purinergic receptor P2Y, G-protein coupled, 14 |
| P2RY4 | NM_002565 | pyrimidinergic receptor P2Y4 |
| P2RY8 | NM_178129 | G-protein coupled purinergic receptor P2Y8 |
| P4HA1 | NM_000917 | prolyl 4-hydroxylase, alpha I subunit isoform 1 |
| P4HA3 | NM_182904 | prolyl 4-hydroxylase, alpha III subunit |
| P53AIP1 | NM_022112 | p53-regulated apoptosis-inducing protein 1 |
| PACRG | NM_152410 | PARK2 co-regulated |
| PACS1 | NM_018026 | phosphofurin acidic cluster sorting protein 1 |
| PAFAH1B2 | NM_002572 | platelet-activating factor acetylhydrolase, |
| PAG1 | NM_018440 | phosphoprotein associated with glycosphingolipid |
| PAICS | NM_006452 | phosphoribosylaminoimidazole carboxylase |
| PALMD | NM_017734 | palmdelphin |
| PAN3 | NM_175854 | PABP1-dependent poly A-specific ribonuclease |
| PAP2D | NM_001010861 | phosphatidic acid phosphatase type 2d isoform 2 |
| PAPLN | NM_173462 | papilin |
| PAPOLB | NM_020144 | poly(A) polymerase beta (testis specific) |
| PAPPA | NM_002581 | pregnancy-associated plasma protein A |
| PAQR5 | NM_017705 | membrane progestin receptor gamma |
| PAQR6 | NM_198406 | progestin and adipoQ receptor family member VI |
| PARD6G | NM_032510 | PAR-6 gamma protein |
| PARP6 | NM_020213 | poly (ADP-ribose) polymerase family, member 6 |
| PARVA | NM_018222 | parvin, alpha |
| PATE | NM_138294 | expressed in prostate and testis |
| PAX5 | NM_016734 | paired box 5 |
| PBK | NM_018492 | T-LAK cell-originated protein kinase |
| PC | NM_000920 | pyruvate carboxylase precursor |
| PCDH11X | NM_032967 | protocadherin 11 X-linked isoform b precursor |
| PCDH11Y | NM_032971 | protocadherin 11 Y-linked isoform a |
| PCDH21 | NM_033100 | protocadherin 21 precursor |
| PCDHA9 | NM_014005 | protocadherin alpha 9 isoform 2 precursor |
| PCDHB10 | NM_018930 | protocadherin beta 10 precursor |
| PCGF3 | NM_006315 | ring finger protein 3 |
| PCGF6 | NM_001011663 | polycomb group ring finger 6 isoform a |
| PCMT1 | NM_005389 | protein-L-isoaspartate (D-aspartate) |
| PCNXL2 | NM_014801 | pecanex-like 2 |
| PCQAP | NM_001003891 | positive cofactor 2, glutamine/Q-rich-associated |
| PCSK2 | NM_002594 | proprotein convertase subtilisin/kexin type 2 |
| PCSK6 | NM_138323 | paired basic amino acid cleaving system 4 |
| PCSK7 | NM_004716 | proprotein convertase subtilisin/kexin type 7 |
| PCSK9 | NM_174936 | proprotein convertase subtilisin/kexin type 9 |
| PCYOX1 | NM_016297 | prenylcysteine oxidase 1 |
| PDAP1 | NM_014891 | PDGFA associated protein 1 |
| PDCD6IP | NM_013374 | programmed cell death 6 interacting protein |
| PDCL | NM_005388 | phosducin-like |
| PDDC1 | NM_182612 | hypothetical protein LOC347862 |
| PDE11A | NM_016953 | phosphodiesterase 11A |
| PDE1B | NM_000924 | phosphodiesterase 1B, calmodulin-dependent |
| PDE4DIP | NM_001002811 | phosphodiesterase 4D interacting protein isoform |
| PDE5A | NM_001083 | phosphodiesterase 5A isoform 1 |
| PDE7A | NM_002604 | phosphodiesterase 7A isoform b |
| PDE8B | NM_001029851 | phosphodiesterase 8B isoform 3 |
| PDGFB | NM_002608 | platelet-derived growth factor beta isoform 1, |
| PDGFRA | NM_006206 | platelet-derived growth factor receptor alpha |
| PDGFRB | NM_002609 | platelet-derived growth factor receptor beta |
| PDIA6 | NM_005742 | protein disulfide isomerase-associated 6 |
| PDK1 | NM_002610 | pyruvate dehydrogenase kinase, isozyme 1 |
| PDLIM2 | NM_176871 | PDZ and LIM domain 2 isoform 1 |
| PDLIM5 | NM_001011513 | PDZ and LIM domain 5 isoform b |
| PDP2 | NM_020786 | pyruvate dehydrogenase phosphatase isoenzyme 2 |
| PDPK1 | NM_002613 | 3-phosphoinositide dependent protein kinase-1 |
| PDPR | NM_017990 | pyruvate dehydrogenase phosphatase regulatory |
| PDXK | NM_003681 | pyridoxal kinase |
| PDYN | NM_024411 | beta-neoendorphin-dynorphin preproprotein |
| PDZD2 | NM_178140 | PDZ domain containing 2 |
| PDZD4 | NM_032512 | PDZ domain containing 4 |
| PEBP1 | NM_002567 | prostatic binding protein |
| PECR | NM_018441 | peroxisomal trans-2-enoyl-CoA reductase |
| PEG3 | NM_006210 | paternally expressed 3 |
| PER2 | NM_022817 | period 2 isoform 1 |
| PEX10 | NM_002617 | peroxisome biogenesis factor 10 isoform 2 |
| PEX5 | NM_000319 | peroxisomal biogenesis factor 5 |
| PFKFB2 | NM_001018053 | 6-phosphofructo-2-kinase/fructose-2, |
| PGAP1 | NM_024989 | GPI deacylase |
| PGBD4 | NM_152595 | piggyBac transposable element derived 4 |
| PGD | NM_002631 | phosphogluconate dehydrogenase |
| PGK1 | NM_000291 | phosphoglycerate kinase 1 |
| PGK2 | NM_138733 | phosphoglycerate kinase 2 |
| PGLYRP2 | NM_052890 | peptidoglycan recognition protein L precursor |
| PGLYRP4 | NM_020393 | peptidoglycan recognition protein-I-beta |
| PGM2L1 | NM_173582 | phosphoglucomutase 2-like 1 |
| PGRMC2 | NM_006320 | progesterone membrane binding protein |
| PHC2 | NM_004427 | polyhomeotic 2-like isoform b |
| PHF11 | NM_016119 | PHD finger protein 11 |
| PHF13 | NM_153812 | PHD finger protein 13 |
| PHF20 | NM_016436 | PHD finger protein 20 |
| PHF20L1 | NM_016018 | PHD finger protein 20-like 1 isoform 1 |
| PHF6 | NM_001015877 | PHD finger protein 6 isoform 1 |
| PHF8 | NM_015107 | PHD finger protein 8 |
| PHGDHL1 | NM_177967 | hypothetical protein LOC337867 |
| PHLDB1 | NM_015157 | pleckstrin homology-like domain, family B, |
| PHTF2 | NM_020432 | putative homeodomain transcription factor 2 |
| PI4KII | NM_018425 | phosphatidylinositol 4-kinase type II |
| PIAS3 | NM_006099 | protein inhibitor of activated STAT, 3 |
| PIGQ | NM_004204 | phosphatidylinositol glycan, class Q isoform 2 |
| PIGW | NM_178517 | phosphatidylinositol glycan, class W |
| PIK3CG | NM_002649 | phosphoinositide-3-kinase, catalytic, gamma |
| PIK3R1 | NM_181504 | phosphoinositide-3-kinase, regulatory subunit, |
| PIK3R3 | NM_003629 | phosphoinositide-3-kinase, regulatory subunit 3 |
| PILRA | NM_013439 | paired immunoglobulin-like type 2 receptor alpha |
| PIP3-E | NM_015553 | phosphoinositide-binding protein PIP3-E |
| PIP5K1C | NM_012398 | phosphatidylinositol-4-phosphate 5-kinase, type |
| PIP5K2B | NM_003559 | phosphatidylinositol-4-phosphate 5-kinase type |
| PIP5KL1 | NM_173492 | phosphatidylinositol-4-phosphate 5-kinase-like |
| PITPNA | NM_006224 | phosphatidylinositol transfer protein, alpha |
| PITX1 | NM_002653 | paired-like homeodomain transcription factor 1 |
| PKD2 | NM_000297 | polycystin 2 |
| PKNOX1 | NM_004571 | PBX/knotted 1 homeobox 1 isoform 1 |
| PKP1 | NM_000299 | plakophilin 1 isoform 1b |
| PLA2G1B | NM_000928 | phospholipase A2, group IB |
| PLA2G2D | NM_012400 | phospholipase A2, group IID |
| PLA2G4D | NM_178034 | phospholipase A2, group IVD |
| PLAGL2 | NM_002657 | pleiomorphic adenoma gene-like 2 |
| PLAU | NM_002658 | urokinase plasminogen activator preproprotein |
| PLAUR | NM_001005376 | plasminogen activator, urokinase receptor |
| PLCB4 | NM_000933 | phospholipase C beta 4 isoform a |
| PLCD3 | NM_133373 | phospholipase C delta 3 |
| PLCXD3 | NM_001005473 | phosphatidylinositol-specific phospholipase C, X |
| PLD5 | NM_152666 | phospholipase D family, member 5 |
| PLEKHA1 | NM_001001974 | pleckstrin homology domain containing, family A |
| PLEKHA6 | NM_014935 | phosphoinositol 3-phosphate-binding protein-3 |
| PLEKHB2 | NM_017958 | pleckstrin homology domain containing, family B |
| PLEKHQ1 | NM_025201 | PH domain-containing protein |
| PLIN | NM_002666 | perilipin |
| PLXNA1 | NM_032242 | plexin A1 |
| PML | NM_033238 | promyelocytic leukemia protein isoform 1 |
| PNKD | NM_015488 | myofibrillogenesis regulator 1 isoform 1 |
| PNMA2 | NM_007257 | paraneoplastic antigen MA2 |
| PNPLA1 | NM_173676 | patatin-like phospholipase domain containing 1 |
| PNPO | NM_018129 | pyridoxine 5′-phosphate oxidase |
| PNRC1 | NM_006813 | proline-rich nuclear receptor coactivator 1 |
| PODXL | NM_001018111 | podocalyxin-like precursor isoform 1 |
| POFUT1 | NM_015352 | protein O-fucosyltransferase 1 isoform 1 |
| POFUT2 | NM_015227 | protein O-fucosyltransferase 2 isoform A |
| POGK | NM_017542 | pogo transposable element with KRAB domain |
| POGZ | NM_145796 | pogo transposable element with ZNF domain |
| POLDIP2 | NM_015584 | DNA polymerase delta interacting protein 2 |
| POLDIP3 | NM_032311 | DNA polymerase delta interacting protein 3 |
| POLH | NM_006502 | polymerase (DNA directed), eta |
| POLR1B | NM_019014 | RNA polymerase I polypeptide B |
| POLR1E | NM_022490 | RNA polymerase I associated factor 53 |
| POLR2L | NM_021128 | DNA directed RNA polymerase II polypeptide L |
| POLR3E | NM_018119 | polymerase (RNA) III (DNA directed) polypeptide |
| POLR3GL | NM_032305 | polymerase (RNA) III (DNA directed) polypeptide |
| POM121 | NM_172020 | nuclear pore membrane protein 121 |
| POU2F2 | NM_002698 | POU domain, class 2, transcription factor 2 |
| POU2F3 | NM_014352 | POU transcription factor |
| PPAPDC2 | NM_203453 | phosphatidic acid phosphatase type 2 domain |
| PPARA | NM_001001928 | peroxisome proliferative activated receptor, |
| PPCDC | NM_021823 | phosphopantothenoylcysteine decarboxylase |
| PPEF2 | NM_152933 | serine/threonine protein phosphatase with |
| PPFIBP2 | NM_003621 | PTPRF interacting protein, binding protein 2 |
| PPIL2 | NM_014337 | peptidylprolyl isomerase-like 2 isoform a |
| PPIL4 | NM_139126 | peptidylprolyl isomerase-like 4 |
| PPL | NM_002705 | periplakin |
| PPM1B | NM_177968 | protein phosphatase 1B isoform 2 |
| PPM1E | NM_014906 | protein phosphatase 1E |
| PPM2C | NM_018444 | pyruvate dehydrogenase phosphatase precursor |
| PPP1R12B | NM_002481 | protein phosphatase 1, regulatory (inhibitor) |
| PPP1R12C | NM_017607 | protein phosphatase 1, regulatory subunit 12C |
| PPP1R13L | NM_006663 | protein phosphatase 1, regulatory (inhibitor) |
| PPP1R15B | NM_032833 | protein phosphatase 1, regulatory subunit 15B |
| PPP1R16B | NM_015568 | protein phosphatase 1 regulatory inhibitor |
| PPP1R3A | NM_002711 | protein phosphatase 1 glycogen-binding |
| PPP1R3B | NM_024607 | protein phosphatase 1, regulatory (inhibitor) |
| PPP2CB | NM_001009552 | protein phosphatase 2, catalytic subunit, beta |
| PPP2R1B | NM_002716 | beta isoform of regulatory subunit A, protein |
| PPP2R2A | NM_002717 | alpha isoform of regulatory subunit B55, protein |
| PPP2R3A | NM_002718 | protein phosphatase 2, regulatory subunit B″, |
| PPP2R4 | NM_021131 | protein phosphatase 2A, regulatory subunit B′ |
| PPP2R5C | NM_002719 | gamma isoform of regulatory subunit B56, protein |
| PPP4R1L | NM_018498 | hypothetical protein LOC55370 |
| PPT2 | NM_005155 | palmitoyl-protein thioesterase 2 isoform a |
| PRC1 | NM_003981 | protein regulator of cytokinesis 1 isoform 1 |
| PRDM12 | NM_021619 | PR domain containing 12 |
| PRDM16 | NM_022114 | PR domain containing 16 isoform 1 |
| PRDM9 | NM_020227 | PR domain containing 9 |
| PREB | NM_013388 | prolactin regulatory element binding protein |
| PRELP | NM_002725 | proline arginine-rich end leucine-rich repeat |
| PREPL | NM_006036 | prolyl endopeptidase-like |
| PRICKLE2 | NM_198859 | prickle-like 2 |
| PRKAA2 | NM_006252 | AMP-activated protein kinase alpha 2 catalytic |
| PRKCA | NM_002737 | protein kinase C, alpha |
| PRKCE | NM_005400 | protein kinase C, epsilon |
| PRKD2 | NM_016457 | protein kinase D2 |
| PRKRIP1 | NM_024653 | PRKR interacting protein 1 (IL11 inducible) |
| PRKRIR | NM_004705 | protein-kinase, interferon-inducible double |
| PRKX | NM_005044 | protein kinase, X-linked |
| PRKY | NM_002760 | protein kinase, Y-linked |
| PRND | NM_012409 | prion-like protein doppel preproprotein |
| PROSC | NM_007198 | proline synthetase co-transcribed homolog |
| PRPF19 | NM_014502 | PRP19/PSO4 pre-mRNA processing factor 19 |
| PRPF4 | NM_004697 | PRP4 pre-mRNA processing factor 4 homolog |
| PRRG4 | NM_024081 | proline rich Gla (G-carboxyglutamic acid) 4 |
| PRRT2 | NM_145239 | hypothetical protein LOC112476 |
| PRRX1 | NM_006902 | paired mesoderm homeobox 1 isoform pmx-1a |
| PRSS23 | NM_007173 | protease, serine, 23 precursor |
| PRX | NM_020956 | periaxin isoform 1 |
| PRY | NM_004676 | PTPN13-lilce, Y-linked |
| PRY2 | NM_001002758 | PTPN13-like, Y-linked 2 |
| PSCD1 | NM_004762 | pleckstrin homology, Sec7 and coiled/coil |
| PSCD4 | NM_013385 | pleckstrin homology, Sec7 and coiled/coil |
| PSD3 | NM_015310 | ADP-ribosylation factor guanine nucleotide |
| PSG4 | NM_002780 | pregnancy specific beta-1-glycoprotein 4 isoform |
| PSG7 | NM_002783 | pregnancy specific beta-1-glycoprotein 7 |
| PSMD5 | NM_005047 | proteasome 26S non-ATPase subunit 5 |
| PSME4 | NM_014614 | proteasome (prosome, macropain) activator |
| PTAFR | NM_000952 | platelet-activating factor receptor |
| PTCH | NM_000264 | patched |
| PTD004 | NM_001011708 | GTP-binding protein PTD004 isoform 2 |
| PTDSS2 | NM_030783 | phosphatidylserine synthase 2 |
| PTGDR | NM_000953 | prostaglandin D2 receptor |
| PTGER3 | NM_198718 | prostaglandin E receptor 3, subtype EP3 isoform |
| PTGES2 | NM_025072 | prostaglandin E synthase 2 isoform 1 |
| PTGES3 | NM_006601 | unactive progesterone receptor, 23 kD |
| PTGIS | NM_000961 | prostaglandin I2 (prostacyclin) synthase |
| PTHB1 | NM_001033604 | parathyroid hormone-responsive B1 isoform 3 |
| PTK6 | NM_005975 | PTK6 protein tyrosine kinase 6 |
| PTK7 | NM_002821 | PTK7 protein tyrosine kinase 7 isoform a |
| PTK9L | NM_007284 | twinfilin-like protein |
| PTPDC1 | NM_152422 | protein tyrosine phosphatase domain containing 1 |
| PTPLB | NM_198402 | protein tyrosine phosphatase-like (proline |
| PTPN11 | NM_002834 | protein tyrosine phosphatase, non-receptor type |
| PTPN2 | NM_002828 | protein tyrosine phosphatase, non-receptor type |
| PTPN23 | NM_015466 | protein tyrosine phosphatase, non-receptor type |
| PTPN4 | NM_002830 | protein tyrosine phosphatase, non-receptor type |
| PTPN7 | NM_002832 | protein tyrosine phosphatase, non-receptor type |
| PTPRE | NM_006504 | protein tyrosine phosphatase, receptor type, E |
| PTPRN | NM_002846 | protein tyrosine phosphatase, receptor type, N |
| PTPRT | NM_007050 | protein tyrosine phosphatase, receptor type, T |
| PTRF | NM_012232 | polymerase I and transcript release factor |
| PTTG1IP | NM_004339 | pituitary tumor-transforming gene 1 |
| PXMP4 | NM_183397 | peroxisomal membrane protein 4 isoform b |
| PXT1 | NM_152990 | peroxisomal, testis specific 1 |
| PYCRL | NM_023078 | pyrroline-5-carboxylate reductase-like |
| QDPR | NM_000320 | quinoid dihydropteridine reductase |
| QKI | NM_006775 | quaking homolog, KH domain RNA binding isoform |
| QPCTL | NM_017659 | glutaminyl-peptide cyclotransferase-like |
| QPRT | NM_014298 | quinolinate phosphoribosyltransferase |
| QRSL1 | NM_018292 | glutaminyl-tRNA synthase |
| QSCN6 | NM_002826 | quiescin Q6 isoform a |
| QSCN6L1 | NM_181701 | quiescin Q6-like 1 |
| RAB11A | NM_004663 | Ras-related protein Rab-11A |
| RAB11FIP1 | NM_001002814 | Rab coupling protein isoform 3 |
| RAB11FIP4 | NM_032932 | RAB11 family interacting protein 4 (class II) |
| RAB15 | NM_198686 | Ras-related protein Rab-15 |
| RAB22A | NM_020673 | RAS-related protein RAB-22A |
| RAB23 | NM_016277 | Ras-related protein Rab-23 |
| RAB27A | NM_004580 | Ras-related protein Rab-27A |
| RAB28 | NM_001017979 | RAB28, member RAS oncogene family isoform 1 |
| RAB33B | NM_031296 | RAB33B, member RAS oncogene family |
| RAB37 | NM_001006638 | RAB37, member RAS oncogene family isoform 2 |
| RAB40B | NM_006822 | RAB40B, member RAS oncogene family |
| RAB40C | NM_021168 | RAR (RAS like GTPASE) like |
| RAB41 | NM_001032726 | RAB41, member RAS homolog family |
| RAB43 | NM_198490 | RAB43 protein |
| RAB6B | NM_016577 | RAB6B, member RAS oncogene family |
| RAB6IP2 | NM_015064 | RAB6-interacting protein 2 isoform alpha |
| RAB7 | NM_004637 | RAB7, member RAS oncogene family |
| RAB7L1 | NM_003929 | RAB7, member RAS oncogene family-like 1 |
| RABEP1 | NM_004703 | rabaptin, RAB GTPase binding effector protein 1 |
| RABIF | NM_002871 | RAB-interacting factor |
| RABL5 | NM_022777 | RAB, member RAS oncogene family-like 5 |
| RAD1 | NM_002853 | RAD1 homolog isoform 1 |
| RAD23B | NM_002874 | UV excision repair protein RAD23 homolog B |
| RAD51 | NM_002875 | RAD51 homolog protein isoform 1 |
| RAD51L3 | NM_002878 | RAD51-like 3 isoform 1 |
| RAE1 | NM_001015885 | RAE1 (RNA export 1, S. pombe) homolog |
| RAF1 | NM_002880 | v-raf-1 murine leukemia viral oncogene homolog |
| RAI17 | NM_020338 | retinoic acid induced 17 |
| RALBP1 | NM_006788 | ralA binding protein 1 |
| RALGPS1 | NM_014636 | Ral GEF with PH domain and SH3 binding motif 1 |
| RANBP10 | NM_020850 | RAN binding protein 10 |
| RAP2B | NM_002886 | RAP2B, member of RAS oncogene family |
| RAPGEF1 | NM_005312 | guanine nucleotide-releasing factor 2 isoform a |
| RAPGEF6 | NM_016340 | PDZ domain-containing guanine nucleotide |
| RARG | NM_000966 | retinoic acid receptor, gamma |
| RARRES1 | NM_206963 | retinoic acid receptor responder (tazarotene |
| RASD2 | NM_014310 | RASD family, member 2 |
| RASGEF1B | NM_152545 | RasGEF domain family, member 1B |
| RASGRP1 | NM_005739 | RAS guanyl releasing protein 1 |
| RASGRP4 | NM_052949 | RAS guanyl releasing protein 4 isoform 3 |
| RASL10B | NM_033315 | RAS-like, family 10, member B |
| RASSF2 | NM_014737 | Ras association domain family 2 |
| RASSF4 | NM_032023 | Ras association domain family 4 isoform a |
| RASSF5 | NM_031437 | Ras association (RalGDS/AF-6) domain family 5 |
| RASSF6 | NM_177532 | Ras association (RalGDS/AF-6) domain family 6 |
| RASSF8 | NM_007211 | Ras association (RalGDS/AF-6) domain family 8 |
| RAVER1 | NM_133452 | RAVER1 |
| RAXLX | NM_001008494 | hypothetical protein LOC91464 |
| RB1 | NM_000321 | retinoblastoma 1 |
| RBBP9 | NM_006606 | retinoblastoma binding protein 9 |
| RBL1 | NM_002895 | retinoblastoma-like protein 1 isoform a |
| RBM14 | NM_006328 | RNA binding motif protein 14 |
| RBM16 | NM_014892 | RNA-binding motif protein 16 |
| RBM17 | NM_032905 | RNA binding motif protein 17 |
| RBM19 | NM_016196 | RNA binding motif protein 19 |
| RBM24 | NM_153020 | hypothetical protein LOC221662 |
| RBM3 | NM_001017430 | RNA binding motif protein 3 isoform b |
| RBM33 | NM_001008408 | hypothetical protein LOC155435 |
| RBM5 | NM_005778 | RNA binding motif protein 5 |
| RCC2 | NM_018715 | RCC1-like |
| RCD-8 | NM_014329 | autoantigen RCD8 |
| RCHY1 | NM_001008925 | ring finger and CHY zinc finger domain |
| RDBP | NM_002904 | RD RNA-binding protein |
| RDH12 | NM_152443 | retinol dehydrogenase 12 (all-trans and 9-cis) |
| RECQL5 | NM_001003715 | RecQ protein-like 5 isoform 2 |
| REEP1 | NM_022912 | receptor expression enhancing protein 1 |
| REEP3 | NM_001001330 | receptor expression enhancing protein 3 |
| REG4 | NM_032044 | regenerating islet-derived family, member 4 |
| REPS1 | NM_031922 | RALBP1 associated Eps domain containing 1 |
| RER1 | NM_007033 | RER1 retention in endoplasmic reticulum 1 |
| RETNLB | NM_032579 | colon and small intestine-specific cysteine-rich |
| REXO1L1 | NM_172239 | exonuclease GOR |
| REXO2 | NM_015523 | small fragment nuclease |
| RFC3 | NM_181558 | replication factor C 3 isoform 2 |
| RFK | NM_018339 | riboflavin kinase |
| RFNG | NM_002917 | radical fringe homolog |
| RFWD3 | NM_018124 | ring finger and WD repeat domain 3 |
| RFX2 | NM_000635 | regulatory factor X2 isoform a |
| RG9MTD3 | NM_144964 | RNA (guanine-9-) methyltransferase domain |
| RGAG1 | NM_020769 | retrotransposon gag domain containing 1 |
| RGL1 | NM_015149 | ral guanine nucleotide dissociation |
| RGMB | NM_001012761 | RGM domain family, member B isoform 1 precursor |
| RGS11 | NM_003834 | regulator of G-protein signalling 11 isoform 2 |
| RGS12 | NM_198432 | regulator of G-protein signalling 12 isoform 5 |
| RGS18 | NM_130782 | regulator of G-protein signalling 18 |
| RGS3 | NM_017790 | regulator of G-protein signalling 3 isoform 3 |
| RGSL1 | NM_181572 | regulator of G-protein signalling like 1 |
| RHBDD1 | NM_032276 | rhomboid domain containing 1 |
| RHBDL3 | NM_138328 | rhomboid, veinlet-like 3 |
| RHCG | NM_016321 | Rhesus blood group, C glycoprotein |
| RHOBTB1 | NM_001032380 | Rho-related BTB domain containing 1 |
| RHOG | NM_001665 | ras homolog gene family, member G |
| RHOJ | NM_020663 | TC10-like Rho GTPase |
| RHOU | NM_021205 | ras homolog gene family, member U |
| RIC8A | NM_021932 | resistance to inhibitors of cholinesterase 8 |
| RICTOR | NM_152756 | rapamycin-insensitive companion of mTOR |
| RIMBP2 | NM_015347 | RIM-binding protein 2 |
| RIMS3 | NM_014747 | regulating synaptic membrane exocytosis 3 |
| RIN2 | NM_018993 | RAB5 interacting protein 2 |
| RIN3 | NM_024832 | Ras and Rab interactor 3 |
| RIPK5 | NM_015375 | receptor interacting protein kinase 5 isoform 1 |
| RKHD2 | NM_016626 | ring finger and KH domain containing 2 |
| RLN2 | NM_005059 | relaxin 2 isoform 2 |
| RMND5A | NM_022780 | hypothetical protein LOC64795 |
| RNASE7 | NM_032572 | ribonuclease 7 |
| RND2 | NM_005440 | Rho family GTPase 2 |
| RNF10 | NM_014868 | ring finger protein 10 |
| RNF11 | NM_014372 | ring finger protein 11 |
| RNF121 | NM_018320 | ring finger protein 121 isoform 1 |
| RNF125 | NM_017831 | ring finger protein 125 |
| RNF135 | NM_197939 | ring finger protein 135 isoform 2 |
| RNF138 | NM_016271 | ring finger protein 138 isoform 1 |
| RNF144 | NM_014746 | ring finger protein 144 |
| RNF165 | NM_152470 | ring finger protein 165 |
| RNF185 | NM_152267 | ring finger protein 185 |
| RNF2 | NM_007212 | ring finger protein 2 |
| RNF24 | NM_007219 | ring finger protein 24 |
| RNF26 | NM_032015 | ring finger protein 26 |
| RNF4 | NM_002938 | ring finger protein 4 |
| RNF40 | NM_014771 | ring finger protein 40 |
| RNF6 | NM_005977 | ring finger protein 6 isoform 1 |
| RNF8 | NM_003958 | ring finger protein 8 isoform 1 |
| RNGTT | NM_003800 | RNA guanylyltransferase and 5′-phosphatase |
| RNMT | NM_003799 | RNA (guanine-7-) methyltransferase |
| RNPC2 | NM_004902 | RNA-binding region containing protein 2 isoform |
| ROBO4 | NM_019055 | roundabout homolog 4, magic roundabout |
| ROD1 | NM_005156 | ROD1 regulator of differentiation 1 |
| RORC | NM_001001523 | RAR-related orphan receptor C isoform b |
| RP11-19J3.3 | NM_001012267 | hypothetical protein LOC401541 |
| RP11-311P8.3 | NM_145052 | hypothetical protein LOC139596 |
| RP13-15M17.2 | NM_001010866 | hypothetical protein LOC199953 |
| RPA1 | NM_002945 | replication protein A1, 70 kDa |
| RPL28 | NM_000991 | ribosomal protein L28 |
| RPL32 | NM_000994 | ribosomal protein L32 |
| RPL34 | NM_000995 | ribosomal protein L34 |
| RPL37 | NM_000997 | ribosomal protein L37 |
| RPL7L1 | NM_198486 | ribosomal protein L7-like 1 |
| RPLP2 | NM_001004 | ribosomal protein P2 |
| RPP25 | NM_017793 | ribonuclease P 25 kDa subunit |
| RPS27 | NM_001030 | ribosomal protein S27 |
| RPS6KA3 | NM_004586 | ribosomal protein S6 kinase, 90 kDa, polypeptide |
| RRAS2 | NM_012250 | related RAS viral (r-ras) oncogene homolog 2 |
| RRH | NM_006583 | peropsin |
| RRM2 | NM_001034 | ribonucleotide reductase M2 polypeptide |
| RRM2B | NM_015713 | ribonucleotide reductase M2 B (TP53 inducible) |
| RRP22 | NM_001007279 | RAS-related on chromosome 22 isoform b |
| RS1 | NM_000330 | X-linked juvenile retinoschisis protein |
| RSAD1 | NM_018346 | radical S-adenosyl methionine domain containing |
| RTEL1 | NM_032957 | regulator of telomere elongation helicase 1 |
| RTF1 | NM_015138 | Paf1/RNA polymerase II complex component |
| RTN2 | NM_206902 | reticulon 2 isoform D |
| RTN4RL1 | NM_178568 | reticulon 4 receptor-like 1 |
| RUNDC1 | NM_173079 | RUN domain containing 1 |
| RUNX3 | NM_001031680 | runt-related transcription factor 3 isoform 1 |
| RWDD4A | NM_152682 | RWD domain containing 4A |
| S100A11 | NM_005620 | S100 calcium binding protein A11 (calgizzarin) |
| S100A14 | NM_020672 | S100 calcium binding protein A14 |
| S100A7L1 | NM_176823 | S100 calcium binding protein A7-like 1 |
| S100PBP | NM_022753 | S100P binding protein Riken isoform a |
| SALL4 | NM_020436 | sal-like 4 |
| SAMD13 | NM_001010971 | dnaj-like protein |
| SAP130 | NM_024545 | mSin3A-associated protein 130 |
| SAP30BP | NM_013260 | transcriptional regulator protein |
| SARM1 | NM_015077 | sterile alpha and TIR motif containing 1 |
| SART1 | NM_005146 | squamous cell carcinoma antigen recognized by T |
| SASH1 | NM_015278 | SAM and SH3 domain containing 1 |
| SATL1 | NM_001012980 | spermidine/spermine N1-acetyl transferase-like |
| SAV1 | NM_021818 | WW45 protein |
| SC65 | NM_006455 | synaptonemal complex protein SC65 |
| SCAMP1 | NM_004866 | secretory carrier membrane protein 1 isoform 1 |
| SCAMP4 | NM_079834 | secretory carrier membrane protein 4 |
| SCAMP5 | NM_138967 | secretory carrier membrane protein 5 |
| SCAND2 | NM_022050 | SCAN domain-containing protein 2 isoform 1 |
| SCAP2 | NM_003930 | src family associated phosphoprotein 2 |
| SCC-112 | NM_015200 | SCC-112 protein |
| SCG3 | NM_013243 | secretogranin III |
| SCML1 | NM_006746 | sex comb on midleg-like 1 isoform b |
| SCML4 | NM_198081 | sex comb on midleg-like 4 |
| SCN11A | NM_014139 | sodium channel, voltage-gated, type XI, alpha |
| SCN2B | NM_004588 | sodium channel, voltage-gated, type II, beta |
| SCN4A | NM_000334 | voltage-gated sodium channel type 4 alpha |
| SCN4B | NM_174934 | sodium channel, voltage-gated, type IV, beta |
| SCOC | NM_032547 | short coiled-coil protein |
| SCRT1 | NM_031309 | scratch |
| SCYL1 | NM_020680 | SCY1-like 1 |
| SDF4 | NM_016176 | calcium binding protein Cab45 precursor |
| SDS | NM_006843 | serine dehydratase |
| SEC14L1 | NM_003003 | SEC14 (S. cerevisiae)-like 1 isoform a |
| SEC14L4 | NM_174977 | SEC14p-like protein TAP3 |
| SEL1L | NM_005065 | sel-1 suppressor of lin-12-like |
| SELI | NM_033505 | selenoprotein I |
| SELL | NM_000655 | selectin L |
| SELP | NM_003005 | selectin P precursor |
| SELT | NM_016275 | selenoprotein T |
| SEMA3E | NM_012431 | semaphorin 3E |
| SEMA3G | NM_020163 | semaphorin sem2 |
| SEMA4F | NM_004263 | semaphorin W |
| SEMA5A | NM_003966 | semaphorin 5A |
| SEMA7A | NM_003612 | semaphorin 7A |
| SEPT10 | NM_144710 | septin 10 isoform 1 |
| SEPT11 | NM_018243 | septin 11 |
| SEPT3 | NM_019106 | septin 3 isoform B |
| SEPT4 | NM_080417 | septin 4 isoform 4 |
| SEPT6 | NM_145799 | septin 6 isoform A |
| SEPT9 | NM_006640 | septin 9 |
| SEPX1 | NM_016332 | selenoprotein X, 1 |
| SERF1A | NM_021967 | small EDRK-rich factor 1A, telomeric |
| SERF1B | NM_022978 | small EDRK-rich factor 1B, centromeric |
| SERPINB13 | NM_012397 | serine (or cysteine) proteinase inhibitor, clade |
| SERPINB8 | NM_002640 | serine (or cysteine) proteinase inhibitor, clade |
| SERPINC1 | NM_000488 | serine (or cysteine) proteinase inhibitor, clade |
| SERPINE1 | NM_000602 | plasminogen activator inhibitor-1 |
| SETD1A | NM_014712 | SET domain containing 1A |
| SF1 | NM_004630 | splicing factor 1 isoform 1 |
| SF3A1 | NM_001005409 | splicing factor 3a, subunit 1, 120 kDa isoform 2 |
| SF3A3 | NM_006802 | splicing factor 3a, subunit 3 |
| SF4 | NM_182812 | splicing factor 4 isoform c |
| SFMBT1 | NM_001005158 | Scm-like with four mbt domains 1 |
| SFMBT2 | NM_001029880 | Scm-like with four mbt domains 2 |
| SFRP4 | NM_003014 | secreted frizzled-related protein 4 |
| SFRS11 | NM_004768 | splicing factor p54 |
| SFRS14 | NM_001017392 | splicing factor, arginine/serine-rich 14 |
| SFTPB | NM_198843 | surfactant, pulmonary-associated protein B |
| SFXN1 | NM_022754 | sideroflexin 1 |
| SFXN5 | NM_144579 | sideroflexin 5 |
| SGCB | NM_000232 | sarcoglycan, beta (43 kDa dystrophin-associated |
| SGEF | NM_015595 | Src homology 3 domain-containing guanine |
| SGK2 | NM_016276 | serum/glucocorticoid regulated kinase 2 isoform |
| SGK3 | NM_001033578 | serum/glucocorticoid regulated kinase 3 isoform |
| SH2BP1 | NM_014633 | SH2 domain binding protein 1 |
| SH2D3A | NM_005490 | SH2 domain containing 3A |
| SH2D3C | NM_170600 | SH2 domain containing 3C isoform 2 |
| SH2D4A | NM_022071 | SH2 domain containing 4A |
| SH2D4B | NM_207372 | SH2 domain containing 4B |
| SH3BGRL2 | NM_031469 | SH3 domain binding glutamic acid-rich protein |
| SH3BP2 | NM_003023 | SH3-domain binding protein 2 |
| SH3GL2 | NM_003026 | SH3-domain GRB2-like 2 |
| SH3PX3 | NM_153271 | SH3 and PX domain containing 3 |
| SH3PXD2A | NM_014631 | SH3 multiple domains 1 |
| SH3PXD2B | NM_001017995 | SH3 and PX domains 2B |
| SHANK2 | NM_012309 | SH3 and multiple ankyrin repeat domains 2 |
| SHE | NM_001010846 | Src homology 2 domain containing E |
| SIDT1 | NM_017699 | SID1 transmembrane family, member 1 |
| SIGLEC11 | NM_052884 | sialic acid binding Ig-like lectin 11 |
| SIGLEC6 | NM_198846 | sialic acid binding Ig-like lectin 6 isoform 3 |
| SIPA1L3 | NM_015073 | signal-induced proliferation-associated 1 like |
| SIRPA | NM_080792 | signal-regulatory protein alpha precursor |
| SIRPB1 | NM_006065 | signal-regulatory protein beta 1 precursor |
| SIRPG | NM_018556 | signal-regulatory protein gamma isoform 1 |
| SIRT2 | NM_012237 | sirtuin 2 isoform 1 |
| SIRT5 | NM_031244 | sirtuin 5 isoform 2 |
| SIT1 | NM_014450 | SHP2-interacting transmembrane adaptor protein |
| SITPEC | NM_016581 | evolutionarily conserved signaling intermediate |
| SIX4 | NM_017420 | sine oculis homeobox homolog 4 |
| SKIP | NM_016532 | skeletal muscle and kidney enriched inositol |
| SLAMF7 | NM_021181 | SLAM family member 7 |
| SLC12A5 | NM_020708 | solute carrier family 12 member 5 |
| SLC13A5 | NM_177550 | solute carrier family 13 (sodium-dependent |
| SLC14A2 | NM_007163 | solute carrier family 14 (urea transporter), |
| SLC15A4 | NM_145648 | solute carrier family 15, member 4 |
| SLC16A12 | NM_213606 | solute carrier family 16 (monocarboxylic acid |
| SLC16A14 | NM_152527 | solute carrier family 16 (monocarboxylic acid |
| SLC16A2 | NM_006517 | solute carrier family 16, member 2 |
| SLC17A5 | NM_012434 | solute carrier family 17 (anion/sugar |
| SLC17A6 | NM_020346 | differentiation-associated Na-dependent |
| SLC17A7 | NM_020309 | solute carrier family 17, member 7 |
| SLC1A1 | NM_004170 | solute carrier family 1, member 1 |
| SLC1A2 | NM_004171 | solute carrier family 1, member 2 |
| SLC1A3 | NM_004172 | solute carrier family 1 (glial high affinity |
| SLC22A15 | NM_018420 | solute carrier family 22 (organic cation |
| SLC22A16 | NM_033125 | solute carrier family 22, member 16 |
| SLC22A3 | NM_021977 | solute carrier family 22 member 3 |
| SLC22A7 | NM_006672 | solute carrier family 22 member 7 isoform a |
| SLC24A1 | NM_004727 | solute carrier family 24 |
| SLC24A4 | NM_153646 | solute carrier family 24 member 4 isoform 1 |
| SLC25A13 | NM_014251 | solute carrier family 25, member 13 (citrin) |
| SLC25A15 | NM_014252 | solute carrier family 25 (mitochondrial carrier; |
| SLC25A23 | NM_024103 | solute carrier family 25 (mitochondrial carrier; |
| SLC25A25 | NM_001006641 | solute carrier family 25, member 25 isoform b |
| SLC25A3 | NM_213612 | solute carrier family 25 member 3 isoform c |
| SLC26A2 | NM_000112 | solute carrier family 26 member 2 |
| SLC26A4 | NM_000441 | pendrin |
| SLC26A8 | NM_052961 | solute carrier family 26, member 8 isoform a |
| SLC27A1 | NM_198580 | solute carrier family 27 (fatty acid |
| SLC27A4 | NM_005094 | solute carrier family 27 (fatty acid |
| SLC2A3 | NM_006931 | solute carrier family 2 (facilitated glucose |
| SLC2A5 | NM_003039 | solute carrier family 2 (facilitated |
| SLC30A3 | NM_003459 | solute carrier family 30 (zinc transporter), |
| SLC30A8 | NM_173851 | solute carrier family 30 member 8 |
| SLC30A9 | NM_006345 | solute carrier family 30 (zinc transporter), |
| SLC31A1 | NM_001859 | solute carrier family 31 (copper transporters), |
| SLC31A2 | NM_001860 | solute carrier family 31 (copper transporters), |
| SLC35A4 | NM_080670 | solute carrier family 35, member A4 |
| SLC35A5 | NM_017945 | solute carrier family 35, member A5 |
| SLC35B1 | NM_005827 | solute carrier family 35, member B1 |
| SLC35B4 | NM_032826 | solute carrier family 35, member B4 |
| SLC35D2 | NM_007001 | solute carrier family 35, member D2 |
| SLC35E1 | NM_024881 | solute carrier family 35, member E1 |
| SLC35F1 | NM_001029858 | solute carrier family 35, member F1 |
| SLC35F5 | NM_025181 | solute carrier family 35, member F5 |
| SLC36A1 | NM_078483 | solute carrier family 36 member 1 |
| SLC37A2 | NM_198277 | solute carrier family 37 (glycerol-3-phosphate |
| SLC38A2 | NM_018976 | solute carrier family 38, member 2 |
| SLC38A3 | NM_006841 | solute carrier family 38, member 3 |
| SLC39A10 | NM_020342 | solute carrier family 39 (zinc transporter), |
| SLC39A11 | NM_139177 | solute carrier family 39 (metal ion |
| SLC39A3 | NM_213568 | solute carrier family 39 (zinc transporter), |
| SLC41A1 | NM_173854 | solute carrier family 41 member 1 |
| SLC45A3 | NM_033102 | prostein |
| SLC5A6 | NM_021095 | solute carrier family 5 (sodium-dependent |
| SLC5A8 | NM_145913 | solute carrier family 5 (iodide transporter), |
| SLC6A1 | NM_003042 | solute carrier family 6 (neurotransmitter |
| SLC6A20 | NM_020208 | solute carrier family 6, member 20 isoform 1 |
| SLC6A6 | NM_003043 | solute carrier family 6 (neurotransmitter |
| SLC6A7 | NM_014228 | solute carrier family 6, member 7 |
| SLC7A5 | NM_003486 | solute carrier family 7 (cationic amino acid |
| SLC7A6 | NM_003983 | solute carrier family 7 (cationic amino acid |
| SLC8A2 | NM_015063 | solute carrier family 8 member 2 |
| SLC8A3 | NM_033262 | solute carrier family 8 member 3 isoform A |
| SLC9A1 | NM_003047 | solute carrier family 9, isoform A1 |
| SLC9A5 | NM_004594 | solute carrier family 9 (sodium/hydrogen |
| SLC9A8 | NM_015266 | Na+/H+ exchanger isoform 8 |
| SLCO2A1 | NM_005630 | solute carrier organic anion transporter family, |
| SLCO2B1 | NM_007256 | solute carrier organic anion transporter family, |
| SLFN13 | NM_144682 | schlafen family member 13 |
| SLFN5 | NM_144975 | schlafen family member 5 |
| SLFNL1 | NM_144990 | hypothetical protein LOC200172 |
| SLITRK3 | NM_014926 | slit and trk like 3 protein |
| SMA4 | NM_021652 | SMA4 |
| SMAD2 | NM_001003652 | Sma- and Mad-related protein 2 |
| SMAD3 | NM_005902 | MAD, mothers against decapentaplegic homolog 3 |
| SMARCB1 | NM_001007468 | SWI/SNF related, matrix associated, actin |
| SMARCD2 | NM_003077 | SWI/SNF-related matrix-associated |
| SMC1L1 | NM_006306 | SMC1 structural maintenance of chromosomes |
| SMC2L1 | NM_006444 | structural maintenance of chromosomes 2-like 1 |
| SMCR7 | NM_139162 | Smith-Magenis syndrome chromosome region, |
| SMG7 | NM_014837 | SMG-7 homolog isoform 3 |
| SMNDC1 | NM_005871 | survival motor neuron domain containing 1 |
| SMO | NM_005631 | smoothened |
| SMPD3 | NM_018667 | sphingomyelin phosphodiesterase 3, neutral |
| SMURF1 | NM_020429 | Smad ubiquitination regulatory factor 1 isoform |
| SMYD4 | NM_052928 | SET and MYND domain containing 4 |
| SNF1LK2 | NM_015191 | SNF1-like kinase 2 |
| SNIP | NM_025248 | SNAP25-interacting protein |
| SNPH | NM_014723 | syntaphilin |
| SNRPN | NM_003097 | small nuclear ribonucleoprotein polypeptide N |
| SNTB2 | NM_130845 | basic beta 2 syntrophin isoform b |
| SNURF | NM_005678 | SNRPN upstream reading frame protein |
| SNX11 | NM_013323 | sorting nexin 11 |
| SNX13 | NM_015132 | sorting nexin 13 |
| SNX27 | NM_030918 | sorting nexin family member 27 |
| SOHLH2 | NM_017826 | hypothetical protein LOC54937 |
| SON | NM_003103 | SON DNA-binding protein isoform G |
| SORBS1 | NM_015385 | sorbin and SH3 domain containing 1 isoform 2 |
| SORCS1 | NM_001013031 | SORCS receptor 1 isoform b |
| SORCS2 | NM_020777 | VPS10 domain receptor protein SORCS 2 |
| SOST | NM_025237 | sclerostin precursor |
| SOX1 | NM_005986 | SRY (sex determining region Y)-box 1 |
| SOX13 | NM_005686 | SRY-box 13 |
| SOX8 | NM_014587 | SRY (sex determining region Y)-box 8 |
| SP1 | NM_138473 | Sp1 transcription factor |
| SP4 | NM_003112 | Sp4 transcription factor |
| SP6 | NM_199262 | Sp6 transcription factor |
| SP7 | NM_152860 | osterix |
| SPACA4 | NM_133498 | sperm acrosomal membrane protein 14 |
| SPAG16 | NM_001025436 | sperm associated antigen 16 isoform 2 |
| SPANXA1 | NM_013453 | sperm protein associated with the nucleus, X |
| SPANXA2 | NM_145662 | sperm protein associated with the nucleus, X |
| SPANXC | NM_022661 | sperm protein associated with the nucleus, X |
| SPANXD | NM_032417 | sperm protein associated with the nucleus, X |
| SPANXE | NM_145665 | sperm protein associated with the nucleus, X |
| SPATA12 | NM_181727 | spermatogenesis associated 12 |
| SPATA18 | NM_145263 | spermatogenesis associated 18 homolog |
| SPATA2 | NM_006038 | spermatogenesis associated 2 |
| SPECC1 | NM_001033554 | spectrin domain with coiled-coils 1 NSP5a3a |
| SPG21 | NM_016630 | acid cluster protein 33 |
| SPIB | NM_003121 | Spi-B transcription factor (Spi-1/PU.1 related) |
| SPINLW1 | NM_020398 | serine peptidase inhibitor-like, with Kunitz and |
| SPIRE1 | NM_020148 | spire homolog 1 |
| SPN | NM_001030288 | sialophorin |
| SPOCK1 | NM_004598 | sparc/osteonectin, cwcv and kazal-like domains |
| SPOCK2 | NM_014767 | sparc/osteonectin, cwcv and kazal-like domains |
| SPRN | NM_001012508 | shadow of prion protein |
| SPRY3 | NM_005840 | sprouty homolog 3 |
| SPRYD3 | NM_032840 | hypothetical protein LOC84926 |
| SPTB | NM_001024858 | spectrin beta isoform a |
| SPTBN2 | NM_006946 | spectrin, beta, non-erythrocytic 2 |
| SPTLC2 | NM_004863 | serine palmitoyltransferase, long chain base |
| SPTY2D1 | NM_194285 | hypothetical protein LOC144108 |
| SRD5A1 | NM_001047 | steroid-5-alpha-reductase 1 |
| SRD5A2L2 | NM_001010874 | steroid 5 alpha-reductase 2-like 2 |
| SRGAP2 | NM_015326 | SLIT-ROBO Rho GTPase activating protein 2 |
| SRM | NM_003132 | spermidine synthase |
| SRP72 | NM_006947 | signal recognition particle 72 kDa |
| SS18L1 | NM_015558 | SS18-like protein 1 |
| SSBP3 | NM_001009955 | single stranded DNA binding protein 3 isoform c |
| SSH2 | NM_033389 | slingshot 2 |
| SSR3 | NM_007107 | signal sequence receptor gamma subunit |
| SSTR1 | NM_001049 | somatostatin receptor 1 |
| SSX1 | NM_005635 | synovial sarcoma, X breakpoint 1 |
| SSX8 | NM_174961 | synovial sarcoma, X breakpoint 8 |
| ST6GAL1 | NM_003032 | sialyltransferase 1 isoform a |
| ST6GALNAC4 | NM_175040 | sialyltransferase 7D isoform b |
| ST7L | NM_017744 | suppression of tumorigenicity 7-like isoform 1 |
| ST8SIA2 | NM_006011 | ST8 alpha-N-acetyl-neuraminide |
| ST8SIA4 | NM_005668 | ST8 alpha-N-acetyl-neuraminide |
| STAB2 | NM_017564 | stabilin 2 precursor |
| STAC | NM_003149 | SH3 and cysteine rich domain |
| STAR | NM_000349 | steroidogenic acute regulator isoform 1 |
| STARD13 | NM_052851 | START domain containing 13 isoform gamma |
| STARD4 | NM_139164 | START domain containing 4, sterol regulated |
| STARD5 | NM_030574 | StAR-related lipid transfer protein 5 isoform 2 |
| STAT5A | NM_003152 | signal transducer and activator of transcription |
| STAU2 | NM_014393 | staufen homolog 2 |
| STCH | NM_006948 | stress 70 protein chaperone, |
| STEAP3 | NM_001008410 | dudulin 2 isoform b |
| STIP1 | NM_006819 | stress-induced-phosphoprotein 1 |
| STK10 | NM_005990 | serine/threonine kinase 10 |
| STK16 | NM_001008910 | serine/threonine kinase 16 |
| STK32B | NM_018401 | serine/threonine kinase 32B |
| STK35 | NM_080836 | serine/threonine kinase 35 |
| STK4 | NM_006282 | serine/threonine kinase 4 |
| STMN3 | NM_015894 | SCG10-like-protein |
| STON1 | NM_006873 | stonin 1 |
| STOX2 | NM_020225 | storkhead box 2 |
| STRN | NM_003162 | striatin, calmodulin binding protein |
| STRN3 | NM_014574 | nuclear autoantigen |
| STS | NM_000351 | steryl-sulfatase precursor |
| STX17 | NM_017919 | syntaxin 17 |
| STXBP1 | NM_001032221 | syntaxin binding protein 1 isoform b |
| STXBP5 | NM_139244 | tomosyn |
| SUFU | NM_016169 | suppressor of fused |
| SUHW1 | NM_080740 | suppressor of hairy wing homolog 1 |
| SULT1A3 | NM_001017387 | sulfotransferase family, cytosolic, 1A, |
| SULT1A4 | NM_001017389 | sulfotransferase family, cytosolic, 1A, |
| SULT1E1 | NM_005420 | sulfotransferase, estrogen-preferring |
| SULT2A1 | NM_003167 | sulfotransferase family, cytosolic, 2A, |
| SUMO3 | NM_006936 | small ubiquitin-like modifier protein 3 |
| SURB7 | NM_004264 | SRB7 suppressor of RNA polymerase B homolog |
| SURF4 | NM_033161 | surfeit 4 |
| SURF5 | NM_133640 | surfeit 5 isoform b |
| SUSD2 | NM_019601 | sushi domain containing 2 |
| SUSD4 | NM_017982 | sushi domain containing 4 isoform a |
| SUV420H1 | NM_016028 | suppressor of variegation 4-20 homolog 1 isoform |
| SV2A | NM_014849 | synaptic vesicle glycoprotein 2 |
| SV2B | NM_014848 | synaptic vesicle protein 2B homolog |
| SVOP | NM_018711 | SV2 related protein |
| SWAP70 | NM_015055 | SWAP-70 protein |
| SYBL1 | NM_005638 | synaptobrevin-like 1 |
| SYN2 | NM_003178 | synapsin II isoform IIb |
| SYN3 | NM_133632 | synapsin III isoform IIIb |
| SYNGR1 | NM_004711 | synaptogyrin 1 isoform 1a |
| SYNJ2 | NM_003898 | synaptojanin 2 |
| SYNJ2BP | NM_018373 | synaptojanin 2 binding protein |
| SYT10 | NM_198992 | synaptotagmin 10 |
| SYT11 | NM_152280 | synaptotagmin 12 |
| SYT3 | NM_032298 | synaptotagmin 3 |
| SYT6 | NM_205848 | synaptotagmin VI |
| SYT7 | NM_004200 | synaptotagmin VII |
| SYT9 | NM_175733 | synaptotagmin IX |
| TACC1 | NM_006283 | transforming, acidic coiled-coil containing |
| TACSTD2 | NM_002353 | tumor-associated calcium signal transducer 2 |
| TADA3L | NM_133480 | transcriptional adaptor 3-like isoform b |
| TAF12 | NM_005644 | TAF12 RNA polymerase II, TATA box binding |
| TAF1L | NM_153809 | TBP-associated factor RNA polymerase 1-like |
| TAOK2 | NM_004783 | TAO kinase 2 isoform 1 |
| TAPBP | NM_003190 | tapasin isoform 1 precursor |
| TARDBP | NM_007375 | TAR DNA binding protein |
| TATDN2 | NM_014760 | TatD DNase domain containing 2 |
| TAZ | NM_181314 | tafazzin isoform 5 |
| TBC1D1 | NM_015173 | TBC1 (tre-2/USP6, BUB2, cdc16) domain family, |
| TBC1D10B | NM_015527 | TBC1 domain family, member 10B |
| TBC1D14 | NM_020773 | TBC1 domain family, member 14 |
| TBC1D20 | NM_144628 | TBC1 domain family, member 20 |
| TBC1D22A | NM_014346 | TBC1 domain family, member 22A |
| TBC1D22B | NM_017772 | TBC1 domain family, member 22B |
| TBC1D2B | NM_015079 | TBC1 domain family, member 2B |
| TBL1X | NM_005647 | transducin beta-like 1X |
| TBX21 | NM_013351 | T-box 21 |
| TBX3 | NM_005996 | T-box 3 protein isoform 1 |
| TCEAL7 | NM_152278 | hypothetical protein LOC56849 |
| TCF15 | NM_004609 | basic helix-loop-helix transcription factor 15 |
| TCF20 | NM_005650 | transcription factor 20 isoform 1 |
| TCF21 | NM_198392 | transcription factor 21 |
| TCF7 | NM_003202 | transcription factor 7 (T-cell specific, |
| TCHHL1 | NM_001008536 | trichohyalin-like 1 |
| TCHP | NM_032300 | trichoplein |
| TCN2 | NM_000355 | transcobalamin II precursor |
| TCP10 | NM_004610 | t-complex 10 |
| TCTA | NM_022171 | T-cell leukemia translocation altered gene |
| TEAD1 | NM_021961 | TEA domain family member 1 |
| TEAD3 | NM_003214 | TEA domain family member 3 |
| TERT | NM_198253 | telomerase reverse transcriptase isoform 3 |
| TEX2 | NM_018469 | testis expressed sequence 2 |
| TEX261 | NM_144582 | testis expressed sequence 261 |
| TFAP2B | NM_003221 | transcription factor AP-2 beta (activating |
| TFF3 | NM_003226 | trefoil factor 3 precursor |
| TGIF2 | NM_021809 | TGFB-induced factor 2 |
| TGM2 | NM_004613 | transglutaminase 2 isoform a |
| THADA | NM_198554 | thyroid adenoma associated isoform 2 |
| THAP6 | NM_144721 | THAP domain containing 6 |
| THBS1 | NM_003246 | thrombospondin 1 precursor |
| THEDC1 | NM_018324 | thioesterase domain containing 1 isoform 1 |
| THEM4 | NM_053055 | thioesterase superfamily member 4 isoform a |
| THEM5 | NM_182578 | thioesterase superfamily member 5 |
| THY1 | NM_006288 | Thy-1 cell surface antigen |
| TIA1 | NM_022037 | TIA1 protein isoform 1 |
| TIGD5 | NM_032862 | tigger transposable element derived 5 |
| TIMM17A | NM_006335 | translocase of inner mitochondrial membrane 17 |
| TK2 | NM_004614 | thymidine kinase 2, mitochondrial |
| TKTL1 | NM_012253 | transketolase-like 1 |
| TKTL2 | NM_032136 | transketolase-like 2 |
| TLK2 | NM_006852 | tousled-like kinase 2 |
| TLN2 | NM_015059 | talin 2 |
| TLR10 | NM_001017388 | toll-like receptor 10 precursor |
| TLX2 | NM_016170 | T-cell leukemia, homeobox 2 |
| TM2D2 | NM_001024380 | TM2 domain containing 2 isoform b |
| TM4SF11 | NM_015993 | plasmolipin |
| TM4SF20 | NM_024795 | transmembrane 4 L six family member 20 |
| TM7SF4 | NM_030788 | dendritic cell-specific transmembrane protein |
| TMBIM1 | NM_022152 | transmembrane BAX inhibitor motif containing 1 |
| TMC5 | NM_024780 | transmembrane channel-like 5 |
| TMCC3 | NM_020698 | transmembrane and coiled-coil domains 3 |
| TMED2 | NM_006815 | coated vesicle membrane protein |
| TMEM1 | NM_001001723 | transmembrane protein 1 isoform b |
| TMEM105 | NM_178520 | hypothetical protein LOC284186 |
| TMEM106A | NM_145041 | hypothetical protein LOC113277 |
| TMEM113 | NM_025222 | hypothetical protein PRO2730 |
| TMEM116 | NM_138341 | hypothetical protein LOC89894 |
| TMEM119 | NM_181724 | hypothetical protein LOC338773 |
| TMEM12 | NM_152311 | transmembrane protein 12 |
| TMEM121 | NM_025268 | hole protein |
| TMEM127 | NM_017849 | hypothetical protein LOC55654 |
| TMEM132D | NM_133448 | hypothetical protein LOC121256 |
| TMEM134 | NM_025124 | hypothetical protein LOC80194 |
| TMEM140 | NM_018295 | hypothetical protein LOC55281 |
| TMEM148 | NM_153238 | hypothetical protein LOC197196 |
| TMEM16B | NM_020373 | transmembrane protein 16B |
| TMEM16F | NM_001025356 | transmembrane protein 16F |
| TMEM16G | NM_001001891 | transmembrane protein 16G isoform NGEP long |
| TMEM19 | NM_018279 | transmembrane protein 19 |
| TMEM29 | NM_014138 | hypothetical protein LOC29057 |
| TMEM30B | NM_001017970 | transmembrane protein 30B |
| TMEM33 | NM_018126 | transmembrane protein 33 |
| TMEM40 | NM_018306 | transmembrane protein 40 |
| TMEM41B | NM_015012 | transmembrane protein 41B |
| TMEM43 | NM_024334 | transmembrane protein 43 |
| TMEM53 | NM_024587 | transmembrane protein 53 |
| TMEM56 | NM_152487 | transmembrane protein 56 |
| TMEM58 | NM_198149 | transmembrane protein 58 |
| TMEM60 | NM_032936 | transmembrane protein 60 |
| TMEM63A | NM_014698 | transmembrane protein 63A |
| TMEM69 | NM_016486 | transmembrane protein 69 |
| TMEM80 | NM_174940 | hypothetical protein LOC283232 |
| TMEM97 | NM_014573 | hypothetical protein MAC30 |
| TMLHE | NM_018196 | trimethyllysine hydroxylase, epsilon |
| TMOD2 | NM_014548 | tropomodulin 2 (neuronal) |
| TMPRSS11B | NM_182502 | transmembrane protease, serine 11B |
| TMPRSS3 | NM_024022 | transmembrane protease, serine 3 isoform 1 |
| TMPRSS4 | NM_019894 | transmembrane protease, serine 4 isoform 1 |
| TNFAIP1 | NM_021137 | tumor necrosis factor, alpha-induced protein 1 |
| TNFAIP8L1 | NM_152362 | tumor necrosis factor, alpha-induced protein |
| TNFAIP8L3 | NM_207381 | tumor necrosis factor, alpha-induced protein |
| TNFRSF10B | NM_003842 | tumor necrosis factor receptor superfamily, |
| TNFRSF10C | NM_003841 | tumor necrosis factor receptor superfamily, |
| TNFRSF10D | NM_003840 | tumor necrosis factor receptor superfamily, |
| TNFRSF19 | NM_148957 | tumor necrosis factor receptor superfamily, |
| TNFRSF8 | NM_001243 | tumor necrosis factor receptor superfamily, |
| TNFSF10 | NM_003810 | tumor necrosis factor (ligand) superfamily, |
| TNFSF4 | NM_003326 | tumor necrosis factor (ligand) superfamily, |
| TNFSF9 | NM_003811 | tumor necrosis factor (ligand) superfamily, |
| TNIP3 | NM_024873 | hypothetical protein LOC79931 |
| TNNI1 | NM_003281 | troponin I, skeletal, slow |
| TNP1 | NM_003284 | transition protein 1 (during histone to |
| TNPO2 | NM_013433 | transportin 2 (importin 3, karyopherin beta 2b) |
| TNRC15 | NM_015575 | trinucleotide repeat containing 15 |
| TNRC6B | NM_001024843 | trinucleotide repeat containing 6B isoform 2 |
| TNS3 | NM_022748 | tensin-like SH2 domain containing 1 |
| TOB2 | NM_016272 | transducer of ERBB2, 2 |
| TOLLIP | NM_019009 | toll interacting protein |
| TOM1L2 | NM_001033551 | target of myb1-like 2 isoform 1 |
| TOMM40L | NM_032174 | translocase of outer mitochondrial membrane 40 |
| TOP2A | NM_001067 | DNA topoisomerase II, alpha isozyme |
| TOR2A | NM_130459 | torsin family 2, member A |
| TOR3A | NM_022371 | torsin family 3, member A |
| TP53 | NM_000546 | tumor protein p53 |
| TP53INP1 | NM_033285 | tumor protein p53 inducible nuclear protein 1 |
| TP53RK | NM_033550 | p53-related protein kinase |
| TPD52L3 | NM_033516 | protein kinase NYD-SP25 isoform 1 |
| TPM3 | NM_153649 | tropomyosin 3 isoform 2 |
| TPM4 | NM_003290 | tropomyosin 4 |
| TPP1 | NM_000391 | tripeptidyl-peptidase I precursor |
| TRAF7 | NM_032271 | ring finger and WD repeat domain 1 isoform 1 |
| TRAIP | NM_005879 | TRAF interacting protein |
| TRAM2 | NM_012288 | translocation-associated membrane protein 2 |
| TRAPPC3 | NM_014408 | BET3 homolog |
| TRIAD3 | NM_207111 | TRIAD3 protein isoform a |
| TRIB3 | NM_021158 | tribbles 3 |
| TRIM10 | NM_006778 | tripartite motif-containing 10 isoform 1 |
| TRIM14 | NM_033220 | tripartite motif protein TRIM14 isoform alpha |
| TRIM22 | NM_006074 | tripartite motif-containing 22 |
| TRIM24 | NM_003852 | transcriptional intermediary factor 1 alpha |
| TRIM25 | NM_005082 | tripartite motif-containing 25 |
| TRIM26 | NM_003449 | tripartite motif-containing 26 |
| TRIM29 | NM_012101 | tripartite motif protein TRIM29 isoform alpha |
| TRIM35 | NM_015066 | tripartite motif-containing 35 isoform 1 |
| TRIM37 | NM_015294 | tripartite motif-containing 37 protein |
| TRIM44 | NM_017583 | DIPB protein |
| TRIM5 | NM_033034 | tripartite motif protein TRIM5 isoform alpha |
| TRIM52 | NM_032765 | hypothetical protein LOC84851 |
| TRIM55 | NM_033058 | ring finger protein 29 isoform 2 |
| TRIM56 | NM_030961 | tripartite motif-containing 56 |
| TRIM58 | NM_015431 | tripartite motif-containing 58 |
| TRIM62 | NM_018207 | tripartite motif-containing 62 |
| TRIM65 | NM_173547 | tripartite motif containing 65 |
| TRIM67 | NM_001004342 | hypothetical protein LOC440730 |
| TRIM73 | NM_198924 | hypothetical protein LOC375593 |
| TRIM74 | NM_198853 | hypothetical protein LOC378108 |
| TRIM9 | NM_052978 | tripartite motif protein 9 isoform 2 |
| TRIO | NM_007118 | triple functional domain (PTPRF interacting) |
| TRIT1 | NM_017646 | tRNA isopentenyltransferase 1 |
| TRMT5 | NM_020810 | tRNA-(N1G37) methyltransferase |
| TRPC5 | NM_012471 | transient receptor potential cation channel, |
| TRPM1 | NM_002420 | transient receptor potential cation channel, |
| TRPM2 | NM_001001188 | transient receptor potential cation channel, |
| TRPS1 | NM_014112 | zinc finger transcription factor TRPS1 |
| TRPV5 | NM_019841 | transient receptor potential cation channel, |
| TRPV6 | NM_018646 | transient receptor potential cation channel, |
| TRUB2 | NM_015679 | TruB pseudouridine (psi) synthase homolog 2 |
| TSC1 | NM_000368 | tuberous sclerosis 1 protein isoform 1 |
| TSC22D3 | NM_001015881 | TSC22 domain family, member 3 isoform 3 |
| TSN | NM_004622 | translin |
| TSNAX | NM_005999 | translin-associated factor X |
| TSPAN13 | NM_014399 | tetraspan NET-6 |
| TSPAN15 | NM_012339 | transmembrane 4 superfamily member 15 |
| TSPAN2 | NM_005725 | tetraspan 2 |
| TSPAN9 | NM_006675 | tetraspanin 9 |
| TSPYL5 | NM_033512 | TSPY-like 5 |
| TTBK1 | NM_032538 | tau tubulin kinase 1 |
| TTBK2 | NM_173500 | tau tubulin kinase 2 |
| TTC12 | NM_017868 | tetratricopeptide repeat domain 12 |
| TTC19 | NM_017775 | tetratricopeptide repeat domain 19 |
| TTC21B | NM_024753 | tetratricopeptide repeat domain 21B |
| TTF2 | NM_003594 | transcription termination factor, RNA polymerase |
| TTL | NM_153712 | tubulin tyrosine ligase |
| TTLL2 | NM_031949 | tubulin tyrosine ligase-like family, member 2 |
| TTLL3 | NM_001025930 | tubulin tyrosine ligase-like family, member 3 |
| TTLL6 | NM_173623 | hypothetical protein LOC284076 |
| TTLL9 | NM_001008409 | tubulin tyrosine ligase-like family, member 9 |
| TTYH2 | NM_032646 | tweety 2 isoform 1 |
| TTYH3 | NM_025250 | tweety 3 |
| TUB | NM_003320 | tubby isoform a |
| TUBB | NM_178014 | tubulin, beta polypeptide |
| TUBB1 | NM_030773 | beta tubulin 1, class VI |
| TUBB4 | NM_006087 | tubulin, beta 4 |
| TUBG1 | NM_001070 | tubulin, gamma 1 |
| TUBG2 | NM_016437 | tubulin, gamma 2 |
| TUBGCP6 | NM_001008658 | tubulin, gamma complex associated protein 6 |
| TUFT1 | NM_020127 | tuftelin 1 |
| TULP3 | NM_003324 | tubby like protein 3 |
| TUSC5 | NM_172367 | LOST1 |
| TXLNA | NM_175852 | taxilin |
| TXLNB | NM_153235 | muscle-derived protein 77 |
| TXNDC13 | NM_021156 | thioredoxin domain containing 13 |
| TXNDC4 | NM_015051 | thioredoxin domain containing 4 (endoplasmic |
| TXNL4B | NM_017853 | thioredoxin-like 4B |
| TXNRD1 | NM_003330 | thioredoxin reductase 1 |
| TYSND1 | NM_173555 | trypsin domain containing 1 isoform a |
| UACA | NM_001008224 | uveal autoantigen with coiled-coil domains and |
| UAP1L1 | NM_207309 | UDP-N-acteylglucosamine pyrophosphorylase 1-like |
| UBE2E1 | NM_003341 | ubiquitin-conjugating enzyme E2E 1 isoform 1 |
| UBE2E3 | NM_006357 | ubiquitin-conjugating enzyme E2E 3 |
| UBE2G1 | NM_003342 | ubiquitin-conjugating enzyme E2G 1 isoform 1 |
| UBE2I | NM_003345 | ubiquitin-conjugating enzyme E2I |
| UBE2J1 | NM_016021 | ubiquitin-conjugating enzyme E2, J1 |
| UBE2Q1 | NM_017582 | ubiquitin-conjugating enzyme E2Q |
| UBE2R2 | NM_017811 | ubiquitin-conjugating enzyme UBC3B |
| UBE3B | NM_183414 | ubiquitin protein ligase E3B isoform b |
| UBE3C | NM_014671 | ubiquitin protein ligase E3C |
| UBL3 | NM_007106 | ubiquitin-like 3 |
| UBL7 | NM_032907 | ubiquitin-like 7 (bone marrow stromal |
| UBN1 | NM_016936 | ubinuclein 1 |
| UBOX5 | NM_014948 | U-box domain containing 5 isoform a |
| UBXD2 | NM_014607 | UBX domain containing 2 |
| UBXD8 | NM_014613 | UBX domain containing 8 |
| UGDH | NM_003359 | UDP-glucose dehydrogenase |
| UGT1A1 | NM_000463 | UDP glycosyltransferase 1 family, polypeptide A1 |
| UGT1A10 | NM_019075 | UDP glycosyltransferase 1 family, polypeptide |
| UGT1A3 | NM_019093 | UDP glycosyltransferase 1 family, polypeptide A3 |
| UGT1A4 | NM_007120 | UDP glycosyltransferase 1 family, polypeptide A4 |
| UGT1A5 | NM_019078 | UDP glycosyltransferase 1 family, polypeptide A5 |
| UGT1A6 | NM_001072 | UDP glycosyltransferase 1 family, polypeptide A6 |
| UGT1A7 | NM_019077 | UDP glycosyltransferase 1 family, polypeptide A7 |
| UGT1A8 | NM_019076 | UDP glycosyltransferase 1 family, polypeptide A8 |
| UGT1A9 | NM_021027 | UDP glycosyltransferase 1 family, polypeptide A9 |
| ULBP1 | NM_025218 | UL16 binding protein 1 |
| UMOD | NM_001008389 | uromodulin precursor |
| UNC13D | NM_199242 | unc-13 homolog D |
| UNC45B | NM_001033576 | cardiomyopathy associated 4 isoform 2 |
| UNC5A | NM_133369 | netrin receptor Unc5h1 |
| UNC5D | NM_080872 | netrin receptor Unc5h4 |
| UNC93A | NM_018974 | unc-93 homolog A |
| UPF1 | NM_002911 | regulator of nonsense transcripts 1 |
| UPF2 | NM_015542 | UPF2 regulator of nonsense transcripts homolog |
| USF1 | NM_007122 | upstream stimulatory factor 1 isoform 1 |
| USP18 | NM_017414 | ubiquitin specific protease 18 |
| USP2 | NM_004205 | ubiquitin specific protease 2 isoform a |
| USP37 | NM_020935 | ubiquitin specific protease 37 |
| USP46 | NM_022832 | ubiquitin specific protease 46 |
| USP47 | NM_017944 | ubiquitin specific protease 47 |
| USP49 | NM_018561 | ubiquitin specific protease 49 |
| UTP14C | NM_021645 | UTP14, U3 small nucleolar ribonucleoprotein, |
| UTS2D | NM_198152 | urotensin 2 domain containing |
| UVRAG | NM_003369 | UV radiation resistance associated gene |
| VANGL2 | NM_020335 | vang-like 2 (van gogh, Drosophila) |
| VAPB | NM_004738 | VAMP-associated protein B/C |
| VASH1 | NM_014909 | vasohibin 1 |
| VAT1 | NM_006373 | vesicle amine transport protein 1 |
| VAX1 | NM_199131 | ventral anterior homeobox 1 |
| VBP1 | NM_003372 | von Hippel-Lindau binding protein 1 |
| VCPIP1 | NM_025054 | valosin containing protein (p97)/p47 complex |
| VDAC1 | NM_003374 | voltage-dependent anion channel 1 |
| VEGF | NM_001025366 | vascular endothelial growth factor isoform a |
| VGLL3 | NM_016206 | colon carcinoma related protein |
| VHL | NM_000551 | von Hippel-Lindau tumor suppressor isoform 1 |
| VIPR1 | NM_004624 | vasoactive intestinal peptide receptor 1 |
| VISA | NM_020746 | virus-induced signaling adapter |
| VMD2L3 | NM_152439 | vitelliform macular dystrophy 2-like 3 |
| VPREB1 | NM_007128 | immunoglobulin iota chain preproprotein |
| VPS13A | NM_001018037 | vacuolar protein sorting 13A isoform C |
| VPS13D | NM_015378 | vacuolar protein sorting 13D isoform 1 |
| VPS16 | NM_022575 | vacuolar protein sorting 16 isoform 1 |
| VPS26A | NM_004896 | vacuolar protein sorting 26 homolog A isoform 1 |
| VPS37A | NM_152415 | hepatocellular carcinoma related protein 1 |
| VPS45A | NM_007259 | vacuolar protein sorting 45A |
| VPS4B | NM_004869 | vacuolar protein sorting factor 4B |
| VPS52 | NM_022553 | suppressor of actin mutations 2-like |
| VPS72 | NM_005997 | transcription factor-like 1 |
| VSIG4 | NM_007268 | V-set and immunoglobulin domain containing 4 |
| VSIG9 | NM_173799 | hypothetical protein LOC201633 |
| VTCN1 | NM_024626 | V-set domain containing T cell activation |
| WASF3 | NM_006646 | WAS protein family, member 3 |
| WASPIP | NM_003387 | WASP-interacting protein |
| WBP2 | NM_012478 | WW domain binding protein 2 |
| WBP5 | NM_001006612 | WW domain binding protein 5 |
| WBSCR17 | NM_022479 | UDP-GalNAc:polypeptide |
| WDFY3 | NM_014991 | WD repeat and FYVE domain containing 3 isoform |
| WDR17 | NM_170710 | WD repeat domain 17 isoform 1 |
| WDR22 | NM_003861 | Breakpoint cluster region protein, uterine |
| WDR23 | NM_025230 | WD repeat domain 23 isoform 1 |
| WDR33 | NM_018383 | WD repeat domain 33 isoform 1 |
| WDR36 | NM_139281 | WD repeat domain 36 |
| WDR42B | NM_001017930 | WD repeat domain 42B |
| WDR48 | NM_020839 | WD repeat domain 48 |
| WDR50 | NM_016001 | WD repeat domain 50 |
| WDR6 | NM_018031 | WD repeat domain 6 protein |
| WDR64 | NM_144625 | hypothetical protein LOC128025 |
| WDR68 | NM_005828 | WD-repeat protein |
| WDR7 | NM_015285 | rabconnectin-3 beta isoform 1 |
| WDR81 | NM_152348 | alpha-2-plasmin inhibitor |
| WDTC1 | NM_015023 | WD and tetratricopeptide repeats 1 |
| WFDC1 | NM_021197 | WAP four-disulfide core domain 1 precursor |
| WFS1 | NM_006005 | wolframin |
| WHSC1 | NM_014919 | Wolf-Hirschhorn syndrome candidate 1 protein |
| WIG1 | NM_022470 | p53 target zinc finger protein isoform 1 |
| WIRE | NM_133264 | WIRE protein |
| WNK4 | NM_032387 | WNK lysine deficient protein kinase 4 |
| WNT2 | NM_003391 | wingless-type MMTV integration site family |
| WNT5B | NM_030775 | wingless-type MMTV integration site family, |
| WSB1 | NM_015626 | WD repeat and SOCS box-containing 1 isoform 1 |
| WWC3 | NM_015691 | hypothetical protein LOC55841 |
| WWP2 | NM_007014 | WW domain containing E3 ubiquitin protein ligase |
| XK | NM_021083 | McLeod syndrome-associated, Kell blood group |
| XKR5 | NM_207411 | XK-related protein 5a |
| XLKD1 | NM_006691 | extracellular link domain containing 1 |
| XPO4 | NM_022459 | exportin 4 |
| XPO5 | NM_020750 | exportin 5 |
| XRCC2 | NM_005431 | X-ray repair cross complementing protein 2 |
| XRN1 | NM_019001 | 5′-3′ exoribonuclease 1 |
| XYLB | NM_005108 | xylulokinase homolog |
| YAF2 | NM_001012424 | YY1 associated factor 2 isoform b |
| YARS | NM_003680 | tyrosyl-tRNA synthetase |
| YEATS2 | NM_018023 | YEATS domain containing 2 |
| YIF1B | NM_033557 | Yip1 interacting factor homolog B isoform 2 |
| YPEL1 | NM_013313 | yippee-like 1 |
| YPEL2 | NM_001005404 | yippee-like 2 |
| YPEL5 | NM_016061 | yippee-like 5 |
| YTHDC2 | NM_022828 | YTH domain containing 2 |
| YWHAB | NM_003404 | tyrosine 3-monooxygenase/tryptophan |
| ZADH1 | NM_152444 | zinc binding alcohol dehydrogenase, domain |
| ZADH2 | NM_175907 | zinc binding alcohol dehydrogenase, domain |
| ZAK | NM_016653 | MLK-related kinase isoform 1 |
| ZBTB40 | NM_014870 | zinc finger and BTB domain containing 40 |
| ZBTB41 | NM_194314 | zinc finger and BTB domain containing 41 |
| ZBTB5 | NM_014872 | zinc finger and BTB domain containing 5 |
| ZBTB6 | NM_006626 | zinc finger protein 482 |
| ZC3H12A | NM_025079 | zinc finger CCCH-type containing 12A |
| ZCCHC14 | NM_015144 | zinc finger, CCHC domain containing 14 |
| ZDHHC11 | NM_024786 | zinc finger, DHHC domain containing 11 |
| ZDHHC2 | NM_016353 | rec |
| ZDHHC23 | NM_173570 | zinc finger, DHHC domain containing 23 |
| ZDHHC4 | NM_018106 | zinc finger, DHHC domain containing 4 |
| ZDHHC9 | NM_001008222 | zinc finger, DHHC domain containing 9 |
| ZFAND2B | NM_138802 | zinc finger, AN1-type domain 2B |
| ZFP30 | NM_014898 | zinc finger protein 30 homolog |
| ZFP36L1 | NM_004926 | butyrate response factor 1 |
| ZFP41 | NM_173832 | zinc finger protein 41 homolog |
| ZFP91 | NM_053023 | zinc finger protein 91 isoform 1 |
| ZFP95 | NM_014569 | zinc finger protein 95 homolog |
| ZFYVE16 | NM_014733 | endosome-associated FYVE-domain protein |
| ZFYVE27 | NM_001002261 | zinc finger, FYVE domain containing 27 isoform |
| ZFYVE28 | NM_020972 | zinc finger, FYVE domain containing 28 |
| ZGPAT | NM_181484 | zinc finger, CCCH-type with G patch domain |
| ZHX3 | NM_015035 | zinc fingers and homeoboxes 3 |
| ZIC1 | NM_003412 | zinc finger protein of the cerebellum 1 |
| ZIC3 | NM_003413 | zinc finger protein of the cerebellum 3 |
| ZIC4 | NM_032153 | zinc finger protein of the cerebellum 4 |
| ZIM3 | NM_052882 | zinc finger, imprinted 3 |
| ZKSCAN1 | NM_003439 | zinc finger protein 36 |
| ZMYM3 | NM_005096 | zinc finger protein 261 |
| ZMYM4 | NM_005095 | zinc finger protein 262 |
| ZMYND11 | NM_006624 | zinc finger, MYND domain containing 11 isoform |
| ZMYND19 | NM_138462 | zinc finger, MYND domain containing 19 |
| ZNF132 | NM_003433 | zinc finger protein 132 (clone pHZ-12) |
| ZNF136 | NM_003437 | zinc finger protein 136 (clone pHZ-20) |
| ZNF137 | NM_003438 | zinc finger protein 137 (clone pHZ-30) |
| ZNF157 | NM_003446 | zinc finger protein 157 |
| ZNF160 | NM_033288 | zinc finger protein 160 |
| ZNF167 | NM_018651 | zinc finger protein ZFP isoform 1 |
| ZNF17 | NM_006959 | zinc finger protein 17 |
| ZNF182 | NM_001007088 | zinc finger protein 21 isoform 2 |
| ZNF187 | NM_001023560 | zinc finger protein 187 |
| ZNF192 | NM_006298 | zinc finger protein 192 |
| ZNF200 | NM_003454 | zinc finger protein 200 isoform 1 |
| ZNF202 | NM_003455 | zinc finger protein 202 |
| ZNF217 | NM_006526 | zinc finger protein 217 |
| ZNF226 | NM_001032374 | zinc finger protein 226 isoform b |
| ZNF236 | NM_007345 | zinc finger protein 236 |
| ZNF264 | NM_003417 | zinc finger protein 264 |
| ZNF265 | NM_005455 | zinc finger protein 265 isoform 2 |
| ZNF272 | NM_006635 | zinc finger protein 272 |
| ZNF276 | NM_152287 | zinc finger protein 276 homolog |
| ZNF294 | NM_015565 | zinc finger protein 294 |
| ZNF300 | NM_052860 | zinc finger protein 300 |
| ZNF31 | NM_145238 | zinc finger protein 31 |
| ZNF313 | NM_018683 | zinc finger protein 313 |
| ZNF317 | NM_020933 | zinc finger protein 317 |
| ZNF318 | NM_014345 | zinc finger protein 318 |
| ZNF320 | NM_207333 | zinc finger protein 320 |
| ZNF322A | NM_024639 | zinc finger protein 322A |
| ZNF322B | NM_199005 | zinc finger protein 322B |
| ZNF329 | NM_024620 | zinc finger protein 329 |
| ZNF333 | NM_032433 | zinc finger protein 333 |
| ZNF33A | NM_006974 | zinc finger protein 33A |
| ZNF33B | NM_006955 | zinc finger protein 33B |
| ZNF346 | NM_012279 | zinc finger protein 346 |
| ZNF365 | NM_199451 | zinc finger protein 365 isoform C |
| ZNF37A | NM_001007094 | zinc finger protein 37a |
| ZNF384 | NM_133476 | nuclear matrix transcription factor 4 isoform a |
| ZNF385 | NM_015481 | zinc finger protein 385 |
| ZNF394 | NM_032164 | zinc finger protein 99 |
| ZNF397 | NM_032347 | zinc finger protein 397 |
| ZNF41 | NM_007130 | zinc finger protein 41 |
| ZNF425 | NM_001001661 | zinc finger protein 425 |
| ZNF426 | NM_024106 | zinc finger protein 426 |
| ZNF43 | NM_003423 | zinc finger protein 43 (HTF6) |
| ZNF430 | NM_025189 | zinc finger protein 430 |
| ZNF445 | NM_181489 | zinc finger protein 445 |
| ZNF471 | NM_020813 | zinc finger protein 471 |
| ZNF480 | NM_144684 | zinc finger protein 480 |
| ZNF483 | NM_001007169 | zinc finger protein 483 isoform b |
| ZNF485 | NM_145312 | zinc finger protein 485 |
| ZNF490 | NM_020714 | zinc finger protein 490 |
| ZNF493 | NM_175910 | zinc finger protein 493 |
| ZNF497 | NM_198458 | zinc finger protein 497 |
| ZNF498 | NM_145115 | zinc finger protein 498 |
| ZNF500 | NM_021646 | zinc finger protein 500 |
| ZNF514 | NM_032788 | zinc finger protein 514 |
| ZNF526 | NM_133444 | zinc finger protein 526 |
| ZNF529 | NM_020951 | zinc finger protein 529 |
| ZNF543 | NM_213598 | zinc finger protein 543 |
| ZNF545 | NM_133466 | zinc finger protein 545 |
| ZNF547 | NM_173631 | zinc finger protein 547 |
| ZNF562 | NM_017656 | zinc finger protein 562 |
| ZNF565 | NM_152477 | zinc finger protein 565 |
| ZNF570 | NM_144694 | zinc finger protein 570 |
| ZNF571 | NM_016536 | zinc finger protein 571 |
| ZNF577 | NM_032679 | zinc finger protein 577 |
| ZNF581 | NM_016535 | zinc finger protein 581 |
| ZNF583 | NM_152478 | zinc finger protein 583 |
| ZNF592 | NM_014630 | zinc finger protein 592 |
| ZNF599 | NM_001007247 | zinc finger protein 599 isoform b |
| ZNF600 | NM_198457 | zinc finger protein 600 |
| ZNF605 | NM_183238 | zinc finger protein 605 |
| ZNF607 | NM_032689 | zinc finger protein 607 |
| ZNF621 | NM_198484 | zinc finger protein 621 |
| ZNF622 | NM_033414 | zinc finger protein 622 |
| ZNF623 | NM_014789 | zinc finger protein 623 |
| ZNF650 | NM_172070 | zinc finger protein 650 |
| ZNF651 | NM_145166 | zinc finger protein 651 |
| ZNF652 | NM_014897 | zinc finger protein 652 |
| ZNF660 | NM_173658 | zinc finger protein 660 |
| ZNF662 | NM_207404 | zinc finger protein 662 |
| ZNF677 | NM_182609 | zinc finger protein 677 |
| ZNF694 | NM_001012981 | zinc finger protein 694 |
| ZNF696 | NM_030895 | zinc finger protein 696 |
| ZNF702 | NM_024924 | zinc finger protein 702 |
| ZNF705A | NM_001004328 | hypothetical protein LOC440077 |
| ZNF708 | NM_021269 | zinc finger protein 15-like 1 (KOX 8) |
| ZNF81 | NM_007137 | zinc finger protein 81 (HFZ20) |
| ZNF93 | NM_001004126 | zinc finger protein 93 isoform b |
| ZNRF2 | NM_147128 | zinc finger/RING finger 2 |
| ZSCAN2 | NM_181877 | zinc finger protein 29 isoform 1 |
| ZSWIM4 | NM_023072 | zinc finger, SWIM domain containing 4 |
| ZWILCH | NM_017975 | Zwilch |
| ZWINT | NM_001005414 | ZW10 interactor isoform c |
| ZYG11A | NM_001004339 | hypothetical protein LOC440590 |
| ZYG11B | NM_024646 | hypothetical protein LOC79699 |

TABLE 4
hsa-miR-143 targets that exhibited altered mRNA expression levels
in human cancer cells after transfection with pre-miR hsa-miR-143.
for Ref Seq ID reference - Pruitt et at., 2005.

| Gene | RefSeq | |
|---|---|---|
| Symbol | Transcript ID | Description |
| ATP6V1A | NM_001690 | ATPase, H+ transporting, lysosomal |
| | | 70 kD, V1 |
| ATXN1 | NM_000332 | ataxin 1 |
| CCND1 | NM_053056 | cyclin D1 |
| CLIC4 | NM_013943 | chloride intracellular channel 4 |
| DDAH1 | NM_012137 | dimethylarginine |
| | | dimethylaminohydrolase 1 |
| GALC | NM_000153 | galactosylceramidase |
| | | isoform a precursor |
| GATM | NM_001482 | glycine amidinotransferase |
| | | (L-arginine:glycine |
| GOLPH2 | NM_016548 | golgi phosphoprotein 2 |
| IGFBP3 | NM_000598 | insulin-like growth factor binding |
| | | protein 3 |
| LMO4 | NM_006769 | LIM domain only 4 |
| MCL1 | NM_021960 | myeloid cell leukemia sequence |
| | | 1 isoform 1 |
| PROSC | NM_007198 | proline synthetase co-transcribed |
| | | homolog |
| RAB11FIP1 | NM_001002814 | Rab coupling protein isoform 3 |
| RBL1 | NM_002895 | retinoblastoma-like protein 1 isoform a |
| RHOBTB1 | NM_001032380 | Rho-related BTB domain containing 1 |
| SERPINE1 | NM_000602 | plasminogen activator inhibitor-1 |
| SLC35B1 | NM_005827 | solute carrier family 35, member B1 |
| WASPIP | NM_003387 | WASP-interacting protein |
| WDR50 | NM_016001 | WD repeat domain 50 |

The predicted gene targets of hsa-miR-143 whose mRNA expression levels are affected by hsa-miR-143 represent particularly useful candidates for cancer therapy and therapy of other diseases through manipulation of their expression levels.

Certain embodiments of the invention include determining expression of one or more marker, gene, or nucleic acid segment representative of one or more genes, by using an amplification assay, a hybridization assay, or protein assay, a variety of which are well known to one of ordinary skill in the art. In certain aspects, an amplification assay can be a quantitative amplification assay, such as quantitative RT-PCR or the like. In still further aspects, a hybridization assay can include array hybridization assays or solution hybridization assays. The nucleic acids from a sample may be labeled from the sample and/or hybridizing the labeled nucleic acid to one or more nucleic acid probes. Nucleic acids, mRNA, and/or nucleic acid probes may be coupled to a support. Such supports are well known to those of ordinary skill in the art and include, but are not limited to glass, plastic, metal, or latex. In particular aspects of the invention, the support can be planar or in the form of a bead or other geometric shapes or configurations known in the art. Proteins are typically assayed by immunoblotting, chromatography, or mass spectrometry or other methods known to those of ordinary skill in the art.

The present invention also concerns kits containing compositions of the invention or compositions to implement methods of the invention. In some embodiments, kits can be used to evaluate one or more marker molecules, and/or express one or more miRNA or miRNA inhibitor. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 100, 150, 200 or more probes, recombinant nucleic acid, or synthetic nucleic acid molecules related to the markers to be assessed or an miRNA or miRNA inhibitor to be expressed or modulated, and may include any range or combination derivable therein. Kits may comprise components, which may be individually packaged or placed in a container, such as a tube, bottle, vial, syringe, or other suitable container means. Individual components may also be provided in a kit in concentrated amounts; in some embodiments, a component is provided individually in the same concentration as it would be in a solution with other components. Concentrations of components may be provided as 1×, 2×, 5×, 10×, or 20× or more. Kits for using probes, synthetic nucleic acids, recombinant nucleic acids, or non-synthetic nucleic acids of the invention for therapeutic, prognostic, or diagnostic applications are included as part of the invention. Specifically contemplated are any such molecules corresponding to any miRNA reported to influence biological activity or expression of one or more marker gene or gene pathway described herein. In certain aspects, negative and/or positive controls are included in some kit embodiments. The control molecules can be used to verify transfection efficiency and/or control for transfection-induced changes in cells.

Certain embodiments are directed to a kit for assessment of a pathological condition or the risk of developing a pathological condition in a patient by nucleic acid profiling of a sample comprising, in suitable container means, two or more nucleic acid hybridization or amplification reagents. The kit can comprise reagents for labeling nucleic acids in a sample and/or nucleic acid hybridization reagents. The hybridization reagents typically comprise hybridization probes. Amplification reagents include, but are not limited to amplification primers, reagents, and enzymes.

In some embodiments of the invention, an expression profile is generated by steps that include: (a) labeling nucleic acid in the sample; (b) hybridizing the nucleic acid to a number of probes, or amplifying a number of nucleic acids, and (c) determining and/or quantitating nucleic acid hybridization to the probes or detecting and quantitating amplification products, wherein an expression profile is generated. See U.S. Provisional Patent Application 60/575,743 and the U.S. Provisional Patent Application 60/649,584, and U.S. patent application Ser. No. 11/141,707 and U.S. patent application Ser. No. 11/273,640, all of which are hereby incorporated by reference.

Methods of the invention involve diagnosing and/or assessing the prognosis of a patient based on a miRNA and/or a marker nucleic acid expression profile. In certain embodiments, the elevation or reduction in the level of expression of a particular gene or genetic pathway or set of nucleic acids in a cell is correlated with a disease state or pathological condition compared to the expression level of the same in a normal or non-pathologic cell or tissue sample. This correlation allows for diagnostic and/or prognostic methods to be carried out when the expression level of one or more nucleic acid is measured in a biological sample being assessed and then compared to the expression level of a normal or non-pathologic cell or tissue sample. It is specifically contemplated that expression profiles for patients, particularly those suspected of having or having a propensity for a particular disease or condition such as cancer, can be generated by evaluating any of or sets of the miRNAs and/or nucleic acids discussed in this application. The expression profile that is generated from the patient will be one that provides information regarding the particular disease or condition. In many embodiments, the profile is generated using nucleic acid hybridization or amplification, (e.g., array hybridization or RT-PCR). In certain aspects, an expression profile can be used in conjunction with other diagnostic and/or prognostic tests, such as histology, protein profiles in the serum and/or cytogenetic assessment.

TABLE 5
Tumor associated mRNAs altered by hsa-miR-143 having prognostic or therapeutic
value for the treatment of various malignancies.

| Gene | | Cellular | | |
|---|---|---|---|---|
| Symbol | Gene Title | Process | Cancer Type | Reference |
| AKAP12 | Akap-12/ | signal | CRC, PC, LC, GC, AML, CML | (Xia et al., 2001; Wikman et al., 2002; Boultwood |
| | SSeCKS/ | transduction | | et al., 2004; Choi et al., 2004; Mori et al., 2006) |
| | Gravin | | | |
| BCL2L1 | BCL-XL | Apoptosis | NSCLC, SCLC, CRC, BC, BldC, RCC, HL, NHL, | (Manion and Hockenbery, 2003) |
| | | | AML, ALL, HCC, OC, MB, G, ODG, My, OepC | |
| CCND1 | cyclin D1 | cell cycle | MCL, BC, SCCHN, OepC, HCC, CRC, BldC, EC, | (Donnellan and Chetty, 1998) |
| | | | OC, M, AC, GB, GC, PaC | |
| CCNG1 | cyclin G1 | cell cycle | OS, BC, PC | (Skotzko et al., 1995; Reimer et al., 1999) |
| IGFBP3 | IGFBP-3 | signal | BC, PC, LC, CRC | (Firth and Baxter, 2002) |
| | | transduction | | |
| IL8 | IL-8 | signal | BC, CRC, PaC, NSCLC, PC, HCC | (Akiba et al., 2001; Sparmann and Bar-Sagi, 2004) |
| | | transduction | | |
| LMO4 | Lmo-4 | transcription | BC, SCCHN, SCLC | (Visvader et al., 2001; Mizunuma et al., 2003; |
| | | | | Taniwaki et al., 2006) |
| MCL1 | Mcl-1 | apoptosis | HCC, MM, TT, CLL, ALCL, BCL, PC | (Krajewska et al., 1996; Kitada et al., 1998; Cho- |
| | | | | Vega et al., 2004; Rust et al., 2005; Sano et al., |
| | | | | 2005; Wuilleme-Toumi et al., 2005; Fleischer et |
| | | | | al., 2006; Sieghart et al., 2006) |
| PDCD4 | Pdcd-4 | apoptosis | G, HCC, L, RCC | (Chen et al., 2003; Jansen et al., 2004; Zhang et |
| | | | | al., 2006; Gao et al., 2007) |
| RBL1 | p107 | cell cycle | BCL, PC, CRC, TC | (Takimoto et al., 1998; Claudio et al., 2002; Wu et |
| | | | | al., 2002; Ito et al., 2003) |
| TGFBR2 | TGF beta | signal | BC, CRC | (Markowitz, 2000; Lucke et al., 2001; Biswas et |
| | receptor type | transduction | | al., 2004) |
| | II | | | |
| TXN | thioredoxin | thioredoxin | LC, PaC, CeC, HCC | (Marks, 2006) |
| | (trx) | redox system | | |
| WEE1 | Wee-1 kinase | cell cycle | NSCLC | (Yoshida et al., 2004) |

Abbreviations:
AC, astrocytoma;
ALCL, anaplastic large cell lymphoma;
ALL, acute lymphoblastic leukemia;
AML, acute myelogenous leukemia;
BC, breast carcinoma;
BCL, B-cell lymphoma;
BldC, bladder carcinoma;
CeC, cervical carcinoma;
CLL, chronic lymphoblastic leukemia;
CRC, colorectal carcinoma;
EC, endometrial carcinoma;
G, glioma;
GB, glioblastoma;
GC, gastric carcinoma;
HCC, hepatocellular carcinoma;
HL, Hodgkin lymphoma;
L, leukemia;
LC, lung carcinoma;
M, melanoma;
MB, medulloblastoma;
MCL, mantle cell lymphoma;
MM, multiple myeloma;
My, myeloma;
NHL, non-Hodgkin lymphoma;
NSCLC, non-small cell lung carcinoma;
OC, ovarian carcinoma;
ODG, oligodendroglioma;
OepC, oesophageal carcinoma;
OS, osteosarcoma;
PaC, pancreatic carcinoma;
PC, prostate carcinoma;
RCC, renal cell carcinoma;
SCCHN, squamous cell carcinoma of the head and neck;
SCLC, small cell lung carcinoma;
TC, thyroid carcinoma;
TT, testicular tumor.

The methods can further comprise one or more of the steps including: (a) obtaining a sample from the patient, (b) isolating nucleic acids from the sample, (c) labeling the nucleic acids isolated from the sample, and (d) hybridizing the labeled nucleic acids to one or more probes. Nucleic acids of the invention include one or more nucleic acid comprising at least one segment having a sequence or complementary sequence of to a nucleic acid representative of one or more of genes or markers in Table 1, 3, 4, and/or 5.

It is contemplated that any method or composition described herein can be implemented with respect to any other method or composition described herein and that different embodiments may be combined. It is specifically contemplated that any methods and compositions discussed herein with respect to miRNA molecules, miRNA, genes, and nucleic acids representative of genes may be implemented with respect to synthetic nucleic acids. In some embodiments the synthetic nucleic acid is exposed to the proper conditions to allow it to become a processed or mature nucleic acid, such as a miRNA under physiological circumstances. The claims originally filed are contemplated to cover claims that are multiply dependent on any filed claim or combination of filed claims.

Also, any embodiment of the invention involving specific genes (including representative fragments there of), mRNA, or miRNAs by name is contemplated also to cover embodiments involving miRNAs whose sequences are at least 80, 81, 82, 83, 84, 85, 86, 87, 88, 89, 90, 91, 92, 93, 94, 95, 96, 97, 98, 99% identical to the mature sequence of the specified miRNA.

It will be further understood that shorthand notations are employed such that a generic description of a gene or marker thereof, or of an miRNA refers to any of its gene family members (distinguished by a number) or representative fragments thereof, unless otherwise indicated. It is understood by those of skill in the art that a “gene family” refers to a group of genes having the same coding sequence or miRNA coding sequence. Typically, miRNA members of a gene family are identified by a number following the initial designation. For example, miR-16-1 and miR-16-2 are members of the miR-16 gene family and “mir-7” refers to miR-7-1, miR-7-2 and miR-7-3. Moreover, unless otherwise indicated, a shorthand notation refers to related miRNAs (distinguished by a letter). Exceptions to these shorthand notations will be otherwise identified.

Other embodiments of the invention are discussed throughout this application. Any embodiment discussed with respect to one aspect of the invention applies to other aspects of the invention as well and vice versa. The embodiments in the Example and Detailed Description section are understood to be embodiments of the invention that are applicable to all aspects of the invention.

The terms “inhibiting,” “reducing,” or “prevention,” or any variation of these terms, when used in the claims and/or the specification includes any measurable decrease or complete inhibition to achieve a desired result.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWING

The following drawing forms part of the present specification and is included to further demonstrate certain aspects of the present invention. The invention may be better understood by reference to this drawing in combination with the detailed description of specific embodiments presented herein.

FIG. 1. Average tumor volumes in mice harboring xenografts of A549 lung cancer cells treated with hsa-miR-143 (white squares; n=5) or treated with a negative control miRNA (black diamonds; n=5). Standard deviations are shown in the graph. Data points with p values less than 0.05 are indicated by an asterisk. Abbreviation: miR-143, hsa-miR-143; NC, negative control miRNA.

### DETAILED DESCRIPTION OF THE INVENTION

The present invention is directed to compositions and methods relating to the identification and characterization of genes and biological pathways related to these genes as represented by the expression of the identified genes, as well as use of miRNAs related to such, for therapeutic, prognostic, and diagnostic applications, particularly those methods and compositions related to assessing and/or identifying pathological conditions directly or indirectly related to miR-143 expression or the aberrant expression thereof.

In certain aspects, the invention is directed to methods for the assessment, analysis, and/or therapy of a cell or subject where certain genes have a reduced or increased expression (relative to normal) as a result of an increased or decreased expression of any one or a combination of miR-143 family members (including, but not limited to lla-mir-143 M10002552; xtr-mir-143 MI0004937; dre-mir-143-2 MI0002008; rno-mir-143 MI0000916; ptr-mir-143 MI0002549; ppy-mir-143 MI0002551; ggo-mir-143 MI0002550; dre-mir-143-1 MI0002007; hsa-mir-143 MI0000459; ppa-mir-143 MI0002553; mdo-mir-143 MI0005302; and mmu-mir-143 MI0000257) and/or genes with an increased expression (relative to normal) as a result of decreased expression thereof. The expression profile and/or response to miR-143 expression or lack of expression may be indicative of an individual with a pathological condition, e.g., cancer.

Prognostic assays featuring any one or combination of the miRNAs listed or the markers listed (including nucleic acids representative thereof) could be used in assessment of a patient to determine what if any treatment regimen is justified. As with the diagnostic assays mentioned above, the absolute values that define low expression will depend on the platform used to measure the miRNA(s). The same methods described for the diagnostic assays could be used for prognostic assays.

### II. THERAPEUTIC METHODS

Embodiments of the invention concern nucleic acids that perform the activities of or inhibit endogenous miRNAs when introduced into cells. In certain aspects, nucleic acids are synthetic or non-synthetic miRNA. Sequence-specific miRNA inhibitors can be used to inhibit sequentially or in combination the activities of one or more endogenous miRNAs in cells, as well those genes and associated pathways modulated by the endogenous miRNA.

The present invention concerns, in some embodiments, short nucleic acid molecules that function as miRNAs or as inhibitors of miRNA in a cell. The term “short” refers to a length of a single polynucleotide that is 15, 16, 17, 18, 19, 20, 21, 22, 23, 24, 25, 50, 100, or 150 nucleotides or fewer, including all integers or ranges derivable there between. The nucleic acid molecules are typically synthetic. The term “synthetic” refers to a nucleic acid molecule that is not produced naturally in a cell. In certain aspects the chemical structure deviates from a naturally-occurring nucleic acid molecule, such as an endogenous precursor miRNA or miRNA molecule or complement thereof. While in some embodiments, nucleic acids of the invention do not have an entire sequence that is identical or complementary to a sequence of a naturally-occurring nucleic acid, such molecules may encompass all or part of a naturally-occurring sequence or a complement thereof. It is contemplated, however, that a synthetic nucleic acid administered to a cell may subsequently be modified or altered in the cell such that its structure or sequence is the same as non-synthetic or naturally occurring nucleic acid, such as a mature miRNA sequence. For example, a synthetic nucleic acid may have a sequence that differs from the sequence of a precursor miRNA, but that sequence may be altered once in a cell to be the same as an endogenous, processed miRNA or an inhibitor thereof. The term “isolated” means that the nucleic acid molecules of the invention are initially separated from different (in terms of sequence or structure) and unwanted nucleic acid molecules such that a population of isolated nucleic acids is at least about 90% homogenous, and may be at least about 95, 96, 97, 98, 99, or 100% homogenous with respect to other polynucleotide molecules. In many embodiments of the invention, a nucleic acid is isolated by virtue of it having been synthesized in vitro separate from endogenous nucleic acids in a cell. It will be understood, however, that isolated nucleic acids may be subsequently mixed or pooled together. In certain aspects, synthetic miRNA of the invention are RNA or RNA analogs. miRNA inhibitors may be DNA or RNA, or analogs thereof. miRNA and miRNA inhibitors of the invention are collectively referred to as “synthetic nucleic acids.”

In some embodiments, there is a miRNA or a synthetic miRNA having a length of between 17 and 130 residues. The present invention concerns miRNA or synthetic miRNA molecules that are, are at least, or are at most 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 140, 145, 150, 160, 170, 180, 190, 200 or more residues in length, including any integer or any range there between.

In certain embodiments, synthetic miRNA have (a) a “miRNA region” whose sequence or binding region from 5′ to 3′ is identical or complementary to all or a segment of a mature miRNA sequence, and (b) a “complementary region” whose sequence from 5′ to 3′ is between 60% and 100% complementary to the miRNA sequence in (a). In certain embodiments, these synthetic miRNA are also isolated, as defined above. The term “miRNA region” refers to a region on the synthetic miRNA that is at least 75, 80, 85, 90, 95, or 100% identical, including all integers there between, to the entire sequence of a mature, naturally occurring miRNA sequence or a complement thereof. In certain embodiments, the miRNA region is or is at least 90, 91, 92, 93, 94, 95, 96, 97, 98, 99, 99.1, 99.2, 99.3, 99.4, 99.5, 99.6, 99.7, 99.8, 99.9 or 100% identical to the sequence of a naturally-occurring miRNA or complement thereof.

The term “complementary region” or “complement” refers to a region of a nucleic acid or mimetic that is or is at least 60% complementary to the mature, naturally occurring miRNA sequence. The complementary region is or is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein. With single polynucleotide sequences, there may be a hairpin loop structure as a result of chemical bonding between the miRNA region and the complementary region. In other embodiments, the complementary region is on a different nucleic acid molecule than the miRNA region, in which case the complementary region is on the complementary strand and the miRNA region is on the active strand.

In other embodiments of the invention, there are synthetic nucleic acids that are miRNA inhibitors. A miRNA inhibitor is between about 17 to 25 nucleotides in length and comprises a 5′ to 3′ sequence that is at least 90% complementary to the 5′ to 3′ sequence of a mature miRNA. In certain embodiments, a miRNA inhibitor molecule is 17, 18, 19, 20, 21, 22, 23, 24, or 25 nucleotides in length, or any range derivable therein. Moreover, an miRNA inhibitor may have a sequence (from 5′ to 3′) that is or is at least 70, 75, 80, 85, 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the 5′ to 3′ sequence of a mature miRNA, particularly a mature, naturally occurring miRNA. One of skill in the art could use a portion of the miRNA sequence that is complementary to the sequence of a mature miRNA as the sequence for a miRNA inhibitor. Moreover, that portion of the nucleic acid sequence can be altered so that it is still comprises the appropriate percentage of complementarity to the sequence of a mature miRNA.

In some embodiments, of the invention, a synthetic miRNA or inhibitor contains one or more design element(s). These design elements include, but are not limited to: (i) a replacement group for the phosphate or hydroxyl of the nucleotide at the 5′ terminus of the complementary region; (ii) one or more sugar modifications in the first or last 1 to 6 residues of the complementary region; or, (iii) noncomplementarity between one or more nucleotides in the last 1 to 5 residues at the 3′ end of the complementary region and the corresponding nucleotides of the miRNA region. A variety of design modifications are known in the art, see below.

In certain embodiments, a synthetic miRNA has a nucleotide at its 5′ end of the complementary region in which the phosphate and/or hydroxyl group has been replaced with another chemical group (referred to as the “replacement design”). In some cases, the phosphate group is replaced, while in others, the hydroxyl group has been replaced. In particular embodiments, the replacement group is biotin, an amine group, a lower alkylamine group, an aminohexyl phosphate group, an acetyl group, 2′O-Me (2′oxygen-methyl), DMTO (4,4′-dimethoxytrityl with oxygen), fluorescein, a thiol, or acridine, though other replacement groups are well known to those of skill in the art and can be used as well. This design element can also be used with a miRNA inhibitor.

Additional embodiments concern a synthetic miRNA having one or more sugar modifications in the first or last 1 to 6 residues of the complementary region (referred to as the “sugar replacement design”). In certain cases, there is one or more sugar modifications in the first 1, 2, 3, 4, 5, 6 or more residues of the complementary region, or any range derivable therein. In additional cases, there are one or more sugar modifications in the last 1, 2, 3, 4, 5, 6 or more residues of the complementary region, or any range derivable therein, have a sugar modification. It will be understood that the terms “first” and “last” are with respect to the order of residues from the 5′ end to the 3′ end of the region. In particular embodiments, the sugar modification is a 2′O-Me modification, a 2′F modification, a 2′H modification, a 2′amino modification, a 4′thioribose modification or a phosphorothioate modification on the carboxy group linked to the carbon at position 6′. In further embodiments, there are one or more sugar modifications in the first or last 2 to 4 residues of the complementary region or the first or last 4 to 6 residues of the complementary region. This design element can also be used with a miRNA inhibitor. Thus, a miRNA inhibitor can have this design element and/or a replacement group on the nucleotide at the 5′ terminus, as discussed above.

In other embodiments of the invention, there is a synthetic miRNA or inhibitor in which one or more nucleotides in the last 1 to 5 residues at the 3′ end of the complementary region are not complementary to the corresponding nucleotides of the miRNA region (“noncomplementarity”) (referred to as the “noncomplementarity design”). The noncomplementarity may be in the last 1, 2, 3, 4, and/or 5 residues of the complementary miRNA. In certain embodiments, there is noncomplementarity with at least 2 nucleotides in the complementary region.

It is contemplated that synthetic miRNA of the invention have one or more of the replacement, sugar modification, or noncomplementarity designs. In certain cases, synthetic RNA molecules have two of them, while in others these molecules have all three designs in place.

The miRNA region and the complementary region may be on the same or separate polynucleotides. In cases in which they are contained on or in the same polynucleotide, the miRNA molecule will be considered a single polynucleotide. In embodiments in which the different regions are on separate polynucleotides, the synthetic miRNA will be considered to be comprised of two polynucleotides.

When the RNA molecule is a single polynucleotide, there can be a linker region between the miRNA region and the complementary region. In some embodiments, the single polynucleotide is capable of forming a hairpin loop structure as a result of bonding between the miRNA region and the complementary region. The linker constitutes the hairpin loop. It is contemplated that in some embodiments, the linker region is, is at least, or is at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 residues in length, or any range derivable therein. In certain embodiments, the linker is between 3 and 30 residues (inclusive) in length.

In addition to having a miRNA or inhibitor region and a complementary region, there may be flanking sequences as well at either the 5′ or 3′ end of the region. In some embodiments, there is or is at least 1, 2, 3, 4, 5, 6, 7, 8, 9, 10 nucleotides or more, or any range derivable therein, flanking one or both sides of these regions.

Methods of the invention include reducing or eliminating activity of one or more miRNAs in a cell comprising introducing into a cell a miRNA inhibitor (which may be described generally herein as an miRNA, so that a description of miRNA, where appropriate, also will refer to a miRNA inhibitor); or supplying or enhancing the activity of one or more miRNAs in a cell. The present invention also concerns inducing certain cellular characteristics by providing to a cell a particular nucleic acid, such as a specific synthetic miRNA molecule or a synthetic miRNA inhibitor molecule. However, in methods of the invention, the miRNA molecule or miRNA inhibitor need not be synthetic. They may have a sequence that is identical to a naturally occurring miRNA or they may not have any design modifications. In certain embodiments, the miRNA molecule and/or the miRNA inhibitor are synthetic, as discussed above.

The particular nucleic acid molecule provided to the cell is understood to correspond to a particular miRNA in the cell, and thus, the miRNA in the cell is referred to as the “corresponding miRNA.” In situations in which a named miRNA molecule is introduced into a cell, the corresponding miRNA will be understood to be the induced or inhibited miRNA or induced or inhibited miRNA function. It is contemplated, however, that the miRNA molecule introduced into a cell is not a mature miRNA but is capable of becoming or functioning as a mature miRNA under the appropriate physiological conditions. In cases in which a particular corresponding miRNA is being inhibited by a miRNA inhibitor, the particular miRNA will be referred to as the “targeted miRNA.” It is contemplated that multiple corresponding miRNAs may be involved. In particular embodiments, more than one miRNA molecule is introduced into a cell. Moreover, in other embodiments, more than one miRNA inhibitor is introduced into a cell. Furthermore, a combination of miRNA molecule(s) and miRNA inhibitor(s) may be introduced into a cell. The inventors contemplate that a combination of miRNA may act at one or more points in cellular pathways of cells with aberrant phenotypes and that such combination may have increased efficacy on the target cell while not adversely effecting normal cells. Thus, a combination of miRNA may have a minimal adverse effect on a subject or patient while supplying a sufficient therapeutic effect, such as amelioration of a condition, growth inhibition of a cell, death of a targeted cell, alteration of cell phenotype or physiology, slowing of cellular growth, sensitization to a second therapy, sensitization to a particular therapy, and the like.

Methods include identifying a cell or patient in need of inducing those cellular characteristics. Also, it will be understood that an amount of a synthetic nucleic acid that is provided to a cell or organism is an “effective amount,” which refers to an amount needed (or a sufficient amount) to achieve a desired goal, such as inducing a particular cellular characteristic(s). Certain embodiments of the methods include providing or introducing to a cell a nucleic acid molecule corresponding to a mature miRNA in the cell in an amount effective to achieve a desired physiological result.

Moreover, methods can involve providing synthetic or nonsynthetic miRNA molecules. It is contemplated that in these embodiments, that the methods may or may not be limited to providing only one or more synthetic miRNA molecules or only one or more nonsynthetic miRNA molecules. Thus, in certain embodiments, methods may involve providing both synthetic and nonsynthetic miRNA molecules. In this situation, a cell or cells are most likely provided a synthetic miRNA molecule corresponding to a particular miRNA and a nonsynthetic miRNA molecule corresponding to a different miRNA. Furthermore, any method articulated using a list of miRNAs using Markush group language may be articulated without the Markush group language and a disjunctive article (i.e., or) instead, and vice versa.

Typically, an endogenous gene, miRNA or mRNA is modulated in the cell. In particular embodiments, the nucleic acid sequence comprises at least one segment that is at least 70, 75, 80, 85, 90, 95, or 100% identical in nucleic acid sequence to one or more miRNA or gene sequence. Modulation of the expression or processing of an endogenous gene, miRNA, or mRNA can be through modulation of the processing of a mRNA, such processing including transcription, transportation and/or translation with in a cell. Modulation may also be effected by the inhibition or enhancement of miRNA activity with a cell, tissue, or organ. Such processing may affect the expression of an encoded product or the stability of the mRNA. In still other embodiments, a nucleic acid sequence can comprise a modified nucleic acid sequence. In certain aspects, one or more miRNA sequence may include or comprise a modified nucleobase or nucleic acid sequence.

It will be understood in methods of the invention that a cell or other biological matter such as an organism (including patients) can be provided a miRNA or miRNA molecule corresponding to a particular miRNA by administering to the cell or organism a nucleic acid molecule that functions as the corresponding miRNA once inside the cell. The form of the molecule provided to the cell may not be the form that acts a miRNA once inside the cell. Thus, it is contemplated that in some embodiments, a synthetic miRNA or a nonsynthetic miRNA is provided such that it becomes processed into a mature and active miRNA once it has access to the cell's miRNA processing machinery. In certain embodiments, it is specifically contemplated that the miRNA molecule provided is not a mature miRNA molecule but a nucleic acid molecule that can be processed into the mature miRNA once it is accessible to miRNA processing machinery. The term “nonsynthetic” in the context of miRNA means that the miRNA is not “synthetic,” as defined herein. Furthermore, it is contemplated that in embodiments of the invention that concern the use of synthetic miRNAs, the use of corresponding nonsynthetic miRNAs is also considered an aspect of the invention, and vice versa. It will be understand that the term “providing” an agent is used to include “administering” the agent to a patient.

In certain embodiments, methods also include targeting a miRNA to modulate in a cell or organism. The term “targeting a miRNA to modulate” means a nucleic acid of the invention will be employed so as to modulate the selected miRNA. In some embodiments the modulation is achieved with a synthetic or non-synthetic miRNA that corresponds to the targeted miRNA, which effectively provides the targeted miRNA to the cell or organism (positive modulation). In other embodiments, the modulation is achieved with a miRNA inhibitor, which effectively inhibits the targeted miRNA in the cell or organism (negative modulation).

In some embodiments, the miRNA targeted to be modulated is a miRNA that affects a disease, condition, or pathway. In certain embodiments, the miRNA is targeted because a treatment can be provided by negative modulation of the targeted miRNA. In other embodiments, the miRNA is targeted because a treatment can be provided by positive modulation of the targeted miRNA or its targets.

In certain methods of the invention, there is a further step of administering the selected miRNA modulator to a cell, tissue, organ, or organism (collectively “biological matter”) in need of treatment related to modulation of the targeted miRNA or in need of the physiological or biological results discussed herein (such as with respect to a particular cellular pathway or result like decrease in cell viability). Consequently, in some methods of the invention there is a step of identifying a patient in need of treatment that can be provided by the miRNA modulator(s). It is contemplated that an effective amount of a miRNA modulator can be administered in some embodiments. In particular embodiments, there is a therapeutic benefit conferred on the biological matter, where a “therapeutic benefit” refers to an improvement in the one or more conditions or symptoms associated with a disease or condition or an improvement in the prognosis, duration, or status with respect to the disease. It is contemplated that a therapeutic benefit includes, but is not limited to, a decrease in pain, a decrease in morbidity, a decrease in a symptom. For example, with respect to cancer, it is contemplated that a therapeutic benefit can be inhibition of tumor growth, prevention of metastasis, reduction in number of metastases, inhibition of cancer cell proliferation, induction of cell death in cancer cells, inhibition of angiogenesis near cancer cells, induction of apoptosis of cancer cells, reduction in pain, reduction in risk of recurrence, induction of chemo- or radiosensitivity in cancer cells, prolongation of life, and/or delay of death directly or indirectly related to cancer.

Furthermore, it is contemplated that the miRNA compositions may be provided as part of a therapy to a patient, in conjunction with traditional therapies or preventative agents. Moreover, it is contemplated that any method discussed in the context of therapy may be applied preventatively, particularly in a patient identified to be potentially in need of the therapy or at risk of the condition or disease for which a therapy is needed.

In addition, methods of the invention concern employing one or more nucleic acids corresponding to a miRNA and a therapeutic drug. The nucleic acid can enhance the effect or efficacy of the drug, reduce any side effects or toxicity, modify its bioavailability, and/or decrease the dosage or frequency needed. In certain embodiments, the therapeutic drug is a cancer therapeutic. Consequently, in some embodiments, there is a method of treating cancer in a patient comprising administering to the patient the cancer therapeutic and an effective amount of at least one miRNA molecule that improves the efficacy of the cancer therapeutic or protects non-cancer cells. Cancer therapies also include a variety of combination therapies with both chemical and radiation based treatments. Combination chemotherapies include but are not limited to, for example, 5-fluorouracil, alemtuzumab, amrubicin, bevacizumab, bleomycin, bortezomib, busulfan, camptothecin, capecitabine, carboplatin, cetuximab, chlorambucil, cisplatin (CDDP), COX-2 inhibitors (e.g., celecoxib), cyclophosphamide, cytarabine, dactinomycin, dasatinib, daunorubicin, dexamethasone, docetaxel, doxorubicin (adriamycin), EGFR inhibitors (gefitinib and cetuximab), erlotinib, estrogen receptor binding agents, etoposide (VP16), everolimus, farnesyl-protein transferase inhibitors, gefitinib, gemcitabine, gemtuzumab, ibritumomab, ifosfamide, imatinib mesylate, larotaxel, lapatinib, lonafarnib, mechlorethamine, melphalan, methotrexate, mitomycin, navelbine, nitrosurea, nocodazole, oxaliplatin, paclitaxel, plicomycin, procarbazine, raloxifene, rituximab, sirolimus, sorafenib, sunitinib, tamoxifen, taxol, taxotere, temsirolimus, tipifamib, tositumomab, transplatinum, trastuzumab, vinblastin, vincristin, or vinorelbine or any analog or derivative variant of the foregoing.

Generally, inhibitors of miRNAs can be given to decrease the activity of an endogenous miRNA. For example, inhibitors of miRNA molecules that increase cell proliferation can be provided to cells to decrease cell proliferation. The present invention contemplates these embodiments in the context of the different physiological effects observed with the different miRNA molecules and miRNA inhibitors disclosed herein. These include, but are not limited to, the following physiological effects: increase and decreasing cell proliferation, increasing or decreasing apoptosis, increasing transformation, increasing or decreasing cell viability, activating or inhibiting a kinase (e.g., Erk), activating/inducing or inhibiting hTert, inhibit stimulation of growth promoting pathway (e.g., Stat 3 signaling), reduce or increase viable cell number, and increase or decrease number of cells at a particular phase of the cell cycle. Methods of the invention are generally contemplated to include providing or introducing one or more different nucleic acid molecules corresponding to one or more different miRNA molecules. It is contemplated that the following, at least the following, or at most the following number of different nucleic acid or miRNA molecules may be provided or introduced: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or any range derivable therein. This also applies to the number of different miRNA molecules that can be provided or introduced into a cell.

### III. PHARMACEUTICAL FORMULATIONS AND DELIVERY

Methods of the present invention include the delivery of an effective amount of a miRNA or an expression construct encoding the same. An “effective amount” of the pharmaceutical composition, generally, is defined as that amount sufficient to detectably and repeatedly to achieve the stated desired result, for example, to ameliorate, reduce, minimize or limit the extent of the disease or its symptoms. Other more rigorous definitions may apply, including elimination, eradication or cure of disease.

B. Administration

In certain embodiments, it is desired to kill cells, inhibit cell growth, inhibit metastasis, decrease tumor or tissue size, and/or reverse or reduce the malignant or disease phenotype of cells. The routes of administration will vary, naturally, with the location and nature of the lesion or site to be targeted, and include, e.g., intradermal, subcutaneous, regional, parenteral, intravenous, intramuscular, intranasal, systemic, and oral administration and formulation. Direct injection, intratumoral injection, or injection into tumor vasculature is specifically contemplated for discrete, solid, accessible tumors, or other accessible target areas. Local, regional, or systemic administration also may be appropriate. For tumors of >4 cm, the volume to be administered will be about 4-10 ml (preferably 10 ml), while for tumors of <4 cm, a volume of about 1-3 ml will be used (preferably 3 ml).

Multiple injections delivered as a single dose comprise about 0.1 to about 0.5 ml volumes. Compositions of the invention may be administered in multiple injections to a tumor or a targeted site. In certain aspects, injections may be spaced at approximately 1 cm intervals.

In the case of surgical intervention, the present invention may be used preoperatively, to render an inoperable tumor subject to resection. Alternatively, the present invention may be used at the time of surgery, and/or thereafter, to treat residual or metastatic disease. For example, a resected tumor bed may be injected or perfused with a formulation comprising a miRNA or combinations thereof. Administration may be continued post-resection, for example, by leaving a catheter implanted at the site of the surgery. Periodic post-surgical treatment also is envisioned. Continuous perfusion of an expression construct or a viral construct also is contemplated.

Continuous administration also may be applied where appropriate, for example, where a tumor or other undesired affected area is excised and the tumor bed or targeted site is treated to eliminate residual, microscopic disease. Delivery via syringe or catherization is contemplated. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs.

Treatment regimens may vary as well and often depend on tumor type, tumor location, immune condition, target site, disease progression, and health and age of the patient. Certain tumor types will require more aggressive treatment. The clinician will be best suited to make such decisions based on the known efficacy and toxicity (if any) of the therapeutic formulations.

In certain embodiments, the tumor or affected area being treated may not, at least initially, be resectable. Treatments with compositions of the invention may increase the resectability of the tumor due to shrinkage at the margins or by elimination of certain particularly invasive portions. Following treatments, resection may be possible. Additional treatments subsequent to resection may serve to eliminate microscopic residual disease at the tumor or targeted site.

Treatments may include various “unit doses.” A unit dose is defined as containing a predetermined quantity of a therapeutic composition(s). The quantity to be administered, and the particular route and formulation, are within the skill of those in the clinical arts. A unit dose need not be administered as a single injection but may comprise continuous infusion over a set period of time. With respect to a viral component of the present invention, a unit dose may conveniently be described in terms of μg or mg of miRNA or miRNA mimetic. Alternatively, the amount specified may be the amount administered as the average daily, average weekly, or average monthly dose.

miRNA can be administered to the patient in a dose or doses of about or of at least about 0.5, 1, 5, 10, 15, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 μg or mg, or more, or any range derivable therein. Alternatively, the amount specified may be the amount administered as the average daily, average weekly, or average monthly dose, or it may be expressed in terms of mg/kg, where kg refers to the weight of the patient and the mg is specified above. In other embodiments, the amount specified is any number discussed above but expressed as mg/m² (with respect to tumor size or patient surface area).

C. Injectable Compositions and Formulations

In some embodiments, the method for the delivery of a miRNA or an expression construct encoding such or combinations thereof is via systemic administration. However, the pharmaceutical compositions disclosed herein may also be administered parenterally, subcutaneously, directly, intratracheally, intravenously, intradermally, intramuscularly, or even intraperitoneally as described in U.S. Pat. Nos. 5,543,158; 5,641,515 and 5,399,363 (each specifically incorporated herein by reference in its entirety).

Injection of nucleic acids may be delivered by syringe or any other method used for injection of a solution, as long as the nucleic acid and any associated components can pass through the particular gauge of needle required for injection. A syringe system has also been described for use in gene therapy that permits multiple injections of predetermined quantities of a solution precisely at any depth (U.S. Pat. No. 5,846,225).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, mixtures thereof, and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In certain formulations, a water-based formulation is employed while in others, it may be lipid-based. In particular embodiments of the invention, a composition comprising a tumor suppressor protein or a nucleic acid encoding the same is in a water-based formulation. In other embodiments, the formulation is lipid based.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral, intralesional, and intraperitoneal administration. In this connection, sterile aqueous media which can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

As used herein, a “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically acceptable” refers to molecular entities and compositions that do not produce an allergic or similar untoward reaction when administered to a human.

The nucleic acid(s) are administered in a manner compatible with the dosage formulation, and in such amount as will be therapeutically effective. The quantity to be administered depends on the subject to be treated, including, e.g., the aggressiveness of the disease or cancer, the size of any tumor(s) or lesions, the previous or other courses of treatment. Precise amounts of active ingredient required to be administered depend on the judgment of the practitioner. Suitable regimes for initial administration and subsequent administration are also variable, but are typified by an initial administration followed by other administrations. Such administration may be systemic, as a single dose, continuous over a period of time spanning 10, 20, 30, 40, 50, 60 minutes, and/or 1, 2, 3, 4, 5, 6, 7, 8, 9, 10, 11, 12, 13, 14, 15, 16, 17, 18, 19, 20, 21, 22, 23, 24 or more hours, and/or 1, 2, 3, 4, 5, 6, 7, days or more. Moreover, administration may be through a time release or sustained release mechanism, implemented by formulation and/or mode of administration.

Various methods for nucleic acid delivery are described, for example in Sambrook et al., 1989 and Ausubel et al., 1994. Such nucleic acid delivery systems comprise the desired nucleic acid, by way of example and not by limitation, in either “naked” form as a “naked” nucleic acid, or formulated in a vehicle suitable for delivery, such as in a complex with a cationic molecule or a liposome forming lipid, or as a component of a vector, or a component of a pharmaceutical composition. The nucleic acid delivery system can be provided to the cell either directly, such as by contacting it with the cell, or indirectly, such as through the action of any biological process. By way of example, and not by limitation, the nucleic acid delivery system can be provided to the cell by endocytosis; receptor targeting; coupling with native or synthetic cell membrane fragments; physical means such as electroporation; combining the nucleic acid delivery system with a polymeric carrier, such as a controlled release film or nanoparticle or microparticle or biocompatible molecules or biodegradable molecules; with vector. The nucleic acid delivery system can be injected into a tissue or fluid surrounding the cell, or administered by diffusion of the nucleic acid delivery system across the cell membrane, or by any active or passive transport mechanism across the cell membrane. Additionally, the nucleic acid delivery system can be provided to the cell using techniques such as antibody-related targeting and antibody-mediated immobilization of a viral vector.

D. Combination Treatments

In certain embodiments, the compositions and methods of the present invention involve a miRNA, or expression construct encoding such. These miRNA composition can be used in combination with a second therapy to enhance the effect of the miRNA therapy, or increase the therapeutic effect of another therapy being employed. These compositions would be provided in a combined amount effective to achieve the desired effect, such as the killing of a cancer cell and/or the inhibition of cellular hyperproliferation. This process may involve contacting the cells with the miRNA or second therapy at the same or different time. This may be achieved by contacting the cell with one or more compositions or pharmacological formulation that includes or more of the agents, or by contacting the cell with two or more distinct compositions or formulations, wherein one composition provides (1) miRNA; and/or (2) a second therapy. A second composition or method may be administered that includes a chemotherapy, radiotherapy, surgical therapy, immunotherapy or gene therapy.

It is contemplated that one may provide a patient with the miRNA therapy and the second therapy within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

In certain embodiments, a course of treatment will last 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 days or more. It is contemplated that one agent may be given on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and/or 90, any combination thereof, and another agent is given on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and/or 90, or any combination thereof. Within a single day (24-hour period), the patient may be given one or multiple administrations of the agent(s). Moreover, after a course of treatment, it is contemplated that there is a period of time at which no treatment is administered. This time period may last 1, 2, 3, 4, 5, 6, 7 days, and/or 1, 2, 3, 4, 5 weeks, and/or 1, 2, 3, 4, 5, 6, 7, 8, 9, 10, 11, 12 months or more, depending on the condition of the patient, such as their prognosis, strength, health, etc.

Various combinations may be employed, for example miRNA therapy is “A” and a second therapy is “B”:

| | | | | | |
|---|---|---|---|---|---|
| A/B/A | B/A/B | B/B/A | A/A/B | A/B/B | B/A/A |
| A/B/B/B | B/A/B/B | B/B/B/A | B/B/A/B | A/A/B/B | A/B/A/B |
| A/B/B/A | B/B/A/A | B/A/B/A | B/A/A/B | A/A/A/B | B/A/A/A |
| A/B/A/A | A/A/B/A | | | | |

Administration of any compound or therapy of the present invention to a patient will follow general protocols for the administration of such compounds, taking into account the toxicity, if any, of the vector or any protein or other agent. Therefore, in some embodiments there is a step of monitoring toxicity that is attributable to combination therapy. It is expected that the treatment cycles would be repeated as necessary. It also is contemplated that various standard therapies, as well as surgical intervention, may be applied in combination with the described therapy.

In specific aspects, it is contemplated that a second therapy, such as chemotherapy, radiotherapy, immunotherapy, surgical therapy or other gene therapy, is employed in combination with the miRNA therapy, as described herein.

2. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

b. Alkylating Agents

Alkylating agents are drugs that directly interact with genomic DNA to prevent the cancer cell from proliferating. This category of chemotherapeutic drugs represents agents that affect all phases of the cell cycle, that is, they are not phase-specific. Alkylating agents can be implemented to treat chronic leukemia, non-Hodgkin's lymphoma, Hodgkin's disease, multiple myeloma, and particular cancers of the breast, lung, and ovary. They include: busulfan, chlorambucil, cisplatin, cyclophosphamide (cytoxan), dacarbazine, ifosfamide, mechlorethamine (mustargen), and melphalan. Troglitazaone can be used to treat cancer in combination with any one or more of these alkylating agents.

c. Antimetabolites

Antimetabolites disrupt DNA and RNA synthesis. Unlike alkylating agents, they specifically influence the cell cycle during S phase. They have been used to combat chronic leukemias in addition to tumors of breast, ovary and the gastrointestinal tract. Antimetabolites include 5-fluorouracil (5-FU), cytarabine (Ara-C), fludarabine, gemcitabine, and methotrexate.

5-Fluorouracil (5-FU) has the chemical name of 5-fluoro-2,4(1H,3H)-pyrimidinedione. Its mechanism of action is thought to be by blocking the methylation reaction of deoxyuridylic acid to thymidylic acid. Thus, 5-FU interferes with the synthesis of deoxyribonucleic acid (DNA) and to a lesser extent inhibits the formation of ribonucleic acid (RNA). Since DNA and RNA are essential for cell division and proliferation, it is thought that the effect of 5-FU is to create a thymidine deficiency leading to cell death. Thus, the effect of 5-FU is found in cells that rapidly divide, a characteristic of metastatic cancers.

d. Antitumor Antibiotics

Antitumor antibiotics have both antimicrobial and cytotoxic activity. These drugs also interfere with DNA by chemically inhibiting enzymes and mitosis or altering cellular membranes. These agents are not phase specific so they work in all phases of the cell cycle. Thus, they are widely used for a variety of cancers. Examples of antitumor antibiotics include bleomycin, dactinomycin, daunorubicin, doxorubicin (Adriamycin), and idarubicin, some of which are discussed in more detail below. Widely used in clinical setting for the treatment of neoplasms, these compounds are administered through bolus injections intravenously at doses ranging from 25-75 mg/m² at 21 day intervals for adriamycin, to 35-100 mg/m² for etoposide intravenously or orally.

e. Mitotic Inhibitors

Mitotic inhibitors include plant alkaloids and other natural agents that can inhibit either protein synthesis required for cell division or mitosis. They operate during a specific phase during the cell cycle. Mitotic inhibitors comprise docetaxel, etoposide (VP16), paclitaxel, taxol, taxotere, vinblastine, vincristine, and vinorelbine.

f. Nitrosureas

Nitrosureas, like alkylating agents, inhibit DNA repair proteins. They are used to treat non-Hodgkin's lymphomas, multiple myeloma, malignant melanoma, in addition to brain tumors. Examples include carmustine and lomustine.

3. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly. Radiotherapy may be used to treat localized solid tumors, such as cancers of the skin, tongue, larynx, brain, breast, or cervix. It can also be used to treat leukemia and lymphoma (cancers of the blood-forming cells and lymphatic system, respectively).

Radiation therapy used according to the present invention may include, but is not limited to, the use of γ-rays, X-rays, and/or the directed delivery of radioisotopes to tumor cells. Other forms of DNA damaging factors are also contemplated such as microwaves, proton beam irradiation (U.S. Pat. Nos. 5,760,395 and 4,870,287) and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells. Radiotherapy may comprise the use of radiolabeled antibodies to deliver doses of radiation directly to the cancer site (radioimmunotherapy). Once injected into the body, the antibodies actively seek out the cancer cells, which are destroyed by the cell-killing (cytotoxic) action of the radiation. This approach can minimize the risk of radiation damage to healthy cells.

Stereotactic radio-surgery (gamma knife) for brain and other tumors does not use a knife, but very precisely targeted beams of gamma radiotherapy from hundreds of different angles. Only one session of radiotherapy, taking about four to five hours, is needed. For this treatment a specially made metal frame is attached to the head. Then, several scans and x-rays are carried out to find the precise area where the treatment is needed. During the radiotherapy for brain tumors, the patient lies with their head in a large helmet, which has hundreds of holes in it to allow the radiotherapy beams through. Related approaches permit positioning for the treatment of tumors in other areas of the body.

4. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

In one aspect of immunotherapy, the tumor or disease cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor such as MDA-7 has been shown to enhance anti-tumor effects (Ju et al., 2000). Moreover, antibodies against any of these compounds can be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy e.g., interferons α, β and γ; IL-1, GM-CSF and TNF (Bukowski et al., 1998; Davidson et al., 1998; Hellstrand et al., 1998) gene therapy e.g., TNF, IL-1, IL-2, p53 (Qin et al., 1998; Austin-Ward and Villaseca, 1998; U.S. Pat. Nos. 5,830,880 and 5,846,945) and monoclonal antibodies e.g., anti-ganglioside GM2, anti-HER-2, anti-p185; Pietras et al., 1998; Hanibuchi et al., 1998; U.S. Pat. No. 5,824,311). Herceptin (trastuzumab) is a chimeric (mouse-human) monoclonal antibody that blocks the HER2-neu receptor. It possesses anti-tumor activity and has been approved for use in the treatment of malignant tumors (Dillman, 1999). Table 6 is a non-limiting list of several known anti-cancer immunotherapeutic agents and their targets. It is contemplated that one or more of these therapies may be employed with the miRNA therapies described herein.

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

TABLE 6

| Generic Name | Target |
|---|---|
| Cetuximab | EGFR |
| Panitumumab | EGFR |
| Trastuzumab | erbB2 receptor |
| Bevacizumab | VEGF |
| Alemtuzumab | CD52 |
| Gemtuzumab ozogamicin | CD33 |
| Rituximab | CD20 |
| Tositumomab | CD20 |
| Matuzumab | EGFR |
| Ibritumomab tiuxetan | CD20 |
| Tositumomab | CD20 |
| HuPAM4 | MUC1 |
| MORAb-009 | Mesothelin |
| G250 | carbonic anhydrase IX |
| mAb 8H9 | 8H9 antigen |
| M195 | CD33 |
| Ipilimumab | CTLA4 |
| HuLuc63 | CS1 |
| Alemtuzumab | CD53 |
| Epratuzumab | CD22 |
| BC8 | CD45 |
| HuJ591 | Prostate specific membrane antigen |
| hA20 | CD20 |
| Lexatumumab | TRAIL receptor-2 |
| Pertuzumab | HER-2 receptor |
| Mik-beta-1 | IL-2R |
| RAV12 | RAAG12 |
| SGN-30 | CD30 |
| AME-133v | CD20 |
| HeFi-1 | CD30 |
| BMS-663513 | CD137 |
| Volociximab | anti-α5β1 integrin |
| GC1008 | TGFβ |
| HCD122 | CD40 |
| Siplizumab | CD2 |
| MORAb-003 | Folate receptor alpha |
| CNTO 328 | IL-6 |
| MDX-060 | CD30 |
| Ofatumumab | CD20 |
| SGN-33 | CD33 |

5. Gene Therapy

In yet another embodiment, a combination treatment involves gene therapy in which a therapeutic polynucleotide is administered before, after, or at the same time as one or more therapeutic miRNA. Delivery of a therapeutic polypeptide or encoding nucleic acid in conjunction with a miRNA may have a combined therapeutic effect on target tissues. A variety of proteins are encompassed within the invention, some of which are described below. Various genes that may be targeted for gene therapy of some form in combination with the present invention include, but are not limited to inducers of cellular proliferation, inhibitors of cellular proliferation, regulators of programmed cell death, cytokines and other therapeutic nucleic acids or nucleic acid that encode therapeutic proteins.

The tumor suppressor oncogenes function to inhibit excessive cellular proliferation. The inactivation of these genes destroys their inhibitory activity, resulting in unregulated proliferation. The tumor suppressors (e.g., therapeutic polypeptides) p53, FHIT, p16 and C-CAM can be employed.

In addition to p53, another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G1. The activity of this enzyme may be to phosphorylate Rb at late G1. The activity of CDK4 is controlled by an activating subunit, D-type cyclin, and by an inhibitory subunit, the p161NK4 has been biochemically characterized as a protein that specifically binds to and inhibits CDK4, and thus may regulate Rb phosphorylation (Serrano et al., 1993; Serrano et al., 1995). Since the p161NK4 protein is a CDK4 inhibitor (Serrano, 1993), deletion of this gene may increase the activity of CDK4, resulting in hyperphosphorylation of the Rb protein. p16 also is known to regulate the function of CDK6.

p161NK4 belongs to a newly described class of CDK-inhibitory proteins that also includes p16B, p19, p21 WAF1, and p27KIP1. The p161NK4 gene maps to 9p21, a chromosome region frequently deleted in many tumor types. Homozygous deletions and mutations of the p161NK4 gene are frequent in human tumor cell lines. This evidence suggests that the p161NK4 gene is a tumor suppressor gene. This interpretation has been challenged, however, by the observation that the frequency of the p161NK4 gene alterations is much lower in primary uncultured tumors than in cultured cell lines (Caldas et al., 1994; Cheng et al., 1994; Hussussian et al., 1994; Kamb et al, 1994; Mori et al., 1994; Okamoto et al., 1994; Nobori et al., 1995; Orlow et al., 1994; Arap et al., 1995). Restoration of wild-type p161NK4 function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-1, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

6. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

7. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Apo2 ligand (Apo2L, also called TRAIL) is a member of the tumor necrosis factor (TNF) cytokine family. TRAIL activates rapid apoptosis in many types of cancer cells, yet is not toxic to normal cells. TRAIL mRNA occurs in a wide variety of tissues. Most normal cells appear to be resistant to TRAIL's cytotoxic action, suggesting the existence of mechanisms that can protect against apoptosis induction by TRAIL. The first receptor described for TRAIL, called death receptor 4 (DR4), contains a cytoplasmic “death domain”; DR4 transmits the apoptosis signal carried by TRAIL. Additional receptors have been identified that bind to TRAIL. One receptor, called DR5, contains a cytoplasmic death domain and signals apoptosis much like DR4. The DR4 and DR5 mRNAs are expressed in many normal tissues and tumor cell lines. Recently, decoy receptors such as DcR1 and DcR2 have been identified that prevent TRAIL from inducing apoptosis through DR4 and DR5. These decoy receptors thus represent a novel mechanism for regulating sensitivity to a pro-apoptotic cytokine directly at the cell's surface. The preferential expression of these inhibitory receptors in normal tissues suggests that TRAIL may be useful as an anticancer agent that induces apoptosis in cancer cells while sparing normal cells. (Marsters et al, 1999).

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

This application incorporates U.S. application Ser. No. 11/349,727 filed on Feb. 8, 2006 claiming priority to U.S. Provisional Application Ser. No. 60/650,807 filed Feb. 8, 2005 herein by references in its entirety.

### IV. miRNA MOLECULES

MicroRNA molecules (“miRNAs”) are generally 21 to 22 nucleotides in length, though lengths of 19 and up to 23 nucleotides have been reported. The miRNAs are each processed from a longer precursor RNA molecule (“precursor miRNA”). Precursor miRNAs are transcribed from non-protein-encoding genes. The precursor miRNAs have two regions of complementarity that enables them to form a stem-loop- or fold-back-like structure, which is cleaved in animals by a ribonuclease III-like nuclease enzyme called Dicer. The processed miRNA is typically a portion of the stem.

The processed miRNA (also referred to as “mature miRNA”) becomes part of a large complex to down-regulate a particular target gene or its gene product. Examples of animal miRNAs include those that imperfectly basepair with the target, which halts translation (Olsen et al., 1999; Seggerson et al., 2002). siRNA molecules also are processed by Dicer, but from a long, double-stranded RNA molecule. siRNAs are not naturally found in animal cells, but they can direct the sequence-specific cleavage of an mRNA target through a RNA-induced silencing complex (RISC) (Denli et al, 2003).

B. Array Preparation

Certain embodiments of the present invention concerns the preparation and use of mRNA or nucleic acid arrays, miRNA or nucleic acid arrays, and/or miRNA or nucleic acid probe arrays, which are macroarrays or microarrays of nucleic acid molecules (probes) that are fully or nearly complementary (over the length of the prove) or identical (over the length of the prove) to a plurality of nucleic acid, mRNA or miRNA molecules, precursor miRNA molecules, or nucleic acids derived from the various genes and gene pathways modulated by miR-143 miRNAs and that are positioned on a support or support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of marker RNA and/or miRNA-complementing nucleic acid samples, the position of each sample can be tracked and linked to the original sample.

A variety of different array devices in which a plurality of distinct nucleic acid probes are stably associated with the surface of a solid support are known to those of skill in the art. Useful substrates for arrays include nylon, glass, metal, plastic, latex, and silicon. Such arrays may vary in a number of different ways, including average probe length, sequence or types of probes, nature of bond between the probe and the array surface, e.g. covalent or non-covalent, and the like. The labeling and screening methods of the present invention and the arrays are not limited in its utility with respect to any parameter except that the probes detect miRNA, or genes or nucleic acid representative of genes; consequently, methods and compositions may be used with a variety of different types of nucleic acid arrays.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610,287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO0138580; WO 0168255; WO 03020898; WO 03040410; WO 03053586; WO 03087297; WO 03091426; WO03100012; WO 04020085; WO 04027093; EP 373 203; EP 785 280; EP 799 897 and UK 8 803 000; the disclosures of which are all herein incorporated by reference.

It is contemplated that the arrays can be high density arrays, such that they contain 2, 20, 25, 50, 80, 100 or more different probes. It is contemplated that they may contain 1000, 16,000, 65,000, 250,000 or 1,000,000 or more different probes. The probes can be directed to mRNA and/or miRNA targets in one or more different organisms or cell types. The oligonucleotide probes range from 5 to 50, 5 to 45, 10 to 40, 9 to 34, or 15 to 40 nucleotides in length in some embodiments. In certain embodiments, the oligonucleotide probes are 5, 10, 15, 20 to 20, 25, 30, 35, 40 nucleotides in length including all integers and ranges there between.

The location and sequence of each different probe sequence in the array are generally known. Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm². The surface area of the array can be about or less than about 1, 1.6, 2, 3, 4, 5, 6, 7, 8, 9, or 10 cm².

Moreover, a person of ordinary skill in the art could readily analyze data generated using an array. Such protocols are disclosed above, and include information found in WO 9743450; WO 03023058; WO 03022421; WO 03029485; WO 03067217; WO 03066906; WO 03076928; WO 03093810; WO 03100448A1, all of which are specifically incorporated by reference.

C. Sample Preparation

It is contemplated that the RNA and/or miRNA of a wide variety of samples can be analyzed using the arrays, index of probes, or array technology of the invention. While endogenous miRNA is contemplated for use with compositions and methods of the invention, recombinant miRNA—including nucleic acids that are complementary or identical to endogenous miRNA or precursor miRNA—can also be handled and analyzed as described herein. Samples may be biological samples, in which case, they can be from biopsy, fine needle aspirates, exfoliates, blood, tissue, organs, semen, saliva, tears, other bodily fluid, hair follicles, skin, or any sample containing or constituting biological cells, particularly cancer or hyperproliferative cells. In certain embodiments, samples may be, but are not limited to, biopsy, or cells purified or enriched to some extent from a biopsy or other bodily fluids or tissues. Alternatively, the sample may not be a biological sample, but be a chemical mixture, such as a cell-free reaction mixture (which may contain one or more biological enzymes).

D. Hybridization

After an array or a set of probes is prepared and/or the nucleic acid in the sample or probe is labeled, the population of target nucleic acids is contacted with the array or probes under hybridization conditions, where such conditions can be adjusted, as desired, to provide for an optimum level of specificity in view of the particular assay being performed. Suitable hybridization conditions are well known to those of skill in the art and reviewed in Sambrook et al. (2001) and WO 95/21944. Of particular interest in many embodiments is the use of stringent conditions during hybridization. Stringent conditions are known to those of skill in the art.

It is specifically contemplated that a single array or set of probes may be contacted with multiple samples. The samples may be labeled with different labels to distinguish the samples. For example, a single array can be contacted with a tumor tissue sample labeled with Cy3, and normal tissue sample labeled with Cy5. Differences between the samples for particular miRNAs corresponding to probes on the array can be readily ascertained and quantified.

The small surface area of the array permits uniform hybridization conditions, such as temperature regulation and salt content. Moreover, because of the small area occupied by the high density arrays, hybridization may be carried out in extremely small fluid volumes (e.g., about 250 μl or less, including volumes of about or less than about 5, 10, 25, 50, 60, 70, 80, 90, 100 μl, or any range derivable therein). In small volumes, hybridization may proceed very rapidly.

E. Differential Expression Analyses

Arrays of the invention can be used to detect differences between two samples. Specifically contemplated applications include identifying and/or quantifying differences between miRNA or gene expression from a sample that is normal and from a sample that is not normal, between a disease or condition and a cell not exhibiting such a disease or condition, or between two differently treated samples. Also, miRNA or gene expression may be compared between a sample believed to be susceptible to a particular disease or condition and one believed to be not susceptible or resistant to that disease or condition. A sample that is not normal is one exhibiting phenotypic or genotypic trait(s) of a disease or condition, or one believed to be not normal with respect to that disease or condition. It may be compared to a cell that is normal with respect to that disease or condition. Phenotypic traits include symptoms of, or susceptibility to, a disease or condition of which a component is or may or may not be genetic, or caused by a hyperproliferative or neoplastic cell or cells.

An array comprises a solid support with nucleic acid probes attached to the support. Arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., (1991), each of which is incorporated by reference in its entirety for all purposes. Techniques for the synthesis of these arrays using mechanical synthesis methods are described in, e.g., U.S. Pat. No. 5,384,261, incorporated herein by reference in its entirety for all purposes. Although a planar array surface is used in certain aspects, the array may be fabricated on a surface of virtually any shape or even a multiplicity of surfaces. Arrays may be nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, which are hereby incorporated in their entirety for all purposes. Arrays may be packaged in such a manner as to allow for diagnostics or other manipulation of an all inclusive device, see for example, U.S. Pat. Nos. 5,856,174 and 5,922,591 incorporated in their entirety by reference for all purposes. See also U.S. patent application Ser. No. 09/545,207, filed Apr. 7, 2000 for additional information concerning arrays, their manufacture, and their characteristics, which is incorporated by reference in its entirety for all purposes.

Particularly, arrays can be used to evaluate samples with respect to pathological condition such as cancer and related conditions. It is specifically contemplated that the invention can be used to evaluate differences between stages or sub-classifications of disease, such as between benign, cancerous, and metastatic tissues or tumors.

Phenotypic traits to be assessed include characteristics such as longevity, morbidity, expected survival, susceptibility or receptivity to particular drugs or therapeutic treatments (drug efficacy), and risk of drug toxicity. Samples that differ in these phenotypic traits may also be evaluated using the compositions and methods described.

In certain embodiments, miRNA and/or expression profiles may be generated to evaluate and correlate those profiles with pharmacokinetics or therapies. For example, these profiles may be created and evaluated for patient tumor and blood samples prior to the patient's being treated or during treatment to determine if there are miRNA or genes whose expression correlates with the outcome of the patient's treatment. Identification of differential miRNAs or genes can lead to a diagnostic assay for evaluation of tumor and/or blood samples to determine what drug regimen the patient should be provided. In addition, it can be used to identify or select patients suitable for a particular clinical trial. If an expression profile is determined to be correlated with drug efficacy or drug toxicity that profile is relevant to whether that patient is an appropriate patient for receiving a drug, for receiving a combination of drugs, or for a particular dosage of the drug.

In addition to the above prognostic assay, samples from patients with a variety of diseases can be evaluated to determine if different diseases can be identified based on miRNA and/or related gene expression levels. A diagnostic assay can be created based on the profiles that doctors can use to identify individuals with a disease or who are at risk to develop a disease. Alternatively, treatments can be designed based on miRNA profiling. Examples of such methods and compositions are described in the U.S. Provisional Patent Application entitled “Methods and Compositions Involving miRNA and miRNA Inhibitor Molecules” filed on May 23, 2005, which is hereby incorporated by reference in its entirety.

F. Other Assays

In addition to the use of arrays and microarrays, it is contemplated that a number of different assays could be employed to analyze miRNAs or related genes, their activities, and their effects. Such assays include, but are not limited to, nucleic acid amplification, polymerase chain reaction, quantitative PCR, RT-PCR, in situ hybridization, Northern hybridization, hybridization protection assay (HPA)(GenProbe), branched DNA (bDNA) assay (Chiron), rolling circle amplification (RCA), single molecule hybridization detection (US Genomics), Invader assay (ThirdWave Technologies), and/or Bridge Litigation Assay (Genaco).

### V. NUCLEIC ACIDS

The present invention concerns nucleic acids, modified or mimetic nucleic acids, miRNAs, mRNAs, genes, and representative fragments thereof that can be labeled, used in array analysis, or employed in diagnostic, therapeutic, or prognostic applications, particularly those related to pathological conditions such as cancer. The molecules may have been endogenously produced by a cell, or been synthesized or produced chemically or recombinantly. They may be isolated and/or purified. Each of the miRNAs described herein and include the corresponding SEQ ID NO and accession numbers for these miRNA sequences. The name of a miRNA is often abbreviated and referred to without a “hsa-” prefix and will be understood as such, depending on the context. Unless otherwise indicated, miRNAs referred to in the application are human sequences identified as miR-X or let-X, where X is a number and/or letter.

In certain aspects, a miRNA probe designated by a suffix “5P” or “3P” can be used. “5P” indicates that the mature miRNA derives from the 5′ end of the precursor and a corresponding “3P” indicates that it derives from the 3′ end of the precursor, as described on the world wide web at sanger.ac.uk. Moreover, in some embodiments, a miRNA probe is used that does not correspond to a known human miRNA. It is contemplated that these non-human miRNA probes may be used in embodiments of the invention or that there may exist a human miRNA that is homologous to the non-human miRNA. In other embodiments, any mammalian cell, biological sample, or preparation thereof may be employed.

In some embodiments of the invention, methods and compositions involving miRNA may concern miRNA, markers (mRNAs), and/or other nucleic acids. Nucleic acids may be, be at least, or be at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides, or any range derivable therein, in length. Such lengths cover the lengths of processed miRNA, miRNA probes, precursor miRNA, miRNA containing vectors, mRNA, mRNA probes, control nucleic acids, and other probes and primers.

In many embodiments, miRNA are 19-24 nucleotides in length, while miRNA probes are 19-35 nucleotides in length, depending on the length of the processed miRNA and any flanking regions added. miRNA precursors are generally between 62 and 110 nucleotides in humans.

Nucleic acids of the invention may have regions of identity or complementarity to another nucleic acid. It is contemplated that the region of complementarity or identity can be at least 5 contiguous residues, though it is specifically contemplated that the region is, is at least, or is at most 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 contiguous nucleotides. It is further understood that the length of complementarity within a precursor miRNA or other nucleic acid or between a miRNA probe and a miRNA or a miRNA gene are such lengths. Moreover, the complementarity may be expressed as a percentage, meaning that the complementarity between a probe and its target is 90% or greater over the length of the probe. In some embodiments, complementarity is or is at least 90%, 95% or 100%. In particular, such lengths may be applied to any nucleic acid comprising a nucleic acid sequence identified in any of SEQ ID NO:1-13, accession number, or any other sequence disclosed herein. Typically, the commonly used name of the miRNA is given (with its identifying source in the prefix, for example, “hsa” for human sequences) and the processed miRNA sequence. Unless otherwise indicated, a miRNA without a prefix will be understood to refer to a human miRNA. Moreover, a lowercase letter in a miRNA name may or may not be lowercase; for example, hsa-mir-130b can also be referred to as miR-130B. The term “miRNA probe” refers to a nucleic acid probe that can identify a particular miRNA or structurally related miRNAs.

It is understood that some nucleic acids are derived from genomic sequences or a gene. In this respect, the term “gene” is used for simplicity to refer to the genomic sequence encoding the precursor nucleic acid or miRNA for a given miRNA or gene. However, embodiments of the invention may involve genomic sequences of a miRNA that are involved in its expression, such as a promoter or other regulatory sequences.

The term “recombinant” may be used and this generally refers to a molecule that has been manipulated in vitro or that is a replicated or expressed product of such a molecule.

The term “nucleic acid” is well known in the art. A “nucleic acid” as used herein will generally refer to a molecule (one or more strands) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompasses the terms “oligonucleotide” and “polynucleotide,” each as a subgenus of the term “nucleic acid.”

The term “miRNA” generally refers to a single-stranded molecule, but in specific embodiments, molecules implemented in the invention will also encompass a region or an additional strand that is partially (between 10 and 50% complementary across length of strand), substantially (greater than 50% but less than 100% complementary across length of strand) or fully complementary to another region of the same single-stranded molecule or to another nucleic acid. Thus, miRNA may encompass a molecule that comprises one or more complementary or self-complementary strand(s) or “complement(s)” of a particular sequence. For example, precursor miRNA may have a self-complementary region, which is up to 100% complementary. miRNA probes or nucleic acids of the invention can include, can be or can be at least 60, 65, 70, 75, 80, 85, 90, 95, 96, 97, 98, 99 or 100% complementary to their target.

It is understood that a “synthetic nucleic acid” of the invention means that the nucleic acid does not have all or part of a chemical structure or sequence of a naturally occurring nucleic acid. Consequently, it will be understood that the term “synthetic miRNA” refers to a “synthetic nucleic acid” that functions in a cell or under physiological conditions as a naturally occurring miRNA.

While embodiments of the invention may involve synthetic miRNAs or synthetic nucleic acids, in some embodiments of the invention, the nucleic acid molecule(s) need not be “synthetic.” In certain embodiments, a non-synthetic nucleic acid or miRNA employed in methods and compositions of the invention may have the entire sequence and structure of a naturally occurring mRNA or miRNA precursor or the mature mRNA or miRNA. For example, non-synthetic miRNAs used in methods and compositions of the invention may not have one or more modified nucleotides or nucleotide analogs. In these embodiments, the non-synthetic miRNA may or may not be recombinantly produced. In particular embodiments, the nucleic acid in methods and/or compositions of the invention is specifically a synthetic miRNA and not a non-synthetic miRNA (that is, not an miRNA that qualifies as “synthetic”); though in other embodiments, the invention specifically involves a non-synthetic miRNA and not a synthetic miRNA. Any embodiments discussed with respect to the use of synthetic miRNAs can be applied with respect to non-synthetic miRNAs, and vice versa.

It will be understood that the term “naturally occurring” refers to something found in an organism without any intervention by a person; it could refer to a naturally-occurring wildtype or mutant molecule. In some embodiments a synthetic miRNA molecule does not have the sequence of a naturally occurring miRNA molecule. In other embodiments, a synthetic miRNA molecule may have the sequence of a naturally occurring miRNA molecule, but the chemical structure of the molecule, particularly in the part unrelated specifically to the precise sequence (non-sequence chemical structure) differs from chemical structure of the naturally occurring miRNA molecule with that sequence. In some cases, the synthetic miRNA has both a sequence and non-sequence chemical structure that are not found in a naturally-occurring miRNA. Moreover, the sequence of the synthetic molecules will identify which miRNA is effectively being provided or inhibited; the endogenous miRNA will be referred to as the “corresponding miRNA.” Corresponding miRNA sequences that can be used in the context of the invention include, but are not limited to, all or a portion of those sequences in the SEQ IDs provided herein, as well as any other miRNA sequence, miRNA precursor sequence, or any sequence complementary thereof. In some embodiments, the sequence is or is derived from or contains all or part of a sequence identified herein to target a particular miRNA (or set of miRNAs) that can be used with that sequence.

As used herein, “hybridization”, “hybridizes” or “capable of hybridizing” is understood to mean the forming of a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but preclude hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.5 M NaCl at temperatures of about 42° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification of a related target nucleic acid that does not hybridize to a nucleic acid under stringent conditions may be achieved by hybridization at low temperature and/or high ionic strength. Such conditions are termed “low stringency” or “low stringency conditions,” and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

B. Nucleobase, Nucleoside, Nucleotide, and Modified Nucleotides

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in a manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those a purine or pyrimidine substituted by one or more of an alkyl, caboxyalkyl, amino, hydroxyl, halogen (i.e., fluoro, chloro, bromo, or iodo), thiol or alkylthiol moiety. Preferred alkyl (e.g., alkyl, caboxyalkyl, etc.) moieties comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like. Other examples are well known to those of skill in the art.

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring. Different types of covalent attachment(s) of a nucleobase to a nucleobase linker moiety are known in the art (Kornberg and Baker, 1992).

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

A nucleic acid may comprise, or be composed entirely of, a derivative or analog of a nucleobase, a nucleobase linker moiety and/or backbone moiety that may be present in a naturally occurring nucleic acid. RNA with nucleic acid analogs may also be labeled according to methods of the invention. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, 1980, incorporated herein by reference).

Additional non-limiting examples of nucleosides, nucleotides or nucleic acids include those in: U.S. Pat. Nos. 5,681,947, 5,652,099 and 5,763,167, 5,614,617, 5,670,663, 5,872,232, 5,859,221, 5,446,137, 5,886,165, 5,714,606, 5,672,697, 5,466,786, 5,792,847, 5,223,618, 5,470,967, 5,378,825, 5,777,092, 5,623,070, 5,610,289, 5,602,240, 5,858,988, 5,214,136, 5,700,922, 5,708,154, 5,728,525, 5,637,683, 6,251,666, 5,480,980, and 5,728,525, each of which is incorporated herein by reference in its entirety.

Labeling methods and kits of the invention specifically contemplate the use of nucleotides that are both modified for attachment of a label and can be incorporated into a miRNA molecule. Such nucleotides include those that can be labeled with a dye, including a fluorescent dye, or with a molecule such as biotin. Labeled nucleotides are readily available; they can be acquired commercially or they can be synthesized by reactions known to those of skill in the art.

Modified nucleotides for use in the invention are not naturally occurring nucleotides, but instead, refer to prepared nucleotides that have a reactive moiety on them. Specific reactive functionalities of interest include: amino, sulfhydryl, sulfoxyl, aminosulfhydryl, azido, epoxide, isothiocyanate, isocyanate, anhydride, monochlorotriazine, dichlorotriazine, mono- or dihalogen substituted pyridine, mono- or disubstituted diazine, maleimide, epoxide, aziridine, sulfonyl halide, acid halide, alkyl halide, aryl halide, alkylsulfonate, N-hydroxysuccinimide ester, imido ester, hydrazine, azidonitrophenyl, azide, 3-(2-pyridyl dithio)-propionamide, glyoxal, aldehyde, iodoacetyl, cyanomethyl ester, p-nitrophenyl ester, o-nitrophenyl ester, hydroxypyridine ester, carbonyl imidazole, and the other such chemical groups. In some embodiments, the reactive functionality may be bonded directly to a nucleotide, or it may be bonded to the nucleotide through a linking group. The functional moiety and any linker cannot substantially impair the ability of the nucleotide to be added to the miRNA or to be labeled. Representative linking groups include carbon containing linking groups, typically ranging from about 2 to 18, usually from about 2 to 8 carbon atoms, where the carbon containing linking groups may or may not include one or more heteroatoms, e.g. S, O, N etc., and may or may not include one or more sites of unsaturation. Of particular interest in many embodiments are alkyl linking groups, typically lower alkyl linking groups of 1 to 16, usually 1 to 4 carbon atoms, where the linking groups may include one or more sites of unsaturation. The functionalized nucleotides (or primers) used in the above methods of functionalized target generation may be fabricated using known protocols or purchased from commercial vendors, e.g., Sigma, Roche, Ambion, Biosearch Technologies and NEN. Functional groups may be prepared according to ways known to those of skill in the art, including the representative information found in U.S. Pat. Nos. 4,404,289; 4,405,711; 4,337,063 and 5,268,486, and U.K. Patent 1,529,202, which are all incorporated by reference.

Amine-modified nucleotides are used in several embodiments of the invention. The amine-modified nucleotide is a nucleotide that has a reactive amine group for attachment of the label. It is contemplated that any ribonucleotide (G, A, U, or C) or deoxyribonucleotide (G, A, T, or C) can be modified for labeling. Examples include, but are not limited to, the following modified ribo- and deoxyribo-nucleotides: 5-(3-aminoallyl)-UTP; 8-[(4-amino)butyl]-amino-ATP and 8-[(6-amino)butyl]-amino-ATP; N6-(4-amino)butyl-ATP, N6-(6-amino)butyl-ATP, N4-[2,2-oxy-bis-(ethylamine)]-CTP; N6-(6-Amino)hexyl-ATP; 8-[(6-Amino)hexyl]-amino-ATP; 5-propargylamino-CTP, 5-propargylamino-UTP; 5-(3-aminoallyl)-dUTP; 8-[(4-amino)butyl]-amino-dATP and 8-[(6-amino)butyl]-amino-dATP; N6-(4-amino)butyl-dATP, N6-(6-amino)butyl-dATP, N4-[2,2-oxy-bis-(ethylamine)]-dCTP; N6-(6-Amino)hexyl-dATP; 8-[(6-Amino)hexyl]-amino-dATP; 5-propargylamino-dCTP, and 5-propargylamino-dUTP. Such nucleotides can be prepared according to methods known to those of skill in the art. Moreover, a person of ordinary skill in the art could prepare other nucleotide entities with the same amine-modification, such as a 5-(3-aminoallyl)-CTP, GTP, ATP, dCTP, dGTP, dTTP, or dUTP in place of a 5-(3-aminoallyl)-UTP.

C. Preparation of Nucleic Acids

A nucleic acid may be made by any technique known to one of ordinary skill in the art, such as for example, chemical synthesis, enzymatic production, or biological production. It is specifically contemplated that miRNA probes of the invention are chemically synthesized.

In some embodiments of the invention, miRNAs are recovered or isolated from a biological sample. The miRNA may be recombinant or it may be natural or endogenous to the cell (produced from the cell's genome). It is contemplated that a biological sample may be treated in a way so as to enhance the recovery of small RNA molecules such as miRNA. U.S. patent application Ser. No. 10/667,126 describes such methods and it is specifically incorporated by reference herein. Generally, methods involve lysing cells with a solution having guanidinium and a detergent.

Alternatively, nucleic acid synthesis is performed according to standard methods. See, for example, Itakura and Riggs (1980) and U.S. Pat. Nos. 4,704,362, 5,221,619, and 5,583,013, each of which is incorporated herein by reference. Non-limiting examples of a synthetic nucleic acid (e.g., a synthetic oligonucleotide), include a nucleic acid made by in vitro chemically synthesis using phosphotriester, phosphite, or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. Nos. 4,683,202 and 4,682,195, each incorporated herein by reference), or the synthesis of an oligonucleotide described in U.S. Pat. No. 5,645,897, incorporated herein by reference. See also Sambrook et al., 2001, incorporated herein by reference).

Oligonucleotide synthesis is well known to those of skill in the art. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

Recombinant methods for producing nucleic acids in a cell are well known to those of skill in the art. These include the use of vectors (viral and non-viral), plasmids, cosmids, and other vehicles for delivering a nucleic acid to a cell, which may be the target cell (e.g., a cancer cell) or simply a host cell (to produce large quantities of the desired RNA molecule). Alternatively, such vehicles can be used in the context of a cell free system so long as the reagents for generating the RNA molecule are present. Such methods include those described in Sambrook, 2003, Sambrook, 2001 and Sambrook, 1989, which are hereby incorporated by reference.

D. Isolation of Nucleic Acids

Nucleic acids may be isolated using techniques well known to those of skill in the art, though in particular embodiments, methods for isolating small nucleic acid molecules, and/or isolating RNA molecules can be employed. Chromatography is a process often used to separate or isolate nucleic acids from protein or from other nucleic acids. Such methods can involve electrophoresis with a gel matrix, filter columns, alcohol precipitation, and/or other chromatography. If miRNA from cells is to be used or evaluated, methods generally involve lysing the cells with a chaotropic (e.g., guanidinium isothiocyanate) and/or detergent (e.g., N-lauroyl sarcosine) prior to implementing processes for isolating particular populations of RNA.

In particular methods for separating miRNA from other nucleic acids, a gel matrix is prepared using polyacrylamide, though agarose can also be used. The gels may be graded by concentration or they may be uniform. Plates or tubing can be used to hold the gel matrix for electrophoresis. Usually one-dimensional electrophoresis is employed for the separation of nucleic acids. Plates are used to prepare a slab gel, while the tubing (glass or rubber, typically) can be used to prepare a tube gel. The phrase “tube electrophoresis” refers to the use of a tube or tubing, instead of plates, to form the gel. Materials for implementing tube electrophoresis can be readily prepared by a person of skill in the art or purchased, such as from C.B.S. Scientific Co., Inc. or Scie-Plas.

Methods may involve the use of organic solvents and/or alcohol to isolate nucleic acids, particularly miRNA used in methods and compositions of the invention. Some embodiments are described in U.S. patent application Ser. No. 10/667,126, which is hereby incorporated by reference. Generally, this disclosure provides methods for efficiently isolating small RNA molecules from cells comprising: adding an alcohol solution to a cell lysate and applying the alcohol/lysate mixture to a solid support before eluting the RNA molecules from the solid support. In some embodiments, the amount of alcohol added to a cell lysate achieves an alcohol concentration of about 55% to 60%. While different alcohols can be employed, ethanol works well. A solid support may be any structure, and it includes beads, filters, and columns, which may include a mineral or polymer support with electronegative groups. A glass fiber filter or column has worked particularly well for such isolation procedures.

In specific embodiments, miRNA isolation processes include: a) lysing cells in the sample with a lysing solution comprising guanidinium, wherein a lysate with a concentration of at least about 1 M guanidinium is produced; b) extracting miRNA molecules from the lysate with an extraction solution comprising phenol; c) adding to the lysate an alcohol solution for forming a lysate/alcohol mixture, wherein the concentration of alcohol in the mixture is between about 35% to about 70%; d) applying the lysate/alcohol mixture to a solid support; e) eluting the miRNA molecules from the solid support with an ionic solution; and, f) capturing the miRNA molecules. Typically the sample is dried and resuspended in a liquid and volume appropriate for subsequent manipulation.

### VI. LABELS AND LABELING TECHNIQUES

In some embodiments, the present invention concerns miRNA that are labeled. It is contemplated that miRNA may first be isolated and/or purified prior to labeling. This may achieve a reaction that more efficiently labels the miRNA, as opposed to other RNA in a sample in which the miRNA is not isolated or purified prior to labeling. In many embodiments of the invention, the label is non-radioactive. Generally, nucleic acids may be labeled by adding labeled nucleotides (one-step process) or adding nucleotides and labeling the added nucleotides (two-step process).

B. Labeling Techniques

In some embodiments, nucleic acids are labeled by catalytically adding to the nucleic acid an already labeled nucleotide or nucleotides. One or more labeled nucleotides can be added to miRNA molecules. See U.S. Pat. No. 6,723,509, which is hereby incorporated by reference.

In other embodiments, an unlabeled nucleotide or nucleotides is catalytically added to a miRNA, and the unlabeled nucleotide is modified with a chemical moiety that enables it to be subsequently labeled. In embodiments of the invention, the chemical moiety is a reactive amine such that the nucleotide is an amine-modified nucleotide. Examples of amine-modified nucleotides are well known to those of skill in the art, many being commercially available such as from Ambion, Sigma, Jena Bioscience, and TriLink.

In contrast to labeling of cDNA during its synthesis, the issue for labeling miRNA is how to label the already existing molecule. The present invention concerns the use of an enzyme capable of using a di- or tri-phosphate ribonucleotide or deoxyribonucleotide as a substrate for its addition to a miRNA. Moreover, in specific embodiments, it involves using a modified di- or tri-phosphate ribonucleotide, which is added to the 3′ end of a miRNA. Enzymes capable of adding such nucleotides include, but are not limited to, poly(A) polymerase, terminal transferase, and polynucleotide phosphorylase. In specific embodiments of the invention, a ligase is contemplated as not being the enzyme used to add the label, and instead, a non-ligase enzyme is employed. Terminal transferase catalyzes the addition of nucleotides to the 3′ terminus of a nucleic acid. Polynucleotide phosphorylase can polymerize nucleotide diphosphates without the need for a primer.

C. Labels

Labels on miRNA or miRNA probes may be colorimetric (includes visible and UV spectrum, including fluorescent), luminescent, enzymatic, or positron emitting (including radioactive). The label may be detected directly or indirectly. Radioactive labels include ¹²⁵I, ³²P, ³³P, and ³⁵S. Examples of enzymatic labels include alkaline phosphatase, luciferase, horseradish peroxidase, and β-galactosidase. Labels can also be proteins with luminescent properties, e.g., green fluorescent protein and phycoerythrin.

The colorimetric and fluorescent labels contemplated for use as conjugates include, but are not limited to, Alexa Fluor dyes, BODIPY dyes, such as BODIPY FL; Cascade Blue; Cascade Yellow; coumarin and its derivatives, such as 7-amino-4-methylcoumarin, aminocoumarin and hydroxycoumarin; cyanine dyes, such as Cy3 and Cy5; eosins and erythrosins; fluorescein and its derivatives, such as fluorescein isothiocyanate; macrocyclic chelates of lanthanide ions, such as Quantum Dye™; Marina Blue; Oregon Green; rhodamine dyes, such as rhodamine red, tetramethylrhodamine and rhodamine 6G; Texas Red; fluorescent energy transfer dyes, such as thiazole orange-ethidium heterodimer; and, TOTAB.

Specific examples of dyes include, but are not limited to, those identified above and

the following: Alexa Fluor 350, Alexa Fluor 405, Alexa Fluor 430, Alexa Fluor 488, Alexa Fluor 500. Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, and, Alexa Fluor 750; amine-reactive BODIPY dyes, such as BODIPY 493/503, BODIPY 530/550, BODIPY 558/568, BODIPY 564/570, BODIPY 576/589, BODIPY 581/591, BODIPY 630/650, BODIPY 650/655, BODIPY FL, BODIPY R6G, BODIPY TMR, and, BODIPY-TR; Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, 2′,4′,5′,7′-Tetrabromosulfonefluorescein, and TET.

Specific examples of fluorescently labeled ribonucleotides are available from Molecular Probes, and these include, Alexa Fluor 488-5-UTP, Fluorescein-12-UTP, BODIPY FL-14-UTP, BODIPY TMR-14-UTP, Tetramethylrhodamine-6-UTP, Alexa Fluor 546-14-UTP, Texas Red-5-UTP, and BODIPY TR-14-UTP. Other fluorescent ribonucleotides are available from Amersham Biosciences, such as Cy3-UTP and Cy5-UTP.

Examples of fluorescently labeled deoxyribonucleotides include Dinitrophenyl (DNP)-1-dUTP, Cascade Blue-7-dUTP, Alexa Fluor 488-5-dUTP, Fluorescein-12-dUTP, Oregon Green 488-5-dUTP, BODIPY FL-14-dUTP, Rhodamine Green-5-dUTP, Alexa Fluor 532-5-dUTP, BODIPY TMR-14-dUTP, Tetramethylrhodamine-6-dUTP, Alexa Fluor 546-14-dUTP, Alexa Fluor 568-5-dUTP, Texas Red-12-dUTP, Texas Red-5-dUTP, BODIPY TR-14-dUTP, Alexa Fluor 594-5-dUTP, BODIPY 630/650-14-dUTP, BODIPY 650/665-14-dUTP; Alexa Fluor 488-7-OBEA-dCTP, Alexa Fluor 546-16-OBEA-dCTP, Alexa Fluor 594-7-OBEA-dCTP, Alexa Fluor 647-12-OBEA-dCTP.

It is contemplated that nucleic acids may be labeled with two different labels. Furthermore, fluorescence resonance energy transfer (FRET) may be employed in methods of the invention (e.g., Klostermeier et al., 2002; Emptage, 2001; Didenko, 2001, each incorporated by reference).

Alternatively, the label may not be detectable per se, but indirectly detectable or allowing for the isolation or separation of the targeted nucleic acid. For example, the label could be biotin, digoxigenin, polyvalent cations, chelator groups and the other ligands, include ligands for an antibody.

D. Visualization Techniques

A number of techniques for visualizing or detecting labeled nucleic acids are readily available. Such techniques include, microscopy, arrays, Fluorometry, Light cyclers or other real time PCR machines, FACS analysis, scintillation counters, Phosphoimagers, Geiger counters, MRI, CAT, antibody-based detection methods (Westerns, immunofluorescence, immunohistochemistry), histochemical techniques, HPLC (Griffey et al., 1997), spectroscopy, capillary gel electrophoresis (Cummins et al., 1996), spectroscopy; mass spectroscopy; radiological techniques; and mass balance techniques.

When two or more differentially colored labels are employed, fluorescent resonance energy transfer (FRET) techniques may be employed to characterize association of one or more nucleic acid. Furthermore, a person of ordinary skill in the art is well aware of ways of visualizing, identifying, and characterizing labeled nucleic acids, and accordingly, such protocols may be used as part of the invention. Examples of tools that may be used also include fluorescent microscopy, a BioAnalyzer, a plate reader, Storm (Molecular Dynamics), Array Scanner, FACS (fluorescent activated cell sorter), or any instrument that has the ability to excite and detect a fluorescent molecule.

### VII. KITS

Any of the compositions described herein may be comprised in a kit. In a non-limiting example, reagents for isolating miRNA, labeling miRNA, and/or evaluating a miRNA population using an array, nucleic acid amplification, and/or hybridization can be included in a kit, as well reagents for preparation of samples from blood samples. The kit may further include reagents for creating or synthesizing miRNA probes. The kits will thus comprise, in suitable container means, an enzyme for labeling the miRNA by incorporating labeled nucleotide or unlabeled nucleotides that are subsequently labeled. In certain aspects, the kit can include amplification reagents. In other aspects, the kit may include various supports, such as glass, nylon, polymeric beads, and the like, and/or reagents for coupling any probes and/or target nucleic acids. It may also include one or more buffers, such as reaction buffer, labeling buffer, washing buffer, or a hybridization buffer, compounds for preparing the miRNA probes, and components for isolating miRNA. Other kits of the invention may include components for making a nucleic acid array comprising miRNA, and thus, may include, for example, a solid support.

Kits for implementing methods of the invention described herein are specifically contemplated. In some embodiments, there are kits for preparing miRNA for multi-labeling and kits for preparing miRNA probes and/or miRNA arrays. In these embodiments, kit comprise, in suitable container means, 1, 2, 3, 4, 5, 6, 7, 8, 9, 10, 11, 12 or more of the following: (1) poly(A) polymerase; (2) unmodified nucleotides (G, A, T, C, and/or U); (3) a modified nucleotide (labeled or unlabeled); (4) poly(A) polymerase buffer; and, (5) at least one microfilter; (6) label that can be attached to a nucleotide; (7) at least one miRNA probe; (8) reaction buffer; (9) a miRNA array or components for making such an array; (10) acetic acid; (11) alcohol; (12) solutions for preparing, isolating, enriching, and purifying miRNAs or miRNA probes or arrays. Other reagents include those generally used for manipulating RNA, such as formamide, loading dye, ribonuclease inhibitors, and DNase.

In specific embodiments, kits of the invention include an array containing miRNA probes, as described in the application. An array may have probes corresponding to all known miRNAs of an organism or a particular tissue or organ in particular conditions, or to a subset of such probes. The subset of probes on arrays of the invention may be or include those identified as relevant to a particular diagnostic, therapeutic, or prognostic application. For example, the array may contain one or more probes that is indicative or suggestive of (1) a disease or condition (acute myeloid leukemia), (2) susceptibility or resistance to a particular drug or treatment; (3) susceptibility to toxicity from a drug or substance; (4) the stage of development or severity of a disease or condition (prognosis); and (5) genetic predisposition to a disease or condition.

For any kit embodiment, including an array, there can be nucleic acid molecules that contain or can be used to amplify a sequence that is a variant of, identical to or complementary to all or part of any of SEQ IDs described herein. In certain embodiments, a kit or array of the invention can contain one or more probes for the miRNAs identified by the SEQ IDs described herein. Any nucleic acid discussed above may be implemented as part of a kit.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit (labeling reagent and label may be packaged together), the kit also will generally contain a second, third or other additional container into which the additional components may be separately placed. However, various combinations of components may be comprised in a vial. The kits of the present invention also will typically include a means for containing the nucleic acids, and any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred.

However, the components of the kit may be provided as dried powder(s). When reagents and/or components are provided as a dry powder, the powder can be reconstituted by the addition of a suitable solvent. It is envisioned that the solvent may also be provided in another container means. In some embodiments, labeling dyes are provided as a dried power. It is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 μg or at least or at most those amounts of dried dye are provided in kits of the invention. The dye may then be resuspended in any suitable solvent, such as DMSO.

Such kits may also include components that facilitate isolation of the labeled miRNA. It may also include components that preserve or maintain the miRNA or that protect against its degradation. Such components may be RNAse-free or protect against RNAses. Such kits generally will comprise, in suitable means, distinct containers for each individual reagent or solution.

A kit will also include instructions for employing the kit components as well the use of any other reagent not included in the kit. Instructions may include variations that can be implemented.

Kits of the invention may also include one or more of the following: Control RNA; nuclease-free water; RNase-free containers, such as 1.5 ml tubes; RNase-free elution tubes; PEG or dextran; ethanol; acetic acid; sodium acetate; ammonium acetate; guanidinium; detergent; nucleic acid size marker; RNase-free tube tips; and RNase or DNase inhibitors.

It is contemplated that such reagents are embodiments of kits of the invention. Such kits, however, are not limited to the particular items identified above and may include any reagent used for the manipulation or characterization of miRNA.

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Gene Expression Analysis Following Transfection with HSA-miR-143

miRNAs are believed to regulate gene expression by binding to target mRNA transcripts and (1) initiating transcript degradation or (2) altering protein translation from the transcript. Translational regulation leading to an up or down change in protein expression may lead to changes in activity and expression of downstream gene products and genes that are in turn regulated by those proteins. These numerous regulatory effects may be revealed as changes in the global mRNA expression profile. Microarray gene expression analyses were performed to identify genes that are mis-regulated by hsa-miR-143 expression.

Synthetic Pre-miR-143 (Ambion) or two negative control miRNAs (pre-miR-NC1, Ambion cat. no. AM17110 and pre-miR-NC2, Ambion, cat. no. AM17111) were reverse transfected into quadruplicate samples of A549 cells for each of three time points. Cells were transfected using siPORT NeoFX (Ambion) according to the manufacturer's recommendations using the following parameters: 200,000 cells per well in a 6 well plate, 5.0 μl of NeoFX, 30 nM final concentration of miRNA in 2.5 ml. Cells were harvested at 4 h, 24 h, and 72 h post transfection. Total RNA was extracted using RNAqueous-4PCR (Ambion) according to the manufacturer's recommended protocol.

mRNA array analyses were performed by Asuragen Services (Austin, Tex.), according to the company's standard operating procedures. Using the MessageAmp™ II-96 aRNA Amplification Kit (Ambion, cat #1819) 2 μg of total RNA were used for target preparation and labeling with biotin. cRNA yields were quantified using an Agilent Bioanalyzer 2100 capillary electrophoresis protocol. Labeled target was hybridized to Affymetrix mRNA arrays (Human HG-U133A 2.0 arrays) using the manufacturer's recommendations and the following parameters. Hybridizations were carried out at 45° C. for 16 hr in an Affymetrix Model 640 hybridization oven. Arrays were washed and stained on an Affymetrix FS450 Fluidics station, running the wash script Midi_euk2v3_{—}450. The arrays were scanned on a Affymetrix GeneChip Scanner 3000. Summaries of the image signal data, group mean values, p-values with significance flags, log ratios and gene annotations for every gene on the array were generated using the Affymetrix Statistical Algorithm MAS 5.0 (GCOS v1.3). Data were reported in a file (cabinet) containing the Affymetrix data and result files and in files (.cel) containing the primary image and processed cell intensities of the arrays. Data were normalized for the effect observed by the average of two negative control microRNA sequences and then were averaged together for presentation. A list of genes whose expression levels varied by at least 0.7 log₂ from the average negative control was assembled. Results of the microarray gene expression analysis are shown in Table 1 above.

Manipulation of the expression levels of the genes listed in Table 1 represents a potentially useful therapy for cancer and other diseases in which increased or reduced expression of hsa-miR-143 has a role in the disease.

### Example 2

### Cellular Pathways Affected by HSA-miR-143

The mis-regulation of gene expression by hsa-miR-143 (Table 1) affects many cellular pathways that represent potential therapeutic targets for the control of cancer and other diseases and disorders. The inventors determined the identity and nature of the cellular genetic pathways affected by the regulatory cascade induced by hsa-miR-143 expression. Cellular pathway analyses were performed using Ingenuity Pathways Analysis (Version 4.0, Ingenuity® Systems, Redwood City, Calif.). Alteration of a given pathway was determined by Fisher's Exact test (Fisher, 1922). The most significantly affected pathways following over-expression of hsa-miR-143 in A549 cells are shown in Table 2.

These data demonstrate that hsa-miR-143 directly or indirectly affects the expression of several, cellular proliferation-, development-, and cell growth-related genes and thus primarily affects functional pathways related to cellular growth, cellular development, and cell proliferation. Those cellular processes have integral roles in the development and progression of various cancers. Manipulation of the expression levels of genes in the cellular pathways shown in Table 2 represents a potentially useful therapy for cancer and other diseases in which increased or reduced expression of hsa-miR-143 has a role in the disease.

### Example 3

### Predicted Gene Targets of HSA-miR-143

Gene targets for binding of and regulation by hsa-miR-143 were predicted using the proprietary algorithm miRNATarget™ (Asuragen), which is an implementation of the method proposed by Krek et al. (2005). Predicted target genes are shown in Table 3.

The predicted gene targets that exhibited altered mRNA expression levels in human cancer cells, following transfection with pre-miR hsa-miR-143, are shown in Table 4.

The predicted gene targets of hsa-miR-143 whose mRNA expression levels are affected by hsa-miR-143 represent particularly useful candidates for cancer therapy and therapy of other diseases through manipulation of their expression levels.

### Example 4

### Cancer Related Gene Expression Altered by HSA-miR-143

Cell proliferation, survival, and growth pathways are commonly altered in tumors (Hanahan and Weinberg, 2000). The inventors have shown that hsa-miR-143 directly or indirectly regulates the transcripts of proteins that are critical in the regulation of these pathways. Many of these targets have inherent oncogenic or tumor suppressor activity. Hsa-miR-143 targets that have prognostic and/or therapeutic value for the treatment of various malignancies are shown in Table 5.

Hsa-miR-143 targeted cancer genes are regulators of the cell cycle, transcription, intracellular signaling, apoptosis and the thioredoxin redox pathway. Hsa-miR-143 regulates cell cycle progression by altering the expression of Wee1, the retinoblastoma-like 1 protein (RBL1) as well as the cyclins D1 and G1. RBL1, also known as p107, is a member of the retinoblastoma tumor suppressor protein family that includes the pocket proteins p107, p130 and pRb. Similar to the pRb prototype, RBL1 interacts with the E2F family of transcription factors and blocks cell cycle progression and DNA replication (Sherr and McCormick, 2002). A subset of cancers show deregulated expression of RBL1 (Takimoto et al., 1998; Claudio et al., 2002; Wu et al., 2002; Ito et al., 2003). Transient transfection of hsa-miR-143 leads to a decrease in RBL1 mRNA levels which may suggest a proliferative function for hsa-miR-143. In contrast, negative regulation of cyclin D1 and positive regulation of cyclin G1 are indicators of a growth-inhibitory role for hsa-miR-143. Cyclins are co-factors of cyclin-dependent kinases (CDKs) and function in the progression of the cell cycle. Cyclin D1 is required for the transition from G1 into S phase and is overexpressed in numerous cancer types (Donnellan and Chetty, 1998). (Donnellan and Chetty, 1998). Hsa-miR-143 negatively regulates cyclin D1 expression and therefore might interfere with abnormal cell growth that depends on high levels of cyclin D1. In accordance, cyclin G1 has growth inhibitory activity and is upregulated by hsa-miR-143 (Zhao et al., 2003). Wee1 is a tyrosine kinase that functions as a mitotic inhibitor by phosphorylating the CDK1(cdc2)/cyclinB1 complex (Parker and Piwnica-Worms, 1992; McGowan and Russell, 1993). Lack of Wee1 expression in lung cancer is correlated with a higher proliferation index, a higher relapse rate and poor prognosis (Yoshida et al., 2004). Another hsa-miR-143 target is LMO-4 (LIM domain only 4), a zinc finger protein regulating transcription. LMO-4 is inherently oncogenic and inactivates the BRCA-1 tumor suppressor protein (breast cancer 1) (Sum et al., 2002; Sum et al., 2005). LMO-4 is frequently overexpressed in multiple cancer types and predicts poor outcome in breast cancer (Visvader et al., 2001; Mizunuma et al., 2003; Sum et al., 2005; Taniwaki et al., 2006). Accordingly, RNAi directed against LMO-4 leads to reduced breast cancer cell growth and migration (Sum et al., 2005). Our data indicate that hsa-miR-143 diminishes LMO-4 transcripts and therefore may intercept with the oncogenic properties of LMO-4.

Hsa-miR-143 also governs the expression of PDCD4, BCL2L1 and MCL1, all of which are functionally linked to the apoptotic pathway. Pdcd-4 (programmed cell death 4) is a tumor suppressor that is induced in response to apoptosis in normal cells. The growth inhibitory properties of Pdcd-4 are due to Pdcd-4 mediated inhibition of the c-Jun proto-oncoprotein, inhibition of cap-dependent mRNA translation and activation of the p21Waf1/Cip1 CDK inhibitor (Yang et al., 2003; Bitomsky et al., 2004; Goke et al., 2004). Pdcd-4 frequently shows reduced or lost expression in various human malignancies, such as gliomas, hepatocellular carcinomas, lung and renal cell carcinomas (Jansen et al., 2004; Zhang et al., 2006; Gao et al., 2007). Expression of Pdcd-4 interferes with skin carcinogenesis in a mouse model and suppresses growth of human colon carcinoma cells (Jansen et al., 2005; Yang et al., 2006). Loss of Pdcd-4 also correlates with lung tumor progression (Chen et al., 2003). Since hsa-miR-143 positively regulates Pdcd-4 expression, a hsa-miR-143 based therapy may reconstitute Pdcd-4 function. BCL2L1 and MCL1 are members of the anti-apoptotic BCL-2 (B cell lymphoma 2) gene family that give rise to two alternatively spliced gene products with opposing functions (Boise et al., 1993; Bae et al., 2000). The predominantly expressed protein encoded by BCL2L1 is Bcl-XL which—next to BCL-2—is a major inhibitor of programmed cell death. Overexpression of Bcl-XL is detected in numerous cancer types and correlates with tumor progression as well as poor survival (Manion and Hockenbery, 2003). Increased levels of Bcl-XL are also associated with resistance to chemo- and radiotherapy (Fesik, 2005). Transient transfection of hsa-miR-143 leads to a reduction of Bcl-XL transcripts and therefore might provide a therapeutic benefit to oncogenic cells with increased expression of Bcl-XL. Mcl-1 (myeloid leukemia 1) is overexpressed in hepatocellular carcinoma, prostate cancer, testicular tumor, multiple myeloma and various leukemias [see refs in Table 5]. Similar to Bcl-XL, high levels of Mcl-1 is correlated with poor prognosis of patients with ovarian carcinoma and is indicative for leukemic relapse (Kaufmann et al., 1998; Shigemasa et al., 2002). RNA interference against Mcl-1 induces a therapeutic response in gastric and hepatocellular carcinoma cells (Schulze-Bergkamen et al., 2006; Zangemeister-Wittke and Huwiler, 2006).

Molecules regulated by hsa-miR-143 that function in intracellular signal transduction include the inflammatory interleukin 8 (IL-8), transforming growth factor beta (TGF-β) receptor 2 (TGFBR2) and A-kinase anchor protein 12 (AKAP12). IL-8 is frequently upregulated in various cancers and correlates with tumor vascularization, metastasis and poor prognosis (Rosenkilde and Schwartz, 2004; Sparmann and Bar-Sagi, 2004). TGFBR-2 forms a functional complex with TGFBR-1 and is the primary receptor for TGF-β (Massague et al., 2000). Central role of TGF-β is inhibition of cellular growth of numerous cell types, such as epithelial, endothelial, hematopoietic neural and mesenchymal cells. Many mammary and colorectal carcinomas with microsatellite instability harbor inactivating mutations of TGFBR-2, and therefore escape the growth-inhibitory function of TGF-β (Markowitz et al., 1995; Lucke et al., 2001). AKAP12, also referred to as gravin or SSeCKS (Src suppressed C kinase substrate), functions as a kinase scaffold protein that tethers the enzyme-substrate interaction (Nauert et al, 1997). Expression of AKAP12 interferes with oncogenic cell transformation induced by the Src or Jun. oncoproteins in vitro and is lost or reduced in numerous cancers, such as leukemia and carcinomas of the rectum, lung and stomach (Lin and Gelman, 1997; Cohen et al., 2001; Xia et al., 2001; Wikman et al., 2002; Boultwood et al., 2004; Choi et al., 2004; Mori et al., 2006). An apparent anti-oncogenic activity of AKAP12 in prostate and gastric cancers marks this protein as a putative tumor suppressor (Xia et al., 2001; Choi et al., 2004).

Based on the functions for most of these targets and how they are regulated by hsa-miR-143, hsa-miR-143 appears to have tumor suppressor potential. This view is supported by our observation that most cancers show reduced expression of miR-143. However, hsa-miR-143 also regulates gene expression in a manner that suggests a role for hsa-miR-143 in the development or progression of disease. For instance, hsa-miR-143 stimulates the expression of thioredoxin (TXN), a 12-kDa thiol reductase targeting various proteins and multiple pathways. Thioredoxin modulates the activity of transcription factors, induces the expression of angiogenic Hif-1α (hypoxia induced factor 1α) as well as VEGF (vascular endothelial growth factor) and can act as a proliferative and anti-apoptotic agent (Marks, 2006). In accord, carcinomas of the lung, pancreas, cervix, and liver show increased levels of thioredoxin. Thioredoxin expression is also correlated with aggressive tumor growth, poor prognosis, and chemoresistance (Marks, 2006). Therefore, a hsa-miR-143 antagonist may have therapeutic potential in cancers that show altered expression of thioredoxin.

In summary and not intending to limit the invention by any particular theory, hsa-miR-143 governs the activity of proteins that are critical regulators of cell proliferation and survival. These targets are frequently deregulated in human cancer. Based on this review of the genes and related pathways that are regulated by miR-143, introduction of hsa-miR-143 or an anti-hsa-miR-143 into a variety of cancer cell types would likely result in a therapeutic response.

### Example 5

### Delivery of Synthetic HSA-miR-143 Inhibits Tumor Growth of Lung Cancer Cells in Mice

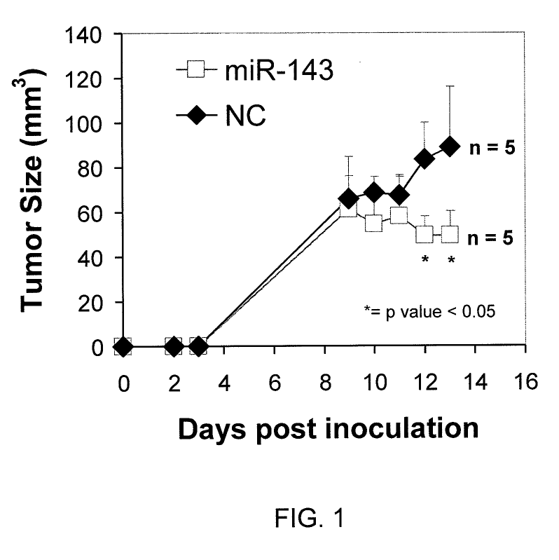

The inventors assessed the therapeutic activity of hsa-miR-143 in human lung cancer xenografts grown in immunodeficient mice. Hsa-miR-143 (Pre-miR™ microRNA Precursor Molecule; Ambion cat. no. AM17100) was delivered into A549 lung cancer cells via electroporation using the Gene Pulser Xcell™ (BioRad) with the following settings: 15×10⁶ cells with 5 μg miRNA in 200 μl OptiMEM (Invitrogen Corp., Carlsbad, Calif., USA), square wave pulse at 150 V for 10 ms. Electroporated cells (5×10⁶) were mixed with BD Matrigel™, (BD Biosciences; San Jose, Calif., USA; cat. no. 356237) in a 1:1 ratio and injected subcutaneously into the flank of female NOD/SCID mice (Charles River Laboratories, Inc.; Wilmington, Mass., USA). As a negative control, A549 cells were electroporated with negative control miRNA (NC; Pre-miR™ microRNA Precursor Molecule-Negative Control #2; Ambion cat. no. AM17111) as described above. To assess the anti-oncogenic activity of miR-143, a group of five animals was injected with A549 cells. NC-treated cells were injected into the opposite flank of the same animal to control for animal-to-animal variability. Once tumors reached a measurable size (9 days post injection), the length and width of tumors were determined every day until day 13 after xenograft implantation. Tumor volumes were calculated using the formula, Volume=(length X width X width)/2, in which the length is greater than the width. Tumor volumes derived from NC-treated cells and miR-143-treated cells were averaged and plotted over time (FIG. 1). Data points with p values less than 0.05 are indicated in the graph.

Administration of miR-143 into the A549 lung cancer cells inhibited tumor growth in vivo (FIG. 1). Cancer cells that received negative control miRNA developed more rapidly than cells treated with hsa-miR-143. These data suggest that hsa-miR-143 represents a particularly useful candidate in the treatment of lung cancer and potentially other diseases.

### REFERENCES

The following references, to the extent that they provide exemplary procedural or other details supplementary to those set forth herein, are specifically incorporated herein by reference.
U.S. Pat. No. 4,337,063
U.S. Pat. No. 4,404,289
U.S. Pat. No. 4,405,711
U.S. Pat. No. 4,659,774
U.S. Pat. No. 4,682,195
U.S. Pat. No. 4,683,202
U.S. Pat. No. 4,704,362
U.S. Pat. No. 4,816,571
U.S. Pat. No. 4,870,287
U.S. Pat. No. 4,959,463
U.S. Pat. No. 5,141,813
U.S. Pat. No. 5,143,854
U.S. Pat. No. 5,202,231
U.S. Pat. No. 5,214,136
U.S. Pat. No. 5,221,619
U.S. Pat. No. 5,223,618
U.S. Pat. No. 5,242,974
U.S. Pat. No. 5,264,566
U.S. Pat. No. 5,268,486
U.S. Pat. No. 5,288,644
U.S. Pat. No. 5,324,633
U.S. Pat. No. 5,378,825
U.S. Pat. No. 5,384,261
U.S. Pat. No. 5,399,363
U.S. Pat. No. 5,405,783
U.S. Pat. No. 5,412,087
U.S. Pat. No. 5,424,186
U.S. Pat. No. 5,428,148
U.S. Pat. No. 5,429,807
U.S. Pat. No. 5,432,049
U.S. Pat. No. 5,436,327
U.S. Pat. No. 5,445,934
U.S. Pat. No. 5,446,137
U.S. Pat. No. 5,466,468
U.S. Pat. No. 5,466,786
U.S. Pat. No. 5,468,613
U.S. Pat. No. 5,470,710
U.S. Pat. No. 5,470,967
U.S. Pat. No. 5,472,672
U.S. Pat. No. 5,480,980
U.S. Pat. No. 5,492,806
U.S. Pat. No. 5,503,980
U.S. Pat. No. 5,510,270
U.S. Pat. No. 5,525,464
U.S. Pat. No. 5,527,681
U.S. Pat. No. 5,529,756
U.S. Pat. No. 5,532,128
U.S. Pat. No. 5,543,158
U.S. Pat. No. 5,545,531
U.S. Pat. No. 5,574,146
U.S. Pat. No. 5,580,726
U.S. Pat. No. 5,580,732
U.S. Pat. No. 5,583,013
U.S. Pat. No. 5,593,839
U.S. Pat. No. 5,599,672
U.S. Pat. No. 5,599,695
U.S. Pat. No. 5,602,240
U.S. Pat. No. 5,602,244
U.S. Pat. No. 5,610,287
U.S. Pat. No. 5,610,289
U.S. Pat. No. 5,614,617
U.S. Pat. No. 5,623,070
U.S. Pat. No. 5,624,711
U.S. Pat. No. 5,631,134
U.S. Pat. No. 5,637,683
U.S. Pat. No. 5,639,603
U.S. Pat. No. 5,641,515
U.S. Pat. No. 5,645,897
U.S. Pat. No. 5,652,099
U.S. Pat. No. 5,654,413
U.S. Pat. No. 5,658,734
U.S. Pat. No. 5,661,028
U.S. Pat. No. 5,665,547
U.S. Pat. No. 5,667,972
U.S. Pat. No. 5,670,663
U.S. Pat. No. 5,672,697
U.S. Pat. No. 5,677,195
U.S. Pat. No. 5,681,947
U.S. Pat. No. 5,695,940
U.S. Pat. No. 5,700,637
U.S. Pat. No. 5,700,922
U.S. Pat. No. 5,705,629
U.S. Pat. No. 5,708,153
U.S. Pat. No. 5,708,154
U.S. Pat. No. 5,714,606
U.S. Pat. No. 5,728,525
U.S. Pat. No. 5,728,525
U.S. Pat. No. 5,739,169
U.S. Pat. No. 5,744,305
U.S. Pat. No. 5,744,305
U.S. Pat. No. 5,760,395
U.S. Pat. No. 5,763,167
U.S. Pat. No. 5,770,358
U.S. Pat. No. 5,777,092
U.S. Pat. No. 5,789,162
U.S. Pat. No. 5,792,847
U.S. Pat. No. 5,800,992
U.S. Pat. No. 5,801,005
U.S. Pat. No. 5,807,522
U.S. Pat. No. 5,824,311
U.S. Pat. No. 5,830,645
U.S. Pat. No. 5,830,880
U.S. Pat. No. 5,837,196
U.S. Pat. No. 5,846,225
U.S. Pat. No. 5,846,945
U.S. Pat. No. 5,847,219
U.S. Pat. No. 5,856,174
U.S. Pat. No. 5,858,988
U.S. Pat. No. 5,859,221
U.S. Pat. No. 5,871,928
U.S. Pat. No. 5,872,232
U.S. Pat. No. 5,876,932
U.S. Pat. No. 5,886,165
U.S. Pat. No. 5,919,626
U.S. Pat. No. 5,922,591
U.S. Pat. No. 6,004,755
U.S. Pat. No. 6,040,193
U.S. Pat. No. 6,040,193
U.S. Pat. No. 6,087,102
U.S. Pat. No. 6,251,666
U.S. Pat. No. 6,368,799
U.S. Pat. No. 6,383,749
U.S. Pat. No. 6,617,112
U.S. Pat. No. 6,638,717
U.S. Pat. No. 6,720,138
U.S. Pat. No. 6,723,509
U.S. patent Ser. No. 09/545,207
U.S. patent Ser. No. 10/667,126
U.S. patent Ser. No. 11/141,707
U.S. patent Ser. No. 11/273,640
U.S. patent Ser. No. 11/349,727
U.S. Patent Ser. 60/575,743
U.S. Patent Ser. 60/649,584
U.S. Patent Ser. 60/650,807
Akao et al., Oncol Reports, 16:845-850, 2006.
Akiba et al., Int J Oncol, 18(2):257-264, 2001.
Arap et al., Cancer Res., 55(6):1351-1354, 1995.
Austin-Ward and Villaseca, Revista Medica de Chile, 126(7):838-845, 1998.
Bae et al., J Biol Chem, 275(33):25255-25261, 2000.
Bagga et al., Cell, 122(4):553-563, 2005.
Biswas et al., Cancer Res, 64(14):4687-4692, 2004.
Bitomsky et al., Oncogene, 23(45):7484-7493, 2004.
Boise et al., Cell, 74(4):597-608, 1993.
Boultwood et al., Br J Haematol, 126(4):508-511, 2004.
Bukowski et al, Clinical Cancer Res., 4(10):2337-2347, 1998.
Caldas et al., Cancer Res., 54:3568-3573, 1994.
Calin and Croce, Nat Rev Cancer, 6(11):857-866, 2006.
Carrington and Ambros, Science, 301(5631):336-338, 2003.
Chen et al., J Pathol, 200(5):640-646, 2003.
Cheng et al., Cancer Res., 54(21):5547-5551, 1994.
Choi et al., Oncogene, 23(42):7095-7103, 2004.
Cho-Vega et al., Hum Pathol, 35(9):1095-1100, 2004.
Christodoulides et al., Microbiology, 144(Pt 11):3027-3037, 1998.
Claudio et al., Clin Cancer Res, 8(6):1808-1815, 2002.
Cohen et al., Oncogene, 20(2):141-146, 2001.
Cummins et al., In: IRT: Nucleosides and nucleosides, La Jolla Calif., 72, 1996.
Davidson et al, J. Immunother., 21(5):389-398, 1998.
Denli et al., Trends Biochem. Sci., 28:196, 2003.
Didenko, Biotechniques, 31(5):1106-1116, 1118, 1120-1121, 2001.
Dillman, Cancer Biother. Radiopharm., 14(1):5-10, 1999.
Donnellan and Chetty, Mol Pathol, 51(1):1-7, 1998.
Emptage et al, Neuron, 29(1):197-208, 2001.
EP 266,032
EP 373 203
EP 785 280
EP 799 897
Esau et al., J Biol Chem, 279(50):52361-52365, 2004.
Esquela-Kerscher and Slack, Nat Rev Cancer, 6(4):259-269, 2006.
Fesik, Nat Rev Cancer, 5(11):876-885, 2005.
Firth and Baxter, Endocr Rev, 23(6):824-854, 2002.
Fisher, J Royal Statistical Soc 85(1):87-94, 1922.
Fleischer et al., Int J Oncol, 28(1):25-32, 2006.
Fodor et al., Biochemistry, 30(33):8102-8108, 1991.
Froehler et al., Nucleic Acids Res., 14(13):5399-5407, 1986.
Gao et al., Oncol Rep, 17(1):123-128, 2007.
Goke et al., Am J Physiol Cell Physiol, 287(6):C1541-1546, 2004.
Griffey et al., J. Mass Spectrom, 32(3):305-13, 1997.
Hanahan and Weinberg, Cell, 100(1):57-70, 2000.
Hanibuchi et al., Int. J. Cancer, 78(4):480-485, 1998.
Hellstrand et al., Acta Oncologica, 37(4):347-353, 1998.
Hui and Hashimoto, Infection Immun., 66(11):5329-5336, 1998.
Hussussian et al., Nat. Genet., 8(1):15-21, 1994.
Itakura and Riggs, Science, 209:1401-1405, 1980.
Ito et al, Anticancer Res, 23(5A):3819-3824, 2003.
Jansen et al., Cancer Res, 65(14):6034-6041, 2005.
Jansen et al., Mol Cancer Ther, 3(2):103-110, 2004.
Ju et al., Gene Ther., 7(19):1672-1679, 2000.
Kamb et al., Nat. Genet., 8(1):23-26, 1994.
Kamb et al., Science, 2674:436-440, 1994.
Kaufmann et al., Blood, 91(3):991-1000, 1998.
Kitada et al., Blood, 91(9):3379-3389, 1998.
Klostermeier and Millar, Biopolymers, 61(3):159-79, 2001-2002.
Kornberg and Baker, In: DNA Replication, 2d Ed., Freeman, San Francisco, 1992.
Krajewska et al., Am J Pathol, 148(5):1567-1576, 1996.
Krek et al., Nature Genet. 37:495-500, 2005.
Lagos-Quintana et al., Science, 294(5543):853-858, 2001.
Lau et al., Science, 294(5543):858-862, 2001.
Lee and Ambros, Science, 294(5543):862-864, 2001.
Lim et al., Nature, 433(7027):769-773, 2005.
Lin and Gelman, Cancer Res, 57(11):2304-2312, 1997.
Lucke et al., Cancer Res, 61(2):482-485, 2001.
Manion and Hockenbery, Cancer Biol Ther, 2(4 Suppl 1): S105-114, 2003.
Markowitz, Biochim Biophys Acta, 1470(1):M13-20, 2000.
Marks, Semin Cancer Biol, 16(6):436-443, 2006.
Marsters et al., Recent Prog. Horm. Res., 54:225-234, 1999.
Massague et al., Cell, 103(2):295-309, 2000.
McGowan and Russell, Embo J, 12(1):75-85, 1993.
Michael et al., Mol Cancer Res, 1:882-891, 2003.
Mizunuma et al., Br J Cancer, 88(10):1543-1548, 2003.
Mori et al., Cancer Res., 54(13):3396-3397, 1994.
Mori et al., Gastroenterology, 131(3):797-808, 2006.
Nauert et al., Curr Biol, 7(1):52-62, 1997.
Nobori et al., Nature, 368(6473):753-756, 1994.
Okamoto et al., Proc. Natl. Acad. Sci. USA, 91(23):11045-11049, 1994.
Olsen et al., Dev. Biol., 216:671, 1999.
Orlow et al., Cancer Res, 54(11):2848-2851, 1994.
Orlow et al., Int. J. Oncol., 15(1):17-24, 1994.
Parker and Piwnica-Worms, Science, 257(5078):1955-1957, 1992.
PCT Appln. WO 0138580
PCT Appln. WO 0168255
PCT Appln. WO 03020898
PCT Appln. WO 03022421
PCT Appln. WO 03023058
PCT Appln. WO 03029485
PCT Appln. WO 03040410
PCT Appln. WO 03053586
PCT Appln. WO 03066906
PCT Appln. WO 03067217
PCT Appln. WO 03076928
PCT Appln. WO 03087297
PCT Appln. WO 03091426
PCT Appln. WO 03093810
PCT Appln. WO 03100012
PCT Appln. WO 03100448A1
PCT Appln. WO 04020085
PCT Appln. WO 04027093
PCT Appln. WO 09923256
PCT Appln. WO 09936760
PCT Appln. WO 93/17126
PCT Appln. WO 95/11995
PCT Appln. WO 95/21265
PCT Appln. WO 95/21944
PCT Appln. WO 95/21944
PCT Appln. WO 95/35505
PCT Appln. WO 96/31622
PCT Appln. WO 97/10365
PCT Appln. WO 97/27317
PCT Appln. WO 9743450
PCT Appln. WO 99/35505
Pietras et al., Oncogene, 17(17):2235-2249, 1998.
Pruitt et al., Nucleic Acids Res, 33(1):D501-D504, 2005.
Qin et al., Proc. Natl. Acad. Sci. USA, 95(24):14411-14416, 1998.
Reimer et al., J Biol Chem, 274(16):11022-11029, 1999.
Remington's Pharmaceutical Sciences” 15th Edition, pages 1035-1038 and 1570-1580, 1990.
Rosenkilde and Schwartz, Apmis, 112(7-8):481-495, 2004.
Rust et al., J Clin Pathol, 58(5):520-524, 2005.
Sambrook and Russell, Molecular Cloning: A Laboratory Manual 3^{rd} Ed., Cold Spring Harbor Laboratory Press, 2001.
Sambrook et al, In: DNA microarrays: a molecular cloning manual, Cold Spring Harbor Laboratory Press, Cold Spring Harbor, N.Y., 2003.
Sambrook et al, In: Molecular cloning: a laboratory manual, 2^{nd} Ed., Cold Spring Harbor Laboratory Press, Cold Spring Harbor, N.Y., 1989.
Sano et al., Histopathology, 46(5):532-539, 2005.
Scheit, In: Synthesis and Biological Function, Wiley-Interscience, New York, 171-172, 1980.
Schulze-Bergkamen et al., BMC Cancer, 6:232, 2006.
Seggerson et al., Dev. Biol., 243:215, 2002.
Serrano et al., Nature, 366:704-707, 1993.
Serrano et al., Science, 267(5195):249-252, 1995.
Sherr and McCormick, Cancer Cell, 2(2):103-112, 2002.
Shigemasa et al., Jpn J Cancer Res, 93(5):542-550, 2002.
Sieghart et al., J Hepatol, 44(1):151-157, 2006.
Skotzko et al., Cancer Res, 55(23):5493-5498, 1995.
Sparmann and Bar-Sagi, Cancer Cell, 6(5):447-458, 2004.
Sum et al., J Biol Chem, 277(10):7849-7856, 2002.
Sum et al., Proc Natl Acad Sci USA, 102(21):7659-7664, 2005.
Takimoto et al., Biochem Biophys Res Commun, 251(1):264-268, 1998.
Taniwaki et al., Int J Oncol, 29(3):567-575, 2006.
U.K. Patent 1,529,202
U.K. Patent 8 803 000
Visvader et al., Proc Natl Acad Sci USA, 98(25):14452-14457, 2001.
Wikman et al., Oncogene, 21(37):5804-5813, 2002.
Wu et al., Eur J Cancer, 38(14):1838-1848, 2002.
Wuilleme-Toumi et al., Leukemia, 19(7):1248-1252, 2005.
Xia et al., Cancer Res, 61(14):5644-5651, 2001a.
Xia et al., Cancer, 91(8):1429-1436, 2001b.
Yang et al., Cancer Cell, 9(6):445-457, 2006a.
Yang et al., Mol Cell Biol, 23(1):26-37, 2003.
Yang et al., Mol Cell Biol, 26(4):1297-1306, 2006b.
Yoshida et al., Ann Oncol, 15(2):252-256, 2004.
Zangemeister-Wittke and Huwiler, Cancer Biol Ther, 5(10):1355-1356, 2006.
Zhang et al., Oncogene, 25(45):6101-6112, 2006.
Zhao et al, Mol Cancer Res, 1(3):195-206, 2003.

1. A method of modulating gene expression in a cell comprising administering to the cell an amount of an isolated nucleic acid comprising a miR-143 nucleic acid sequence in an amount sufficient to modulate the expression of one or more genes identified in Table 1, 3, 4, or 5.

2. The method of claim 1, wherein the cell is in a subject having, suspected of having, or at risk of developing a metabolic, an immunologic, an infectious, a cardiovascular, a digestive, an endocrine, an ocular, a genitourinary, a blood, a musculoskeletal, a nervous system, a congenital, a respiratory, a skin, or a cancerous disease or condition.

3. (canceled)

4. The method of claim 2, wherein the cancerous condition is astrocytoma, anaplastic large cell lymphoma, acute lymphoblastic leukemia, acute myelogenous leukemia, breast carcinoma, B-cell lymphoma, bladder carcinoma, cervical carcinoma, chronic lymphoblastic leukemia, colorectal carcinoma, endometrial carcinoma, glioma, glioblastoma, gastric carcinoma, hepatocellular carcinoma, Hodgkin lymphoma, leukemia, lung carcinoma, melanoma, medulloblastoma, mantle cell lymphoma, multiple myeloma, myeloma, non-Hodgkin lymphoma, non-small cell lung carcinoma, ovarian carcinoma, oligodendroglioma, oesophageal carcinoma, osteosarcoma, pancreatic carcinoma, prostate carcinoma, renal cell carcinoma, squamous cell carcinoma of the head and neck, small cell lung carcinoma, thyroid carcinoma, or testicular tumor, wherein the modulation of one or more gene is sufficient for a therapeutic response.

5. The method of claim 4, wherein the cancerous condition is lung carcinoma.

6. The method of claim 5, wherein lung carcinoma is adenocarcinoma, squamous cell carcinoma, large cell carcinoma or bronchioalveolar carcinoma.

7. The method of claim 1, wherein the expression of a gene is up-regulated.

8. The method of claim 1, wherein the expression of a gene is down-regulated.

9. The method of claim 1, wherein the cell is an endothelial, a mesothelial, an epithelial, a stromal, or a mucosal cell.

10. The method of claim 1, wherein the cell is a brain, a neuronal, a blood, an esophageal, a lung, a cardiovascular, a liver, a breast, a bone, a thyroid, a glandular, an adrenal, a pancreatic, a stomach, an intestinal, a kidney, a bladder, a prostate, a cervical, a uterine, an ovarian, a testicular, a splenic, a skin, a smooth muscle, a cardiac muscle, or a striated muscle cell.

11. The method of claim 1, wherein the cell is a cancer cell.

12. The method of claim 11, wherein the cancer cell is a neuronal, glial, lung, liver, brain, breast, bladder, blood, leukemic, colon, endometrial, stomach, skin, ovarian, fat, bone, cervical, esophageal, pancreatic, prostate, kidney, testicular, intestinal, lymphoid, colorectal, or thyroid cell.

13. The method of claim 1, wherein the isolated miR-143 nucleic acid is a recombinant nucleic acid.

14-18. (canceled)

19. The method of claim 1, wherein the miR-143 nucleic acid is a synthetic nucleic acid.

20. (canceled)

21. The method of claim 1, wherein the miR-143 is a hsa-miR-143.

22-24. (canceled)

25. The method of claim 1, wherein the nucleic acid is comprised in a pharmaceutical formulation.

26. The method of claim 25, wherein the pharmaceutical formulation is a lipid composition.

27. The method of claim 25 wherein the pharmaceutical formulation is a nanoparticle composition.

28. The method of claim 25 wherein the pharmaceutical formulation consists of biocompatible and biodegradable molecules.

29-44. (canceled)

45. A method of treating a patient diagnosed with or suspected of having or suspected of developing a pathological condition or disease related to a gene modulated by a miRNA comprising the steps of:
(a) administering to the patient an amount of an isolated nucleic acid comprising a miR-143 nucleic acid sequence in an amount sufficient to modulate a cellular pathway or a physiologic pathway; and
(b) administering a second therapy, wherein the modulation of the cellular pathway or physiologic pathway sensitizes the patient to the second therapy.

46. (canceled)

47. A method of selecting a miRNA to be administered to a subject with, suspected of having, or having a propensity for developing a pathological condition or disease comprising:
(a) determining an expression profile of one or more genes selected from Table 1, 3, 4, or 5;
(b) assessing the sensitivity of the subject to miRNA therapy based on the expression profile; and
(c) selecting one or more miRNA based on the assessed sensitivity.

48-52. (canceled)
